(19) United States
(12) Patent Application Publication
(10) Pub. No.: US 2024/0150402 A1
(43) Pub. Date: May 9, 2024

(54) MATRIPTASE AND U-PLASMINOGEN ACTIVATOR SUBSTRATES AND OTHER CLEAVABLE MOIETIES AND METHODS OF USE THEREOF

(71) Applicant: CytomX Therapeutics, Inc., South San Francisco, CA (US)

(72) Inventors: Stephen James MOORE, Danville, CA (US); Margaret Thy Luu NGUYEN, San Francisco, CA (US); Daniel Robert HOSTETTER, Rocklin, CA (US); Olga VASILJEVA, Highlands Ranch, CO (US)

(21) Appl. No.: 18/534,263

(22) Filed: Dec. 8, 2023

Related U.S. Application Data
(62) Division of application No. 17/240,278, filed on Apr. 26, 2021, now Pat. No. 11,884,746.
Application No. 17/240,278 is a division of application No. 16/160,846, filed on Oct. 15, 2018, now Pat. No. 11,028,126.
Application No. 16/160,846 is a continuation of application No. 15/390,975, filed on Dec. 27, 2016, now Pat. No. 10,138,272.
Application No. 15/390,975 is a division of application No. 14/610,468, filed on Jan. 30, 2015, now Pat. No. 9,562,073.
(60) Provisional application No. 61/971,009, filed on Mar. 27, 2014.
(60) Provisional application No. 61/934,619, filed on Jan. 31, 2014.

(51) Int. Cl. C07K 7/06; A61K 47/65; A61K 47/68; A61K 49/00; C07K 7/08; C07K 16/00; C07K 16/28

(52) U.S. Cl. CPC C07K 7/06; A61K 47/65; A61K 47/6803; A61K 47/6835; A61K 47/6889; A61K 49/0056; C07K 7/08; C07K 16/00; C07K 16/2809; C07K 16/2863; A61K 2039/505

(57) ABSTRACT

The invention relates generally to polypeptides that include a cleavable moiety that is a substrate for at least one protease selected from matriptase and u-plasminogen activator (uPA), to activatable antibodies and other larger molecules that include the cleavable moiety that is a substrate for at least one protease selected from matriptase and u-plasminogen activator, and to methods of making and using these polypeptides that include a cleavable moiety that is a substrate for at least one protease selected from matriptase and u-plasminogen activator in a variety of therapeutic, diagnostic and prophylactic indications.

### RELATED APPLICATIONS

This application is a divisional of U.S. Ser. No. 17/240,278 filed Apr. 26, 2021, which is a divisional of U.S. Ser. No. 16/160,846 filed Oct. 15, 2018, now U.S. Pat. No. 11,028,126, issued Jun. 8, 2021, which is a continuation of U.S. patent application Ser. No. 15/390,975, filed Dec. 27, 2016, now U.S. Pat. No. 10,138,272, issued Nov. 27, 2018; which is a divisional of U.S. patent application Ser. No. 14/610,468, filed Jan. 30, 2015, now U.S. Pat. No. 9,562,073, issued Feb. 7, 2017, which claims the benefit of U.S. Provisional Application No. 61/934,619, filed Jan. 31, 2014 and U.S. Provisional Application No. 61/971,009, filed Mar. 27, 2014, the contents of each of which are incorporated herein by reference in their entireties.

### INCORPORATION OF SEQUENCE LISTING

The instant application contains a Sequence Listing which has been submitted electronically in XML format and is hereby incorporated by reference in its entirety. Said XML copy, created on Dec. 4, 2023, is named 4862_107US7_SL.XML and is 684,032 bytes in size.

### FIELD OF THE INVENTION

The invention relates generally to polypeptides that include a cleavable moiety that is a substrate for at least one protease selected from matriptase and u-plasminogen activator (uPA), to activatable antibodies and other larger molecules that include the cleavable moiety that is a substrate for at least one protease selected from matriptase and uPA, and to methods of making and using these polypeptides that include a cleavable moiety that is a substrate for at least one protease selected from matriptase and uPA in a variety of therapeutic, diagnostic and prophylactic indications.

### BACKGROUND OF THE INVENTION

Proteases are enzymes that cleave the peptide bonds between amino acid residues. Some proteases are known to break specific peptide bonds based on the presence of a particular amino acid sequence within a protein. Proteases occur naturally in all organisms and are involved in a variety of physiological reactions from simple degradation to highly regulated pathways. However, many pathological conditions are associated with deregulated expression and/or activity of proteases. As such, inappropriate proteolysis can have a major role in development and progression of cancer as well as cardiovascular, inflammatory, neurodegenerative, eukaryotic, bacterial and viral and parasitic diseases.

Accordingly, there exists a need to identify new substrates for proteases and to use these substrates in a variety of therapeutic, diagnostic and prophylactic indications.

### SUMMARY OF THE INVENTION

The disclosure provides amino acid sequences that include a cleavable moiety (CM) that is a substrate for at least one protease selected from matriptase (also referred to herein as MT-SP1, matriptase-1, and similar terms denoting matriptase) and u-plasminogen activator (also referred to herein as uPA, urokinase, urokinase-type plasminogen activator, and similar terms denoting uPA). These CMs are useful in a variety of therapeutic, diagnostic and prophylactic indications.

In some embodiments, the CM is linked or otherwise attached to an antibody. For example, the CM is used to link one or more agents to the antibody or antigen binding fragment thereof that binds a given target, such that the CM is cleaved when exposed to the protease, i.e., matriptase and/or uPA, and the agent is released from the antibody or antigen-binding fragment. Exemplary targets include, but are not limited to the targets shown in Table 1. Exemplary antibodies or antigen-binding fragments thereof include, but are not limited to, the targets shown in Table 2. In some embodiments, the antibody in the uncleaved state has the structural arrangement from N-terminus to C-terminus as follows: Agent-CM-(Antibody or Antigen-Binding Fragment) or (Antibody or Antigen-Binding Fragment)-CM-Agent. In some embodiments, the antibody comprises a linking peptide between the antibody or antigen-binding fragment and the CM. In some embodiments, the antibody or antigen-binding fragment comprises a linking peptide between the CM and the conjugated agent.

In some embodiments, the antibody comprises a first linking peptide (LP1) and a second linking peptide (LP2), wherein the antibody in the uncleaved state has the structural arrangement from N-terminus to C-terminus as follows: Agent-LP1-CM-LP2—(Antibody or Antigen-Binding Fragment) or (Antibody or Antigen-Binding Fragment)-LP2-CM-LP1-Agent. In some embodiments, the two linking peptides need not be identical to each other.

In some embodiments, at least one of LP1 or LP2 comprises an amino acid sequence selected from the group consisting of (GS)ₙ, (GGS)ₙ, (GSGGS)ₙ (SEQ ID NO: 385) and (GGGS)ₙ (SEQ ID NO: 386), where n is an integer of at least one.

In some embodiments, at least one of LP1 or LP2 comprises an amino acid sequence selected from the group consisting of GGSG (SEQ ID NO: 387), GGSGG (SEQ ID NO: 388), GSGSG (SEQ ID NO: 389), GSGGG (SEQ ID NO: 390), GGGSG (SEQ ID NO: 391), and GSSSG (SEQ ID NO: 392).

In some embodiments, LP1 comprises the amino acid sequence

(SEQ ID NO: 393)
GSSGGSGGSGGSG,
(SEQ ID NO: 394)
GSSGGSGGSGG,
(SEQ ID NO: 395)
GSSGGSGGSGGS,
(SEQ ID NO: 396)
GSSGGSGGSGGSGGGS,
(SEQ ID NO: 397)
GSSGGSGGSG, or
(SEQ ID NO: 398)
GSSGGSGGSGS.

In some embodiments, LP2 comprises the amino acid sequence GSS, GGS, GGGS (SEQ ID NO: 399), GSSGT (SEQ ID NO: 400) or GSSG (SEQ ID NO: 401).

In some embodiments, the antibody or antigen-binding fragment has an equilibrium dissociation constant of about 100 nM or less for binding to the target.

In some embodiments, the antibody or antigen-binding fragment thereof specifically binds a target. In some embodiments, the antibody or immunologically active fragment thereof that binds the target is a monoclonal antibody, domain antibody, single chain, Fab fragment, a F(ab′)₂ fragment, a scFv, a scab, a dAb, a single domain heavy chain antibody, or a single domain light chain antibody. In some embodiments, such an antibody or immunologically active fragment thereof that binds the target is a mouse, other rodent, chimeric, humanized or fully human monoclonal antibody.

In some embodiments, the protease, i.e., matriptase and/or uPA is co-localized with the target in a tissue, and the protease cleaves the CM in the antibody when the antibody is exposed to the protease.

In some embodiments, the CM is a polypeptide of up to 15 amino acids in length.

In some embodiments, the CM is a substrate for at least matriptase. In some embodiments, the CM is a substrate for at least uPA. In some embodiments, the CM is a substrate for at least matriptase and uPA.

In some embodiments, the CM is a substrate for matriptase and/or uPA, and is resistant to cleavage by at least one other protease. In some embodiments, the CM is a substrate for matriptase and/or uPA, and is resistant to cleavage by at least plasmin. In some embodiments, the CM is a substrate for matriptase and/or uPA, and is resistant to cleavage by at least tissue plasminogen activator (tPA).

In some embodiments, the CM is a substrate for matriptase and/or uPA and includes a motif sequence that is recognized by matriptase and/or uPA, provided that for any given motif sequence of the disclosure:
(i) the CM does not comprise any of the following amino acid sequences TGRGPSWV (SEQ ID NO: 402), SARGPSRW (SEQ ID NO: 403), or TARGPSFK (SEQ ID NO: 404); and the CM does not comprise a consensus amino acid sequence based on these amino acid sequences, such as for example, TARGPSW (SEQ ID NO: 405);
(ii) the CM does not comprise any of the following amino acid sequences LSGRSDNH (SEQ ID NO: 406), GGWHTGRN (SEQ ID NO: 407), HTGRSGAL (SEQ ID NO: 408), or PLTGRSGG (SEQ ID NO: 409); and the CM does not comprise a consensus amino acid sequence based on these amino acid sequences, such as for example, LTGRSGA (SEQ ID NO: 410); and/or
(iii) the CM does not comprise any of the following amino acid sequences AARGPAIH (SEQ ID NO: 411), RGPAFNPM (SEQ ID NO: 412), SSRGPAYL (SEQ ID NO: 413), or RGPATPIM (SEQ ID NO: 414); and the CM does not comprise a consensus amino acid sequence based on these amino acid sequences, such as for example, RGPA (SEQ ID NO: 415).

In some embodiments, the motif sequence is a substrate for at least matriptase and includes a core CM consensus sequence shown in Tables 8A-8J below. In some embodiments, the motif sequence includes a subgenus, i.e., a subset, of the core CM consensus sequence shown in Tables 8A-8J below.

TABLE 8A
Matriptase Cleavable Core CM Consensus Sequence 1

| Core CM Consensus 1 | Subgenus of Core CM Consensus 1 |
|---|---|
| X₁X₂X₃X₄X₅ (SEQ ID NO: 1), wherein: | X₁X₂X₃X₄X₅ (SEQ ID NO: 2), wherein X₁ is A, |
| X₁ is A, G, H, K, L, N, P, R, S, or V; | G, P, R, S, or V; X₂ is A, L, M, P, S, or V; X₃ is |
| X₂ is A, H, L, M, P, Q, R, S, or V; | G, L, or P; X₄ is R; and X₅ is A, G, R, S, or V |
| X₃ is A, E, F, G, I, L, P, R, S, T, or V; | X₁X₂X₃X₄X₅ (SEQ ID NO: 3), wherein X₁ is A, |
| X₄ is A, I, K, N, R, T, or W; and | P, R, S, or V; X₂ is A, L, M, S, or V; X₃ is P; X₄ |
| X₅ is A, G, I, L, M, Q, R, S, or V | is R; and X₅ is A, G, S, or V |
| | X₁X₂X₃X₄X₅ (SEQ ID NO: 4), wherein: X₁ is A, |
| | P, or R; X₂ is A, S, or V; X₃ is P; X₄ is R; and X₅ |
| | is S or V |
| | X₁X₂X₃X₄X₅ (SEQ ID NO: 5), wherein: X₁ is A, |
| | P, or R; X₂ is A or S; X₃ is P; X₄ iS R; and X₅ is S |

TABLE 8B
Matriptase Cleavable Core CM Consensus Sequence 2

| Core CM Consensus 2 | Subgenus of Core CM Consensus 2 |
|---|---|
| X₁₀X₁₁X₁₂X₁₃X₁₄ (SEQ ID NO: 6), wherein: | X₁₀X₁₁X₁₂X₁₃X₁₄ (SEQ ID NO: 7), wherein: X₁₀ |
| X₁₀ is A, L, P, R, S, T, or V; | is A, R, S, or T; X₁₁ is K or R; X₁₂ is D or R; X₁₃ |
| X₁₁ is K or R; | is L or V; and X₁₄ is F or P |
| X₁₂ iS D or R; | X₁₀X₁₁X₁₂X₁₃X₁₄ (SEQ ID NO: 8), wherein: X₁₀ |
| X₁₃ is A, G, L, M, S, T, V, or W; and | is A, S, or T; X₁₁ is K or R; X₁₂ is R; X₁₃ is L or |
| X₁₄ is F, G, M, P, or V | V; and X₁₄ is F or P |
| | X₁₀X₁₁X₁₂X₁₃X₁₄ (SEQ ID NO: 9), wherein: X₁₀ |
| | is S or T; X₁₁ is R; X₁₂ is R; X₁₃ is L or V; and |
| | X₁₄ is P |

TABLE 8C
Matriptase Cleavable Core CM Consensus Sequence 3

| Core CM Consensus 3 | Subgenus of Core CM Consensus 3 |
|---|---|
| X₂₀X₂₁X₂₂X₂₃X₂₄ (SEQ ID NO: 10), wherein: | X₂₀X₂₁X₂₂X₂₃X₂₄ (SEQ ID NO: 11), wherein: |
| X₂₀ is E, G, P, R, S, V, or W; | X₂₀ is G, P, R, S, or V; X₂₁ is P or V; X₂₂ is L or |
| X₂₁ is A, G, L, M, P, S, or V; | R; X₂₃ is G; and X₂₄ is G or R |
| X₂₂ is A, I, L, or R; | X₂₀X₂₁X₂₂X₂₃X₂₄ (SEQ ID NO: 12), wherein: |
| X₂₃ is A, G, I, or P; and | X₂₀ is P or R; X₂₁ is P; X₂₂ is L; X₂₃ is G; and |
| X₂₄ is G or R | X₂₄ is R |

TABLE 8D
Matriptase Cleavable Core CM Consensus Sequence 4

| Core CM Consensus 4 | Subgenus of Core CM Consensus 4 |
|---|---|
| X₂₆X₂₇X₂₈X₂₉X₃₀ (SEQ ID NO: 13), wherein: | X₂₆X₂₇X₂₈X₂₉X₃₀ (SEQ ID NO: 14), wherein: |
| X₂₆ is A, G, H, L, R, or S; | X₂₆ is A, G, H, L, R, or S; X₂₇ is D, H, R, or T; |
| X₂₇ is D, H, N, R, S, T, or W; | X₂₈ is A, P, R, S, T, or V; X₂₉ is F, G, L, M, P, or |
| X₂₈ is A, N, P, R, S, T, or V; | S; and X₃₀ is G, L, P, S, V, or W |
| X₂₉ is F, G, L, M, P, Q, R, S, or Y; and | X₂₆X₂₇X₂₈X₂₉X₃₀ (SEQ ID NO: 15), wherein: |
| X₃₀ is I, G, L, P, S, V, or W | X₂₆ is G, L, or S; X₂₇ is R or T; X₂₈ is A, P, or S; |
| | X₂₉ is F, G, M, or S; and X₃₀ is G, P, S, V, or W |
| | X₂₆X₂₇X₂₈X₂₉X₃₀ (SEQ ID NO: 16), wherein: |
| | X₂₆ is G, L, or S; X₂₇ is R; X₂₈ is A or S; X₂₉ is |
| | G or M; and X₃₀ is G, P, S, or W |
| | X₂₆X₂₇X₂₈X₂₉X₃₀ (SEQ ID NO: 17), wherein: |
| | X₂₆ is L; X₂₇ is R; X₂₈ is A or S; X₂₉ is G; and |
| | X₃₀ iS W |

TABLE 8E
Matriptase Cleavable Core CM Consensus Sequence 5

| Core CM Consensus 5 | Subgenus of Core CM Consensus 5 |
|---|---|
| X₃₆X₃₇X₃₈X₃₉X₄₀ (SEQ ID NO: 18), wherein: | X₃₆X₃₇X₃₈X₃₉X₄₀ (SEQ ID NO: 19), wherein: |
| X₃₆ is G, K, L, S, V, or W; | X₃₆ is G, L, S, V, or W; X₃₇ is G, Q, R, or S; X₃₈ |
| X₃₇ is G, I, P, Q, R, or S; | is R; X₃₉ is G, S, or V; and X₄₀ is A, G, L, S, and |
| X₃₈ is R; | V |
| X₃₉ is G, K, R, S, or V; and | X₃₆X₃₇X₃₈X₃₉X₄₀ (SEQ ID NO: 20), wherein: |
| X₄₀ is A, C, G, L, M, P, S, V, or Y | X₃₆ is V; X₃₇ is S; X₃₈ is R; X₃₉ is S; and X₄₀ is |
| | A and V |

TABLE 8F
Matriptase Cleavable Core CM Consensus Sequence 6

| Core CM Consensus 6 | Subgenus of Core CM Consensus 6 |
|---|---|
| X₄₂X₄₃X₄₄X₄₅X₄₆ (SEQ ID NO: 21), wherein: | X₄₂X₄₃X₄₄X₄₅X₄₆ (SEQ ID NO: 22), wherein: |
| X₄₂ is A, E, G, I, L, M, R, or S; | X₄₂ is A, E, G, L, M, R, or S; X₄₃ is G, K, L, N, |
| X₄₃ is A, G, K, L, N, R, S, or V; | R, S, or V; X₄₄ is R or Y; X₄₅ is A, F, G, P, or S; |
| X₄₄ is F, H, L, R, or Y; | and X₄₆ is F, G, M, P, R, S, or V |
| X₄₅ is A, F, G, H, P, or S; and | X₄₂X₄₃X₄₄X₄₅X₄₆ (SEQ ID NO: 23), wherein: |
| X₄₆ is F, G, M, N, P, R, S, or V | X₄₂ is A, E, G, M, or S; X₄₃ is G, L, S, or V; X₄₄ |
| | is R or Y; X₄₅ is A, G, P, or S; and X₄₆ is F, G, |
| | M, P, R, S, or V |
| | X₄₂X₄₃X₄₄X₄₅X₄₆ (SEQ ID NO: 24), wherein: |
| | X₄₂ is A, G, or S; X₄₃ is L, S, or V; X₄₄ is R; X₄₅ |
| | is A; and X46 is M or P |
| | X₄₂X₄₃X₄₄X₄₅X₄₆ (SEQ ID NO: 25), wherein: |
| | X₄₂ is A; X₄₃ is L, S or V; X₄₄ is R; X₄₅ is A; and |
| | X₄₆ is M or P |

TABLE 8G
Matriptase Cleavable Core CM Consensus Sequence 7

| Core CM Consensus 7 | Subgenus of Core CM Consensus 7 |
|---|---|
| X₅₀X₅₁X₅₂X₅₃X₅₄ (SEQ ID NO: 26), wherein: | X₅₀X₅₁X₅₂X₅₃X₅₄ (SEQ ID NO: 27), wherein: |
| X₅₀ is A, E, K, L, P, S, T, V, W, or Y; | X₅₀ is E, P, S, V, or W; X₅₁ is A, P, R, S, V, or |
| X₅₁ is A, I, L, P, R, S, V, or Y; | Y; X₅₂ is E, G, H, L, P, or V; X₅₃ is G, K, L, or |
| X₅₂ is E, G, H, L, P, or V; | R; and X₅₄ is Q or R |
| X₅₃ is G, K, L, or R; and | X₅₀X₅₁X₅₂X₅₃X₅₄ (SEQ ID NO: 28), wherein: |
| X₅₄ is Q or R | X₅₀ is P or V; X₅1 is A, P, or R; X₅₂ is E, G, P, or |
| | V; X₅₃ is G or R; and X₅₄ is R |
| | X₅₀X₅₁X₅₂X₅₃X₅₄ (SEQ ID NO: 29), wherein: |
| | X₅₀ is P or V; X₅₁ is A or R; X₅₂ is G or V; X₅₃ is |
| | G or R; and X₅₄ is R |
| | X₅₀X₅₁X₅₂X₅₃X₅₄ (SEQ ID NO: 30), wherein: |
| | X₅₀ is P or V; X₅₁ is A; X₅₂ is G or V; X₅₃ is R; |
| | and X₅₄ iS R |

TABLE 8H
Matriptase Cleavable Core CM Consensus Sequence 8

| Core CM Consensus 8 | Subgenus of Core CM Consensus 8 |
|---|---|
| X₅₇X₅₈X₅₉X₆₀X₆₁ (SEQ ID NO: 31), wherein: | X₅₇X₅₈X₅₉X₆₀X₆₁ (SEQ ID NO: 32), wherein: |
| X₅₇ is A, G, I, K, P, S, or T; | X₅₇ is A, G, I, K, S, or T; X₅₈ is R; X₅₉ is 5; X₆₀ |
| X₅₈ is R or T; | is F, M, or R; and X₆₁ is A, I, L, R, or W |
| X₅₉ is H, M, or S; | X₅₇X₅₈X₅₉X₆₀X₆₁ (SEQ ID NO: 33), wherein: |
| X₆₀ is F, M, or R; and | X₅₇ is G or K; X₅₈ is R; X₅₉ is 5; X₆₀ is M; and |
| X₆₁ is A, G, I, L, P, Q, R, S, V, or W | X₆₁ is A, L, R, or W |
| | X₅₇X₅₈X₅₉X₆₀X₆₁ (SEQ ID NO: 34), wherein: |
| | X₅₇ is G; X₅₈ is R; X₅₉ is 5; X₆₀ is M; and X₆₁ is |
| | A or L |

TABLE 81
Matriptase Cleavable Core CM Consensus Sequence 9

| Core CM Consensus 9 | Subgenus of Core CM Consensus 9 |
|---|---|
| X₆₇X₆₈X₆₉X₇₀X₇₁ (SEQ ID NO: 35), wherein: | X₆₇X₆₈X₆₉X₇₀X₇₁ (SEQ ID NO: 36), wherein: |
| X₆₇ is I, L, or S; | X₆₇ is I or L; X₆₈ is A, G, P, or V; X₆₉ is R; X₇₀ |
| X₆₈ is A, G, K, P, R, or V; | is A, M, P, R, S, or T; and X₇₁ is G, K, L, R, S, |
| X₆₉ is L, R or S; | or V |
| X₇₀ is A, K, M, P, R, S, or T; and | X₆₇X₆₈X₆₉X₇₀X₇₁ (SEQ ID NO: 37), wherein: |
| X₇₁ is F, G, H, I, K, L, M, P, R, S, or V | X67 is L; X₆₈ is A, G, P, or V; X₆₉ is R; X₇₀ is A, |
| | M, P, R, or S; and X₇₁ is G, K, L, S, or V |
| | X₆₇X₆₈X₆₉X₇₀X₇₁ (SEQ ID NO: 38), wherein: |
| | X₆₇ is L; X₆₈ is A, G, or P; X₆₉ is R; X₇₀ is A or |
| | S; and X₇₁ is G or L |
| | X₆₇X₆₈X₆₉X₇₀X₇₁ (SEQ ID NO: 39), wherein: |
| | X₆₇ is L; X₆₈ is A or P; X₆₉ is R; X₇₀ is A; and |
| | X₇₁ is G or L |

TABLE 8J
Matriptase Cleavable Core CM Consensus Sequence 10

| Core CM Consensus 10 | Subgenus of Core CM Consensus 10 |
|---|---|
| X₇₄X₇₅X₇₆X₇₇X₇₈ (SEQ ID NO: 40), wherein: | X₇₄X₇₅X₇₆X₇₇X₇₈ (SEQ ID NO: 41), wherein: |
| X₇₄ is E, L, Q, S, T or V; | X₇₄ is E, L, T or V; X₇₅ is R or S; X₇₆ is K or R; |
| X₇₅ is L, R, or S; | X₇₇ is M, R, or S; and X₇₈ is G, L, M, S, or W |
| X₇₆ is H, K, or R; | X₇₄X₇₅X₇₆X₇₇X₇₈ (SEQ ID NO: 42), wherein: |
| X₇₇ is A, M, R, or S; and | X₇₄ is E, T or V; X₇₅ is R or S; X₇₆ is K or R; X₇₇ |
| X₇₈ is G, L, M, R, S, or W | is M or R; and X₇₈ is G, L, M, S, or W |
| | X₇₄X₇₅X₇₆X₇₇X₇₈ (SEQ ID NO: 43), wherein: |
| | X₇₄ is E or V; X₇₅ is 5; X₇₆ is K or R; X₇₇ is R; |
| | and X₇₈ is L, S, or W |
| | X₇₄X₇₅X₇₆X₇₇X₇₈ (SEQ ID NO: 44), wherein: |
| | X₇₄ is E; X₇₅ is 5; X₇₆ is K or R; X₇₇ is R; and |
| | X₇₈ is L or W |

In some embodiments, the motif sequence is a substrate for at least matriptase and includes an expanded consensus sequence based on one of the core CM consensus sequence shown in Tables 8A-8J. In some embodiments, the expanded consensus sequence is a consensus sequence shown in Tables 9A-9J-3 below.

TABLE 9A
Matriptase Cleavable Expanded Core CM Consensus Sequence 1

| Expanded Core CM Consensus 1 | Subgenus of Expanded Core CM Consensus 1 |
|---|---|
| X₁X₂X₃X₄X₅X₆ (SEQ ID NO: 45) | X₁X₂X₃X₄X₅X₆ (SEQ ID NO: 46), wherein: X1 is |
| wherein: | A, G, P, R, S, or V; X₂ is A, L, M, P, S, or V; X₃ is |
| X₁ is A, G, H, K, L, N, P, R, S, or V; | G, L, or P; X₄ is R; X₅ is A, G, R, S, or V; and X₆ |
| X₂ is A, H, L, M, P, Q, R, S, or V; | is F, G, H, L, M, S, or W, |
| X₃ is A, E, F, G, I, L, P, R, S, T, or V; | X₁X₂X₃X₄X₅X₆ (SEQ ID NO: 47), wherein: X₁ is |
| X₄ is A, I, K, N, R, T, or W; | A, P, R, S, or V; X₂ is A, L, M, S, or V; X₃ is P; |
| X₅ is A, G, I, L, M, Q, R, S, or V; and | X₄ is R; X₅ is A, G, S, or V; and X₆ is F, G, H, M, |
| X₆ is F, G, H, L, M, R, S, or W | S, or W |
| | X₁X₂X₃X₄X₅X₆ (SEQ ID NO: 48), wherein: X₁ is |
| | A, P, or R; X₂ is A, S, or V; X₃ is P; X₄ is R; X₅ is |
| | S or V; and X₆ is F, G, H, M, or S |
| | X₁X₂X₃X₄X₅X₆ (SEQ ID NO: 49), wherein: X₁ is |
| | A, P, or R; X₂ is A or S; X₃ is P; X₄ is R; X₅ is S; |
| | and X₆ is F, G, H, or S |

TABLE 9B-1
Matriptase Cleavable Expanded Core CM Consensus Sequence 2A

| Expanded Core CM Consensus 2A | Subgenus of Expanded Core CM Consensus 2A |
|---|---|
| X₉X₁₀X₁₁X₁₂X₁₃X₁₄ (SEQ ID NO: 50), | X₉X₁₀X₁₁X₁₂X₁₃X₁₄ (SEQ ID NO: 51), wherein: X₉ |
| wherein: | is E, G, L, P, Q or S; X₁₀ is A, R, S, or T; X₁₁ is K or |
| X₉ is A, E, G, L, P, Q, 5, T or V; | R; X₁₂ is D or R; X₁₃ is L or V; and X₁₄ is F or P |
| X₁₀ is A, L, P, R, S, T, or V; | X₉X₁₀X₁₁X₁₂X₁₃X₁₄ (SEQ ID NO: 52), wherein: X₉ |
| X₁₁ is K or R; | is E, L, P or Q; X₁₀ is A, S, or T; X₁₁ is K or R; X₁₂ |
| X₁₂ iS D or R; | is R; X₁₃ is L or V; and X₁₄ is F or P |
| X₁₃ is A, G, L, M, S, T, V, or W; and | X₉X₁₀X₁₁X₁₂X₁₃X₁₄ (SEQ ID NO: 53), wherein: X₉ |
| X₁₄ is F, G, M, P, or V | is E, P or Q; X₁₀ is S or T; X₁₁ is R; X₁₂ is R; X₁₃ is |
| | L or V; and X₁₄ is P |

TABLE 9B-2
Matriptase Cleavable Expanded Core CM Consensus Sequence 2B

| Expanded Core CM Consensus 2B | Subgenus of Expanded Core CM Consensus 2B |
|---|---|
| X₁₀X₁₁X₁₂X₁₃X₁₄X₁₅ (SEQ ID NO: 54), | X₁₀X₁₁X₁₂X₁₃X₁₄X₁₅ (SEQ ID NO: 55), wherein: X₁₀ |
| wherein: | is A, R, S, or T; X₁₁ is K or R; X₁₂ is D or R; X₁₃ is |
| X₁₀ is A, L, P, R, S, T, or V; | L or V; X₁₄ is F or P; and X₁₅ is G, L, S or V |
| X₁₁ is K or R; | X₁₀X₁₁X₁₂X₁₃X₁₄X₁₅ (SEQ ID NO: 56), wherein: X₁₀ |
| X₁₂ is D or R; | is A, S, or T; X₁₁ is K or R; X₁₂ is R; X₁₃ is L or V; |
| X₁₃ is A, G, L, M, S, T, V, or W; | X₁₄ is F or P; and X₁₅ is G, L, S or V |
| X₁₄ is F, G, M, P, or V; and | X₁₀X₁₁X₁₂X₁₃X₁₄X₁₅ (SEQ ID NO: 57), wherein: X₁₀ |
| X₁₅ is G, L, M, N, P, S, V, or Y | is S or T; X₁₁ is R; X₁₂ is R; X₁₃ is L or V; X₁₄ is P; |
| | and X₁₅ is L or V |

TABLE 9B-3
Matriptase Cleavable Expanded Core CM Consensus Sequence 2C

| Expanded Core CM Consensus 2C | Subgenus of Expanded Core CM Consensus 2C |
|---|---|
| X₉X₁₀X₁₁X₁₂X₁₃X₁₄X₁₅ (SEQ ID | X₉X₁₀X₁₁X₁₂X₁₃X₁₄X₁₅ (SEQ ID NO: 59), wherein: |
| NO: 58), wherein: | X₉ is E, G, L, P, Q or S; X₁₀ is A, R, S, or T; X₁₁ is |
| X₉ is A, E, G, L, P, Q, 5, T or V; | K or R; X₁₂ is D or R; X₁₃ is L or V; X₁₄ is F or P; |
| X₁₀ is A, L, P, R, S, T, or V; | and X₁₄ is G, L, S or V |
| X₁₁ is K or R; | X₉X₁₀X₁₁X₁₂X₁₃X₁₄X₁₅ (SEQ ID NO: 60), wherein: |
| X₁₂ is D or R; | X₉ is E, L, P or Q; X₁₀ is A, S, or T; X₁₁ is K or R; |
| X₁₃ is A, G, L, M, S, T, V, or W; | X₁₂ is R; X₁₃ is L or V; X₁₄ is F or P; and X₁₄ is G, |
| X₁₄ is F, G, M, P, or V; and | L, S or V |
| X₁₅ is G, L, M, N, P, S, V, or Y | X₉X₁₀X₁₁X₁₂X₁₃X₁₄X₁₅ (SEQ ID NO: 61), wherein: |
| | X₉ is E, P or Q; X₁₀ is S or T; X₁₁ is R; X₁₂ is R; X₁₃ |
| | is L or V; X₁₄ is P; and X₁₄ is L or V |

TABLE 9C-1
Matriptase Cleavable Expanded Core CM Consensus Sequence 3A

| Expanded Core CM Consensus 3A | Subgenus of Expanded Core CM Consensus 3A |
|---|---|
| X₁₉X₂₀X₂₁X₂₂X₂₃X₂₄ (SEQ ID NO: 62), | X₁₉X₂₀X₂₁X₂₂X₂₃X₂₄ (SEQ ID NO: 63), wherein: |
| wherein: | X₁₉ is G, K, or S; X₂₀ is G, P, R, S, or V; X₂₁ is P or |
| X₁₉ is D, G, K, S, T, or V; | V; X₂₂ is L or R; X₂₃ is G; and X₂₄ is G or R |
| X₂₀ is E, G, P, R, S, V, or W; | X₁₉X₂₀X₂₁X₂₂X₂₃X₂₄ (SEQ ID NO: 64), wherein: |
| X₂₁ is A, G, L, M, P, S, or V; | X₁₉ is G or S; X₂₀ is P or R; X₂₁ is P; X₂₂ is L; X₂₃ is |
| X₂₂ is A, I, L, or R; | G; X₂₄ is R |
| X₂₃ is A, G, I, or P; and | |
| X₂₄ is G or R | |

TABLE 9C-2
Matriptase Cleavable Expanded Core CM Consensus Sequence 3B

| Expanded Core CM Consensus 3B | Subgenus of Expanded Core CM Consensus 3B |
|---|---|
| X₁₈X₁₉X₂₀X₂₁X₂₂X₂₃X₂₄ (SEQ ID | X₁₈X₁₉X₂₀X₂₁X₂₂X₂₃X₂₄ (SEQ ID NO: 66), wherein: |
| NO: 65), wherein: | X₁₈ is C, G, or S; X₁₉ is G, K, or S; X₂₀ is G, P, R, S, |
| X₁₈ is C, G, I, L or S; | or V; X₂₁ is P or V; X₂₂ is L or R; X₂₃ is G; and X₂₄ |
| X₁₉ is D, G, K, S, T, or V; | is G or R |
| X₂₀ is E, G, P, R, S, V, or W; | X₁₈X₁₉X₂₀X₂₁X₂₂X₂₃X₂₄ (SEQ ID NO: 67), wherein: |
| X₂₁ is A, G, L, M, P, S, or V; | X₁₈ is C, G, or S; X₁₉ is G or S; X₂₀ is P or R; X₂₁ is |
| X₂₂ is A, I, L, or R; | P; X₂₂ is L; X₂₃ is G; X₂₄ is R |
| X₂₃ is A, G, I, or P; and | |
| X₂₄ is G or R | |

TABLE 9C-3
Matriptase Cleavable Expanded Core CM Consensus Sequence 3C

| Expanded Core CM Consensus 3C | Subgenus of Expanded Core CM Consensus 3C |
|---|---|
| X₁₇X₁₈X₁₉X₂₀X₂₁X₂₂X₂₃X₂₄ (SEQ ID | X₁₇X₁₈X₁₉X₂₀X₂₁X₂₂X₂₃X₂₄ (SEQ ID NO: 69), |
| NO: 68), wherein: | wherein: X₁₇ is G or S; X₁₈ is C, G, or S; X₁₉ is G, |
| X₁₇ is G or S; | K, or S; X₂₀ is G, P, R, S, or V; X₂₁ is P or V; X₂₂ is |
| X₁₈ is C, G, I, L or S; | L or R; X₂₃ is G; and X₂₄ is G or R |
| X₁₉ is D, G, K, S, T, or V; | X₁₇X₁₈X₁₉X₂₀X₂₁X₂₂X₂₃X₂₄ (SEQ ID NO: 70), |
| X₂₀ is E, G, P, R, S, V, or W; | wherein: X₁₇ is G or S; X₁₈ is C, G, or S; X₁₉ is G or |
| X₂₁ is A, G, L, M, P, S, or V; | S; X₂₀ is P or R; X₂₁ is P; X₂₂ is L; X₂₃ is G; X₂₄ is R |
| X₂₂ is A, I, L, or R; | |
| X₂₃ is A, G, I, or P; and | |
| X₂₄ is G or R | |

TABLE 9D-1
Matriptase Cleavable Expanded Core CM Consensus Sequence 4A

| Expanded Core CM Consensus 4A | Subgenus of Expanded Core CM Consensus 4A |
|---|---|
| X₂₅X₂₆X₂₇X₂₈X₂₉X₃₀ (SEQ ID NO: 71), | X₂₅X₂₆X₂₇X₂₈X₂₉X₃₀ (SEQ ID NO: 72), wherein: |
| wherein: | X₂₅ is G, M, R, or S; X₂₆ is A, G, H, L, R, or S; X₂₇ |
| X₂₅ is G, M, R, or S; | is D, H, R, or T; X₂₈ is A, P, R, S, T, or V; X₂₉ is F, |
| X₂₆ is A, G, H, L, R, or S; | G, L, M, P, or S; and X₃₀ is G, L, P, S, V, or W |
| X₂₇ is D, H, N, R, S, T, or W; | X₂₅X₂₆X₂₇X₂₈X₂₉X₃₀ (SEQ ID NO: 73), wherein: |
| X₂₈ is A, N, P, R, S, T, or V; | X₂₅ is G, M, R, or S; X₂₆ is G, L, or S; X₂₇ is R or T; |
| X₂₉ is F, G, L, M, P, Q, R, S, or Y; and | X₂₈ is A, P, or S; X₂₉ is F, G, M, or S; and X₃₀ is G, |
| X₃₀ is I, G, L, P, S, V, or W | P, S, V, or W |
| | X₂₅X₂₆X₂₇X₂₈X₂₉X₃₀ (SEQ ID NO: 74), wherein: |
| | X₂₅ is G, M, R, or S; X₂₆ is G, L, or S; X₂₇ is R; X₂₈ |
| | is A or S; X₂₉ is G or M; and X₃₀ is G, P, S, or W |
| | X₂₅X₂₆X₂₇X₂₈X₂₉X₃₀ (SEQ ID NO: 75), wherein: |
| | X₂₅ is M; X₂₆ is L; X₂₇ is R; X₂₈ is A or S; X₂₉ is G; |
| | and X₃₀ is W |

TABLE 9D-2
Matriptase Cleavable Expanded Core CM Consensus Sequence 4B

| Expanded Core CM Consensus 4B | Subgenus of Expanded Core CM Consensus 4B |
|---|---|
| X₂₅X₂₆X₂₇X₂₈X₂₉X₃₀X₃₁ (SEQ ID | X₂₅X₂₆X₂₇X₂₈X₂₉X₃₀X₃₁ (SEQ ID NO: 77), wherein: |
| NO: 76), wherein: | X₂₅ is G, M, R, or S; X₂₆ is A, G, H, L, R, or S; X₂₇ |
| X₂₅ is G, M, R, or S; | is D, H, R, or T; X₂₈ is A, P, R, S, T, or V; X₂₉ is F, |
| X₂₆ is A, G, H, L, R, or S; | G, L, M, P, or S; X₃₀ is G, L, P, S, V, or W; and X₃₁ |
| X₂₇ is D, H, N, R, S, T, or W; | is G, P, R, or S |
| X₂₈ is A, N, P, R, S, T, or V; | X₂₅X₂₆X₂₇X₂₈X₂₉X₃₀X₃₁ (SEQ ID NO: 78), wherein: |
| X₂₉ is F, G, L, M, P, Q, R, S, or Y; | X₂₅ is G, M, R, or S; X₂₆ is G, L, or S; X₂₇ is R or T; |
| X₃₀ is I, G, L, P, S, V, or W; and | X₂₈ is A, P, or S; X₂₉ is F, G, M, or S; X₃₀ is G, P, S, |
| X₃₁ is G, P, R, or S | V, or W; and X₃₁ is G, R, or S |
| X₃₁ is G, R, or S | X₂₅X₂₆X₂₇X₂₈X₂₉X₃₀X₃₁ (SEQ ID NO: 79), wherein: |
| | X₂₅ is G, M, R, or S; X₂₆ is G, L, or S; X₂₇ is R; X₂₈ |
| | is A or S; X₂₉ is G or M; X₃₀ is G, P, S, or W; and |
| | X₂₅X₂₆X₂₇X₂₈X₂₉X₃₀X₃₁ (SEQ ID NO: 80), wherein: |
| | X₂₅ is M; X₂₆ is L; X₂₇ is R; X₂₈ is A or S; X₂₉ is G; |
| | X₃₀ is W; and X₃₁ is R |

TABLE 9D-3
Matriptase Cleavable Expanded Core CM Consensus Sequence 4C

| Expanded Core CM Consensus 4C | Subgenus of Expanded Core CM Consensus 4C |
|---|---|
| X₂₅X₂₆X₂₇X₂₈X₂₉X₃₀X₃₁X₃₂ (SEQ ID | X₂₅X₂₆X₂₇X₂₈X₂₉X₃₀X₃₁X₃₂ (SEQ ID NO: 82), |
| NO: 81), wherein: | wherein: X₂₅ is G, M, R, or S; X₂₆ is A, G, H, L, R, |
| X₂₅ is G, M, R, or S; | or S; X₂₇ is D, H, R, or T; X₂₈ is A, P, R, S, T, or V; |
| X₂₆ is A, G, H, L, R, or S; | X₂₉ is F, G, L, M, P, or S; X₃₀ is G, L, P, S, V, or W; |
| X₂₇ is D, H, N, R, S, T, or W; | X₃₁ is G, P, R, or S; and X₃₂ is G, L, R, S, or V |
| X₂₈ is A, N, P, R, S, T, or V; | X₂₅X₂₆X₂₇X₂₈X₂₉X₃₀X₃₁X₃₂ (SEQ ID NO: 83), |
| X₂₉ is F, G, L, M, P, Q, R, S, or Y; | wherein: X₂₅ is G, M, R, or S; X₂₆ is G, L, or S; X₂₇ |
| X₃₀ is I, G, L, P, S, V, or W; | is R or T; X₂₈ is A, P, or S; X₂₉ is F, G, M, or S; X₃₀ |
| X₃₁ is G, P, R, or S; and | is G, P, S, V, or W; X₃₁ is G, R, or S; and X₃₂ is G, |
| X₃₂ is G, L, R, S, or V | L, S, or V |
| | X₂₅X₂₆X₂₇X₂₈X₂₉X₃₀X₃₁X₃₂ (SEQ ID NO: 84), |
| | wherein: X₂₅ is G, M, R, or S; X₂₆ is G, L, or S; X₂₇ |
| | is R; X₂₈ is A or S; X₂₉ is G or M; X₃₀ is G, P, S, or |
| | W; X₃₁ is G, R, or S; and X₃₂ is G, L, S, or V |
| | X₂₅X₂₆X₂₇X₂₈X₂₉X₃₀X₃₁X₃₂ (SEQ ID NO: 85), |
| | wherein: X₂₅ is M; X₂₆ is L; X₂₇ is R; X₂₈ is A or S; |
| | X₂₉ is G; X₃₀ is W; X₃₁ is R; and X₃₂ is G, L, or S |

TABLE 9E-1
Matriptase Cleavable Expanded Core CM Consensus Sequence 5A

| Expanded Core CM Consensus 5A | Subgenus of Expanded Core CM Consensus 5A |
|---|---|
| X₃₅X₃₆X₃₇X₃₈X₃₉X₄₀ (SEQ ID NO: 86), | X₃₅X₃₆X₃₇X₃₈X₃₉X₄₀ (SEQ ID NO: 87), wherein: |
| wherein: | X₃₅ is G, I, S, or V X₃₆ is G, L, S, V, or W; X₃₇ is G, |
| X₃₅ is A, E, G, H, I, L, N, P, S, or V; | Q, R, or S; X₃₈ is R; X₃₉ is G, S, or V; and X₄₀ is A, |
| X₃₆ is G, K, L, S, V, or W; | G, L, S, or V |
| X₃₇ is G, I, P, Q, R, or S; | X₃₅X₃₆X₃₇X₃₈X₃₉X₄₀ (SEQ ID NO: 88), wherein: |
| X₃₈ is R; | X₃₅ is G, I, S, or V X₃₆ is V; X₃₇ is S; X₃₈ is R; X₃₉ is |
| X₃₉ is G, K, R, S, or V; and | S; and X₄₀ is A or V |
| X₄₀ is A, C, G, L, M, P, S, V, or Y | X₃₅X₃₆X₃₇X₃₈X₃₉X₄₀ (SEQ ID NO: 89), wherein: |
| | X₃₅ is I; X₃₆ is V; X₃₇ is S; X₃₈ is R; X₃₉ is S; and |
| | X₄₀ is A or V |

TABLE 9E-2
Matriptase Cleavable Expanded Core CM Consensus Sequence 5B

| Expanded Core CM Consensus 5B | Subgenus of Expanded Core CM Consensus 5B |
|---|---|
| X₃₄X₃₅X₃₆X₃₇X₃₈X₃₉X₄₀ (SEQ ID | X₃₄X₃₅X₃₆X₃₇X₃₈X₃₉X₄₀ (SEQ ID NO: 91), wherein: |
| NO: 90), wherein: | X₃₄ is A, G, K, S, V, or Y; and X₃₅ is G, I, S, or V |
| X₃₄ is A, G, K, M, P, Q, S, V, or Y; | X₃₆ is G, L, S, V, or W; X₃₇ is G, Q, R, or S; X₃₈ is |
| X₃₅ is A, E, G, H, I, L, N, P, S, or V; | R; X₃₉ is G, S, or V; and X₄₀ is A, G, L, S, or V |
| X₃₆ is G, K, L, S, V, or W; | X₃₄X₃₅X₃₆X₃₇X₃₈X₃₉X₄₀ (SEQ ID NO: 92), wherein: |
| X₃₇ is G, I, P, Q, R, or S; | X₃₄ is G, S, V, or Y; X₃₅ is G, I, S, or V X₃₆ is V; |
| X₃₈ is R; | X₃₇ is S; X₃₈ is R; X₃₉ is S; and X₄₀ is A or V |
| X₃₉ is G, K, R, S, or V; and | X₃₄X₃₅X₃₆X₃₇X₃₈X₃₉X₄₀ (SEQ ID NO: 93), wherein: |
| X₄₀ is A, C, G, L, M, P, S, V, or Y | X₃₄ is Y; X₃₅ is I; X₃₆ is V; X₃₇ is S; X₃₈ is R; X₃₉ is |
| | S; and X₄₀ is A or V |

TABLE 9E-3
Matriptase Cleavable Expanded Core CM Consensus Sequence 5C

| Expanded Core CM Consensus 5C | Subgenus of Expanded Core CM Consensus 5C |
|---|---|
| X₃₃X₃₄X₃₅X₃₆X₃₇X₃₈X₃₉X₄₀ (SEQ ID | X₃₃X₃₄X₃₅X₃₆X₃₇X₃₈X₃₉X₄₀ (SEQ ID NO: 95), |
| NO: 94), wherein: | wherein: X₃₃ is G, P, Q, S, or T; X₃₄ is A, G, K, S, |
| X₃₃ is G, K, P, Q, S, or T; | V, or Y; and X₃₅ is G, I, S, or V X₃₆ is G, L, S, V, or |
| X₃₄ is A, G, K, M, P, Q, S, V, or Y; | W; X₃₇ is G, Q, R, or S; X₃₈ is R; X₃₉ is G, S, or V; |
| X₃₅ is A, E, G, H, I, L, N, P, S, or V; | and X₄₀ is A, G, L, S, or V |
| X₃₆ is G, K, L, S, V, or W; | X₃₃X₃₄X₃₅X₃₆X₃₇X₃₈X₃₉X₄₀ (SEQ ID NO: 96), |
| X₃₇ is G, I, P, Q, R, or S; | wherein: X₃₃ is G, Q, or S; X₃₄ is G, S, V, or Y; X₃₅ |
| X₃₈ is R; | is G, I, S, or V X₃₆ is V; X₃₇ is S; X₃₈ is R; X₃₉ is S; |
| X₃₉ is G, K, R, S, or V; and | and X₄₀ is A or V |
| X₄₀ is A, C, G, L, M, P, S, V, or Y | X₃₃X₃₄X₃₅X₃₆X₃₇X₃₈X₃₉X₄₀ (SEQ ID NO: 97), |
| | wherein: X₃₃ is G, Q, or S; X₃₄ is Y; X₃₅ is I; X₃₆ is |
| | V; X₃₇ is S; X₃₈ is R; X₃₉ is S; and X₄₀ is A or V |

TABLE 9F
Matriptase Cleavable Expanded Core CM Consensus Sequence 6

| Expanded Core CM Consensus 6 | Subgenus of Expanded Core CM Consensus 6 |
|---|---|
| X₄₁X₄₂X₄₃X₄₄X₄₅X₄₆ (SEQ ID NO: 98), | X₄₁X₄₂X₄₃X₄₄X₄₅X₄₆ (SEQ ID NO: 99), wherein: |
| wherein: | X₄₁ is G, K, R, S, or T; X₄₂ is A, E, G, L, M, R, or S; |
| X₄₁ is G, K, P, R, S, or T; | X₄₃ is G, K, L, N, R, S, or V; X₄₄ is R or Y; X₄₅ is A, |
| X₄₂ is A, E, G, I, L, M, R, or S; | F, G, P, or S; and X₄₆ is F, G, M, P, R, S, or V |
| X₄₃ is A, G, K, L, N, R, S, or V; | X₄₁X₄₂X₄₃X₄₄X₄₅X₄₆ (SEQ ID NO: 100), wherein: |
| X₄₄ is F, H, L, R, or Y; | X₄₁ is G, R, S, or T; X₄₂ is A, E, G, M, or S; X₄₃ is |
| X₄₅ is A, F, G, H, P, or S; and | G, L, S, or V; X₄₄ is R or Y; X₄₅ is A, G, P, or S; and |
| X₄₆ is F, G, M, N, P, R, S, or V | X₄₆ is F, G, M, P, R, S, or V |
| | X₄₁X₄₂X₄₃X₄₄X₄₅X₄₆ (SEQ ID NO: 101), wherein: |
| | X₄₁ is G, R, or S; X₄₂ is A, G, or S; X₄₃ is L, S, or V; |
| | X₄₄ is R; X₄₅ is A; and X₄₆ is M or P |
| | X₄₁X₄₂X₄₃X₄₄X₄₅X₄₆ (SEQ ID NO: 102), wherein: |
| | X₄₁ is G, R, or S; X₄₂ is A; X₄₃ is L, S or V; X₄₄ is R; |
| | X₄₅ is A; and X₄₆ is M or P |

TABLE 9G-1
Matriptase Cleavable Expanded Core CM Consensus Sequence 7A

| Expanded Core CM Consensus 7A | Subgenus of Expanded Core CM Consensus 7A |
|---|---|
| X₅₀X₅₁X₅₂X₅₃X₅₄X₅₅ (SEQ ID NO: 103), | X₅₀X₅₁X₅₂X₅₃X₅₄X₅₅ (SEQ ID NO: 104), wherein: |
| wherein: | X₅₀ is E, P, S, V, or W; X₅₁ is A, P, R, S, V, or Y; |
| X₅₀ is A, E, K, L, P, S, T, V, W, or Y; | X₅₂ is E, G, H, L, P, or V; X₅₃ is G, K, L, or R; X₅₄ |
| X₅₁ is A, I, L, P, R, S, V, or Y; | is Q or R; and X₅₅ is A, G, H, M, R, or S |
| X₅₂ is E, G, H, L, P, or V; | X₅₀X₅₁X₅₂X₅₃X₅₄X₅₅ (SEQ ID NO: 105), wherein: |
| X₅₃ is G, K, L, or R; | X₅₀ is P or V; X₅₁ is A, P, or R; X₅₂ is E, G, P, or V; |
| X₅₄ is Q or R; and | and X₅₃ is G or R; X₅₄ is R; X₅₅ is G, M, or S |
| X₅₅ is A, G, H, M, R, or S | X₅₀X₅₁X₅₂X₅₃X₅₄X₅₅ (SEQ ID NO: 106), wherein: |
| | X₅₀ is P or V; X₅₁ is A or R; X₅₂ is G or V; X₅₃ is G |
| | or R; X₅₄ is R; and X₅₅ is M or S |
| | X₅₀X₅₁X₅₂X₅₃X₅₄X₅₅ (SEQ ID NO: 107), wherein: |
| | X₅₀ is P or V; X₅₁ is A; X₅₂ is G or V; X₅₃ is R; X₅₄ |
| | is R; and X₅₅ is M or S |

TABLE 9G-2
Matriptase Cleavable Expanded Core CM Consensus Sequence 7B

| Expanded Core CM Consensus 7B | Subgenus of Expanded Core CM Consensus 7B |
|---|---|
| X₄₉X₅₀X₅₁X₅₂X₅₃X₅₄X₅₅ (SEQ ID | X₄₉X₅₀X₅₁X₅₂X₅₃X₅₄X₅₅ (SEQ ID NO: 109), |
| NO: 108), wherein: | wherein: X₄₉ is G, K, P, Q, S, or V; X₅₀ is E, P, S, |
| X₄₉ is E, G, K, P, Q, S, T, or V; | V, or W; X₅₁ is A, P, R, S, V, or Y; X₅₂ is E, G, H, |
| X₅₀ is A, E, K, L, P, S, T, V, W, or Y; | L, P, or V; X₅₃ is G, K, L, or R; X₅₄ is Q or R; and |
| X₅₁ is A, I, L, P, R, S, V, or Y; | X₅₅ is A, G, H, M, R, or S |
| X₅₂ is E, G, H, L, P, or V; | X₄₉X₅₀X₅₁X₅₂X₅₃X₅₄X₅₅ (SEQ ID NO:: 110), |
| X₅₃ is G, K, L, or R; | wherein: X₄₉ is G, P, S, or V; X₅₀ is P or V; X₅₁ is |
| X₅₄ is Q or R; and | A, P, or R; X₅₂ is E, G, P, or V; X₅₃ is G or R; X₅₄ is |
| X₅₅ is A, G, H, M, R, or S | R; and X₅₅ is G, M, or S |
| | X₄₉X₅₀X₅₁X₅₂X₅₃X₅₄X₅₅ (SEQ ID NO: 111), |
| | wherein: X₄₉ is G, P, S, or V; X₅₀ is P or V; X₅₁ is A |
| | or R; X₅₂ is G or V; X₅₃ is G or R; X₅₄ is R; and X₅₅ |
| | is M or S |
| | X₄₉X₅₀X₅₁X₅₂X₅₃X₅₄X₅₅ (SEQ ID NO: 112), |
| | wherein: X₄₉ is G, S, or V; X₅₀ is P or V; X₅₁ is A; |
| | X₅₂ is G or V; X₅₃ is R; X₅₄ is R; and X₅₅ is M or S |

TABLE 9G-3
Matriptase Cleavable Expanded Core CM Consensus Sequence 7C

| Expanded Core CM Consensus 7C | Subgenus of Expanded Core CM Consensus 7C |
|---|---|
| X₄₉X₅₀X₅₁X₅₂X₅₃X₅₄X₅₅X₅₆ (SEQ ID | X₄₉X₅₀X₅₁X₅₂X₅₃X₅₄X₅₅X₅₆ (SEQ ID NO: 114), |
| NO: 113), wherein: | wherein: X₄₉ is G, K, P, Q, S, or V; X₅₀ is E, P, S, |
| X₄₉ is E, G, K, P, Q, S, T, or V; | V, or W; X₅₁ is A, P, R, S, V, or Y; X₅₂ is E, G, H, |
| X₅₀ is A, E, K, L, P, S, T, V, W, or Y; | L, P, or V; X₅₃ is G, K, L, or R; X₅₄ is Q or R; X₅₅ is |
| X₅₁ is A, I, L, P, R, S, V, or Y; | A, G, H, M, R, or S; and X₅₆ is G, L, M, P, S, or W |
| X₅₂ is E, G, H, L, P, or V; | X₄₉X₅₀X₅₁X₅₂X₅₃X₅₄X₅₅X₅₆ (SEQ ID NO: 115), |
| X₅₃ is G, K, L, or R; | wherein: X₄₉ is G, P, S, or V; X₅₀ is P or V; X₅₁ is |
| X₅₄ is Q or R; | A, P, or R; X₅₂ is E, G, P, or V; X₅₃ is G or R; X₅₄ is |
| X₅₅ is A, G, H, M, R, or S; and | R; X₅₅ is G, M, or S; and X₅₆ is G, L, M, P, S, or W |
| X₅₆ is F, G, L, M, P, S, or W | X₄₉X₅₀X₅₁X₅₂X₅₃X₅₄X₅₅X₅₆ (SEQ ID NO: 116), |
| | wherein: X₄₉ is G, P, S, or V; X₅₀ is P or V; X₅₁ is A |
| | or R; X₅₂ is G or V; X₅₃ is G or R; X₅₄ is R; X₅₅ is M |
| | or S; and X₅₆ is G, L, P, S, or W |
| | X₄₉X₅₀X₅₁X₅₂X₅₃X₅₄X₅₅X₅₆ (SEQ ID NO: 117), |
| | wherein: X₄₉ is G, S, or V; X₅₀ is P or V; X₅₁ is A; |
| | X₅₂ is G or V; X₅₃ is R; X₅₄ is R; X₅₅ is M or S; and |
| | X₅₆ is G, L, or S |

TABLE 9H-1
Matriptase Cleavable Expanded Core CM Consensus Sequence 8A

| Expanded Core CM Consensus 8A | Subgenus of Expanded Core CM Consensus 8A |
|---|---|
| X₅₇X₅₈X₅₉X₆₀X₆₁X₆₂ (SEQ ID NO: 118), | X₅₇X₅₈X₅₉X₆₀X₆₁X₆₂ (SEQ ID NO: 119), wherein: |
| wherein: | X₅₇ is A, G, I, K, S, or T; X₅₈ is R; X₅₉ is 5; X₆₀ is |
| X₅₇ is A, G, I, K, P, S, or T; | F, M, or R; X₆₁ is A, I, L, R, or W; and X₆₂ is G, L, |
| X₅₈ is R or T; | M, P, Q, R, S, or V |
| X₅₉ is H, M, or S; | X₅₇X₅₈X₅₉X₆₀X₆₁X₆₂ (SEQ ID NO: 120), wherein: |
| X₆₀ is F, M, or R; | X₅₇ is G or K; X₅₈ is R; X₅₉ is S; X₆₀ is M; X₆₁ is A, |
| X₆₁ is A, G, I, L, P, Q, R, S, V, or W; and | L, R, or W; and X₆₂ is G, L, M, P, R, or S |
| X₆₂ is A, G, L, M, P, Q, R, S, T, V, or W | X₅₇X₅₈X₅₉X₆₀X₆₁X₆₂ (SEQ ID NO: 121), wherein: |
| | X₅₇ is G; X₅₈ is R; X₅₉ is S; X₆₀ is M; X₆₁ is A or L; |
| | and X₆₂ is G, L, M, R, or S |
| | X₅₇X₅₈X₅₉X₆₀X₆₁X₆₂ (SEQ ID NO: 122), wherein: |
| | X₅₇ is G; X₅₈ is R; X₅₉ is S; X₆₀ is M; X₆₁ is A or L; |
| | and X₆₂ is L or M |

TABLE 9H-2
Matriptase Cleavable Expanded Core CM Consensus Sequence 8B

| Expanded Core CM Consensus 8B | Subgenus of Expanded Core CM Consensus 8B |
|---|---|
| X₅₇X₅₈X₅₉X₆₀X₆₁X₆₂X₆₃ (SEQ ID | X₅₇X₅₈X₅₉X₆₀X₆₁X₆₂X₆₃ (SEQ ID NO: 124), |
| NO: 123), wherein: | wherein: X₅₇ is A, G, I, K, S, or T; X₅₈ is R; X₅₉ is |
| X₅₇ is A, G, I, K, P, S, or T; | S; X₆₀ is F, M, or R; X₆₁ is A, I, L, R, or W; X₆₂ is |
| X₅₈ is R or T; | G, L, M, P, Q, R, S, or V; and X₆₃ is A, G, P, R, S, |
| X₅₉ is H, M, or S; | W, or Y |
| X₆₀ is F, M, or R; | X₅₇X₅₈X₅₉X₆₀X₆₁X₆₂X₆₃ (SEQ ID NO: 125), |
| X₆₁ is A, G, I, L, P, Q, R, S, V, or W; | wherein: X₅₇ is G or K; X₅₈ is R; X₅₉ is S; X₆₀ is M; |
| X₆₂ is A, G, L, M, P, Q, R, S, T, V, or | X₆₁ is A, L, R, or W; X₆₂ is G, L, M, P, R, or S; and |
| W; and | X₆₃ is A, G, P, R, S, or W |
| X₆₃ is A, G, K, M, P, R, S, W, or Y | X₅₇X₅₈X₅₉X₆₀X₆₁X₆₂X₆₃ (SEQ ID NO: 126), |
| | wherein: X₅₇ is G; X₅₈ is R; X₅₉ is S; X₆₀ is M; X₆₁ |
| | is A or L; X₆₂ is G, L, M, R, or S; and X₆₃ is A, G, P, |
| | R, or S |
| | X₅₇X₅₈X₅₉X₆₀X₆₁X₆₂X₆₃ (SEQ ID NO: 127), |
| | wherein: X₅₇ is G; X₅₈ is R; X₅₉ is 5; X₆₀ is M; X₆₁ |
| | is A or L; X₆₂ is L or M; and X₆₃ is G, P, or S |

TABLE 9H-3
Matriptase Cleavable Expanded Core CM Consensus Sequence 8C

| Expanded Core CM Consensus 8C | Subgenus of Expanded Core CM Consensus 8C |
|---|---|
| X₅₇X₅₈X₅₉X₆₀X₆₁X₆₂X₆₃X₆₄ (SEQ ID | X₅₇X₅₈X₅₉X₆₀X₆₁X₆₂X₆₃X₆₄ (SEQ ID NO: 129), |
| NO: 128), wherein: | wherein: X₅₇ is A, G, I, K, S, or T; X₅₈ is R; X₅₉ is |
| X₅₇ is A, G, I, K, P, S, or T; | S; X₆₀ is F, M, or R; X₆₁ is A, I, L, R, or W; X₆₂ is |
| X₅₈ is R or T; | G, L, M, P, Q, R, S, or V; X₆₃ is A, G, P, R, S, W, or |
| X₅₉ is H, M, or S; | Y; and X₆₄ is F, G, L, P, or S |
| X₆₀ is F, M, or R; | X₅₇X₅₈X₅₉X₆₀X₆₁X₆₂X₆₃X₆₄ (SEQ ID NO: 130), |
| X₆₁ is A, G, I, L, P, Q, R, S, V, or W; | wherein: X₅₇ is G or K; X₅₈ is R; X₅₉ is S; X₆₀ is M; |
| X₆₂ is A, G, L, M, P, Q, R, S, T, V, or W; | X₆₁ is A, L, R, or W; X₆₂ is G, L, M, P, R, or S; X₆₃ |
| X₆₃ is A, G, K, M, P, R, S, W, or Y; and | is A, G, P, R, S, or W; and X₆₄ is F, G, L, P, or S |
| X₆₄ is F, G, I, L, P, Q, S, or Y | X₅₇X₅₈X₅₉X₆₀X₆₁X₆₂X₆₃X₆₄ (SEQ ID NO: 131), |
| | wherein: X₅₇ is G; X₅₈ is R; X₅₉ is S; X₆₀ is M; X₆₁ |
| | is A or L; X₆₂ is G, L, M, R, or S; X₆₃ is A, G, P, R, |
| | or S; and X₆₄ is F, G, P, or S |
| | X₅₇X₅₈X₅₉X₆₀X₆₁X₆₂X₆₃X₆₄ (SEQ ID NO: 132), |
| | wherein: X₅₇ is G; X₅₈ is R; X₅₉ is S; X₆₀ is M; X₆₁ |
| | is A or L; X₆₂ is L or M; X₆₃ is G, P, or S; and X₆₄ is |
| | G, P, or S |

TABLE 9I-1
Matriptase Cleavable Expanded Core CM Consensus Sequence 9A

| Expanded Core CM Consensus 9A | Subgenus of Expanded Core CM Consensus 9A |
|---|---|
| X₆₇X₆₈X₆₉X₇₀X₇₁X₇₂ (SEQ ID NO: 133), | X₆₇X₆₈X₆₉X₇₀X₇₁X₇₂ (SEQ ID NO: 134), wherein: |
| wherein: | X₆₇ is I or L; X₆₈ is A, G, P, or V; X₆₉ is R; X₇₀ is A, |
| X₆₇ is I, L, or S; | M, P, R, S, or T; X₇₁ is G, K, L, R, S, or V; and X₇₂ |
| X₆₈ is A, G, K, P, R, or V; | is F, G, I, L, M, S, or V |
| X₆₉ is L, R or S; | X₆₇X₆₈X₆₉X₇₀X₇₁X₇₂ (SEQ ID NO: 135), wherein: |
| X₇₀ is A, K, M, P, R, S, or T; | X₆₇ is L; X₆₈ is A, G, P, or V; X₆₉ is R; X₇₀ is A, M, |
| X₇₁ is F, G, H, I, K, L, M, P, R, S, or V; | P, R, or S; X₇₁ is G, K, L, S, or V; and X₇₂ is F, G, I, |
| and | L, S, or V |
| X₇₂ is F, G, I, L, M, P, R, S, T, V, W, or | X₆₇X₆₈X₆₉X₇₀X₇₁X₇₂ (SEQ ID NO: 136), wherein: |
| Y | X₆₇ is L; X₆₈ is A, G, or P; X₆₉ is R; X₇₀ is A or S; |
| | X₇₁ is G or L; and X₇₂ is I or L |
| | X₆₇X₆₈X₆₉X₇₀X₇₁X₇₂ (SEQ ID NO: 137), wherein: |
| | X₆₇ is L; X₆₈ is A or P; X₆₉ is R; X₇₀ is A; X₇₁ is G |
| | or L; and X₇₂ is I or L |

TABLE 9I-2
Matriptase Cleavable Expanded Core CM Consensus Sequence 9B

| Expanded Core CM Consensus 9B | Subgenus of Expanded Core CM Consensus 9B |
|---|---|
| X₆₆X₆₇X₆₈X₆₉X₇₀X₇₁X₇₂ (SEQ ID | X₆₆X₆₇X₆₈X₆₉X₇₀X₇₁X₇₂ (SEQ ID NO: 139), |
| NO: 138), wherein: | wherein: X₆₆ is G, P, R, S, or T; X₆₇ is I or L; X₆₈ is |
| X₆₆ is G, K, P, Q, R, S, or T; | A, G, P, or V; X₆₉ is R; X₇₀ is A, M, P, R, S, or T; |
| X₆₇ is I, L, or S; | X₇₁ is G, K, L, R, S, or V; and X₇₂ is F, G, I, L, M, |
| X₆₈ is A, G, K, P, R, or V; | S, or V |
| X₆₉ is L, R or S; | X₆₆X₆₇X₆₈X₆₉X₇₀X₇₁X₇₂ (SEQ ID NO: 140), |
| X₇₀ is A, K, M, P, R, S, or T; | wherein: X₆₆ is G, P, R, or S; X₆₇ is L; X₆₈ is A, G, |
| X₇₁ is F, G, H, I, K, L, M, P, R, S, or | P, or V; X₆₉ is R; X₇₀ is A, M, P, R, or S; X₇₁ is G, |
| V; and | K, L, S, or V; and X₇₂ is F, G, I, L, S, or V |
| X₇₂ is F, G, I, L, M, P, R, S, T, V, W, | X₆₆X₆₇X₆₈X₆₉X₇₀X₇₁X₇₂ (SEQ ID NO: 141), |
| or Y | wherein: X₆₆ is P; X₆₇ is L; X₆₈ is A, G, or P; X₆₉ is |
| | R; X₇₀ is A or S; X₇₁ is G or L; and X₇₂ is I or L |
| | X₆₆X₆₇X₆₈X₆₉X₇₀X₇₁X₇₂ (SEQ ID NO: 142), |
| | wherein: X₆₆ is P; X₆₇ is L; X₆₈ is A or P; X₆₉ is R; |
| | X₇₀ is A; X₇₁ is G or L; and X₇₂ is I or L |

TABLE 9I-3
Matriptase Cleavable Expanded Core CM Consensus Sequence 9C

| Expanded Core CM Consensus 9C | Subgenus of Expanded Core CM Consensus 9C |
|---|---|
| X₆₅X₆₆X₆₇X₆₈X₆₉X₇₀X₇₁X₇₂ (SEQ ID | X₆₅X₆₆X₆₇X₆₈X₆₉X₇₀X₇₁X₇₂ (SEQ ID NO: 144), |
| NO: 143), wherein: | wherein: X₆₅ is G, K, P, R, S, or V; X₆₆ is G, P, R, |
| X₆₅ is A, G, I, K, P, R, S, or V; | S, or T; X₆₇ is I or L; X₆₈ is A, G, P, or V; X₆₉ is R; |
| X₆₆ is G, K, P, Q, R, S, or T; | X₇₀ is A, M, P, R, S, or T; X₇₁ is G, K, L, R, S, or V; |
| X₆₇ is I, L, or S; | and X₇₂ is F, G, I, L, M, S, or V |
| X₆₈ is A, G, K, P, R, or V; | X₆₅X₆₆X₆₇X₆₈X₆₉X₇₀X₇₁X₇₂ (SEQ ID NO: 145), |
| X₆₉ is L, R or S; | wherein: X₆₅ is G, P, R, or S; X₆₆ is G, P, R, or S; |
| X₇₀ is A, K, M, P, R, S, or T; | X₆₇ is L; X₆₈ is A, G, P, or V; X₆₉ is R; X₇₀ is A, M, |
| X₇₁ is F, G, H, I, K, L, M, P, R, S, or | P, R, or S; X₇₁ is G, K, L, S, or V; and X₇₂ is F, G, I, |
| V; and | L, S, or V |
| X₇₂ is F, G, I, L, M, P, R, S, T, V, W, | X₆₅X₆₆X₆₇X₆₈X₆₉X₇₀X₇₁X₇₂ (SEQ ID NO: 146), |
| or Y | wherein: X₆₅ is G, P, R, or S; X₆₆ is P; X₆₇ is L; X₆₈ |
| | is A, G, or P; X₆₉ is R; X₇₀ is A or S; X₇₁ is G or L; |
| | and X₇₂ is I or L |
| | X₆₅X₆₆X₆₇X₆₈X₆₉X₇₀X₇₁X₇₂ (SEQ ID NO: 147), |
| | wherein: X₆₅ is R; X₆₆ is P; X₆₇ is L; X₆₈ is A or P; |
| | X₆₉ is R; X₇₀ is A; X₇₁ is G or L; and X₇₂ is I or L |

TABLE 9J-1
Matriptase Cleavable Expanded Core CM Consensus Sequence 10A

| Expanded Core CM Consensus 10A | Subgenus of Expanded Core CM Consensus 10A |
|---|---|
| X₇₃X₇₄X₇₅X₇₆X₇₇X₇₈X₇₉ (SEQ ID | X₇₃X₇₄X₇₅X₇₆X₇₇X₇₈X₇₉ (SEQ ID NO: 149), |
| NO: 148), wherein: | wherein: X₇₃ is G, N, P, S, T, or V; X₇₄ is E, L, T or |
| X₇₃ is G, H, N, P, R, S, T, or V; | V; X₇₅ is R or S; X₇₆ is K or R; X₇₇ is M, R, or S; |
| X₇₄ is E, L, Q, S, T or V; | X₇₈ is G, L, M, S, or W; and X₇₉ is A, G, I, M, P, S, |
| X₇₅ is L, R, or S; | or V |
| X₇₆ is H, K, or R; | X₇₃X₇₄X₇₅X₇₆X₇₇X₇₈X₇₉ (SEQ ID NO: 150), |
| X₇₇ is A, M, R, or S; | wherein: X₇₃ is G, N, P, S, or V; X₇₄ is E, T or V; |
| X₇₈ is G, L, M, R, S, or W; and | X₇₅ is R or S; X₇₆ is K or R; X₇₇ is M or R; X₇₈ is G, |
| X₇₉ is A, G, I, M, N, P, S, V, or Y | L, M, S, or W; and X₇₉ is A, G, I, M, P, S, or V |
| | X₇₃X₇₄X₇₅X₇₆X₇₇X₇₈X₇₉ (SEQ ID NO: 151), |
| | wherein: X₇₃ is G, P, S, or V; X₇₄ is E or V; X₇₅ is |
| | S; X₇₆ is K or R; X₇₇ is R; X₇₈ is L, S, or W; and X₇₉ |
| | is G, I, M, P, S, or V |
| | X₇₃X₇₄X₇₅X₇₆X₇₇X₇₈X₇₉ (SEQ ID NO: 152), |
| | wherein: X₇₃ is G, P, or S; X₇₄ is E; X₇₅ is S; X₇₆ is |
| | K or R; X₇₇ is R; X₇₈ is L or W; and X₇₉ is M |

TABLE 9J-2
Matriptase Cleavable Expanded Core CM Consensus Sequence 10B

| Expanded Core CM Consensus 10B | Subgenus of Expanded Core CM Consensus 10B |
|---|---|
| X₇₄X₇₅X₇₆X₇₇X₇₈X₇₉X₈₀ (SEQ ID | X₇₄X₇₅X₇₆X₇₇X₇₈X₇₉X₈₀ (SEQ ID NO: 154), |
| NO: 153), wherein: | wherein: X₇₄ is E, L, T or V; X₇₅ is R or S; X₇₆ is K |
| X₇₄ is E, L, Q, S, T or V; | or R; X₇₇ is M, R, or S; X₇₈ is G, L, M, S, or W; X₇₉ |
| X₇₅ is L, R, or S; | is A, G, I, M, P, S, or V; and X₈₀ is G, I, L, N, P, S, |
| X₇₆ is H, K, or R; | or V |
| X₇₇ is A, M, R, or S; | X₇₄X₇₅X₇₆X₇₇X₇₈X₇₉X₈₀ (SEQ ID NO: 155), |
| X₇₈ is G, L, M, R, S, or W; | wherein: X₇₄ is E, T or V; X₇₅ is R or S; X₇₆ is K or |
| X₇₉ is A, G, I, M, N, P, S, V, or Y; | R; X₇₇ is M or R; X₇₈ is G, L, M, S, or W; X₇₉ is A, |
| and | G, I, M, P, S, or V; and X₈₀ is G, I, L, N, P, S, or V |
| X₈₀ is G, I, L, N, P, S, V, or W | X₇₄X₇₅X₇₆X₇₇X₇₈X₇₉X₈₀ (SEQ ID NO: 156), |
| | wherein: X₇₄ is E or V; X₇₅ is S; X₇₆ is K or R; X₇₇ |
| | is R; X₇₈ is L, S, or W; X₇₉ is G, I, M, P, S, or V; |
| | and X₈₀ is G, L, N, P, S, or V |
| | X₇₄X₇₅X₇₆X₇₇X₇₈X₇₉X₈₀ (SEQ ID NO: 157), |
| | wherein: X₇₄ is E; X₇₅ is S; X₇₆ is K or R; X₇₇ is R; |
| | X₇₈ is L or W; X₇₉ is M; and X₈₀ is G, P, or S |

TABLE 9J-3
Matriptase Cleavable Expanded Core CM Consensus Sequence 10C

| Expanded Core CM Consensus 10C | Subgenus of Expanded Core CM Consensus 10C |
|---|---|
| X₇₃X₇₄X₇₅X₇₆X₇₇X₇₈X₇₉X₈₀ (SEQ ID | X₇₃X₇₄X₇₅X₇₆X₇₇X₇₈X₇₉X₈₀ (SEQ ID NO: 159), |
| NO: 158), wherein: | wherein: X₇₃ is G, N, P, S, T, or V; X₇₄ is E, L, T or |
| X₇₃ is G, H, N, P, R, S, T, or V; | V; X₇₅ is R or S; X₇₆ is K or R; X₇₇ is M, R, or S; |
| X₇₄ is E, L, Q, S, T or V; | X₇₈ is G, L, M, S, or W; X₇₉ is A, G, I, M, P, S, or |
| X₇₅ is L, R, or S; | V; and X₈₀ is G, I, L, N, P, S, or V |
| X₇₆ is H, K, or R; | X₇₃X₇₄X₇₅X₇₆X₇₇X₇₈X₇₉X₈₀ (SEQ ID NO: 160), |
| X₇₇ is A, M, R, or S; | wherein: X₇₃ is G, N, P, S, or V; X₇₄ is E, T or V; |
| X₇₈ is G, L, M, R, S, or W; | X₇₅ is R or S; X₇₆ is K or R; X₇₇ is M or R; X₇₈ is G, |
| X₇₉ is A, G, I, M, N, P, S, V, or Y; | L, M, S, or W; X₇₉ is A, G, I, M, P, S, or V; and X₈₀ |
| and | is G, I, L, N, P, S, or V |
| X₈₀ is G, I, L, N, P, S, V, or W | X₇₃X₇₄X₇₅X₇₆X₇₇X₇₈X₇₉X₈₀ (SEQ ID NO: 161), |
| | wherein: X₇₃ is G, P, S, or V; X₇₄ is E or V; X₇₅ is |
| | S; X₇₆ is K or R; X₇₇ is R; X₇₈ is L, S, or W; X₇₉ is |
| | G, I, M, P, S, or V; and X₈₀ is G, L, N, P, S, or V |
| | X₇₃X₇₄X₇₅X₇₆X₇₇X₇₈X₇₉X₈₀ (SEQ ID NO: 162), |
| | wherein: X₇₃ is G, P, or S; X₇₄ is E; X₇₅ is S; X₇₆ is |
| | K or R; X₇₇ is R; X₇₈ is L or W; X₇₉ is M; and X₈₀ is |
| | G, P, or S |

In some embodiments, the CM comprises a core CM consensus 1 sequence comprising the amino acid sequence AAPRS (SEQ ID NO: 163). In some embodiments, the CM comprises an expanded core CM consensus 1 sequence comprising the amino acid sequence AAPRSF (SEQ ID NO: 164).

In some embodiments, the CM comprises a core CM consensus 2 sequence comprising the amino acid sequence SRRVP (SEQ ID NO: 165). In some embodiments, the CM comprises an expanded core CM consensus 2 sequence comprising an amino acid sequence selected from the group consisting of QSRRVP (SEQ ID NO: 166), QTRRVP (SEQ ID NO: 167), SRRVPL (SEQ ID NO: 168), SRRVPV (SEQ ID NO: 169), QSRRVPL (SEQ ID NO: 170), QSRRVPV (SEQ ID NO: 171), QTRRVPL (SEQ ID NO: 172), and QTRRVPV (SEQ ID NO: 173).

In some embodiments, the CM comprises the amino acid sequence QSRRVP (SEQ ID NO: 166). In some embodiments, the CM comprises the amino acid sequence QTRRVP (SEQ ID NO: 167). In some embodiments, the CM comprises the amino acid sequence SRRVPL (SEQ ID NO: 168). In some embodiments, the CM comprises the amino acid sequence SRRVPV (SEQ ID NO: 169). In some embodiments, the CM comprises the amino acid sequence QSRRVPL (SEQ ID NO: 170). In some embodiments, the CM comprises the amino acid sequence QSRRVPV (SEQ ID NO: 171). In some embodiments, the CM comprises the amino acid sequence QTRRVPL (SEQ ID NO: 172). In some embodiments, the CM comprises the amino acid sequence QTRRVPV (SEQ ID NO: 173).

In some embodiments, the CM comprises a core CM consensus 3 sequence comprising the amino acid sequence PPLGR (SEQ ID NO: 174). In some embodiments, the CM comprises an expanded core CM consensus 3 sequence comprising an amino acid sequence selected from the group consisting of GPPLGR (SEQ ID NO: 175), SPPLGR (SEQ ID NO: 176), CGPPLGR (SEQ ID NO: 177), CSPPLGR (SEQ ID NO: 178), GGPPLGR (SEQ ID NO: 179), GSPPLGR (SEQ ID NO: 180), SGPPLGR (SEQ ID NO: 181), SSPPLGR (SEQ ID NO: 182), GCGPPLGR (SEQ ID NO: 183), GCSPPLGR (SEQ ID NO: 184), GGGPPLGR (SEQ ID NO: 185), GGSPPLGR (SEQ ID NO: 186), GSGPPLGR (SEQ ID NO: 187), GSSPPLGR (SEQ ID NO: 188), SCGPPLGR (SEQ ID NO: 189), SCSPPLGR (SEQ ID NO: 190), SGGPPLGR (SEQ ID NO: 191), SGSPPLGR (SEQ ID NO: 192), SSGPPLGR (SEQ ID NO: 193), and SSSPPLGR (SEQ ID NO: 194).

In some embodiments, the CM comprises the amino acid sequence GPPLGR (SEQ ID NO: 175). In some embodiments, the CM comprises the amino acid sequence SPPLGR (SEQ ID NO: 176). In some embodiments, the CM comprises the amino acid sequence CGPPLGR (SEQ ID NO: 177). In some embodiments, the CM comprises the amino acid sequence CSPPLGR (SEQ ID NO: 178). In some embodiments, the CM comprises the amino acid sequence GGPPLGR (SEQ ID NO: 179). In some embodiments, the CM comprises the amino acid sequence GSPPLGR (SEQ ID NO: 180). In some embodiments, the CM comprises the amino acid sequence SGPPLGR (SEQ ID NO: 181). In some embodiments, the CM comprises the amino acid sequence SSPPLGR (SEQ ID NO: 182). In some embodiments, the CM comprises the amino acid sequence GCGPPLGR (SEQ ID NO: 183). In some embodiments, the CM comprises the amino acid sequence GCSPPLGR (SEQ ID NO: 184). In some embodiments, the CM comprises the amino acid sequence GGGPPLGR (SEQ ID NO: 185). In some embodiments, the CM comprises the amino acid sequence GGSPPLGR (SEQ ID NO: 186). In some embodiments, the CM comprises the amino acid sequence GSGPPLGR (SEQ ID NO: 187). In some embodiments, the CM comprises the amino acid sequence GSSPPLGR (SEQ ID NO: 188). In some embodiments, the CM comprises the amino acid sequence SCGPPLGR (SEQ ID NO: 189). In some embodiments, the CM comprises the amino acid sequence SCSPPLGR (SEQ ID NO: 190). In some embodiments, the CM comprises the amino acid sequence SGGPPLGR (SEQ ID NO: 191). In some embodiments, the CM comprises the amino acid sequence SGSPPLGR (SEQ ID NO: 192). In some embodiments, the CM comprises the amino acid sequence SSGPPLGR (SEQ ID NO: 193). In some embodiments, the CM comprises the amino acid sequence SSSPPLGR (SEQ ID NO: 194).

In some embodiments, the CM comprises a core CM consensus 4 sequence comprising the amino acid sequence LRSGW (SEQ ID NO: 195). In some embodiments, the CM comprises an expanded core CM consensus 4 sequence comprising an amino acid sequence selected from the group consisting of MLRSGW (SEQ ID NO: 196), MLRSGWR (SEQ ID NO: 197), MLRSGWRG (SEQ ID NO: 198), MLRSGWRL (SEQ ID NO: 199), and MLRSGWRS (SEQ ID NO: 200).

In some embodiments, the CM comprises the amino acid sequence MLRSGW, (SEQ ID NO: 196). In some embodiments, the CM comprises the amino acid sequence MLRSGWR (SEQ ID NO: 197). In some embodiments, the CM comprises the amino acid sequence MLRSGWRG (SEQ ID NO: 198). In some embodiments, the CM comprises the amino acid sequence MLRSGWRL (SEQ ID NO: 199). In some embodiments, the CM comprises the amino acid sequence MLRSGWRS (SEQ ID NO: 200).

In some embodiments, the CM comprises a core CM consensus 5 sequence comprising the amino acid sequence VSRSA (SEQ ID NO: 201). In some embodiments, the CM comprises an expanded core CM consensus 5 sequence comprising an amino acid sequence selected from the group consisting of IVSRSA (SEQ ID NO: 202), YIVSRSA (SEQ ID NO: 203), and QYIVSRSA (SEQ ID NO: 204).

In some embodiments, the CM comprises the amino acid sequence IVSRSA (SEQ ID NO: 202). In some embodiments, the CM comprises the amino acid sequence YIVSRSA (SEQ ID NO: 203). In some embodiments, the CM comprises the amino acid sequence QYIVSRSA (SEQ ID NO: 204).

In some embodiments, the CM comprises a core CM consensus 6 sequence comprising the amino acid sequence ALRAP (SEQ ID NO: 205). In some embodiments, the CM comprises an expanded core CM consensus 6 sequence comprising the amino acid sequence RALRAP (SEQ ID NO: 206).

In some embodiments, the CM comprises a core CM consensus 7 sequence comprising the amino acid sequence PAGRR (SEQ ID NO: 207). In some embodiments, the CM comprises an expanded core CM consensus 7 sequence comprising an amino acid sequence selected from the group consisting of PAGRRS (SEQ ID NO: 208), PAGRRSL (SEQ ID NO: 209), VPAGRRS (SEQ ID NO: 210), and VPAGRRSL (SEQ ID NO: 211).

In some embodiments, the CM comprises the amino acid sequence PAGRRS (SEQ ID NO: 208). In some embodiments, the CM comprises the amino acid sequence PAGRRSL (SEQ ID NO: 209). In some embodiments, the CM comprises the amino acid sequence VPAGRRS (SEQ ID NO: 210). In some embodiments, the CM comprises the amino acid sequence VPAGRRSL (SEQ ID NO: 211).

In some embodiments, the CM comprises a core CM consensus 8 sequence comprising the amino acid sequence GRSML (SEQ ID NO: 212). In some embodiments, the CM comprises an expanded core CM consensus 8 sequence comprising an amino acid sequence selected from the group consisting of GRSMLL (SEQ ID NO: 213), GRSMLM (SEQ ID NO: 214), GRSMLLG (SEQ ID NO: 215), GRSMLLP (SEQ ID NO: 216), GRSMLLS (SEQ ID NO: 217), GRSMLMG (SEQ ID NO: 218), GRSMLMP (SEQ ID NO: 219), GRSMLMS (SEQ ID NO: 220), GRSMLLGG (SEQ ID NO: 221), GRSMLLPG (SEQ ID NO: 222), GRSMLLSG (SEQ ID NO: 223), GRSMLMGG (SEQ ID NO: 224), GRSMLMPG (SEQ ID NO: 225), GRSMLMSG (SEQ ID NO: 226), GRSMLLGP (SEQ ID NO: 227), GRSMLLPP (SEQ ID NO: 228), GRSMLLSP (SEQ ID NO: 229), GRSMLMGP (SEQ ID NO: 230), GRSMLMPP (SEQ ID NO: 231), GRSMLMSP (SEQ ID NO: 232), GRSMLLGS (SEQ ID NO: 233), GRSMLLPS (SEQ ID NO: 234), GRSMLLSS (SEQ ID NO: 235), GRSMLMGS (SEQ ID NO: 236), GRSMLMPS (SEQ ID NO: 237), and GRSMLMSS (SEQ ID NO: 238).

In some embodiments, the CM comprises the amino acid sequence GRSMLL (SEQ ID NO: 213). In some embodiments, the CM comprises the amino acid sequence GRSMLM (SEQ ID NO: 214). In some embodiments, the CM comprises the amino acid sequence GRSMLLG (SEQ ID NO: 215). In some embodiments, the CM comprises the amino acid sequence GRSMLLP (SEQ ID NO: 216). In some embodiments, the CM comprises the amino acid sequence GRSMLLS (SEQ ID NO: 217). In some embodiments, the CM comprises the amino acid sequence GRSMLMG (SEQ ID NO: 218). In some embodiments, the CM comprises the amino acid sequence GRSMLMP (SEQ ID NO: 219). In some embodiments, the CM comprises the amino acid sequence GRSMLMS (SEQ ID NO: 220). In some embodiments, the CM comprises the amino acid sequence GRSMLLGG (SEQ ID NO: 221). In some embodiments, the CM comprises the amino acid sequence GRSMLLPG (SEQ ID NO: 222). In some embodiments, the CM comprises the amino acid sequence GRSMLLSG (SEQ ID NO: 223). In some embodiments, the CM comprises the amino acid sequence GRSMLMGG (SEQ ID NO: 224). In some embodiments, the CM comprises the amino acid sequence GRSMLMPG (SEQ ID NO: 225). In some embodiments, the CM comprises the amino acid sequence GRSMLMSG (SEQ ID NO: 226). In some embodiments, the CM comprises the amino acid sequence GRSMLLGP (SEQ ID NO: 227). In some embodiments, the CM comprises the amino acid sequence GRSMLLPP (SEQ ID NO: 228). In some embodiments, the CM comprises the amino acid sequence GRSMLLSP (SEQ ID NO: 229). In some embodiments, the CM comprises the amino acid sequence GRSMLMGP (SEQ ID NO: 230). In some embodiments, the CM comprises the amino acid sequence GRSMLMPP (SEQ ID NO: 231). In some embodiments, the CM comprises the amino acid sequence GRSMLMSP (SEQ ID NO: 232). In some embodiments, the CM comprises the amino acid sequence GRSMLLGS (SEQ ID NO: 233). In some embodiments, the CM comprises the amino acid sequence GRSMLLPS (SEQ ID NO: 234). In some embodiments, the CM comprises the amino acid sequence GRSMLLSS (SEQ ID NO: 235). In some embodiments, the CM comprises the amino acid sequence GRSMLMGS (SEQ ID NO: 236). In some embodiments, the CM comprises the amino acid sequence GRSMLMPS (SEQ ID NO: 237). In some embodiments, the CM comprises the amino acid sequence GRSMLMSS (SEQ ID NO: 238).

In some embodiments, the CM comprises a core CM consensus 9 sequence comprising the amino acid sequence LARAG (SEQ ID NO: 239). In some embodiments, the CM comprises an expanded core CM consensus 9 sequence comprising an amino acid sequence selected from the group consisting of LARAGI (SEQ ID NO: 240), LARAGL (SEQ ID NO: 241), PLARAGI (SEQ ID NO: 242), PLARAGL (SEQ ID NO: 243), RPLARAGI (SEQ ID NO: 244), and RPLARAGL (SEQ ID NO: 245).

In some embodiments, the CM comprises the amino acid sequence LARAGI (SEQ ID NO: 240). In some embodiments, the CM comprises the amino acid sequence LARAGL (SEQ ID NO: 241). In some embodiments, the CM comprises the amino acid sequence PLARAGI (SEQ ID NO: 242). In some embodiments, the CM comprises the amino acid sequence PLARAGL (SEQ ID NO: 243). In some embodiments, the CM comprises the amino acid sequence RPLARAGI (SEQ ID NO: 244). In some embodiments, the CM comprises the amino acid sequence RPLARAGL (SEQ ID NO: 245).

In some embodiments, the CM comprises a core CM consensus 10 sequence comprising the amino acid sequence ESRRW (SEQ ID NO: 246). In some embodiments, the CM comprises an expanded core CM consensus 10 sequence comprising an amino acid sequence selected from the group consisting of ESRRWM (SEQ ID NO: 247), ESRRWMP (SEQ ID NO: 248), and PESRRWMP (SEQ ID NO: 249).

In some embodiments, the CM comprises the amino acid sequence ESRRWM (SEQ ID NO: 247). In some embodiments, the CM comprises the amino acid sequence ESRRWMP (SEQ ID NO: 248). In some embodiments, the CM comprises the amino acid sequence PESRRWMP (SEQ ID NO: 249).

In some embodiments, the CM comprises an amino acid sequence selected from the group consisting of ILPRSPAF (SEQ ID NO: 250), VAGRSMRP (SEQ ID NO: 251), VVPEGRRS (SEQ ID NO: 252), QGRAITFI (SEQ ID NO: 253), VLSKQMSF (SEQ ID NO: 254), LKGRSYYY (SEQ ID NO: 255), KRMPVQFL (SEQ ID NO: 256), PQHRIVSF (SEQ ID NO: 257), YKKFVGSL (SEQ ID NO: 258), HMMQYARH (SEQ ID NO: 259), IPFSWSRF (SEQ ID NO: 260), LSQARWRK (SEQ ID NO: 261), DISHWRRS (SEQ ID NO: 262), RKTVQHWW (SEQ ID NO: 263), RFYRNQFF (SEQ ID NO: 264), RSLVFAPI (SEQ ID NO: 265), RSPSRLKC (SEQ ID NO: 266), and RKMPNITV (SEQ ID NO: 267).

In some embodiments, the CM comprises the amino acid sequence ILPRSPAF (SEQ ID NO: 250). In some embodiments, the CM comprises the amino acid sequence VAGRSMRP (SEQ ID NO: 251). In some embodiments, the CM comprises the amino acid sequence VVPEGRRS (SEQ ID NO: 252). In some embodiments, the CM comprises the amino acid sequence QGRAITFI (SEQ ID NO: 253). In some embodiments, the CM comprises the amino acid sequence VLSKQMSF (SEQ ID NO: 254). In some embodiments, the CM comprises the amino acid sequence LKGRSYYY (SEQ ID NO: 255). In some embodiments, the CM comprises the amino acid sequence KRMPVQFL (SEQ ID NO: 256). In some embodiments, the CM comprises the amino acid sequence PQHRIVSF (SEQ ID NO: 257). In some embodiments, the CM comprises the amino acid sequence YKKFVGSL (SEQ ID NO: 258). In some embodiments, the CM comprises the amino acid sequence HMMQYARH (SEQ ID NO: 259). In some embodiments, the CM comprises the amino acid sequence IPFSWSRF (SEQ ID NO: 260). In some embodiments, the CM comprises the amino acid sequence LSQARWRK (SEQ ID NO: 261). In some embodiments, the CM comprises the amino acid sequence DISHWRRS (SEQ ID NO: 262). In some embodiments, the CM comprises the amino acid sequence RKTVQHWW (SEQ ID NO: 263_). In some embodiments, the CM comprises the amino acid sequence RFYRNQFF (SEQ ID NO: 264). In some embodiments, the CM comprises the amino acid sequence RSLVFAPI (SEQ ID NO: 265). In some embodiments, the CM comprises the amino acid sequence RSPSRLKC (SEQ ID NO: 266). In some embodiments, the CM comprises the amino acid sequence RKMPNITV (SEQ ID NO: 267).

In some embodiments, the CM includes a motif sequence that is a substrate for at least uPA and/or matriptase and includes a core CM consensus sequence shown in Tables 10A-10J below. In some embodiments, the motif sequence includes a subgenus, i.e., a subset, of the core CM consensus sequence shown in Tables 10A-10D below.

TABLE 10A
uPA and/or Matriptase Cleavable Core CM Consensus Sequence 11

| Core CM Consensus 11 | Subgenus of Core CM Consensus 11 |
|---|---|
| X₈₇X₈₈X₈₉X₉₀X₉₁X₉₂X₉₃X₉₄ (SEQ ID | X₈₇X₈₈X₈₉X₉₀X₉₁X₉₂X₉₃X₉₄ (SEQ ID NO: 269), |
| NO: 268), wherein: | wherein: X₈₇ is D, L, S, or V; X₈₈ is C, G, N, R, S, or T; |
| X₈₇ is D, I, L, R, S, or V; | X₈₉ is D, G, or S; X₉₀ is R; X₉₁ is F or S; X₉₂ is A, G, I, |
| X₈₈ is C, G, H, I, K, N, R, S, T, or Y; | L, T, or V; X₉₃ is H, I, N, or S; and X₉₄ is H, M, R, or |
| X₈₉ is D, G, or S; | Y |
| X₉₀ is R; | X₈₇X₈₈X₈₉X₉₀X₉₁X₉₂X₉₃X₉₄ (SEQ ID NO: 270), |
| X₉₁ is F or S; | wherein: X₈₇ is L or V; X₈₈ is G, H, K, N, S, or T; X₈₉ |
| X₉₂ is A, D, G, H, I, L, T, or V; | is G; X₉₀ is R; X₉₁ is S; X₉₂ is A or D; X₉₃ is N or R; |
| X₉₃ is H, I, N, R, S, or T; and | and X₉₄ is H or Y |
| X₉₄ is H, L, M, R, V or Y | X₈₇X₈₈X₈₉X₉₀X₉₁X₉₂X₉₃X₉₄ (SEQ ID NO: 271), |
| | wherein: X₈₇ is L or V; X₈₈ is G, H, N or S; X₈₉ is G; |
| | X₉₀ is R; X₉₁ is S; X₉₂ is A or D; X₉₃ is N; and X₉₄ is H |
| | X₈₇X₈₈X₈₉X₉₀X₉₁X₉₂X₉₃X₉₄ (SEQ ID NO: 272), |
| | wherein: X₈₇ is L or V; X₈₈ is S; X₈₉ is G; X₉₀ is R; X₉₁ |
| | is S; X₉₂ is A or D; X₉₃ is N; and X₉₄ is H |
| | X₈₇X₈₈X₈₉X₉₀X₉₁X₉₂X₉₃X₉₄ (SEQ ID NO: 273), |
| | wherein: X₈₇ is L or V; X₈₈ is G or S; X₈₉ is G; X₉₀ is |
| | R; X₉₁ is S; X₉₂ is A; X₉₃ is N; and X₉₄ is H |

TABLE 10B
uPA and/or Matriptase Cleavable Core CM Consensus Sequence 12

| Core CM Consensus 12 | Subgenus of Core CM Consensus 12 |
|---|---|
| X₉₉X₁₀₀X₁₀₁X₁₀₂X₁₀₃X₁₀₄X₁₀₅X₁₀₆ | X₉₉X₁₀₀X₁₀₁X₁₀₂X₁₀₃X₁₀₄X₁₀₅X₁₀₆ (SEQ ID NO: 275), |
| (SEQ ID NO: 274), wherein: | wherein: X₉₉ is L; X₁₀₀ is N, S, or T; X₁₀₁ is G; X₁₀₂ is |
| X₉₉ is D, I, L, R, S, or V; | R; X₁₀₃ is S; X₁₀₄ is A, D, or H; X₁₀₅ is N or R; and |
| X₁₀₀ is C, G, H, I, K, N, R, S, T or Y; | X₁₀₆ is H, L, V, or Y |
| X₁₀₁ is D, G, or S; | X₉₉X₁₀₀X₁₀₁X₁₀₂X₁₀₃X₁₀₄X₁₀₅X₁₀₆ (SEQ ID NO: 276), |
| X₁₀₂ is R; | wherein: X₉₉ is L or V; X₁₀₀ is G, H, K, N, S, or T; X₁₀₁ |
| X₁₀₃ is F or S; | is G; X₁₀₂ is R; X₁₀₃ is S; X₁₀₄ is A or D; X₁₀₅ is N or |
| X₁₀₄ is A, D, G, H, I, L, T, or V; | R; and X₁₀₆ is H or Y |
| X₁₀₅ is H, I, N, R, S, or T; and | X₉₉X₁₀₀X₁₀₁X₁₀₂X₁₀₃X₁₀₄X₁₀₅X₁₀₆ (SEQ ID NO: 277), |
| X₁₀₆ is H, L, M, R, V, or Y | wherein: X₉₉ is L or V; X₁₀₀ is G, H, N, or S; X₁₀₁ is G; |
| | X₁₀₂ is R; X₁₀₃ is S; X₁₀₄ is A or D; X₁₀₅ is N; and X₁₀₆ |
| | is H |
| | X₉₉X₁₀₀X₁₀₁X₁₀₂X₁₀₃X₁₀₄X₁₀₅X₁₀₆ (SEQ ID NO: 278), |
| | wherein: X₉₉ is L or V; X₁₀₀ is S; X₁₀₁ is G; X₁₀₂ is R; |
| | X₁₀₃ is S; X₁₀₄ is A or D; X₁₀₅ is N; and X₁₀₆ is H |
| | X₉₉X₁₀₀X₁₀₁X₁₀₂X₁₀₃X₁₀₄X₁₀₅X₁₀₆ (SEQ ID NO: 279), |
| | wherein: X₉₉ is L; X₁₀₀ is N or S; X₁₀₁ is G; X₁₀₂ is R; |
| | X₁₀₃ is S; X₁₀₄ is A or D; X₁₀₅ is N or R; and X₁₀₆ is H |

TABLE 10C
uPA and/or Matriptase Cleavable Core CM Consensus Sequence 13

| Core CM Consensus 13 | Subgenus of Core CM Consensus 13 |
|---|---|
| X₁₁₁X₁₁₂X₁₁₃X₁₁₄X₁₁₅X₁₁₆X₁₁₇X₁₁₈ | X₁₁₁X₁₁₂X₁₁₃X₁₁₄X₁₁₅X₁₁₆X₁₁₇X₁₁₈ (SEQ ID NO: 281), |
| (SEQ ID NO: 280), wherein: | wherein: X₁₁₁ is C or R; X₁₁₂ is I, S, or Y; X₁₁₃ is G or |
| X₁₁₁ is C, G, H, L, P, R, S, T, or V; | R; X₁₁₄ is R or S; X₁₁₅ is F, P, or S; X₁₁₆ is D, G, or H; |
| X₁₁₂ is I, L, M, N, S, T, V, or Y; | X₁₁₇ is H or N; and X₁₁₈ is H |
| X₁₁₃ is A, D, E, G, K, R, or V; | X₁₁₁X₁₁₂X₁₁₃X₁₁₄X₁₁₅X₁₁₆X₁₁₇X₁₁₈ (SEQ ID NO: 282), |
| X₁₁₄ is A, C, G, H, L, R, S, T, or V; | wherein: X₁₁₁ is R; X₁₁₂ is I; X₁₁₃ is G; X₁₁₄ is R; X₁₁₅ |
| X₁₁₅ is C, F, P, S, T, V, or Y; | is S; X₁₁₆ is D or H; X₁₁₇ is N; and X₁₁₈ is H |
| X₁₁₆ is A, D, E, G, H, N, T, V, or Y; | |
| X₁₁₇ is D, E, H, K, N, Q, R, S, T; and | |
| X₁₁₈ is H, L, N, R, S, V, or Y | |

TABLE 10D
uPA and/or Matriptase Cleavable Core CM Consensus Sequence 14

| Core CM Consensus 14 | Subgenus of Core CM Consensus 14 |
|---|---|
| X₁₂₃X₁₂₄X₁₂₅X₁₂₆X₁₂₇X₁₂₈X₁₂₉X₁₃₀ | X₁₂₃X₁₂₄X₁₂₅X₁₂₆X₁₂₇X₁₂₈X₁₂₉X₁₃₀ (SEQ ID NO: 284), |
| (SEQ ID NO: 283), wherein: | wherein: X₁₂₃ is L or T; X₁₂₄ is E, R, or S; X₁₂₅ is G; |
| X₁₂₃ is L, R, T, or V; | X₁₂₆ is R; X₁₂₇ is P or S; X₁₂₈ is A, G, or Y; X₁₂₉ is E or |
| X₁₂₄ is E, G, I, N, R, or S; | N; and X₁₃₀ is P, Q, or S |
| X₁₂₅ is G; | X₁₂₃X₁₂₄X₁₂₅X₁₂₆X₁₂₇X₁₂₈X₁₂₉X₁₃₀ (SEQ ID NO: 285), |
| X₁₂₆ is R; | wherein: X₁₂₃ is L, T, or V; X₁₂₄ is R or S; X₁₂₅ is G; |
| X₁₂₇ is P or S; | X₁₂₆ is R; X₁₂₇ is S; X₁₂₈ is A or G; X₁₂₉ is K, N or Y; |
| X₁₂₈ is A, G, or Y; | and X₁₃₀ is P |
| X₁₂₉ is E, K, N, or Y; and | X₁₂₃X₁₂₄X₁₂₅X₁₂₆X₁₂₇X₁₂₈X₁₂₉X₁₃₀ (SEQ ID NO: 286), |
| X₁₃₀ is P, Q, or S | wherein: X₁₂₃ is L or T; X₁₂₄ is S; X₁₂₅ is G; X₁₂₆ is R; |
| | X₁₂₇ is S; X₁₂₈ is A or G; X₁₂₉ is N; and X₁₃₀ is P |

In some embodiments, the motif sequence is a substrate for at least uPA and/or matriptase and includes an expanded consensus sequence based on one of the core CM consensus sequence shown in Tables 10A-10D. In some embodiments, the expanded consensus sequence is a consensus sequence shown in Tables 11A-11D below.

TABLE 11A
uPA and/or Matriptase Cleavable Expanded Core CM Consensus Sequence 11

| Expanded Core CM Consensus 11 | Subgenus of Expanded Core CM Consensus 11 |
|---|---|
| X₈₅X₈₆X₈₇X₈₈X₈₉X₉₀X₉₁X₉₂X₉₃X₉₄ | X₈₅X₈₆X₈₇X₈₈X₈₉X₉₀X₉₁X₉₂X₉₃X₉₄X₉₅X₉₆ (SEQ ID |
| X_{95X96} (SEQ ID NO: 287), wherein: | NO: 288), wherein: |
| X₈₅ is A, D, G, K, L, N, R, S, T, | X₈₅ is A, D, G, K, N, S, or V; X₈₆ is A, G, K, M, R, S, or |
| or V; | T; X₈₇ is D, L, S, or V; X₈₈ is C, G, N, R, S, or T; X₈₉ is |
| X₈₆ is A, G, K, M, P, Q, R, S, or | D, G, or S; X₉₀ is R; X₉₁ is F or S; X₉₂ is A, G, I, L, T, |
| T; | or V; X₉₃ is H, I, N, or S; X₉₄ is H, M, R, or Y; X₉₅ is E, |
| X₈₇ is D, I, L, R, S, or V; | G, K, or R; and X₉₆ is K, R, or S |
| X₈₈ is C, G, H, I, K, N, R, S, T, or | X₈₅X₈₆X₈₇X₈₈X₈₉X₉₀X₉₁X₉₂X₉₃X₉₄X₉₅X₉₆ (SEQ ID |
| Y; | NO: 289), wherein: |
| X₈₉ is D, G, or S; | X₈₅ is A, D, G, N, R, or T; X₈₆ is G, K, P, R, S, or T; X₈₇ |
| X₉₀ is R; | is L or V; X₈₈ is G, H, K, N, S, or T; X₈₉ is G; X₉₀ is R; |
| X₉₁ is F or S; | X₉₁ is S; X₉₂ is A or D; X₉₃ is N or R; X₉₄ is H or Y; X₉₅ |
| X₉₂ is A, D, G, H, I, L, T, or V; | is E, K, N, Q, or R; and X₉₆ is A, K, or R |
| X₉₃ is H, I, N, R, S, or T; | X₈₅X₈₆X₈₇X₈₈X₈₉X₉₀X₉₁X₉₂X₉₃X₉₄X₉₅X₉₆ (SEQ ID |
| X₉₄ is H, L, M, R, V or Y; | NO: 290), wherein: |
| X₉₅ is E, G, K, N, Q, R, or V; and | X₈₅ is A, D, G, or R; X₈₆ is K, P, R or T; X₈₇ is L or V; |
| X₉₆ is A, G, K, L, Q, R, or S | X₈₈ is G, H, N or S; X₈₉ is G; X₉₀ is R; X₉₁ is S; X₉₂ is A |
| | or D; X₉₃ is N; X₉₄ is H; X₉₅ is K, N, or R; and X₉₆ is A, |
| | K, or R |
| | X₈₅X₈₆X₈₇X₈₈X₈₉X₉₀X₉₁X₉₂X₉₃X₉₄X₉₅X₉₆ (SEQ ID |
| | NO: 291), wherein: |
| | X₈₅ is A or D; X₈₆ is K, P, or R; X₈₇ is L or V; X₈₈ is S; |
| | X₈₉ is G; X₉₀ is R; X₉₁ is S; X₉₂ is A or D; X₉₃ is N; X₉₄ |
| | is H; X₉₅ is K or R; and X₉₆ is K or R |
| | X₈₅X₈₆X₈₇X₈₈X₈₉X₉₀X₉₁X₉₂X₉₃X₉₄X₉₅X₉₆ (SEQ ID |
| | NO: 292), wherein: |
| | X₈₅ is D, G, or N; X₈₆ is K, R, or S; X₈₇ is L or V; X₈₈ is |
| | G or S; X₈₉ is G; X₉₀ is R; X₉₁ is S; X₉₂ is A; X₉₃ is N; |
| | X₉₄ is H; X₉₅ is K; and X₉₆ is K |

TABLE 11B
uPA and/or Matriptase Cleavable Expanded Core CM Consensus Sequence 12

| Expanded Core CM Consensus 12 | Subgenus of Expanded Core CM Consensus 12 |
|---|---|
| X₉₇X₉₈X₉₉X₁₀₀X₁₀₁X₁₀₂X₁₀₃X₁₀₄X₁₀₅X₁₀₆ | X₉₇X₉₈X₉₉X₁₀₀X₁₀₁X₁₀₂X₁₀₃X₁₀₄X₁₀₅X₁₀₆X₁₀₇X₁₀₈ |
| X₁₀₇X₁₀₈ (SEQ ID NO: 293), | (SEQ ID NO: 294), wherein: X₉₇ is D, G, K, or R; X₉₈ |
| wherein: | is G, P, or R; X₉₉ is L; X₁₀₀ is N, S, or T; X₁₀₁ is G; |
| X₉₇ is A, D, G, K, L, N, R, S, T, or V; | X₁₀₂ is R; X₁₀₃ is S; X₁₀₄ is A, D, or H; X₁₀₅ is N or R; |
| X₉₈ is A, G, K, M, P, Q, R, S, or T; | X₁₀₆ is H, L, V, or Y; X₁₀₇ is E, G, K, N, Q, or R; and |
| X₉₉ is D, I, L, R, S, or V; | X₁₀₈ is A, G, K, L, Q, or R |
| X₁₀₀ is C, G, H, I, K, N, R, S, T or Y; | X₉₇X₉₈X₉₉X₁₀₀X₁₀₁X₁₀₂X₁₀₃X₁₀₄X₁₀₅X₁₀₆X₁₀₇X₁₀₈ |
| X₁₀₁ is D, G, or S; | (SEQ ID NO: 295), wherein: X₉₇ is A, D, G, N, R, or |
| X₁₀₂ is R; | T; X₉₈ is G, K, P, R, S, or T; X₉₉ is L or V; X₁₀₀ is G, |
| X₁₀₃ is F or S; | H, K, N, S, or T; X₁₀₁ is G; X₁₀₂ is R; X₁₀₃ is S; X₁₀₄ is |
| X₁₀₄ is A, D, G, H, I, L, T, or V; | A or D; X₁₀₅ is N or R; X₁₀₆ is H or Y; X₁₀₇ is E, K, N, |
| X₁₀₅ is H, I, N, R, S, or T; | Q, or R; and X₁₀₈ is A, K, or R |
| X₁₀₆ is H, L, M, R, V, or Y; | X₉₇X₉₈X₉₉X₁₀₀X₁₀₁X₁₀₂X₁₀₃X₁₀₄X₁₀₅X₁₀₆X₁₀₇X₁₀₈ |
| X₁₀₇ is E, G, K, N, Q, R, or V; and | (SEQ ID NO: 296), wherein: X₉₇ is A, D, G, or R; X₉₈ |
| X₁₀₈ is A, G, K, L, Q, R, or S | is K, P, R, or T; X₉₉ is L or V; X₁₀₀ is G, H, N, or S; |
| | X₁₀₁ is G; X₁₀₂ is R; X₁₀₃ is S; X₁₀₄ is A or D; X₁₀₅ is N; |
| | X₁₀₆ is H; X₁₀₇ is K, N, or R; and X₁₀₈ is A, K, or R |
| | X₉₇X₉₈X₉₉X₁₀₀X₁₀₁X₁₀₂X₁₀₃X₁₀₄X₁₀₅X₁₀₆X₁₀₇X₁₀₈ |
| | (SEQ ID NO: 297), wherein: X₉₇ is A or D; X₉₈ is K, P, |
| | or R; X₉₉ is L or V; X₁₀₀ is S; X₁₀₁ is G; X₁₀₂ is R; X₁₀₃ |
| | is S; X₁₀₄ is A or D; X₁₀₅ is N; X₁₀₆ is H; X₁₀₇ is K or |
| | R; and X₁₀₈ is K or R |
| | X₉₇X₉₈X₉₉X₁₀₀X₁₀₁X₁₀₂X₁₀₃X₁₀₄X₁₀₅X₁₀₆X₁₀₇X₁₀₈ |
| | (SEQ ID NO: 298), wherein: X₉₇ is G or R; X₉₈ is P; |
| | X₉₉ is L; X₁₀₀ is N or S; X₁₀₁ is G; X₁₀₂ is R; X₁₀₃ is S; |
| | X₁₀₄ is A or D; X₁₀₅ is N or R; X₁₀₆ is H; X₁₀₇ is K, Q, |
| | or R; and X₁₀₈ is A, K, or R |

TABLE 11C
uPA and/or Matriptase Cleavable Expanded Core CM Consensus Sequence 13

| Expanded Core CM Consensus 13 | Subgenus of Expanded Core CM Consensus 13 |
|---|---|
| X₁₀₉X₁₁₀X₁₁₁X₁₁₂X₁₁₃X₁₁₄X₁₁₅X₁₁₆X₁₁₇ | X₁₀₉X₁₁₀X₁₁₁X₁₁₂X₁₁₃X₁₁₄X₁₁₅X₁₁₆X₁₁₇X₁₁₈X₁₁₉X₁₂₀ |
| X₁₁₈X₁₁₉X₁₂₀ (SEQ ID NO: 299), | (SEQ ID NO: 300), wherein: X₁₀₉ is N; X₁₁₀ is H or R; |
| wherein: | X₁₁₁ is C or R; X₁₁₂ is I, S, or Y; X₁₁₃ is G or R; X₁₁₄ is |
| X₁₀₉ is A, D, G, H, I, K, N, R, S, T, | R or S; X₁₁₅ is F, P, or S; X₁₁₆ is D, G, or H; X₁₁₇ is H |
| or Y; | or N; X₁₁₈ is H; X₁₁₉ is E, K, R, or V; and X₁₂₀ is A, G, |
| X₁₁₀ is D, G, H, L, N, Q, R, or Y; | Q, R, or W |
| X₁₁₁ is C, G, H, L, P, R, S, T, or V; | X₁₀₉X₁₁₀X₁₁₁X₁₁₂X₁₁₃X₁₁₄X₁₁₅X₁₁₆X₁₁₇X₁₁₈X₁₁₉X₁₂₀ |
| X₁₁₂ is I, L, M, N, S, T, V, or Y; | (SEQ ID NO: 301), wherein: X₁₀₉ is N; X₁₁₀ is H; X₁₁₁ |
| X₁₁₃ is A, D, E, G, K, R, or V; | is R; X₁₁₂ is I; X₁₁₃ is G; X₁₁₄ is R; X₁₁₅ is S; X₁₁₆ is D |
| X₁₁₄ is A, C, G, H, L, R, S, T, or V; | or H; X₁₁₇ is N; X₁₁₈ is H; X₁₁₉ is R; and X₁₂₀ is G or R |
| X₁₁ₛ is C, F, P, S, T, V, or Y; | |
| X₁₁₆ is A, D, E, G, H, N, T, V, or Y; | |
| X₁₁₇ is D, E, H, K, N, Q, R, S, T; | |
| X₁₁₈ is H, L, N, R, S, V, or Y; | |
| X₁₁₉ is E, G, K, L, N, Q, R, S, V, | |
| or W; and | |
| X₁₂₀ is A, E, G, K, L, N, P, Q, R, | |
| or W | |

TABLE 11D
uPA and/or Matriptase Cleavable Expanded Core CM Consensus Sequence 14

| Expanded Core CM Consensus 14 | Subgenus of Expanded Core CM Consensus 14 |
|---|---|
| X₁₂₁X₁₂₂X₁₂₃X₁₂₄X₁₂₅X₁₂₆X₁₂₇X₁₂₈X₁₂₉ | X₁₂₁X₁₂₂X₁₂₃X₁₂₄X₁₂₅X₁₂₆X₁₂₇X₁₂₈X₁₂₉X₁₃₀X₁₃₁X₁₃₂ |
| X₁₃₀X₁₃₁X₁₃₂ (SEQ ID NO: 302), | (SEQ ID NO: 303), wherein: X₁₂₁ is M, N, P, R, or T; |
| wherein: | X₁₂₂ is A, P, or S; X₁₂₃ is L or T; X₁₂₄ is E, R, or S; X₁₂₅ |
| X₁₂₁ is A, D, G, M, N, P, R, or T; | is G; X₁₂₆ is R; X₁₂₇ is P or S; X₁₂₈ is A, G, or Y; X₁₂₉ |
| X₁₂₂ is A, H, K, P, R, or S; | is E or N; X₁₃₀ is P, Q, or S; X₁₃₁ is E, K, R; and X₁₃₂ is |
| X₁₂₃ is L, R, T, or V; | E, G, or R |
| X₁₂₄ is E, G, I, N, R, or S; | X₁₂₁X₁₂₂X₁₂₃X₁₂₄X₁₂₅X₁₂₆X₁₂₇X₁₂₈X₁₂₉X₁₃₀X₁₃₁X₁₃₂ |
| X₁₂₅ is G; | (SEQ ID NO: 304), wherein: X₁₂₁ is G, N, or T; X₁₂₂ is |
| X₁₂₆ is R; | A, P, or S; X₁₂₃ is L, T, or V; X₁₂₄ is R or S; X₁₂₅ is G; |
| X₁₂₇ is P or S; | X₁₂₆ is R; X₁₂₇ is 5; X₁₂₈ is A or G; X₁₂₉ is K, N or Y; |
| X₁₂₈ is A, G, or Y; | X₁₃₀ is P; X₁₃₁ is K or R; and X₁₃₂ is D, G or H |
| X₁₂₉ is E, K, N, or Y; | X₁₂₁X₁₂₂X₁₂₃X₁₂₄X₁₂₅X₁₂₆X₁₂₇X₁₂₈X₁₂₉X₁₃₀X₁₃₁X₁₃₂ |
| X₁₃₀ is P, Q, or S; | (SEQ ID NO: 305), wherein: X₁₂₁ is T; X₁₂₂ is P or S; |
| X₁₃₁ is E, K, or R; and | X₁₂₃ is L or T; X₁₂₄ is S; X₁₂₅ is G; X₁₂₆ is R; X₁₂₇ is S; |
| X₁₃₂ is D, E, G, H, or R | X₁₂₈ is A or G; X₁₂₉ is N; X₁₃₀ is P; X₁₃₁ is K or R; and |
| | X₁₃₂ is G or H |
| | X₁₂₁X₁₂₂X₁₂₃X₁₂₄X₁₂₅X₁₂₆X₁₂₇X₁₂₈X₁₂₉X₁₃₀X₁₃₁X₁₃₂ |
| | (SEQ ID NO: 306), wherein: X₁₂₁ is T; X₁₂₂ is S; X₁₂₃ |
| | is L or T; X₁₂₄ is S; X₁₂₅ is G; X₁₂₆ is R; X₁₂₇ is S; X₁₂₈ |
| | is A or G; X₁₂₉ is N; X₁₃₀ is P; X₁₃₁ is R; and X₁₃₂ is G |

In some embodiments, the CM comprises a core CM consensus 11 sequence comprising the amino acid sequence LSGRSANH (SEQ ID NO: 307) or LSGRSGNH (SEQ ID NO: 308). In some embodiments, the CM comprises an expanded core CM consensus 11 sequence comprising the amino acid sequence DRLSGRSANHKK (SEQ ID NO: 309), DRLSGRSDNHKK (SEQ ID NO: 310), or NTLSGRSGNHGS (SEQ ID NO: 311).

In some embodiments, the CM comprises the amino acid sequence LSGRSANH (SEQ ID NO: 307). In some embodiments, the CM comprises the amino acid sequence LSGRSGNH (SEQ ID NO: 308). In some embodiments, the CM comprises the amino acid sequence DRLSGRSANHKK (SEQ ID NO: 309). In some embodiments, the CM comprises the amino acid sequence DRLSGRSDNHKK (SEQ ID NO: 310). In some embodiments, the CM comprises the amino acid sequence NTLSGRSGNHGS (SEQ ID NO: 311).

In some embodiments, the CM comprises the amino acid sequence LSGRSANH (SEQ ID NO: 307). In some embodiments, the CM comprises the amino acid sequence LNGRSDNH (SEQ ID NO: 313). In some embodiments, the CM comprises the amino acid sequence LTGRSDRH (SEQ ID NO: 314). In some embodiments, the CM comprises a core CM consensus 12 sequence comprising the amino acid sequence LSGRSANH (SEQ ID NO: 307), LNGRSDNH (SEQ ID NO: 313), and LTGRSDRH (SEQ ID NO: 314). In some embodiments, the CM an expanded core CM consensus 12 sequence comprising an amino acid sequence selected from the group consisting of DRLSGRSANHKK (SEQ ID NO: 309), DRLSGRSDNHKK (SEQ ID NO: 310), GPLNGRSDNHKA (SEQ ID NO: 320), GPLNGRSDNHKK (SEQ ID NO: 321), GPLNGRSDNHKR (SEQ ID NO: 322), GPLNGRSDNHQA (SEQ ID NO: 323), GPLNGRSDNHQK (SEQ ID NO: 324), GPLNGRSDNHQR (SEQ ID NO: 325), GPLNGRSDNHRA (SEQ ID NO: 326), GPLNGRSDNHRK (SEQ ID NO: 327), GPLNGRSDNHRR (SEQ ID NO: 328), RPLNGRSDNHKA (SEQ ID NO: 329), RPLNGRSDNHKK (SEQ ID NO: 330), RPLNGRSDNHKR (SEQ ID NO: 331), RPLNGRSDNHQA (SEQ ID NO: 332), RPLNGRSDNHQK (SEQ ID NO: 333), RPLNGRSDNHQR (SEQ ID NO: 334), RPLNGRSDNHRA (SEQ ID NO: 335), RPLNGRSDNHRK (SEQ ID NO: 336), RPLNGRSDNHRR (SEQ ID NO: 337), GPLSGRSDNHKA (SEQ ID NO: 338), GPLSGRSDNHKK (SEQ ID NO: 339), GPLSGRSDNHKR (SEQ ID NO: 340), GPLSGRSDNHQA (SEQ ID NO: 341), GPLSGRSDNHQK (SEQ ID NO: 342), GPLSGRSDNHQR (SEQ ID NO: 343), GPLSGRSDNHRA (SEQ ID NO: 344), GPLSGRSDNHRK (SEQ ID NO: 345), GPLSGRSDNHRR (SEQ ID NO: 346), RPLSGRSDNHKA (SEQ ID NO: 347), RPLSGRSDNHKK (SEQ ID NO: 348), RPLSGRSDNHKR (SEQ ID NO: 349), RPLSGRSDNHQA (SEQ ID NO: 350), RPLSGRSDNHQK (SEQ ID NO: 351), RPLSGRSDNHQR (SEQ ID NO: 352), RPLSGRSDNHRA (SEQ ID NO: 353), RPLSGRSDNHRK (SEQ ID NO: 354), RPLSGRSDNHRR (SEQ ID NO: 355), and KGLTGRSDRHQA (SEQ ID NO: 356).

In some embodiments, the CM comprises the amino acid sequence DRLSGRSANHKK (SEQ ID NO: 309). In some embodiments, the CM comprises the amino acid sequence DRLSGRSDNHKK (SEQ ID NO: 310). In some embodiments, the CM comprises the amino acid sequence GPLNGRSDNHKA (SEQ ID NO: 320). In some embodiments, the CM comprises the amino acid sequence GPLNGRSDNHKK (SEQ ID NO: 321). In some embodiments, the CM comprises the amino acid sequence GPLNGRSDNHKR (SEQ ID NO: 322). In some embodiments, the CM comprises the amino acid sequence GPLNGRSDNHQA (SEQ ID NO: 323). In some embodiments, the CM comprises the amino acid sequence GPLNGRSDNHQK (SEQ ID NO: 324). In some embodiments, the CM comprises the amino acid sequence GPLNGRSDNHQR (SEQ ID NO: 325). In some embodiments, the CM comprises the amino acid sequence GPLNGRSDNHRA (SEQ ID NO: 326). In some embodiments, the CM comprises the amino acid sequence GPLNGRSDNHRK (SEQ ID NO: 327). In some embodiments, the CM comprises the amino acid sequence GPLNGRSDNHRR (SEQ ID NO: 328). In some embodiments, the CM comprises the amino acid sequence RPLNGRSDNHKA (SEQ ID NO: 329). In some embodiments, the CM comprises the amino acid sequence RPLNGRSDNHKK (SEQ ID NO: 330). In some embodiments, the CM comprises the amino acid sequence RPLNGRSDNHKR (SEQ ID NO: 331). In some embodiments, the CM comprises the amino acid sequence RPLNGRSDNHQA (SEQ ID NO: 332). In some embodiments, the CM comprises the amino acid sequence RPLNGRSDNHQK (SEQ ID NO: 333). In some embodiments, the CM comprises the amino acid sequence RPLNGRSDNHQR (SEQ ID NO: 334). In some embodiments, the CM comprises the amino acid sequence RPLNGRSDNHRA (SEQ ID NO: 335). In some embodiments, the CM comprises the amino acid sequence RPLNGRSDNHRK (SEQ ID NO: 336). In some embodiments, the CM comprises the amino acid sequence RPLNGRSDNHKR (SEQ ID NO: 337). In some embodiments, the CM comprises the amino acid sequence GPLSGRSDNHKA (SEQ ID NO: 338). In some embodiments, the CM comprises the amino acid sequence GPLSGRSDNHKK (SEQ ID NO: 339). In some embodiments, the CM comprises the amino acid sequence GPLSGRSDNHKR (SEQ ID NO: 340). In some embodiments, the CM comprises the amino acid sequence GPLSGRSDNHQA (SEQ ID NO: 341). In some embodiments, the CM comprises the amino acid sequence GPLSGRSDNHQK (SEQ ID NO: 342). In some embodiments, the CM comprises the amino acid sequence GPLSGRSDNHQR (SEQ ID NO: 343). In some embodiments, the CM comprises the amino acid sequence GPLSGRSDNHRA (SEQ ID NO: 344). In some embodiments, the CM comprises the amino acid sequence GPLSGRSDNHKK (SEQ ID NO: 345). In some embodiments, the CM comprises the amino acid sequence GPLSGRSDNHKR (SEQ ID NO: 346). In some embodiments, the CM comprises the amino acid sequence RPLSGRSDNHKA (SEQ ID NO: 347). In some embodiments, the CM comprises the amino acid sequence RPLSGRSDNHKK (SEQ ID NO: 348). In some embodiments, the CM comprises the amino acid sequence RPLSGRSDNHKR (SEQ ID NO: 349). In some embodiments, the CM comprises the amino acid sequence RPLSGRSDNHQA (SEQ ID NO: 350). In some embodiments, the CM comprises the amino acid sequence RPLSGRSDNHQK (SEQ ID NO: 351). In some embodiments, the CM comprises the amino acid sequence RPLSGRSDNHQR (SEQ ID NO: 352). In some embodiments, the CM comprises the amino acid sequence RPLSGRSDNHRA (SEQ ID NO: 353). In some embodiments, the CM comprises the amino acid sequence RPLSGRSDNHQK (SEQ ID NO: 354). In some embodiments, the CM comprises the amino acid sequence RPLSGRSDNHRR (SEQ ID NO: 355). In some embodiments, the CM comprises the amino acid sequence KGLTGRSDRHQA (SEQ ID NO: 356).

In some embodiments, the CM comprises a core CM consensus 13 sequence comprising the amino acid sequence RIGRSDNH (SEQ ID NO: 357) or RLGRSDNN (SEQ ID NO: 358). In some embodiments, the CM comprises an expanded core CM consensus 13 sequence comprising the amino acid sequence NHRIGRSDNHRR (SEQ ID NO: 359) or TLRLGRSDNNKN (SEQ ID NO: 360).

In some embodiments, the CM comprises the amino acid sequence RIGRSDNH (SEQ ID NO: 357). In some embodiments, the CM comprises the amino acid sequence RLGRSDNN (SEQ ID NO: 358). In some embodiments, the CM comprises the amino acid sequence NHRIGRSDNHRR (SEQ ID NO: 359). In some embodiments, the CM comprises the amino acid sequence TLRLGRSDNNKN (SEQ ID NO: 360).

In some embodiments, the CM comprises a core CM consensus 14 sequence comprising an amino acid sequence selected from the group consisting of TSGRSANP (SEQ ID NO: 361), TSGRSGNP (SEQ ID NO: 362), LSGRSANP (SEQ ID NO: 363), and LSGRSGNP (SEQ ID NO: 364). In some embodiments, the CM comprises an expanded core CM consensus 14 sequence comprising an amino acid sequence selected from the group consisting of TSTSGRSANPRG (SEQ ID NO: 365), TSTSGRSGNPRG (SEQ ID NO: 366), TSLSGRSANPRG (SEQ ID NO: 367), and TSLSGRSGNPRG (SEQ ID NO: 368).

In some embodiments, the CM comprises the amino acid sequence TSGRSANP (SEQ ID NO: 361). In some embodiments, the CM comprises the amino acid sequence TSGRSGNP (SEQ ID NO: 362). In some embodiments, the CM comprises the amino acid sequence LSGRSANP (SEQ ID NO: 363). In some embodiments, the CM comprises the amino acid sequence LSGRSGNP (SEQ ID NO: 364). In some embodiments. In some embodiments, the CM comprises the amino acid sequence the CM comprises the amino acid sequence TSTSGRSANPRG (SEQ ID NO: 365). In some embodiments, the CM comprises the amino acid sequence TSTSGRSGNPRG (SEQ ID NO: 366). In some embodiments, the CM comprises the amino acid sequence TSLSGRSANPRG (SEQ ID NO: 367). In some embodiments, the CM comprises the amino acid sequence and TSLSGRSGNPRG (SEQ ID NO: 368).

In some embodiments, the CM comprises an amino acid sequence selected from the group consisting of LSGRSENH (SEQ ID NO: 369), SIARSDNL (SEQ ID NO: 370), LSGRSVTQ (SEQ ID NO: 371), LSGRSGNH (SEQ ID NO: 308), LTGRSDRH (SEQ ID NO: 314), LYGRSENN (SEQ ID NO: 374), RLGRSDNN (SEQ ID NO: 375), TSGRSANP (SEQ ID NO: 376), NTLSGRSENHSG (SEQ ID NO: 377), PPSIARSDNLAN (SEQ ID NO: 378), TGLSGRSVTQTS (SEQ ID NO: 379), NTLSGRSGNHGS (SEQ ID NO: 311), KGLTGRSDRHQA (SEQ ID NO: 381), KNLYGRSENNGN (SEQ ID NO: 382), TLRLGRSDNNKN (SEQ ID NO: 383), and TSTSGRSANPRG (SEQ ID NO: 384).

In some embodiments, the CM comprises the amino acid sequence LSGRSENH (SEQ ID NO: 369). In some embodiments, the CM comprises the amino acid sequence SIARSDNL (SEQ ID NO: 370). In some embodiments, the CM comprises the amino acid sequence LSGRSVTQ (SEQ ID NO: 371). In some embodiments, the CM comprises the amino acid sequence LSGRSGNH (SEQ ID NO: 308). In some embodiments, the CM comprises the amino acid sequence LTGRSDRH (SEQ ID NO: 314). In some embodiments, the CM comprises the amino acid sequence LYGRSENN (SEQ ID NO: 374). In some embodiments, the CM comprises the amino acid sequence RLGRSDNN (SEQ ID NO: 375). In some embodiments, the CM comprises the amino acid sequence TSGRSANP (SEQ ID NO: 376). In some embodiments, the CM comprises the amino acid sequence NTLSGRSENHSG (SEQ ID NO: 377). In some embodiments, the CM comprises the amino acid sequence PPSIARSDNLAN (SEQ ID NO: 378). In some embodiments, the CM comprises the amino acid sequence TGLSGRSVTQTS (SEQ ID NO: 379). In some embodiments, the CM comprises the amino acid sequence NTLSGRSGNHGS (SEQ ID NO: 311). In some embodiments, the CM comprises the amino acid sequence KGLTGRSDRHQA (SEQ ID NO: 381). In some embodiments, the CM comprises the amino acid sequence KNLYGRSENNGN (SEQ ID NO: 382). In some embodiments, the CM comprises the amino acid sequence TLRLGRSDNNKN (SEQ ID NO: 383). In some embodiments, the CM comprises the amino acid sequence TSTSGRSANPRG (SEQ ID NO: 384).

In some embodiments, the CM is a substrate for at least two proteases. In some embodiments, at least one protease is matriptase or uPA and at least one protease is selected from the group consisting of those shown in Table 7.

TABLE 7
Exemplary Proteases and/or Enzymes

ADAMS, ADAMTS, e.g.
ADAM8
ADAM9
ADAM10
ADAM12
ADAM15
ADAM17/TACE
ADAMDEC1
ADAMTS1
ADAMTS4
ADAMTS5
Aspartate proteases, e.g.,
BACE
Renin
Aspartic cathepsins, e.g.,
Cathepsin D
Cathepsin E
Caspases, e.g.,
Caspase 1
Caspase 2
Caspase 3
Caspase 4
Caspase 5
Caspase 6
Caspase 7
Caspase 8
Caspase 9
Caspase 10
Caspase 14
Cysteine cathepsins, e.g.,
Cathepsin B
Cathepsin C
Cathepsin K
Cathepsin L
Cathepsin S
Cathepsin V/L2
Cathepsin X/Z/P
Cysteine proteinases, e.g.,
Cruzipain
Legumain
Otubain-2
KLKs, e.g.,
KLK4
KLK5
KLK6
KLK7
KLK8
KLK10
KLK11
KLK13
KLK14
Metallo proteinases, e.g.,
Meprin
Neprilysin
PSMA
BMP-1
MMPs, e.g.,
MMP1
MMP2
MMP3
MMP7
MMP8
MMP9
MMP10
MMP11
MMP12
MMP13
MMP14
MMP15
MMP16
MMP17
MMP19
MMP20
MMP23
MMP24
MMP26
MMP27
Serine proteases, e.g.,
activated protein C
Cathepsin A
Cathepsin G
Chymase
coagulation factor proteases
(e.g., FVIIa, FIXa, FXa, FXIa,
FXIIa)
Elastase
Granzyme B
Guanidinobenzoatase
HtrA1
Human Neutrophil Elastase
Lactoferrin
Marapsin
NS3/4A
PACE4
Plasmin
PSA
tPA
Thrombin
Tryptase
uPA
Type II Transmembrane
Serine Proteases (TTSPs), e.g.,
DESC1
DPP-4
FAP
Hepsin
Matriptase-2
MT-SP1/Matriptase
TMPRSS2
TMPRSS3
TMPRSS4

In some embodiments, the antibody is attached to at least a first CM and a second CM. In some embodiments, the first CM and the second CM are each polypeptides of no more than 15 amino acids long. In some embodiments, the first CM and the second CM in the antibody in the uncleaved state have the structural arrangement from N-terminus to C-terminus as follows: Agent-CM1-CM2-(Antibody or Antigen-Binding Fragment), (Antibody or Antigen-Binding Fragment)-CM2-CM1-Agent, Agent-CM2-CM1-(Antibody or Antigen-Binding Fragment), or (Antibody or Antigen-Binding Fragment)-CM1-CM2-Agent. In some embodiments, the antibody includes a linking peptide between the agent and CM1. In some embodiments, the antibody includes a linking peptide between CM1 and CM2. In some embodiments, the antibody includes a linking peptide between CM2 and antibody or antigen-binding fragment. In some embodiments, the antibody includes a linking peptide between the agent and CM1 and a linking peptide between CM2 and antibody or antigen-binding fragment. In some embodiments, the antibody includes a linking peptide between agent and CM1 and a linking peptide between CM1 and CM2. In some embodiments, the antibody includes a linking peptide between CM1 and CM2 and a linking peptide between CM2 and antibody or antigen-binding fragment. In some embodiments, the antibody includes a linking peptide between agent and CM1, a linking peptide between CM1 and CM2, and a linking peptide between CM2 and antibody or antigen-binding fragment.

In some embodiments, the antibody includes at least a first CM that includes a substrate for at least one protease selected from matriptase and uPA and a second CM that includes a substrate sequence. Exemplary substrates for the second CM (CM2) include but are not limited to substrates cleavable by one or more of the following enzymes or proteases listed in Table 7.

In some embodiments, the CM2 is selected for use with a specific protease. In some embodiments, the CM2 is a substrate for at least one protease selected from the group consisting of a matrix metalloprotease (MMP), a neutrophil elastase, uPA (also referred to as u-plasminogen activator), legumain, matriptase, thrombin, a cysteine protease such as a cathepsin, ADAM17, BMP-1, HtrA1, and a TMPRSS such as TMPRSS3 or TMPRSS4.

In some embodiments, the CM2 is a substrate for a neutrophil elastase. In some embodiments, the CM2 is a substrate for uPA. In some embodiments, the CM2 is a substrate for legumain. In some embodiments, the CM2 is a substrate for matriptase. In some embodiments, the CM2 is a substrate for thrombin. In some embodiments, the CM2 is a substrate for a cysteine protease. In some embodiments, the CM2 is a substrate for a cathepsin. In some embodiments, the CM2 is a substrate for ADAM17. In some embodiments, the CM2 is a substrate for BMP-1. In some embodiments, the CM2 is a substrate for HtrA1. In some embodiments, the CM2 is a substrate for a TMPRSS. In some embodiments, the CM2 is a substrate for TMPRSS3. In some embodiments, the CM2 is a substrate for TMPRSS4.

For example, suitable CM2 are cleaved by at least one protease and include the sequence TGRGPSWV (SEQ ID NO: 402); SARGPSRW (SEQ ID NO: 403); TARGPSFK (SEQ ID NO: 404); TARGPSW (SEQ ID NO: 405); LSGRSDNH (SEQ ID NO: 406); GGWHTGRN (SEQ ID NO: 407); HTGRSGAL (SEQ ID NO: 408); PLTGRSGG (SEQ ID NO: 409); AARGPAIH (SEQ ID NO: 411); RGPAFNPM (SEQ ID NO: 412); SSRGPAYL (SEQ ID NO: 413); RGPATPIM (SEQ ID NO: 414); RGPA (SEQ ID NO: 415); GGQPSGMWGW (SEQ ID NO: 416); FPRPLGITGL (SEQ ID NO: 417); VHMPLGFLGP (SEQ ID NO: 418); SPLTGRSG (SEQ ID NO: 419); SAGFSLPA (SEQ ID NO: 126); LAPLGLQRR (SEQ ID NO: 420); SGGPLGVR (SEQ ID NO: 421); PLGL (SEQ ID NO: 422); GPRSFGL (SEQ ID NO: 423) and/or GPRSFG (SEQ ID NO: 424).

In some embodiments, the CM2 comprises the amino acid sequence TGRGPSWV (SEQ ID NO: 402). In some embodiments, the CM2 comprises the amino acid sequence SARGPSRW (SEQ ID NO: 403). In some embodiments, the CM2 comprises the amino acid sequence TARGPSFK (SEQ ID NO: 404). In some embodiments, the CM2 comprises the amino acid sequence TARGPSW (SEQ ID NO: 405). In some embodiments, the CM2 comprises the amino acid sequence LSGRSDNH (SEQ ID NO: 406). In some embodiments, the CM2 comprises the amino acid sequence GGWHTGRN (SEQ ID NO: 407). In some embodiments, the CM2 comprises the amino acid sequence HTGRSGAL (SEQ ID NO: 408). In some embodiments, the CM2 comprises the amino acid sequence PLTGRSGG (SEQ ID NO: 409). In some embodiments, the CM2 comprises the amino acid sequence AARGPAIH (SEQ ID NO: 411). In some embodiments, the CM2 comprises the amino acid sequence RGPAFNPM (SEQ ID NO: 412). In some embodiments, the CM2 comprises the amino acid sequence SSRGPAYL (SEQ ID NO: 413). In some embodiments, the CM2 comprises the amino acid sequence RGPATPIM (SEQ ID NO: 414). In some embodiments, the CM2 comprises the amino acid sequence RGPA (SEQ ID NO: 415). In some embodiments, the CM2 comprises the amino acid sequence GGQPSGMWGW (SEQ ID NO: 416). In some embodiments, the CM2 comprises the amino acid sequence FPRPLGITGL (SEQ ID NO: 417). In some embodiments, the CM2 comprises the amino acid sequence VHMPLGFLGP (SEQ ID NO: 418). In some embodiments, the CM2 comprises the amino acid sequence SPLTGRSG (SEQ ID NO: 419). In some embodiments, the CM2 comprises the amino acid sequence LAPLGLQRR (SEQ ID NO: 420). In some embodiments, the CM2 comprises the amino acid sequence SGGPLGVR (SEQ ID NO: 421). In some embodiments, the CM2 comprises the amino acid sequence PLGL (SEQ ID NO: 422). In some embodiments, the CM2 comprises the amino acid sequence GPRSFGL (SEQ ID NO: 423). In some embodiments, the CM2 comprises the amino acid sequence GPRSFG (SEQ ID NO: 424).

In some embodiments, the CM2 is a substrate for at least one MMP. In some embodiments, the CM2 is a substrate for at least one MMP listed in the Table 7. In some embodiments, the CM2 is a substrate for MMP9. In some embodiments, the CM2 is a substrate for MMP14. In some embodiments, the CM2 is a substrate for two or more MMPs. In some embodiments, the CM2 is a substrate for at least MMP9 or MMP14. In some embodiments, the CM2 is a substrate for two or more MMPs. In some embodiments, the CM2 is a substrate for at least MMP9 and MMP14.

In some embodiments, CM2 is a substrate for an MMP and includes the sequence

```
(SEQ ID NO: 425)
ISSGLLSS;
(SEQ ID NO: 426)
QNQALRMA;
(SEQ ID NO: 427)
AQNLLGMV;
(SEQ ID NO: 428)
STFPFGMF;
(SEQ ID NO: 429)
PVGYTSSL;
(SEQ ID NO: 430)
DWLYWPGI;
(SEQ ID NO: 431)
MIAPVAYR;
(SEQ ID NO: 432)
RPSPMWAY;
(SEQ ID NO: 433)
WATPRPMR;
(SEQ ID NO: 434)
FRLLDWQW;
  (SEQ ID NO: 435)
LKAAPRWA;
(SEQ ID NO: 436)
GPSHLVLT;
(SEQ ID NO: 437)
LPGGLSPW;
      (SEQ ID NO: 438)
MGLFSEAG;
(SEQ ID NO: 439)
SPLPLRVP;
(SEQ ID NO: 440)
RMHLRSLG;
(SEQ ID NO: 441)
LAAPLGLL;
(SEQ ID NO: 442)
AVGLLAPP;
(SEQ ID NO: 443)
LLAPSHRA,
          (SEQ ID NO: 444)
PAGLWLDP; and/or
(SEQ ID NO: 445)
ISSGLSS.
```

In some embodiments, the first cleaving agent and the second cleaving agent are the same protease, and the first CM and the second CM are different substrates for the enzyme. In some embodiments, the first cleaving agent and the second cleaving agent are different proteases. In some embodiments, the first cleaving agent and the second cleaving agent are co-localized in the target tissue. In some embodiments, the first CM and the second CM are cleaved by at least one cleaving agent in the target tissue.

In some embodiments, the CM comprises the non-prime side of the protease cleavage site; that is, the CM comprises at least the P1 and P2 amino acids, and in some embodiments comprises the P1, P2 and P3 amino acids and in some embodiments comprises the P1, P2, P3, and P4 amino acids. In some embodiments, the CM comprises the non-prime side and the prime side of the protease cleavage site. In some embodiments, the CM comprises the non-prime side but lacks at least part of the prime side of the protease cleavage site. In some embodiments, the CM comprises the non-prime side but lacks the prime side of the protease cleavage site. Such a CM can be linked directly or through a linker to an antibody or other molecule as disclosed herein, such as, but not limited to, a detection moiety.

In some embodiments, the agent conjugated to the antibody or antigen-binding fragment is a therapeutic agent. In some embodiments, the agent is an antineoplastic agent. In some embodiments, the agent is a toxin or fragment thereof. As used herein, a fragment of a toxin is a fragment that retains toxic activity. In some embodiments, the agent is conjugated to the AB via a cleavable linker. In some embodiments, the agent is conjugated to the AB via a linker that includes at least one cleavable substrate sequence described herein. In some embodiments, the agent is conjugated to the AB via a noncleavable linker. In some embodiments, the agent is a microtubule inhibitor. In some embodiments, the agent is a nucleic acid damaging agent, such as a DNA alkylator or DNA intercalator, or other DNA damaging agent. In some embodiments, the agent is an agent selected from the group listed in Table 3. In some embodiments, the agent is a dolastatin. In some embodiments, the agent is an auristatin or derivative thereof. In some embodiments, the agent is auristatin E or a derivative thereof. In some embodiments, the agent is monomethyl auristatin E (MMAE). In some embodiments, the agent is monomethyl auristatin D (MMAD). In some embodiments, the agent is a maytansinoid or maytansinoid derivative. In some embodiments, the agent is DM1 or DM4. In some embodiments, the agent is a duocarmycin or derivative thereof. In some embodiments, the agent is a calicheamicin or derivative thereof. In some embodiments, the agent is a pyrrolobenzodiazepine.

In some embodiments, the agent is an anti-inflammatory agent.

In some embodiments, the antibody also includes a detectable moiety. In some embodiments, the detectable moiety is a diagnostic agent.

In some embodiments, the conjugated antibody and/or conjugated activatable antibody includes a detectable label. In some embodiments, the detectable label includes an imaging agent, a contrasting agent, an enzyme, a fluorescent label, a chromophore, a dye, one or more metal ions, or a ligand-based label. In some embodiments, the imaging agent comprises a radioisotope. In some embodiments, the radioisotope is indium or technetium. In some embodiments, the contrasting agent comprises iodine, gadolinium or iron oxide. In some embodiments, the enzyme comprises horseradish peroxidase, alkaline phosphatase, or β-galactosidase. In some embodiments, the fluorescent label comprises yellow fluorescent protein (YFP), cyan fluorescent protein (CFP), green fluorescent protein (GFP), modified red fluorescent protein (mRFP), red fluorescent protein tdimer2 (RFP tdimer2), HCRED, or a europium derivative. In some embodiments, the luminescent label comprises an N-methylacrydium derivative. In some embodiments, the label comprises an Alexa Fluor® label, such as Alex Fluor® 680 or Alexa Fluor® 750. In some embodiments, the ligand-based label comprises biotin, avidin, streptavidin or one or more haptens.

In some embodiments, the antibody naturally contains one or more disulfide bonds. In some embodiments, the antibody or antigen-binding fragment can be engineered to include one or more disulfide bonds.

In some embodiments, the antibody and/or conjugated antibody is monospecific. In some embodiments, the antibody and/or conjugated antibody is multispecific, e.g., by way of non-limiting example, bispecific or trifunctional. In some embodiments, the antibody and/or conjugated antibody is formulated as part of a pro-Bispecific T Cell Engager (pro-BITE) molecule. In some embodiments, the antibody and/or conjugated antibody is formulated as part of a pro-Chimeric Antigen Receptor (pro-CAR) modified T cell or other engineered receptor.

In some embodiments, the activatable antibody and/or conjugated activatable antibody is monospecific. In some embodiments, the activatable antibody and/or conjugated activatable antibody is multispecific, referred to herein as multispecific activatable antibodies and/or conjugated multispecific activatable antibodies. As used herein, terms such as “activatable antibody” and all grammatical variations thereof, unless otherwise noted, are intended to encompass, but are not limited to embodiments where the activatable antibody is a multispecific activatable antibody of the disclosure. As used herein, terms such as “conjugated activatable antibody” and all grammatical variations thereof, unless otherwise noted, are intended to encompass, but are not limited to embodiments where the conjugated activatable antibody is a conjugated multispecific activatable antibody of the disclosure. In some embodiments, the multispecific activatable antibody and/or conjugated multispecific activatable antibody is bispecific or trifunctional.

In some embodiments, the conjugated antibodies described herein are used in conjunction with one or more additional agents or a combination of additional agents. Suitable additional agents include current pharmaceutical and/or surgical therapies for an intended application, such as, for example, cancer. For example, the conjugated antibodies can be used in conjunction with an additional chemotherapeutic or anti-neoplastic agent.

The matriptase and/or uPA substrates of the disclosure are also useful in activatable antibodies. The activatable antibodies described herein in an activated state bind a given target and include (i) an antibody or an antigen binding fragment thereof (AB) that specifically binds to the target; (ii) a masking moiety (MM) that inhibits the binding of the AB to the target in an uncleaved state; and (c) a cleavable moiety (CM) coupled to the AB, wherein the CM is a polypeptide that functions as a substrate for at least one protease selected from matriptase and/or uPA.

In some embodiments, the activatable antibody in the uncleaved state has the structural arrangement from N-terminus to C-terminus as follows: MM-CM-AB or AB-CM-MM.

In some embodiments, the activatable antibody comprises a linking peptide between the MM and the CM.

In some embodiments, the activatable antibody comprises a linking peptide between the CM and the AB.

In some embodiments, the activatable antibody comprises a first linking peptide (LP1) and a second linking peptide (LP2), wherein the activatable antibody in the uncleaved state has the structural arrangement from N-terminus to C-terminus as follows: MM-LP1-CM-LP2-AB or AB-LP2-CM-LP1-MM.

In some embodiments, the two linking peptides need not be identical to each other.

In some embodiments, at least one of LP1 or LP2 comprises an amino acid sequence selected from the group consisting of (GS)ₙ, (GGS)ₙ, (GSGGS)ₙ (SEQ ID NO: 385) and (GGGS)ₙ (SEQ ID NO: 386), where n is an integer of at least one.

In some embodiments, at least one of LP1 or LP2 comprises an amino acid sequence selected from the group consisting of GGSG (SEQ ID NO: 387), GGSGG (SEQ ID NO: 388), GSGSG (SEQ ID NO: 389), GSGGG (SEQ ID NO: 390), GGGSG (SEQ ID NO: 391), and GSSSG (SEQ ID NO: 392).

In some embodiments, LP1 comprises the amino acid sequence

(SEQ ID NO: 393)
GSSGGSGGSGGSG,
(SEQ ID NO: 394)
GSSGGSGGSGG,
(SEQ ID NO: 395)
GSSGGSGGSGGS,
(SEQ ID NO: 396)
GSSGGSGGSGGSGGGS,
(SEQ ID NO: 397)
GSSGGSGGSG, or
(SEQ ID NO: 398)
GSSGGSGGSGS.

In some embodiments, LP2 comprises the amino acid sequence GSS, GGS, GGGS (SEQ ID NO: 399), GSSGT (SEQ ID NO: 400) or GSSG (SEQ ID NO: 401).

In some embodiments, the AB has an equilibrium dissociation constant of about 100 nM or less for binding to the target.

In some embodiments, the activatable antibody includes an antibody or antigen-binding fragment thereof that specifically binds a target. In some embodiments, the antibody or immunologically active fragment thereof that binds the target is a monoclonal antibody, domain antibody, single chain, Fab fragment, a F(ab′)₂ fragment, a scFv, a scab, a dAb, a single domain heavy chain antibody, or a single domain light chain antibody. In some embodiments, such an antibody or immunologically active fragment thereof that binds the target is a mouse, other rodent, chimeric, humanized or fully human monoclonal antibody.

In some embodiments, the MM has an equilibrium dissociation constant for binding to the AB that is greater than the equilibrium dissociation constant of the AB to the target.

In some embodiments, the MM has an equilibrium dissociation constant for binding to the AB that is no more than the equilibrium dissociation constant of the AB to the target.

In some embodiments, the MM does not interfere or compete with the AB for binding to the target in a cleaved state.

In some embodiments, the MM is a polypeptide of about 2 to 40 amino acids in length. For example, the MM is a polypeptide of up to about 40 amino acids in length.

In some embodiments, the MM polypeptide sequence is different from that of any natural binding partner of the AB. In some embodiments, the MM polypeptide sequence is no more than 50% identical to any natural binding partner of the AB. In some embodiments, the MM polypeptide sequence is no more than 40%, 30%, 25%, 20%, 15%, or 10% identical to any natural binding partner of the AB.

In some embodiments, the coupling of the MM to the AB reduces the ability of the AB to bind its target such that the dissociation constant (K_{d}) of the AB when coupled to the MM towards the target is at least two times greater than the K_{d} of the AB when not coupled to the MM towards the target.

In some embodiments, the coupling of the MM to the AB reduces the ability of the AB to bind its target such that the dissociation constant (K_{d}) of the AB when coupled to the MM towards the target is at least three times greater than the K_{d} of the AB when not coupled to the MM towards the target.

In some embodiments, the coupling of the MM to the AB reduces the ability of the AB to bind its target such that the dissociation constant (K_{d}) of the AB when coupled to the MM towards the target is at least five times greater than the K_{d} of the AB when not coupled to the MM towards the target.

In some embodiments, the coupling of the MM to the AB reduces the ability of the AB to bind its target such that the dissociation constant (K_{d}) of the AB when coupled to the MM towards the target is at least 10 times greater than the K_{d} of the AB when not coupled to the MM towards the target.

In some embodiments, the coupling of the MM to the AB reduces the ability of the AB to bind its target such that the dissociation constant (K_{d}) of the AB when coupled to the MM towards the target is at least 20 times greater than the K_{d} of the AB when not coupled to the MM towards the target.

In some embodiments, the coupling of the MM to the AB reduces the ability of the AB to bind the target such that the dissociation constant (K_{d}) of the AB when coupled to the MM towards the target is at least 40 times greater than the K_{d} of the AB when not coupled to the MM towards the target.

In some embodiments, the coupling of the MM to the AB reduces the ability of the AB to bind the target such that the dissociation constant (K_{d}) of the AB when coupled to the MM towards the target is at least 100 times greater than the K_{d} of the AB when not coupled to the MM towards the target.

In some embodiments, the coupling of the MM to the AB reduces the ability of the AB to bind the target such that the dissociation constant (K_{d}) of the AB when coupled to the MM towards the target is at least 1000 times greater than the K_{d} of the AB when not coupled to the MM towards the target.

In some embodiments, the coupling of the MM to the AB reduces the ability of the AB to bind the target such that the dissociation constant (K_{d}) of the AB when coupled to the MM towards the target is at least 10,000 times greater than the K_{d} of the AB when not coupled to the MM towards the target.

In some embodiments, the protease, i.e., matriptase and/or uPA, is co-localized with the target in a tissue, and the protease cleaves the CM in the activatable antibody when the activatable antibody is exposed to the protease.

In some embodiments, in the presence of the target, the MM reduces the ability of the AB to bind the target by at least 90% when the CM is uncleaved, as compared to when the CM is cleaved when assayed in vitro using a target displacement assay such as, for example, the assay described in PCT Publication Nos. WO 2009/025846 and WO 2010/081173.

In some embodiments, the CM is positioned in the activatable antibody such that in the uncleaved state, binding of the activatable antibody to the target is reduced to occur with an equilibrium dissociation constant that is at least twofold greater than the equilibrium dissociation constant of an unmodified AB binding to the target, whereas in the cleaved state (i.e., when the activatable antibody is in the cleaved state), the AB binds the target.

In some embodiments, the CM is positioned in the activatable antibody such that in the uncleaved state, binding of the activatable antibody to the target is reduced to occur with an equilibrium dissociation constant that is at least five-fold greater than the equilibrium dissociation constant of an unmodified AB binding to the target, whereas in the cleaved state (i.e., when the activatable antibody is in the cleaved state), the AB binds the target.

In some embodiments, the CM is positioned in the activatable antibody such that in the uncleaved state, binding of the activatable antibody to the target is reduced to occur with an equilibrium dissociation constant that is at least ten-fold greater than the equilibrium dissociation constant of an unmodified AB binding to the target, whereas in the cleaved state (i.e., when the activatable antibody is in the cleaved state), the AB binds the target.

In some embodiments, the CM is positioned in the activatable antibody such that in the uncleaved state, binding of the activatable antibody to the target is reduced to occur with an equilibrium dissociation constant that is at least 20-fold greater than the equilibrium dissociation constant of an unmodified AB binding to the target, whereas in the cleaved state (i.e., when the activatable antibody is in the cleaved state), the AB binds the target.

In some embodiments, the CM is positioned in the activatable antibody such that in the uncleaved state, binding of the activatable antibody to the target is reduced to occur with an equilibrium dissociation constant that is at least 40-fold greater than the equilibrium dissociation constant of an unmodified AB binding to the target, whereas in the cleaved state, the AB binds the target.

In some embodiments, the CM is positioned in the activatable antibody such that in the uncleaved state, binding of the activatable antibody to the target is reduced to occur with an equilibrium dissociation constant that is at least 50-fold greater than the equilibrium dissociation constant of an unmodified AB binding to the target, whereas in the cleaved state, the AB binds the target.

In some embodiments, the CM is positioned in the activatable antibody such that in the uncleaved state, binding of the activatable antibody to the target is reduced to occur with an equilibrium dissociation constant that is at least 100-fold greater than the equilibrium dissociation constant of an unmodified AB binding to the target, whereas in the cleaved state, the AB binds the target.

In some embodiments, the CM is positioned in the activatable antibody such that in the uncleaved state, binding of the activatable antibody to the target is reduced to occur with an equilibrium dissociation constant that is at least 200-fold greater than the equilibrium dissociation constant of an unmodified AB binding to the target, whereas in the cleaved state, the AB binds the target.

In some embodiments, the CM is a polypeptide of up to 15 amino acids in length.

In some embodiments, the CM is a substrate for at least matriptase. In some embodiments, the CM is a substrate for at least uPA. In some embodiments, the CM is a substrate for at least matriptase and uPA.

In some embodiments, the CM is a substrate for matriptase and/or uPA, and is resistant to cleavage by at least one other protease. In some embodiments, the CM is a substrate for matriptase and/or uPA, and is resistant to cleavage by at least plasmin. In some embodiments, the CM is a substrate for matriptase and/or uPA, and is resistant to cleavage by at least tissue plasminogen activator (tPA).

In some embodiments, the CM is a substrate for matriptase and/or uPA and includes a motif sequence that is recognized by matriptase and/or uPA, provided that for any given motif sequence of the disclosure:
(i) the CM does not comprise any of the following amino acid sequences TGRGPSWV (SEQ ID NO: 402), SARGPSRW (SEQ ID NO: 403), or TARGPSFK (SEQ ID NO: 404); and the CM does not comprise a consensus amino acid sequence based on these amino acid sequences, such as for example, TARGPSW (SEQ ID NO: 405);
(ii) the CM does not comprise any of the following amino acid sequences LSGRSDNH (SEQ ID NO: 406), GGWHTGRN (SEQ ID NO: 407), HTGRSGAL (SEQ ID NO: 408), or PLTGRSGG (SEQ ID NO: 409); and the CM does not comprise a consensus amino acid sequence based on these amino acid sequences, such as for example, LTGRSGA (SEQ ID NO: 410); and/or
(iii) the CM does not comprise any of the following amino acid sequences AARGPAIH (SEQ ID NO: 411), RGPAFNPM (SEQ ID NO: 412), SSRGPAYL (SEQ ID NO: 413), or RGPATPIM (SEQ ID NO: 414); and the CM does not comprise a consensus amino acid sequence based on these amino acid sequences, such as for example, RGPA (SEQ ID NO: 415).

In some embodiments, the motif sequence includes a core CM consensus sequence shown in Tables 8A-8J. In some embodiments, the motif sequence includes a subgenus, i.e., a subset, of the core CM consensus sequence shown in Tables 8A-8J.

In some embodiments, the motif sequence includes an expanded consensus sequence based on one of the core CM consensus sequence shown in Tables 8A-8J. In some embodiments, the expanded consensus sequence is a consensus sequence shown in Tables 9A-9J-3.

In some embodiments, the CM comprises a core CM consensus 1 sequence comprising the amino acid sequence AAPRS (SEQ ID NO: 163). In some embodiments, the CM comprises an expanded core CM consensus 1 sequence comprising the amino acid sequence AAPRSF (SEQ ID NO: 164).

In some embodiments, the CM comprises a core CM consensus 2 sequence comprising the amino acid sequence SRRVP (SEQ ID NO: 165). In some embodiments, the CM comprises an expanded core CM consensus 2 sequence comprising an amino acid sequence selected from the group consisting of QSRRVP (SEQ ID NO: 166), QTRRVP (SEQ ID NO: 167), SRRVPL (SEQ ID NO: 168), SRRVPV (SEQ ID NO: 169), QSRRVPL (SEQ ID NO: 170), QSRRVPV (SEQ ID NO: 171), QTRRVPL (SEQ ID NO: 172), and QTRRVPV (SEQ ID NO: 173).

In some embodiments, the CM comprises the amino acid sequence QSRRVP (SEQ ID NO: 166). In some embodiments, the CM comprises the amino acid sequence QTRRVP (SEQ ID NO: 167). In some embodiments, the CM comprises the amino acid sequence SRRVPL (SEQ ID NO: 168). In some embodiments, the CM comprises the amino acid sequence SRRVPV (SEQ ID NO: 169). In some embodiments, the CM comprises the amino acid sequence QSRRVPL (SEQ ID NO: 170). In some embodiments, the CM comprises the amino acid sequence QSRRVPV (SEQ ID NO: 171). In some embodiments, the CM comprises the amino acid sequence QTRRVPL (SEQ ID NO: 172). In some embodiments, the CM comprises the amino acid sequence QTRRVPV (SEQ ID NO: 173).

In some embodiments, the CM comprises a core CM consensus 3 sequence comprising the amino acid sequence PPLGR (SEQ ID NO: 174). In some embodiments, the CM comprises an expanded core CM consensus 3 sequence comprising an amino acid sequence selected from the group consisting of GPPLGR (SEQ ID NO: 175), SPPLGR (SEQ ID NO: 176), CGPPLGR (SEQ ID NO: 177), CSPPLGR (SEQ ID NO: 178), GGPPLGR (SEQ ID NO: 179), GSPPLGR (SEQ ID NO: 180), SGPPLGR (SEQ ID NO: 181), SSPPLGR (SEQ ID NO: 182), GCGPPLGR (SEQ ID NO: 183), GCSPPLGR (SEQ ID NO: 184), GGGPPLGR (SEQ ID NO: 185), GGSPPLGR (SEQ ID NO: 186), GSGPPLGR (SEQ ID NO: 187), GSSPPLGR (SEQ ID NO: 188), SCGPPLGR (SEQ ID NO: 189), SCSPPLGR (SEQ ID NO: 190), SGGPPLGR (SEQ ID NO: 191), SGSPPLGR (SEQ ID NO: 192), SSGPPLGR (SEQ ID NO: 193), and SSSPPLGR (SEQ ID NO: 194).

In some embodiments, the CM comprises the amino acid sequence GPPLGR (SEQ ID NO: 175). In some embodiments, the CM comprises the amino acid sequence SPPLGR (SEQ ID NO: 176). In some embodiments, the CM comprises the amino acid sequence CGPPLGR (SEQ ID NO: 177). In some embodiments, the CM comprises the amino acid sequence CSPPLGR (SEQ ID NO: 178). In some embodiments, the CM comprises the amino acid sequence GGPPLGR (SEQ ID NO: 179). In some embodiments, the CM comprises the amino acid sequence GSPPLGR (SEQ ID NO: 180). In some embodiments, the CM comprises the amino acid sequence SGPPLGR (SEQ ID NO: 181). In some embodiments, the CM comprises the amino acid sequence SSPPLGR (SEQ ID NO: 182). In some embodiments, the CM comprises the amino acid sequence GCGPPLGR (SEQ ID NO: 183). In some embodiments, the CM comprises the amino acid sequence GCSPPLGR (SEQ ID NO: 184). In some embodiments, the CM comprises the amino acid sequence GGGPPLGR (SEQ ID NO: 185). In some embodiments, the CM comprises the amino acid sequence GGSPPLGR (SEQ ID NO: 186). In some embodiments, the CM comprises the amino acid sequence GSGPPLGR (SEQ ID NO: 187). In some embodiments, the CM comprises the amino acid sequence GSSPPLGR (SEQ ID NO: 188). In some embodiments, the CM comprises the amino acid sequence SCGPPLGR (SEQ ID NO: 189). In some embodiments, the CM comprises the amino acid sequence SCSPPLGR (SEQ ID NO: 190). In some embodiments, the CM comprises the amino acid sequence SGGPPLGR (SEQ ID NO: 191). In some embodiments, the CM comprises the amino acid sequence SGSPPLGR (SEQ ID NO: 192). In some embodiments, the CM comprises the amino acid sequence SSGPPLGR (SEQ ID NO: 193). In some embodiments, the CM comprises the amino acid sequence SSSPPLGR (SEQ ID NO: 194).

In some embodiments, the CM comprises a core CM consensus 4 sequence comprising the amino acid sequence LRSGW (SEQ ID NO: 195). In some embodiments, the CM comprises an expanded core CM consensus 4 sequence comprising an amino acid sequence selected from the group consisting of MLRSGW, (SEQ ID NO: 196), MLRSGWR, (SEQ ID NO: 197), MLRSGWRG, (SEQ ID NO: 198), MLRSGWRL, (SEQ ID NO: 199), and MLRSGWRS (SEQ ID NO: 200).

In some embodiments, the CM comprises the amino acid sequence MLRSGW, (SEQ ID NO: 196). In some embodiments, the CM comprises the amino acid sequence MLRSGWR (SEQ ID NO: 197). In some embodiments, the CM comprises the amino acid sequence MLRSGWRG (SEQ ID NO: 198). In some embodiments, the CM comprises the amino acid sequence MLRSGWRL (SEQ ID NO: 199). In some embodiments, the CM comprises the amino acid sequence MLRSGWRS (SEQ ID NO: 200).

In some embodiments, the CM comprises a core CM consensus 5 sequence comprising the amino acid sequence VSRSA (SEQ ID NO: 201). In some embodiments, the CM comprises an expanded core CM consensus 5 sequence comprising an amino acid sequence selected from the group consisting of IVSRSA (SEQ ID NO: 202), YIVSRSA (SEQ ID NO: 203), and QYIVSRSA (SEQ ID NO: 204).

In some embodiments, the CM comprises the amino acid sequence IVSRSA (SEQ ID NO: 202). In some embodiments, the CM comprises the amino acid sequence YIVSRSA (SEQ ID NO: 203). In some embodiments, the CM comprises the amino acid sequence QYIVSRSA (SEQ ID NO: 204).

In some embodiments, the CM comprises a core CM consensus 6 sequence comprising the amino acid sequence ALRAP (SEQ ID NO: 205). In some embodiments, the CM comprises an expanded core CM consensus 6 sequence comprising the amino acid sequence RALRAP (SEQ ID NO: 206).

In some embodiments, the CM comprises a core CM consensus 7 sequence comprising the amino acid sequence PAGRR (SEQ ID NO: 207). In some embodiments, the CM comprises an expanded core CM consensus 7 sequence comprising an amino acid sequence selected from the group consisting of PAGRRS (SEQ ID NO: 208), PAGRRSL (SEQ ID NO: 209), VPAGRRS (SEQ ID NO: 210), and VPAGRRSL (SEQ ID NO: 211).

In some embodiments, the CM comprises the amino acid sequence PAGRRS (SEQ ID NO: 208). In some embodiments, the CM comprises the amino acid sequence PAGRRSL (SEQ ID NO: 209). In some embodiments, the CM comprises the amino acid sequence VPAGRRS (SEQ ID NO: 210). In some embodiments, the CM comprises the amino acid sequence VPAGRRSL (SEQ ID NO: 211).

In some embodiments, the CM comprises a core CM consensus 8 sequence comprising the amino acid sequence GRSML (SEQ ID NO: 212). In some embodiments, the CM comprises an expanded core CM consensus 8 sequence comprising an amino acid sequence selected from the group consisting of GRSMLL (SEQ ID NO: 213), GRSMLM (SEQ ID NO: 214), GRSMLLG (SEQ ID NO: 215), GRSMLLP (SEQ ID NO: 216), GRSMLLS (SEQ ID NO: 217), GRSMLMG (SEQ ID NO: 218), GRSMLMP (SEQ ID NO: 219), GRSMLMS (SEQ ID NO: 220), GRSMLLGG (SEQ ID NO: 221), GRSMLLPG (SEQ ID NO: 222), GRSMLLSG (SEQ ID NO: 223), GRSMLMGG (SEQ ID NO: 224), GRSMLMPG (SEQ ID NO: 225), GRSMLMSG (SEQ ID NO: 226), GRSMLLGP (SEQ ID NO: 227), GRSMLLPP (SEQ ID NO: 228), GRSMLLSP (SEQ ID NO: 229), GRSMLMGP (SEQ ID NO: 230), GRSMLMPP (SEQ ID NO: 231), GRSMLMSP (SEQ ID NO: 232), GRSMLLGS (SEQ ID NO: 233), GRSMLLPS (SEQ ID NO: 234), GRSMLLSS (SEQ ID NO: 235), GRSMLMGS (SEQ ID NO: 236), GRSMLMPS (SEQ ID NO: 237), and GRSMLMSS (SEQ ID NO: 238).

In some embodiments, the CM comprises the amino acid sequence GRSMLL (SEQ ID NO: 213). In some embodiments, the CM comprises the amino acid sequence GRSMLM (SEQ ID NO: 214). In some embodiments, the CM comprises the amino acid sequence GRSMLLG (SEQ ID NO: 215). In some embodiments, the CM comprises the amino acid sequence GRSMLLP (SEQ ID NO: 216). In some embodiments, the CM comprises the amino acid sequence GRSMLLS (SEQ ID NO: 217). In some embodiments, the CM comprises the amino acid sequence GRSMLMG (SEQ ID NO: 218). In some embodiments, the CM comprises the amino acid sequence GRSMLMP (SEQ ID NO: 219). In some embodiments, the CM comprises the amino acid sequence GRSMLMS (SEQ ID NO: 220). In some embodiments, the CM comprises the amino acid sequence GRSMLLGG (SEQ ID NO: 221). In some embodiments, the CM comprises the amino acid sequence GRSMLLPG (SEQ ID NO: 222). In some embodiments, the CM comprises the amino acid sequence GRSMLLSG (SEQ ID NO: 223). In some embodiments, the CM comprises the amino acid sequence GRSMLMGG (SEQ ID NO: 224). In some embodiments, the CM comprises the amino acid sequence GRSMLMPG (SEQ ID NO: 225). In some embodiments, the CM comprises the amino acid sequence GRSMLMSG (SEQ ID NO: 226). In some embodiments, the CM comprises the amino acid sequence GRSMLLGP (SEQ ID NO: 227). In some embodiments, the CM comprises the amino acid sequence GRSMLLPP (SEQ ID NO: 228). In some embodiments, the CM comprises the amino acid sequence GRSMLLSP (SEQ ID NO: 229). In some embodiments, the CM comprises the amino acid sequence GRSMLMGP (SEQ ID NO: 230). In some embodiments, the CM comprises the amino acid sequence GRSMLMPP (SEQ ID NO: 231). In some embodiments, the CM comprises the amino acid sequence GRSMLMSP (SEQ ID NO: 232). In some embodiments, the CM comprises the amino acid sequence GRSMLLGS (SEQ ID NO: 233). In some embodiments, the CM comprises the amino acid sequence GRSMLLPS (SEQ ID NO: 234). In some embodiments, the CM comprises the amino acid sequence GRSMLLSS (SEQ ID NO: 235). In some embodiments, the CM comprises the amino acid sequence GRSMLMGS (SEQ ID NO: 236). In some embodiments, the CM comprises the amino acid sequence GRSMLMPS (SEQ ID NO: 237). In some embodiments, the CM comprises the amino acid sequence GRSMLMSS (SEQ ID NO: 238).

In some embodiments, the CM comprises a core CM consensus 9 sequence comprising the amino acid sequence LARAG (SEQ ID NO: 239). In some embodiments, the CM comprises an expanded core CM consensus 9 sequence comprising an amino acid sequence selected from the group consisting of LARAGI (SEQ ID NO: 240), LARAGL (SEQ ID NO: 241), PLARAGI (SEQ ID NO: 242), PLARAGL (SEQ ID NO: 243), RPLARAGI (SEQ ID NO: 244), and RPLARAGL (SEQ ID NO: 245).

In some embodiments, the CM comprises the amino acid sequence LARAGI (SEQ ID NO: 240). In some embodiments, the CM comprises the amino acid sequence LARAGL (SEQ ID NO: 241). In some embodiments, the CM comprises the amino acid sequence PLARAGI (SEQ ID NO: 242). In some embodiments, the CM comprises the amino acid sequence PLARAGL (SEQ ID NO: 243). In some embodiments, the CM comprises the amino acid sequence RPLARAGI (SEQ ID NO: 244). In some embodiments, the CM comprises the amino acid sequence RPLARAGL (SEQ ID NO: 245).

In some embodiments, the CM comprises a core CM consensus 10 sequence comprising the amino acid sequence ESRRW (SEQ ID NO: 246). In some embodiments, the CM comprises an expanded core CM consensus 10 sequence comprising an amino acid sequence selected from the group consisting of ESRRWM (SEQ ID NO: 247), ESRRWMP (SEQ ID NO: 248), and PESRRWMP (SEQ ID NO: 249).

In some embodiments, the CM comprises the amino acid sequence ESRRWM (SEQ ID NO: 247). In some embodiments, the CM comprises the amino acid sequence ESRRWMP (SEQ ID NO: 248). In some embodiments, the CM comprises the amino acid sequence PESRRWMP (SEQ ID NO: 249).

In some embodiments, the CM comprises an amino acid sequence selected from the group consisting of ILPRSPAF (SEQ ID NO: 250), VAGRSMRP (SEQ ID NO: 251), VVPEGRRS (SEQ ID NO: 252), QGRAITFI (SEQ ID NO: 253), VLSKQMSF (SEQ ID NO: 254), LKGRSYYY (SEQ ID NO: 255), KRMPVQFL (SEQ ID NO: 256), PQHRIVSF (SEQ ID NO: 257), YKKFVGSL (SEQ ID NO: 258), HMMQYARH (SEQ ID NO: 259), IPFSWSRF (SEQ ID NO: 260), LSQARWRK (SEQ ID NO: 261), DISHWRRS (SEQ ID NO: 262), RKTVQHWW (SEQ ID NO: 263), RFYRNQFF (SEQ ID NO: 264), RSLVFAPI (SEQ ID NO: 265), RSPSRLKC (SEQ ID NO: 266), and RKMPNITV (SEQ ID NO: 267).

In some embodiments, the CM comprises the amino acid sequence ILPRSPAF (SEQ ID NO: 250). In some embodiments, the CM comprises the amino acid sequence VAGRSMRP (SEQ ID NO: 251). In some embodiments, the CM comprises the amino acid sequence VVPEGRRS (SEQ ID NO: 252). In some embodiments, the CM comprises the amino acid sequence QGRAITFI (SEQ ID NO: 253). In some embodiments, the CM comprises the amino acid sequence VLSKQMSF (SEQ ID NO: 254). In some embodiments, the CM comprises the amino acid sequence LKGRSYYY (SEQ ID NO: 255). In some embodiments, the CM comprises the amino acid sequence KRMPVQFL (SEQ ID NO: 256). In some embodiments, the CM comprises the amino acid sequence PQHRIVSF (SEQ ID NO: 257). In some embodiments, the CM comprises the amino acid sequence YKKFVGSL (SEQ ID NO: 258). In some embodiments, the CM comprises the amino acid sequence HMMQYARH (SEQ ID NO: 259). In some embodiments, the CM comprises the amino acid sequence IPFSWSRF (SEQ ID NO: 260). In some embodiments, the CM comprises the amino acid sequence LSQARWRK (SEQ ID NO: 261). In some embodiments, the CM comprises the amino acid sequence DISHWRRS (SEQ ID NO: 262). In some embodiments, the CM comprises the amino acid sequence RKTVQHWW (SEQ ID NO: 263J. In some embodiments, the CM comprises the amino acid sequence RFYRNQFF (SEQ ID NO: 264). In some embodiments, the CM comprises the amino acid sequence RSLVFAPI (SEQ ID NO: 265). In some embodiments, the CM comprises the amino acid sequence RSPSRLKC (SEQ ID NO: 266). In some embodiments, the CM comprises the amino acid sequence RKMPNITV (SEQ ID NO: 267).

In some embodiments, the motif sequence includes a core CM consensus sequence shown in Tables 10A-10D. In some embodiments, the motif sequence includes a subgenus, i.e., a subset, of the core CM consensus sequence shown in Tables 10A-10D.

In some embodiments, the motif sequence includes an expanded consensus sequence based on one of the core CM consensus sequence shown in Tables 10A-10D. In some embodiments, the expanded consensus sequence is a consensus sequence shown in Tables 11A-11D.

In some embodiments, the CM comprises a core CM consensus 11 sequence comprising the amino acid sequence LSGRSANH (SEQ ID NO: 307) or LSGRSGNH (SEQ ID NO: 308). In some embodiments, the CM comprises an expanded core CM consensus 11 sequence comprising the amino acid sequence DRLSGRSANHKK (SEQ ID NO: 309), DRLSGRSDNHKK (SEQ ID NO: 310), or NTLSGRSGNHGS (SEQ ID NO: 311).

In some embodiments, the CM comprises the amino acid sequence LSGRSANH (SEQ ID NO: 307). In some embodiments, the CM comprises the amino acid sequence LSGRSGNH (SEQ ID NO: 308). In some embodiments, the CM comprises the amino acid sequence DRLSGRSANHKK (SEQ ID NO: 309). In some embodiments, the CM comprises the amino acid sequence DRLSGRSDNHKK (SEQ ID NO: 310). In some embodiments, the CM comprises the amino acid sequence NTLSGRSGNHGS (SEQ ID NO: 311).

In some embodiments, the CM comprises the amino acid sequence LSGRSANH (SEQ ID NO: 307). In some embodiments, the CM comprises the amino acid sequence LNGRSDNH (SEQ ID NO: 313). In some embodiments, the CM comprises the amino acid sequence LTGRSDRH (SEQ ID NO: 314). In some embodiments, the CM comprises a core CM consensus 12 sequence comprising the amino acid sequence LSGRSANH (SEQ ID NO: 307), LNGRSDNH (SEQ ID NO: 313), and LTGRSDRH (SEQ ID NO: 314). In some embodiments, the CM an expanded core CM consensus 12 sequence comprising an amino acid sequence selected from the group consisting of DRLSGRSANHKK (SEQ ID NO: 309), DRLSGRSDNHKK (SEQ ID NO: 310), GPLNGRSDNHKA (SEQ ID NO: 320), GPLNGRSDNHKK (SEQ ID NO: 321), GPLNGRSDNHKR (SEQ ID NO: 322), GPLNGRSDNHQA (SEQ ID NO: 323), GPLNGRSDNHQK (SEQ ID NO: 324), GPLNGRSDNHQR (SEQ ID NO: 325), GPLNGRSDNHRA (SEQ ID NO: 326), GPLNGRSDNHRK (SEQ ID NO: 327), GPLNGRSDNHRR (SEQ ID NO: 328), RPLNGRSDNHKA (SEQ ID NO: 329), RPLNGRSDNHKK (SEQ ID NO: 330), RPLNGRSDNHKR (SEQ ID NO: 331), RPLNGRSDNHQA (SEQ ID NO: 332), RPLNGRSDNHQK (SEQ ID NO: 333), RPLNGRSDNHQR (SEQ ID NO: 334), RPLNGRSDNHRA (SEQ ID NO: 335), RPLNGRSDNHRK (SEQ ID NO: 336), RPLNGRSDNHRR (SEQ ID NO: 337), GPLSGRSDNHKA (SEQ ID NO: 338), GPLSGRSDNHKK (SEQ ID NO: 339), GPLSGRSDNHKR (SEQ ID NO: 340), GPLSGRSDNHQA (SEQ ID NO: 341), GPLSGRSDNHQK (SEQ ID NO: 342), GPLSGRSDNHQR (SEQ ID NO: 343), GPLSGRSDNHRA (SEQ ID NO: 344), GPLSGRSDNHRK (SEQ ID NO: 345), GPLSGRSDNHRR (SEQ ID NO: 346), RPLSGRSDNHKA (SEQ ID NO: 347), RPLSGRSDNHKK (SEQ ID NO: 348), RPLSGRSDNHKR (SEQ ID NO: 349), RPLSGRSDNHQA (SEQ ID NO: 350), RPLSGRSDNHQK (SEQ ID NO: 351), RPLSGRSDNHQR (SEQ ID NO: 352), RPLSGRSDNHRA (SEQ ID NO: 353), RPLSGRSDNHRK (SEQ ID NO: 354), RPLSGRSDNHRR (SEQ ID NO: 355), and KGLTGRSDRHQA (SEQ ID NO: 356).

In some embodiments, the CM comprises the amino acid sequence DRLSGRSANHKK (SEQ ID NO: 309). In some embodiments, the CM comprises the amino acid sequence DRLSGRSDNHKK (SEQ ID NO: 310). In some embodiments, the CM comprises the amino acid sequence GPLNGRSDNHKA (SEQ ID NO: 320). In some embodiments, the CM comprises the amino acid sequence GPLNGRSDNHKK (SEQ ID NO: 321). In some embodiments, the CM comprises the amino acid sequence GPLNGRSDNHKR (SEQ ID NO: 322). In some embodiments, the CM comprises the amino acid sequence GPLNGRSDNHQA (SEQ ID NO: 323). In some embodiments, the CM comprises the amino acid sequence GPLNGRSDNHQK (SEQ ID NO: 324). In some embodiments, the CM comprises the amino acid sequence GPLNGRSDNHQR (SEQ ID NO: 325). In some embodiments, the CM comprises the amino acid sequence GPLNGRSDNHRA (SEQ ID NO: 326). In some embodiments, the CM comprises the amino acid sequence GPLNGRSDNHRK (SEQ ID NO: 327). In some embodiments, the CM comprises the amino acid sequence GPLNGRSDNHRR (SEQ ID NO: 328). In some embodiments, the CM comprises the amino acid sequence RPLNGRSDNHKA (SEQ ID NO: 329). In some embodiments, the CM comprises the amino acid sequence RPLNGRSDNHKK (SEQ ID NO: 330). In some embodiments, the CM comprises the amino acid sequence RPLNGRSDNHKR (SEQ ID NO: 331). In some embodiments, the CM comprises the amino acid sequence RPLNGRSDNHQA (SEQ ID NO: 332). In some embodiments, the CM comprises the amino acid sequence RPLNGRSDNHQK (SEQ ID NO: 333). In some embodiments, the CM comprises the amino acid sequence RPLNGRSDNHQR (SEQ ID NO: 334). In some embodiments, the CM comprises the amino acid sequence RPLNGRSDNHRA (SEQ ID NO: 335). In some embodiments, the CM comprises the amino acid sequence RPLNGRSDNHRK (SEQ ID NO: 336). In some embodiments, the CM comprises the amino acid sequence RPLNGRSDNHKR (SEQ ID NO: 337). In some embodiments, the CM comprises the amino acid sequence GPLSGRSDNHKA (SEQ ID NO: 338). In some embodiments, the CM comprises the amino acid sequence GPLSGRSDNHKK (SEQ ID NO: 339). In some embodiments, the CM comprises the amino acid sequence GPLSGRSDNHKR (SEQ ID NO: 340). In some embodiments, the CM comprises the amino acid sequence GPLSGRSDNHQA (SEQ ID NO: 341). In some embodiments, the CM comprises the amino acid sequence GPLSGRSDNHQK (SEQ ID NO: 342). In some embodiments, the CM comprises the amino acid sequence GPLSGRSDNHQR (SEQ ID NO: 343). In some embodiments, the CM comprises the amino acid sequence GPLSGRSDNHKA (SEQ ID NO: 344). In some embodiments, the CM comprises the amino acid sequence GPLSGRSDNHKK (SEQ ID NO: 345). In some embodiments, the CM comprises the amino acid sequence GPLSGRSDNHKR (SEQ ID NO: 346). In some embodiments, the CM comprises the amino acid sequence RPLSGRSDNHKA (SEQ ID NO: 347). In some embodiments, the CM comprises the amino acid sequence RPLSGRSDNHKK (SEQ ID NO: 348). In some embodiments, the CM comprises the amino acid sequence RPLSGRSDNHKR (SEQ ID NO: 349). In some embodiments, the CM comprises the amino acid sequence RPLSGRSDNHQA (SEQ ID NO: 350). In some embodiments, the CM comprises the amino acid sequence RPLSGRSDNHQK (SEQ ID NO: 351). In some embodiments, the CM comprises the amino acid sequence RPLSGRSDNHQR (SEQ ID NO: 352). In some embodiments, the CM comprises the amino acid sequence RPLSGRSDNHKA (SEQ ID NO: 353). In some embodiments, the CM comprises the amino acid sequence RPLSGRSDNHKK (SEQ ID NO: 354). In some embodiments, the CM comprises the amino acid sequence RPLSGRSDNHRR (SEQ ID NO: 355). In some embodiments, the CM comprises the amino acid sequence KGLTGRSDRHQA (SEQ ID NO: 356).

In some embodiments, the CM comprises a core CM consensus 13 sequence comprising the amino acid sequence RIGRSDNH (SEQ ID NO: 357) or RLGRSDNN (SEQ ID NO: 358). In some embodiments, the CM comprises an expanded core CM consensus 13 sequence comprising the amino acid sequence NHRIGRSDNHRR (SEQ ID NO: 359) or TLRLGRSDNNKN (SEQ ID NO: 360).

In some embodiments, the CM comprises the amino acid sequence RIGRSDNH (SEQ ID NO: 357). In some embodiments, the CM comprises the amino acid sequence RLGRSDNN (SEQ ID NO: 358). In some embodiments, the CM comprises the amino acid sequence NHRIGRSDNHRR (SEQ ID NO: 359). In some embodiments, the CM comprises the amino acid sequence TLRLGRSDNNKN (SEQ ID NO: 360).

In some embodiments, the CM comprises a core CM consensus 14 sequence comprising an amino acid sequence selected from the group consisting of TSGRSANP (SEQ ID NO: 361), TSGRSGNP (SEQ ID NO: 362), LSGRSANP (SEQ ID NO: 363), and LSGRSGNP (SEQ ID NO: 364). In some embodiments, the CM comprises an expanded core CM consensus 14 sequence comprising an amino acid sequence selected from the group consisting of TSTSGRSANPRG (SEQ ID NO: 365), TSTSGRSGNPRG (SEQ ID NO: 366), TSLSGRSANPRG (SEQ ID NO: 367), and TSLSGRSGNPRG (SEQ ID NO: 368).

In some embodiments, the CM comprises the amino acid sequence TSGRSANP (SEQ ID NO: 361). In some embodiments, the CM comprises the amino acid sequence TSGRSGNP (SEQ ID NO: 362). In some embodiments, the CM comprises the amino acid sequence LSGRSANP (SEQ ID NO: 363). In some embodiments, the CM comprises the amino acid sequence LSGRSGNP (SEQ ID NO: 364). In some embodiments. In some embodiments, the CM comprises the amino acid sequence the CM comprises the amino acid sequence TSTSGRSANPRG (SEQ ID NO: 365). In some embodiments, the CM comprises the amino acid sequence TSTSGRSGNPRG (SEQ ID NO: 366). In some embodiments, the CM comprises the amino acid sequence TSLSGRSANPRG (SEQ ID NO: 367). In some embodiments, the CM comprises the amino acid sequence and TSLSGRSGNPRG (SEQ ID NO: 368).

In some embodiments, the CM comprises an amino acid sequence selected from the group consisting of LSGRSENH (SEQ ID NO: 369), SIARSDNL (SEQ ID NO: 370), LSGRSVTQ (SEQ ID NO: 371), LSGRSGNH (SEQ ID NO: 308), LTGRSDRH (SEQ ID NO: 314), LYGRSENN (SEQ ID NO: 374), RLGRSDNN (SEQ ID NO: 375), TSGRSANP (SEQ ID NO: 376), NTLSGRSENHSG (SEQ ID NO: 377), PPSIARSDNLAN (SEQ ID NO: 378), TGLSGRSVTQTS (SEQ ID NO: 379), NTLSGRSGNHGS (SEQ ID NO: 311), KGLTGRSDRHQA (SEQ ID NO: 381), KNLYGRSENNGN (SEQ ID NO: 382), TLRLGRSDNNKN (SEQ ID NO: 383), and TSTSGRSANPRG (SEQ ID NO: 384).

In some embodiments, the CM comprises the amino acid sequence LSGRSENH (SEQ ID NO: 369). In some embodiments, the CM comprises the amino acid sequence SIARSDNL (SEQ ID NO: 370). In some embodiments, the CM comprises the amino acid sequence LSGRSVTQ (SEQ ID NO: 371). In some embodiments, the CM comprises the amino acid sequence LSGRSGNH (SEQ ID NO: 308). In some embodiments, the CM comprises the amino acid sequence LTGRSDRH (SEQ ID NO: 314). In some embodiments, the CM comprises the amino acid sequence LYGRSENN (SEQ ID NO: 374). In some embodiments, the CM comprises the amino acid sequence RLGRSDNN (SEQ ID NO: 375). In some embodiments, the CM comprises the amino acid sequence TSGRSANP (SEQ ID NO: 376). In some embodiments, the CM comprises the amino acid sequence NTLSGRSENHSG (SEQ ID NO: 377). In some embodiments, the CM comprises the amino acid sequence PPSIARSDNLAN (SEQ ID NO: 378). In some embodiments, the CM comprises the amino acid sequence TGLSGRSVTQTS (SEQ ID NO: 379). In some embodiments, the CM comprises the amino acid sequence NTLSGRSGNHGS (SEQ ID NO: 311). In some embodiments, the CM comprises the amino acid sequence KGLTGRSDRHQA (SEQ ID NO: 381). In some embodiments, the CM comprises the amino acid sequence KNLYGRSENNGN (SEQ ID NO: 382). In some embodiments, the CM comprises the amino acid sequence TLRLGRSDNNKN (SEQ ID NO: 383). In some embodiments, the CM comprises the amino acid sequence TSTSGRSANPRG (SEQ ID NO: 384).

In some embodiments, the CM is a substrate for at least two proteases. In some embodiments, at least one protease is selected from matriptase and uPA, and at least one protease is selected from the group consisting of those shown in Table 7.

In some embodiments, the activatable antibody includes at least a first CM and a second CM. In some embodiments, the first CM and the second CM are each polypeptides of no more than 15 amino acids long. In some embodiments, the first CM and the second CM in the activatable antibody have the structural arrangement from N-terminus to C-terminus as follows in the uncleaved state: MM-CM1-CM2-AB, AB-CM2-CM1-MM, MM-CM2-CM1-AB, or AB-CM1-CM2-MM. In some embodiments, the activatable antibody includes a linking peptide between MM and CM1. In some embodiments, the activatable antibody includes a linking peptide between CM1 and CM2. In some embodiments, the activatable antibody includes a linking peptide between CM2 and AB. In some embodiments, the activatable antibody includes a linking peptide between MM and CM1 and a linking peptide between CM2 and AB. In some embodiments, the activatable antibody includes a linking peptide between MM and CM1 and a linking peptide between CM1 and CM2. In some embodiments, the activatable antibody includes a linking peptide between CM1 and CM2 and a linking peptide between CM2 and AB. In some embodiments, the activatable antibody includes a linking peptide between MM and CM1, a linking peptide between CM1 and CM2, and a linking peptide between CM2 and AB.

In some embodiments, the CM2 is selected for use with a specific protease. In some embodiments, the CM2 is a substrate for at least one protease selected from the group consisting of a matrix metalloprotease (MMP), a neutrophil elastase, uPA, legumain, matriptase, thrombin, a cysteine protease such as a cathepsin, ADAM17, BMP-1, HtrA1, and a TMPRSS such as TMPRSS3 or TMPRSS4.

In some embodiments, the CM2 is a substrate for a neutrophil elastase. In some embodiments, the CM2 is a substrate for uPA. In some embodiments, the CM2 is a substrate for legumain. In some embodiments, the CM2 is a substrate for matriptase. In some embodiments, the CM2 is a substrate for thrombin. In some embodiments, the CM2 is a substrate for a cysteine protease. In some embodiments, the CM2 is a substrate for a cathepsin. In some embodiments, the CM2 is a substrate for ADAM17. In some embodiments, the CM2 is a substrate for BMP-1. In some embodiments, the CM2 is a substrate for HtrA1. In some embodiments, the CM2 is a substrate for a TMPRSS. In some embodiments, the CM2 is a substrate for TMPRSS3. In some embodiments, the CM2 is a substrate for TMPRSS4.

For example, suitable CM2 are cleaved by at least one protease and include the sequence TGRGPSWV (SEQ ID NO: 402); SARGPSRW (SEQ ID NO: 403); TARGPSFK (SEQ ID NO: 404); TARGPSW (SEQ ID NO: 405); LSGRSDNH (SEQ ID NO: 406); GGWHTGRN (SEQ ID NO: 407); HTGRSGAL (SEQ ID NO: 408); PLTGRSGG (SEQ ID NO: 409); AARGPAIH (SEQ ID NO: 411); RGPAFNPM (SEQ ID NO: 412); SSRGPAYL (SEQ ID NO: 413); RGPATPIM (SEQ ID NO: 414); RGPA (SEQ ID NO: 415); GGQPSGMWGW (SEQ ID NO: 416); FPRPLGITGL (SEQ ID NO: 417); VHMPLGFLGP (SEQ ID NO: 418); SPLTGRSG (SEQ ID NO: 419); SAGFSLPA (SEQ ID NO: 126); LAPLGLQRR (SEQ ID NO: 420); SGGPLGVR (SEQ ID NO: 421); PLGL (SEQ ID NO: 422); GPRSFGL (SEQ ID NO: 423) and/or GPRSFG (SEQ ID NO: 424).

In some embodiments, the CM2 comprises the amino acid sequence TGRGPSWV (SEQ ID NO: 402). In some embodiments, the CM2 comprises the amino acid sequence SARGPSRW (SEQ ID NO: 403). In some embodiments, the CM2 comprises the amino acid sequence TARGPSFK (SEQ ID NO: 404). In some embodiments, the CM2 comprises the amino acid sequence TARGPSW (SEQ ID NO: 405). In some embodiments, the CM2 comprises the amino acid sequence LSGRSDNH (SEQ ID NO: 406). In some embodiments, the CM2 comprises the amino acid sequence GGWHTGRN (SEQ ID NO: 407). In some embodiments, the CM2 comprises the amino acid sequence HTGRSGAL (SEQ ID NO: 408). In some embodiments, the CM2 comprises the amino acid sequence PLTGRSGG (SEQ ID NO: 409). In some embodiments, the CM2 comprises the amino acid sequence AARGPAIH (SEQ ID NO: 411). In some embodiments, the CM2 comprises the amino acid sequence RGPAFNPM (SEQ ID NO: 412). In some embodiments, the CM2 comprises the amino acid sequence SSRGPAYL (SEQ ID NO: 413). In some embodiments, the CM2 comprises the amino acid sequence RGPATPIM (SEQ ID NO: 414). In some embodiments, the CM2 comprises the amino acid sequence RGPA (SEQ ID NO: 415). In some embodiments, the CM2 comprises the amino acid sequence GGQPSGMWGW (SEQ ID NO: 416). In some embodiments, the CM2 comprises the amino acid sequence FPRPLGITGL (SEQ ID NO: 417). In some embodiments, the CM2 comprises the amino acid sequence VHMPLGFLGP (SEQ ID NO: 418). In some embodiments, the CM2 comprises the amino acid sequence SPLTGRSG (SEQ ID NO: 419). In some embodiments, the CM2 comprises the amino acid sequence LAPLGLQRR (SEQ ID NO: 420). In some embodiments, the CM2 comprises the amino acid sequence SGGPLGVR (SEQ ID NO: 421). In some embodiments, the CM2 comprises the amino acid sequence PLGL (SEQ ID NO: 422). In some embodiments, the CM2 comprises the amino acid sequence GPRSFGL (SEQ ID NO: 423). In some embodiments, the CM2 comprises the amino acid sequence GPRSFG (SEQ ID NO: 424).

In some embodiments, the CM2 is a substrate for at least one MMP. In some embodiments, the CM2 is a substrate for at least one MMP listed in the Table 7. In some embodiments, the CM2 is a substrate for MMP9. In some embodiments, the CM2 is a substrate for MMP14. In some embodiments, the CM2 is a substrate for two or more MMPs. In some embodiments, the CM2 is a substrate for at least MMP9 or MMP14. In some embodiments, the CM2 is a substrate for two or more MMPs. In some embodiments, the CM2 is a substrate for at least MMP9 and MMP14.

In some embodiments, CM2 is a substrate for an MMP and includes the sequence

```
(SEQ ID NO: 425)
ISSGLLSS;
(SEQ ID NO: 426)
QNQALRMA;
(SEQ ID NO: 427)
AQNLLGMV;
(SEQ ID NO: 428)
STFPFGMF;
(SEQ ID NO: 429)
PVGYTSSL;
(SEQ ID NO: 430)
DWLYWPGI;
(SEQ ID NO: 431)
MIAPVAYR;
(SEQ ID NO: 432)
RPSPMWAY;
(SEQ ID NO: 433)
WATPRPMR;
(SEQ ID NO: 434)
FRLLDWQW;
(SEQ ID NO: 435)
LKAAPRWA;
(SEQ ID NO: 436)
GPSHLVLT;
(SEQ ID NO: 437)
LPGGLSPW;
(SEQ ID NO: 438)
MGLFSEAG;
        (SEQ ID NO: 439)
SPLPLRVP;
          (SEQ ID NO: 440)
RMHLRSLG;
(SEQ ID NO: 441)
LAAPLGLL;
(SEQ ID NO: 442)
AVGLLAPP;
(SEQ ID NO: 443)
LLAPSHRA,
(SEQ ID NO: 444)
PAGLWLDP; and/or
          (SEQ ID NO: 445)
ISSGLSS.
```

In some embodiments, the first cleaving agent and the second cleaving agent are the protease selected from matriptase and uPA, and the first CM and the second CM are different substrates for the enzyme. In some embodiments, the first cleaving agent and the second cleaving agent are different proteases, where at least one protease is selected from matriptase and uPA. In some embodiments, the first cleaving agent and the second cleaving agent are co-localized in the target tissue. In some embodiments, the first CM and the second CM are cleaved by at least one cleaving agent selected from matriptase and uPA in the target tissue.

In some embodiments, the activatable antibody is exposed to and cleaved by a protease selected from matriptase and uPA such that, in the activated or cleaved state, the activated antibody includes a light chain amino acid sequence that includes at least a portion of LP2 and/or CM sequence after the protease has cleaved the CM.

In some embodiments, the CM comprises the non-prime side of the protease cleavage site; that is, the CM comprises at least the P1 and P2 amino acids, and in some embodiments comprises the P1, P2 and P3 amino acids and in some embodiments comprises the P1, P2, P3, and P4 amino acids. In some embodiments, the CM comprises the non-prime side and the prime side of the protease cleavage site. In some embodiments, the CM comprises the non-prime side but lacks at least part of the prime side of the protease cleavage site. In some embodiments, the CM comprises the non-prime side but lacks the prime side of the protease cleavage site. Such a CM can be linked directly or through a linker to an antibody or other molecule as disclosed herein, such as, but not limited to, a detection moiety.

In some embodiments, the activatable antibody also includes an agent conjugated to the AB. In some embodiments, the agent is a therapeutic agent. In some embodiments, the agent is an antineoplastic agent. In some embodiments, the agent is a toxin or a fragment thereof. In some embodiments, the agent is conjugated to the AB via a linker. In some embodiments, the linker is a cleavable linker. In some embodiments, the agent is a microtubule inhibitor. In some embodiments, the agent is a nucleic acid damaging agent, such as a DNA alkylator or DNA intercalator, or other DNA damaging agent. In some embodiments, the linker is a cleavable linker. In some embodiments, the agent is conjugated to the AB via a linker that includes at least one MMP-cleavable substrate sequence. In some embodiments, the agent is an agent selected from the group listed in Table 3. In some embodiments, the agent is a dolastatin. In some embodiments, the agent is an auristatin or derivative thereof. In some embodiments, the agent is auristatin E or a derivative thereof. In some embodiments, the agent is monomethyl auristatin E (MMAE). In some embodiments, the agent is monomethyl auristatin D (MMAD). In some embodiments, the agent is a maytansinoid or maytansinoid derivative. In some embodiments, the agent is DM1 or DM4. In some embodiments, the agent is a duocarmycin or derivative thereof. In some embodiments, the agent is a calicheamicin or derivative thereof. In some embodiments, the agent is a pyrrolobenzodiazepine.

In some embodiments, the agent is an anti-inflammatory agent.

In some embodiments, the activatable antibody also includes a detectable moiety. In some embodiments, the detectable moiety is a diagnostic agent.

In some embodiments, the conjugated antibody includes a detectable label. In some embodiments, the detectable label includes an imaging agent, a contrasting agent, an enzyme, a fluorescent label, a chromophore, a dye, one or more metal ions, or a ligand-based label. In some embodiments, the imaging agent comprises a radioisotope. In some embodiments, the radioisotope is indium or technetium. In some embodiments, the contrasting agent comprises iodine, gadolinium or iron oxide. In some embodiments, the enzyme comprises horseradish peroxidase, alkaline phosphatase, or β-galactosidase. In some embodiments, the fluorescent label comprises yellow fluorescent protein (YFP), cyan fluorescent protein (CFP), green fluorescent protein (GFP), modified red fluorescent protein (mRFP), red fluorescent protein tdimer2 (RFP tdimer2), HCRED, or a europium derivative. In some embodiments, the luminescent label comprises an N-methylacrydium derivative. In some embodiments, the label comprises an Alexa Fluor® label, such as Alex Fluor® 680 or Alexa Fluor® 750. In some embodiments, the ligand-based label comprises biotin, avidin, streptavidin or one or more haptens.

In some embodiments, the activatable antibody also includes a signal peptide. In some embodiments, the signal peptide is conjugated to the activatable antibody via a spacer. In some embodiments, the spacer is conjugated to the activatable antibody in the absence of a signal peptide. In some embodiments, the spacer is joined directly to the MM of the activatable antibody. In some embodiments, the spacer is joined directly to the MM of the activatable antibody in the structural arrangement from N-terminus to C-terminus of spacer-MM-CM-AB. An example of a spacer joined directly to the N-terminus of MM of the activatable antibody is QGQSGQ (SEQ ID NO: 446). In some embodiments, the spacer includes at least the amino acid sequence QGQSGQ (SEQ ID NO: 446).

In some embodiments, the AB of the activatable antibody naturally contains one or more disulfide bonds. In some embodiments, the AB can be engineered to include one or more disulfide bonds.

In some embodiments, the serum half-life of the activatable antibody is longer than that of the corresponding antibody; e.g., the pK of the activatable antibody is longer than that of the corresponding antibody. In some embodiments, the serum half-life of the activatable antibody is similar to that of the corresponding antibody. In some embodiments, the serum half-life of the activatable antibody is at least 15 days when administered to an organism. In some embodiments, the serum half-life of the activatable antibody is at least 12 days when administered to an organism. In some embodiments, the serum half-life of the activatable antibody is at least 11 days when administered to an organism. In some embodiments, the serum half-life of the activatable antibody is at least 10 days when administered to an organism. In some embodiments, the serum half-life of the activatable antibody is at least 9 days when administered to an organism. In some embodiments, the serum half-life of the activatable antibody is at least 8 days when administered to an organism. In some embodiments, the serum half-life of the activatable antibody is at least 7 days when administered to an organism. In some embodiments, the serum half-life of the activatable antibody is at least 6 days when administered to an organism. In some embodiments, the serum half-life of the activatable antibody is at least 5 days when administered to an organism. In some embodiments, the serum half-life of the activatable antibody is at least 4 days when administered to an organism. In some embodiments, the serum half-life of the activatable antibody is at least 3 days when administered to an organism. In some embodiments, the serum half-life of the activatable antibody is at least 2 days when administered to an organism. In some embodiments, the serum half-life of the activatable antibody is at least 24 hours when administered to an organism. In some embodiments, the serum half-life of the activatable antibody is at least 20 hours when administered to an organism. In some embodiments, the serum half-life of the activatable antibody is at least 18 hours when administered to an organism. In some embodiments, the serum half-life of the activatable antibody is at least 16 hours when administered to an organism. In some embodiments, the serum half-life of the activatable antibody is at least 14 hours when administered to an organism. In some embodiments, the serum half-life of the activatable antibody is at least 12 hours when administered to an organism. In some embodiments, the serum half-life of the activatable antibody is at least 10 hours when administered to an organism. In some embodiments, the serum half-life of the activatable antibody is at least 8 hours when administered to an organism. In some embodiments, the serum half-life of the activatable antibody is at least 6 hours when administered to an organism. In some embodiments, the serum half-life of the activatable antibody is at least 4 hours when administered to an organism. In some embodiments, the serum half-life of the activatable antibody is at least 3 hours when administered to an organism.

In some embodiments, the activatable antibody and/or conjugated activatable antibody is monospecific. In some embodiments, the activatable antibody and/or conjugated activatable antibody is multispecific, e.g., by way of non-limiting example, bispecific or trifunctional. In some embodiments, the activatable antibody and/or conjugated activatable antibody is formulated as part of a pro-Bispecific T Cell Engager (BITE) molecule. In some embodiments, the activatable antibody and/or conjugated activatable antibody is formulated as part of a pro-Chimeric Antigen Receptor (CAR) modified T cell or other engineered receptor.

The disclosure also provides compositions and methods that include an activatable antibody that includes an antibody or antibody fragment (AB) that specifically binds a given target, where the AB is coupled to a masking moiety (MM) that decreases the ability of the AB to bind its target. In some embodiments, the activatable antibody further includes a cleavable moiety (CM) that is a substrate for at least one protease selected from matriptase and uPA. The compositions and methods provided herein enable the attachment of one or more agents to one or more cysteine residues in the AB without compromising the activity (e.g., the masking, activating or binding activity) of the activatable antibody. In some embodiments, the compositions and methods provided herein enable the attachment of one or more agents to one or more cysteine residues in the AB without reducing or otherwise disturbing one or more disulfide bonds within the MM. The compositions and methods provided herein produce an activatable antibody that is conjugated to one or more agents, e.g., any of a variety of therapeutic, diagnostic and/or prophylactic agents, for example, in some embodiments, without any of the agent(s) being conjugated to the MM of the activatable antibody. The compositions and methods provided herein produce conjugated activatable antibodies in which the MM retains the ability to effectively and efficiently mask the AB of the activatable antibody in an uncleaved state. The compositions and methods provided herein produce conjugated activatable antibodies in which the activatable antibody is still activated, i.e., cleaved, in the presence of a protease, i.e., matriptase and/or uPA, that can cleave the CM.

The activatable antibodies have at least one point of conjugation for an agent, but in the methods and compositions provided herein less than all possible points of conjugation are available for conjugation to an agent. In some embodiments, the one or more points of conjugation are sulfur atoms involved in disulfide bonds. In some embodiments, the one or more points of conjugation are sulfur atoms involved in interchain disulfide bonds. In some embodiments, the one or more points of conjugation are sulfur atoms involved in interchain sulfide bonds, but not sulfur atoms involved in intrachain disulfide bonds. In some embodiments, the one or more points of conjugation are sulfur atoms of cysteine or other amino acid residues containing a sulfur atom. Such residues may occur naturally in the antibody structure or may be incorporated into the antibody by site-directed mutagenesis, chemical conversion, or mis-incorporation of non-natural amino acids.

Also provided are methods of preparing a conjugate of an activatable antibody having one or more interchain disulfide bonds in the AB and one or more intrachain disulfide bonds in the MM, and a drug reactive with free thiols is provided. The method generally includes partially reducing interchain disulfide bonds in the activatable antibody with a reducing agent, such as, for example, TCEP; and conjugating the drug reactive with free thiols to the partially reduced activatable antibody. As used herein, the term partial reduction refers to situations where an activatable antibody is contacted with a reducing agent and less than all disulfide bonds, e.g., less than all possible sites of conjugation are reduced. In some embodiments, less than 99%, 98%, 97%, 96%, 95%, 90%, 85%, 80%, 75%, 70%, 65%, 60%, 55%, 50%, 45%, 40%, 35%, 30%, 25%, 20%, 15%, 10% or less than 5% of all possible sites of conjugation are reduced.

In some embodiments, a method of reducing and conjugating an agent, e.g., a drug, to an activatable antibody resulting in selectivity in the placement of the agent is provided. The method generally includes partially reducing the activatable antibody with a reducing agent such that any conjugation sites in the masking moiety or other non-AB portion of the activatable antibody are not reduced, and conjugating the agent to interchain thiols in the AB. The conjugation site(s) are selected so as to allow desired placement of an agent to allow conjugation to occur at a desired site. The reducing agent is, for example, TCEP. The reduction reaction conditions such as, for example, the ratio of reducing agent to activatable antibody, the length of incubation, the temperature during the incubation, the pH of the reducing reaction solution, etc., are determined by identifying the conditions that produce a conjugated activatable antibody in which the MM retains the ability to effectively and efficiently mask the AB of the activatable antibody in an uncleaved state. The ratio of reduction agent to activatable antibody will vary depending on the activatable antibody. In some embodiments, the ratio of reducing agent to activatable antibody will be in a range from about 20:1 to 1:1, from about 10:1 to 1:1, from about 9:1 to 1:1, from about 8:1 to 1:1, from about 7:1 to 1:1, from about 6:1 to 1:1, from about 5:1 to 1:1, from about 4:1 to 1:1, from about 3:1 to 1:1, from about 2:1 to 1:1, from about 20:1 to 1:1.5, from about 10:1 to 1:1.5, from about 9:1 to 1:1.5, from about 8:1 to 1:1.5, from about 7:1 to 1:1.5, from about 6:1 to 1:1.5, from about 5:1 to 1:1.5, from about 4:1 to 1:1.5, from about 3:1 to 1:1.5, from about 2:1 to 1:1.5, from about 1.5:1 to 1:1.5, or from about 1:1 to 1:1.5. In some embodiments, the ratio is in a range of from about 5:1 to 1:1. In some embodiments, the ratio is in a range of from about 5:1 to 1.5:1. In some embodiments, the ratio is in a range of from about 4:1 to 1:1. In some embodiments, the ratio is in a range from about 4:1 to 1.5:1. In some embodiments, the ratio is in a range from about 8:1 to about 1:1. In some embodiments, the ratio is in a range of from about 2.5:1 to 1:1.

In some embodiments, a method of reducing interchain disulfide bonds in the AB of an activatable antibody and conjugating an agent, e.g., a thiol-containing agent such as a drug, to the resulting interchain thiols to selectively locate agent(s) on the AB is provided. The method generally includes partially reducing the AB with a reducing agent to form at least two interchain thiols without forming all possible interchain thiols in the activatable antibody; and conjugating the agent to the interchain thiols of the partially reduced AB. For example, the AB of the activatable antibody is partially reduced for about 1 hour at about 37° C. at a desired ratio of reducing agent:activatable antibody. In some embodiments, the ratio of reducing agent to activatable antibody will be in a range from about 20:1 to 1:1, from about 10:1 to 1:1, from about 9:1 to 1:1, from about 8:1 to 1:1, from about 7:1 to 1:1, from about 6:1 to 1:1, from about 5:1 to 1:1, from about 4:1 to 1:1, from about 3:1 to 1:1, from about 2:1 to 1:1, from about 20:1 to 1:1.5, from about 10:1 to 1:1.5, from about 9:1 to 1:1.5, from about 8:1 to 1:1.5, from about 7:1 to 1:1.5, from about 6:1 to 1:1.5, from about 5:1 to 1:1.5, from about 4:1 to 1:1.5, from about 3:1 to 1:1.5, from about 2:1 to 1:1.5, from about 1.5:1 to 1:1.5, or from about 1:1 to 1:1.5. In some embodiments, the ratio is in a range of from about 5:1 to 1:1. In some embodiments, the ratio is in a range of from about 5:1 to 1.5:1. In some embodiments, the ratio is in a range of from about 4:1 to 1:1. In some embodiments, the ratio is in a range from about 4:1 to 1.5:1. In some embodiments, the ratio is in a range from about 8:1 to about 1:1. In some embodiments, the ratio is in a range of from about 2.5:1 to 1:1.

The thiol-containing reagent can be, for example, cysteine or N-acetyl cysteine. The reducing agent can be, for example, TCEP. In some embodiments, the reduced activatable antibody can be purified prior to conjugation, using for example, column chromatography, dialysis, or diafiltration. In some embodiments, the reduced antibody is not purified after partial reduction and prior to conjugation.

The disclosure also provides partially reduced activatable antibodies in which at least one interchain disulfide bond in the activatable antibody has been reduced with a reducing agent without disturbing any intrachain disulfide bonds in the activatable antibody, wherein the activatable antibody includes an antibody or an antigen binding fragment thereof (AB) that specifically binds to the target, a masking moiety (MM) that inhibits the binding of the AB of the activatable antibody in an uncleaved state to the target, and a cleavable moiety (CM) coupled to the AB, wherein the CM is a polypeptide that functions as a substrate for at least one protease selected from matriptase and uPA. In some embodiments, the MM is coupled to the AB via the CM. In some embodiments, one or more intrachain disulfide bond(s) of the activatable antibody is not disturbed by the reducing agent. In some embodiments, one or more intrachain disulfide bond(s) of the MM within the activatable antibody is not disturbed by the reducing agent. In some embodiments, the activatable antibody in the uncleaved state has the structural arrangement from N-terminus to C-terminus as follows: MM-CM-AB or AB-CM-MM. In some embodiments, reducing agent is TCEP.

The disclosure also provides conjugated activatable antibodies that include an activatable antibody linked to monomethyl auristatin D (MMAD) payload, wherein the activatable antibody includes an antibody or an antigen binding fragment thereof (AB) that specifically binds to a target, a masking moiety (MM) that inhibits the binding of the AB of the activatable antibody in an uncleaved state to the target, and cleavable moiety (CM) coupled to the AB, and the CM is a polypeptide that functions as a substrate for at least one MMP protease.

In some embodiments, the MMAD-conjugated activatable antibody can be conjugated using any of several methods for attaching agents to ABs: (a) attachment to the carbohydrate moieties of the AB, or (b) attachment to sulfhydryl groups of the AB, or (c) attachment to amino groups of the AB, or (d) attachment to carboxylate groups of the AB.

In some embodiments, the MMAD payload is conjugated to the AB via a linker. In some embodiments, the MMAD payload is conjugated to a cysteine in the AB via a linker. In some embodiments, the MMAD payload is conjugated to a lysine in the AB via a linker. In some embodiments, the MMAD payload is conjugated to another residue of the AB via a linker, such as those residues disclosed herein. In some embodiments, the linker is a thiol-containing linker. In some embodiments, the linker is a cleavable linker. In some embodiments, the linker is a non-cleavable linker. In some embodiments, the linker is selected from the group consisting of the linkers shown in Tables 5 and 6. In some embodiments, the activatable antibody and the MMAD payload are linked via a maleimide caproyl-valine-citrulline linker. In some embodiments, the activatable antibody and the MMAD payload are linked via a maleimide PEG-valine-citrulline linker. In some embodiments, the activatable antibody and the MMAD payload are linked via a maleimide caproyl-valine-citrulline-para-aminobenzyloxycarbonyl linker. In some embodiments, the activatable antibody and the MMAD payload are linked via a maleimide PEG-valine-citrulline-para-aminobenzyloxycarbonyl linker. In some embodiments, the MMAD payload is conjugated to the AB using the partial reduction and conjugation technology disclosed herein.

The disclosure also provides polypeptides and other larger molecules that include one or more of the matriptase-cleavable substrate sequences presented herein and/or uPA-cleavable substrate sequences presented herein. By way of non-limiting example, matriptase-cleavable substrate sequences presented herein and/or uPA-cleavable substrate sequences presented herein are useful in prodrug compositions and methods of use thereof. These matriptase-cleavable substrate sequences presented herein and/or uPA-cleavable substrate sequences presented herein are also useful in probes and other detection agents and methods of use thereof. For example, the matriptase-cleavable substrate sequences presented herein and/or uPA-cleavable substrate sequences presented herein can be used in conjunction with fluors and other quenchers to produce detection agents, such as imaging agents and/or other diagnostic agents. Those of ordinary skill in the art will appreciate that the matriptase-cleavable substrate sequences presented herein and/or uPA-cleavable substrate sequences presented herein are useful in any composition and/or method in the art that would use a substrate that is cleavable by matriptase and/or uPA.

In some embodiments, the matriptase and/or uPA substrates of the disclosure are used in larger molecules, for example, isolated polypeptides that include at least one additional moiety (M) selected from the group consisting of (i) at least one moiety that is located amino (N) terminally to the CM (M_{N}), i.e., at a location within the larger molecule that is situated closer to the N-terminus of the larger molecule than the CM; (ii) at least one moiety that is located carboxyl (C) terminally to the CM (M_{C}), i.e., at a location within the larger molecule that is situated closer to the C-terminus of the larger molecule than the CM; and (iii) combinations thereof. In some embodiments, the larger molecule includes at least one M_{N} and at least one M_{C}.

By way of non-limiting examples, suitable M_{N} for use in the larger molecules of the disclosure include at least one of the following: a masking moiety, an antibody, a protein, a therapeutic agent, an antineoplastic agent, a toxic agent, a drug, a detectable moiety, a diagnostic agent, an affinity tag, and combinations thereof.

By way of non-limiting examples, suitable M_{C} for use in the larger molecules of the disclosure include at least one of the following: a masking moiety, an antibody, a protein, a therapeutic agent, an antineoplastic agent, a toxic agent, a drug, a detectable moiety, a diagnostic agent, an affinity tag, and combinations thereof.

The disclosure also provides an isolated nucleic acid molecule encoding a CM-containing molecule of the disclosure, e.g., a CM-containing polypeptide such as, e.g., a CM-containing probe, an antibody and/or an activatable antibody described herein, as well as vectors that include these isolated nucleic acid sequences. The disclosure provides methods of producing CM-containing polypeptide by culturing a cell under conditions that lead to expression of the CM-containing polypeptide, wherein the cell comprises such a vector. The disclosure provides methods of producing an antibody and/or activatable antibody by culturing a cell under conditions that lead to expression of the antibody and/or activatable antibody, wherein the cell comprises such a vector.

The disclosure provides a method of manufacturing a CM-containing polypeptide of the disclosure that binds a given target by (a) culturing a cell comprising a nucleic acid construct that encodes the CM-containing polypeptide under conditions that lead to expression of the polypeptide, (i) wherein the polypeptide includes a cleavable moiety (CM), and (ii) wherein the CM is a polypeptide that functions as a substrate for at least one protease selected from matriptase and uPA; and (b) recovering the polypeptide. These methods can also include the further step of (c) conjugating the recovered polypeptide to one or more additional agents.

The disclosure provides a method of manufacturing a conjugated antibody of the disclosure that binds a given target by (a) culturing a cell comprising a nucleic acid construct that encodes the antibody under conditions that lead to expression of the antibody, (i) wherein the antibody includes a cleavable moiety (CM), and (ii) wherein the CM is a polypeptide that functions as a substrate for at least one protease selected from matriptase and uPA; (b) recovering the antibody; and (c) conjugating the recovered antibody to one or more additional agents.

The disclosure also provides a method of manufacturing the activatable antibodies of the disclosure that bind in an activated state a given target by (a) culturing a cell comprising a nucleic acid construct that encodes the activatable antibody under conditions that lead to expression of the activatable antibody, wherein the activatable antibody comprises a masking moiety (MM), a cleavable moiety (CM), and an antibody or an antigen binding fragment thereof (AB) that specifically binds the target, (i) wherein the CM is a polypeptide that functions as a substrate for a protease selected from matriptase and uPA; and (ii) wherein the CM is positioned in the activatable antibody such that, in an uncleaved state, the MM interferes with specific binding of the AB to the target and in a cleaved state the MM does not interfere or compete with specific binding of the AB to the target; and (b) recovering the activatable antibody.

The disclosure also provides methods of producing non-polypeptide CM-containing molecules, including by way of non-limiting examples, prodrugs, non-peptide probes, etc. These non-polypeptide CM-containing molecules can be made using any of a variety of art-recognized techniques, including standard chemical synthesis and/or conjugation methods.

The disclosure provides methods of preventing, delaying the progression of, treating, alleviating a symptom of, or otherwise ameliorating a target-related disease in a subject by administering a therapeutically effective amount of a conjugated antibody, an activatable antibody and/or a conjugated activatable antibody described herein to a subject in need thereof.

The disclosure provides methods of preventing, delaying the progression of, treating, alleviating a symptom of, or otherwise ameliorating inflammation and/or an inflammatory disorder in a subject by administering a therapeutically effective amount of a conjugated antibody, an activatable antibody and/or a conjugated activatable antibody described herein to a subject in need thereof. The disclosure also provides methods of preventing, delaying the progression of, treating, alleviating a symptom of, or otherwise ameliorating cancer in a subject by administering a therapeutically effective amount of a conjugated antibody, an activatable antibody and/or a conjugated activatable antibody described herein to a subject in need thereof. The disclosure also provides methods of preventing, delaying the progression of, treating, alleviating a symptom of, or otherwise ameliorating an autoimmune disease in a subject by administering a therapeutically effective amount a conjugated antibody, an activatable antibody and/or a conjugated activatable antibody described herein to a subject in need thereof.

A conjugated antibody, an activatable antibody and/or a conjugated activatable antibody used in any of the embodiments of these methods and uses can be administered at any stage of the disease. For example, such a conjugated antibody, activatable antibody and/or conjugated activatable antibody can be administered to a patient suffering cancer of any stage, from early to metastatic. The terms subject and patient are used interchangeably herein.

In some embodiments, the subject is a mammal, such as a human, non-human primate, companion animal (e.g., cat, dog, horse), farm animal, work animal, or zoo animal. In some embodiments, the subject is a human. In some embodiments, the subject is a companion animal. In some embodiments, the subject is an animal in the care of a veterinarian.

The conjugated antibody, activatable antibody and/or conjugated activatable antibody and therapeutic formulations thereof are administered to a subject suffering from or susceptible to a disease or disorder associated with aberrant target expression and/or activity. A subject suffering from or susceptible to a disease or disorder associated with aberrant target expression and/or activity is identified using any of a variety of methods known in the art. For example, subjects suffering from cancer or other neoplastic condition are identified using any of a variety of clinical and/or laboratory tests such as, physical examination and blood, urine and/or stool analysis to evaluate health status. For example, subjects suffering from inflammation and/or an inflammatory disorder are identified using any of a variety of clinical and/or laboratory tests such as physical examination and/or bodily fluid analysis, e.g., blood, urine and/or stool analysis, to evaluate health status.

Administration of a conjugated antibody, an activatable antibody and/or a conjugated activatable antibody to a patient suffering from a disease or disorder associated with aberrant target expression and/or activity is considered successful if any of a variety of laboratory or clinical objectives is achieved. For example, administration of a conjugated antibody, an activatable antibody and/or a conjugated activatable antibody to a patient suffering from a disease or disorder associated with aberrant target expression and/or activity is considered successful if one or more of the symptoms associated with the disease or disorder is alleviated, reduced, inhibited or does not progress to a further, i.e., worse, state. Administration of a conjugated antibody, an activatable antibody and/or a conjugated activatable antibody to a patient suffering from a disease or disorder associated with aberrant target expression and/or activity is considered successful if the disease or disorder enters remission or does not progress to a further, i.e., worse, state.

In some embodiments, the conjugated antibody, activatable antibody and/or conjugated activatable antibody is administered during and/or after treatment in combination with one or more additional agents such as, for example, an anti-inflammatory agent, an immunosuppressive agent, and/or a chemotherapeutic agent. In some embodiments, the conjugated antibody, activatable antibody and/or conjugated activatable antibody and the additional agent(s) are administered simultaneously. For example, the conjugated antibody, activatable antibody and/or conjugated activatable antibody and the additional agent(s) can be formulated in a single composition or administered as two or more separate compositions. In some embodiments, the conjugated antibody, activatable antibody and/or conjugated activatable antibody and the additional agent(s) are administered sequentially, or the antibody and/or conjugated antibodies and the additional agent are administered at different times during a treatment regimen. For example, the antibody and/or conjugated antibodies is administered prior to the administration of the additional agent, the antibody and/or conjugated antibodies is administered subsequent to the administration of the additional agent, or the antibody and/or conjugated antibodies and the additional agent are administered in an alternating fashion. As described herein, the antibody and/or conjugated antibodies and additional agent are administered in single doses or in multiple doses.

In some embodiments, the conjugated antibody, activatable antibody and/or conjugated activatable antibody is administered during and/or after treatment in combination with one or more additional agents such as, by way of non-limiting example, an anti-inflammatory agent, an immunosuppressive agent, a chemotherapeutic agent, such as an alkylating agent, an anti-metabolite, an anti-microtubule agent, a topoisomerase inhibitor, a cytotoxic antibiotic, and/or any other nucleic acid damaging agent. In some embodiments, the additional agent is a taxane, such as paclitaxel (e.g., Abraxane®). In some embodiments, the additional agent is an anti-metabolite, such as gemcitabine. In some embodiments, the additional agent is an alkylating agent, such as platinum-based chemotherapy, such as carboplatin or cisplatin. In some embodiments, the additional agent is a targeted agent, such as a kinase inhibitor, e.g., sorafenib or erlotinib. In some embodiments, the additional agent is a targeted agent, such as another antibody, e.g., a monoclonal antibody (e.g., bevacizumab), a bispecific antibody, or a multispecific antibody. In some embodiments, the additional agent is a proteosome inhibitor, such as bortezomib or carfilzomib. In some embodiments, the additional agent is an immune modulating agent, such as lenolidominde or IL-2. In some embodiments, the additional agent is radiation. In some embodiments, the additional agent is an agent considered standard of care by those skilled in the art. In some embodiments, the additional agent is a chemotherapeutic agent well known to those skilled in the art.

In some embodiments, the additional agent is an antibody, another conjugated antibody, another activatable antibody and/or another conjugated activatable antibody. In some embodiments the additional agent is an antibody, another conjugated antibody, another activatable antibody and/or another conjugated activatable antibody against the same target as the first conjugated antibody, activatable antibody and/or a conjugated activatable antibody. In some embodiments the additional agent is an antibody, another conjugated antibody, another activatable antibody and/or another conjugated activatable antibody against a target different than the target of the first conjugated antibody, activatable antibody and/or a conjugated activatable antibody.

In some embodiments, the conjugated antibody, activatable antibody and/or conjugated activatable antibody and the additional agent(s) are administered simultaneously. For example, the conjugated antibody, activatable antibody and/or conjugated activatable antibody and the additional agent(s) can be formulated in a single composition or administered as two or more separate compositions. In some embodiments, the conjugated antibody, activatable antibody and/or conjugated activatable antibody and the additional agent(s) are administered sequentially, or the antibody and/or conjugated antibodies and the additional agent are administered at different times during a treatment regimen. For example, the antibody and/or conjugated antibodies is administered prior to the administration of the additional agent, the antibody and/or conjugated antibodies is administered subsequent to the administration of the additional agent, or the antibody and/or conjugated antibodies and the additional agent are administered in an alternating fashion. As described herein, the antibody and/or conjugated antibodies and additional agent are in single doses or in multiple doses.

In some embodiments, the CM is linked or otherwise attached to an activatable antibody that includes an antibody or antigen-binding fragment thereof that specifically binds a given target coupled to a masking moiety (MM), such that coupling of the MM to the AB reduces the ability of the antibody or antigen-binding fragment thereof to bind the target. In some embodiments, the MM is coupled via the CM. Exemplary targets include, but are not limited to the targets shown in Table 1. Exemplary ABs include, but are not limited to, the targets shown in Table 2. The activatable antibodies provided herein are stable in circulation, activated at intended sites of therapy and/or diagnosis but not in normal, e.g., healthy tissue or other tissue not targeted for treatment and/or diagnosis, and, when activated, exhibit binding to the target that is at least comparable to the corresponding, unmodified antibody.

The disclosure also provides methods and kits for using the conjugated antibodies, activatable antibodies and/or conjugated activatable antibodies in a variety of diagnostic and/or prophylactic indications.

In some embodiments, the disclosure provides methods and kits for detecting presence or absence of a cleaving agent and a target of interest in a subject or a sample by (i) contacting a subject or sample with an activatable antibody, wherein the activatable antibody comprises a masking moiety (MM), a cleavable moiety (CM) that is cleaved by the cleaving agent, and an antigen binding domain or fragment thereof (AB) that specifically binds the target of interest, wherein the activatable antibody in an uncleaved, non-activated state comprises a structural arrangement from N-terminus to C-terminus as follows: MM-CM-AB or AB-CM-MM; (a) wherein the MM is a peptide that inhibits binding of the AB to the target, and wherein the MM does not have an amino acid sequence of a naturally occurring binding partner of the AB and is not a modified form of a natural binding partner of the AB; and (b) wherein, in an uncleaved, non-activated state, the MA/I interferes with specific binding of the AB to the target, and in a cleaved, activated state the MM does not interfere or compete with specific binding of the AB to the target; and (ii) measuring a level of activated activatable antibody in the subject or sample, wherein a detectable level of activated activatable antibody in the subject or sample indicates that the cleaving agent and the target are present in the subject or sample and wherein no detectable level of activated activatable antibody in the subject or sample indicates that the cleaving agent, the target or both the cleaving agent and the target are absent in the subject or sample.

In some embodiments, the activatable antibody is an activatable antibody to which a therapeutic agent is conjugated. In some embodiments, the activatable antibody is not conjugated to an agent. In some embodiments, the activatable antibody comprises a detectable label. In some embodiments, the detectable label is positioned on the AB. In some embodiments, measuring the level of activatable antibody in the subject or sample is accomplished using a secondary reagent that specifically binds to the activated antibody, wherein the reagent comprises a detectable label. In some embodiments, the secondary reagent is an antibody comprising a detectable label.

In some embodiments of these methods and kits, the activatable antibody includes a detectable label. In some embodiments of these methods and kits, the detectable label includes an imaging agent, a contrasting agent, an enzyme, a fluorescent label, a chromophore, a dye, one or more metal ions, or a ligand-based label. In some embodiments of these methods and kits, the imaging agent comprises a radioisotope. In some embodiments of these methods and kits, the radioisotope is indium or technetium. In some embodiments of these methods and kits, the contrasting agent comprises iodine, gadolinium or iron oxide. In some embodiments of these methods and kits, the enzyme comprises horseradish peroxidase, alkaline phosphatase, or β-galactosidase. In some embodiments of these methods and kits, the fluorescent label comprises yellow fluorescent protein (YFP), cyan fluorescent protein (CFP), green fluorescent protein (GFP), modified red fluorescent protein (mRFP), red fluorescent protein tdimer2 (RFP tdimer2), HCRED, or a europium derivative. In some embodiments of these methods and kits, the luminescent label comprises an N-methylacrydium derivative. In some embodiments of these methods, the label comprises an Alexa Fluor® label, such as Alex Fluor® 680 or Alexa Fluor® 750. In some embodiments of these methods and kits, the ligand-based label comprises biotin, avidin, streptavidin or one or more haptens.

In some embodiments of these methods and kits, the subject is a mammal. In some embodiments of these methods, the subject is a human. In some embodiments, the subject is a non-human mammal, such as a non-human primate, companion animal (e.g., cat, dog, horse), farm animal, work animal, or zoo animal. In some embodiments, the subject is a rodent.

In some embodiments of these methods and kits, the method is an in vivo method. In some embodiments of these methods, the method is an in situ method. In some embodiments of these methods, the method is an ex vivo method. In some embodiments of these methods, the method is an in vitro method.

The disclosure also provides methods of detecting presence or absence of a cleaving agent in a subject or a sample by (i) contacting a subject or biological sample with a probe comprising a cleavable moiety (CM) and a detectable label that is released or activated following cleavage of the CM; and (ii) measuring a level of detectable label in the subject or biological sample. When such release or activation increases detection of the label (e.g., stimulates a detectable signal), a detectable level of the detectable label in the subject or biological sample indicates that the cleaving agent is present in the subject or biological sample, and wherein a reduced detectable level of the detectable label in the subject or biological sample indicates that the cleaving agent is absent and/or not sufficiently present in the subject or biological sample at a detectable level, such that protease cleavage of the CM cannot be detected in the subject or biological sample. When such release or activation reduces detection of the label, a detectable level of the detectable label in the subject or biological sample indicates that the cleaving agent is absent and/or not sufficiently present in the subject or biological sample at a detectable level, such that protease cleavage of the CM cannot be detected in the subject or biological sample, and wherein a reduced detectable level of the detectable label in the subject or biological sample indicates that the cleaving agent is present in the subject or biological sample.

In some embodiments of these methods and kits, the probes comprising CM includes a detectable label. In some embodiments of these methods and kits, the detectable label includes an imaging agent, a contrasting agent, an enzyme, a fluorescent label, a chromophore, a dye, one or more metal ions, or a ligand-based label. In some embodiments of these methods and kits, the imaging agent comprises a radioisotope. In some embodiments of these methods and kits, the radioisotope is indium or technetium. In some embodiments of these methods and kits, the contrasting agent comprises iodine, gadolinium or iron oxide. In some embodiments of these methods and kits, the enzyme comprises horseradish peroxidase, alkaline phosphatase, or β-galactosidase. In some embodiments of these methods and kits, the fluorescent label comprises yellow fluorescent protein (YFP), cyan fluorescent protein (CFP), green fluorescent protein (GFP), modified red fluorescent protein (mRFP), red fluorescent protein tdimer2 (RFP tdimer2), HCRED, or a europium derivative. In some embodiments of these methods and kits, the luminescent label comprises an N-methylacrydium derivative. In some embodiments of these methods, the label comprises an Alexa Fluor® label, such as Alex Fluor® 680 or Alexa Fluor® 750. In some embodiments of these methods and kits, the ligand-based label comprises biotin, avidin, streptavidin or one or more haptens.

In some embodiments of these methods, the method is an in vivo method. In some embodiments of these methods, the method is an in situ method. In some embodiments of these methods, the method is an ex vivo method. In some embodiments of these methods, the method is an in vitro method.

In some embodiments of the methods and kits, the method is used to identify or otherwise refine a patient population suitable for treatment with an activatable antibody of the disclosure, followed by treatment by administering that activatable antibody and/or conjugated activatable antibody to a subject in need thereof. For example, patients that test positive for both the target and at least one protease selected from matriptase and uPA that cleaves the substrate in the cleavable moiety (CM) of the activatable antibody being tested in these methods are identified as suitable candidates for treatment with such an activatable antibody comprising such a CM, and the patient is then administered a therapeutically effective amount of the activatable antibody and/or conjugated activatable antibody that was tested. Likewise, patients that test negative for either or both of the target and the protease, i.e., matriptase and/or uPA that cleaves the substrate in the CM in the activatable antibody being tested using these methods might be identified as suitable candidates for another form of therapy. In some embodiments, such patients can be tested with other activatable antibodies until a suitable activatable antibody for treatment is identified (e.g., an activatable antibody comprising a CM that is cleaved by the patient at the site of disease). In some embodiments, the patient is then administered a therapeutically effective amount of the activatable antibody and/or conjugated for which the patient tested positive.

The disclosure also provides polypeptides and other larger molecules that include one or more of the matriptase-cleavable substrate sequences presented herein and/or uPA-cleavable substrate sequences presented herein. By way of non-limiting example, matriptase-cleavable substrate sequences presented herein and/or uPA-cleavable substrate sequences presented herein are useful in prodrug compositions and methods of use thereof. In some embodiments, the polypeptide comprises a CM joined to a drug, such as a small molecule. Examples of drugs are well known in the art. These matriptase-cleavable substrate sequences presented herein and/or uPA-cleavable substrate sequences presented herein are also useful in probes and other detection agents and methods of use thereof. For example, the matriptase-cleavable substrate sequences presented herein and/or uPA-cleavable substrate sequences presented herein can be used in conjunction with fluors and other quenchers to produce detection agents, such as imaging agents and/or other diagnostic agents. Those of ordinary skill in the art will appreciate that the matriptase-cleavable substrate sequences presented herein and/or uPA-cleavable substrate sequences presented herein are useful in any composition and/or method in the art that would use a substrate that is cleavable by matriptase and/or uPA.

In some embodiments, the matriptase and/or uPA substrates of the disclosure are used in larger molecules, for example, isolated polypeptides that include at least one additional moiety (M) selected from the group consisting of (i) at least one moiety that is located amino (N) terminally to the CM (M_{N}), i.e., at a location within the larger molecule that is situated closer to the N-terminus of the larger molecule than the CM; (ii) at least one moiety that is located carboxyl (C) terminally to the CM (M_{C}), i.e., at a location within the larger molecule that is situated closer to the C-terminus of the larger molecule than the CM; and (iii) combinations thereof. In some embodiments, the larger molecule includes at least one M_{N} and at least one M_{C}.

By way of non-limiting examples, suitable M_{N} for use in the larger molecules of the disclosure include at least one of the following: a masking moiety, an antibody, a protein, a therapeutic agent, an antineoplastic agent, a toxic agent, a drug, a detectable moiety, a diagnostic agent, an affinity tag, and combinations thereof.

By way of non-limiting examples, suitable M_{C} for use in the larger molecules of the disclosure include at least one of the following: a masking moiety, an antibody, a protein, a therapeutic agent, an antineoplastic agent, a toxic agent, a drug, a detectable moiety, a diagnostic agent, an affinity tag, and combinations thereof.

Pharmaceutical compositions according to the disclosure can include an antibody of the disclosure and a carrier. These pharmaceutical compositions can be included in kits, such as, for example, diagnostic kits.

### BRIEF DESCRIPTION OF THE DRAWINGS

FIG. 1 is a series of graphs depicting cleavage of pool SMP30 by matriptase-1.

FIG. 2 is a series of graphs depicting cleavage of pool SMP17 by matriptase-1 and resistance to cleavage by tPA.

FIG. 3 is a series of graphs depicting cleavage of the substrate sequence VAGRSMRP (SEQ ID NO: 251) by matriptase-1.

FIGS. 4A and 4B are a series of schematic representations of the peptide display platforms used in the working examples provided herein. FIG. 4A is a schematic representation of the sequence of the display platform referred to herein as “Display Platform CYTX-DP-XXXXXXXX” or “CYTX-DP-XXXXXXXX” (SEQ ID NO: 694). FIG. 4B is a schematic representation of the sequence of the display platform referred to herein as “Display Platform SP-CYTX-DP-XXXXXXXX” or “SP-CYTX-DP-XXXXXXXX” (SEQ ID NO: 695), where SP-CYTX-DP-XXXXXXXX is the CYTX-DP-XXXXXXXX platform with a signal peptide.

### DETAILED DESCRIPTION OF THE INVENTION

The disclosure provides amino acid sequences that include a cleavable moiety (CM) that is a substrate for at least one protease selected from matriptase and u-plasminogen activator (uPA). These CMs are useful in a variety of therapeutic, diagnostic and prophylactic indications.

The disclosure provides antibodies that include one or more of these matriptase-cleavable substrates and/or uPA-cleavable substrates. For example, these matriptase-cleavable substrates and/or uPA-cleavable substrates are useful when conjugating antibodies to one or more additional agents to produce conjugated antibodies. These matriptase-cleavable substrates and/or uPA-cleavable substrates are useful in activatable antibody constructs.

The conjugated antibodies include an antibody or antigen-binding fragment thereof that specifically binds a target, and the activatable antibodies include an antibody or antigen-binding fragment thereof (AB) that specifically binds a target. Exemplary classes of targets of an antibody or antigen-binding fragment thereof include, but are not necessarily limited to, cell surface receptors and secreted binding proteins (e.g., growth factors), soluble enzymes, structural proteins (e.g. collagen, fibronectin) and the like. In some embodiments, conjugated antibodies and/or activatable antibodies have an antibody or antigen-binding fragment thereof that binds an extracellular target, usually an extracellular protein target. In some embodiments, conjugated antibodies and/or activatable antibodies are designed for cellular uptake and are switchable inside a cell.

As a non-limiting example, the antibody or antigen-binding fragment and/or the AB of an activatable antibody is a binding partner for any target listed in Table 1.

TABLE 1
Exemplary Targets

| | | | |
|---|---|---|---|
| 1-92-LFA-3 | CD166 | HER2/neu | Receptors |
| Alpha-4 | CD172A | HGF | Notch 1 |
| integrin | CD248 | hGH | Notch 2 |
| Alpha-V | CDH6 | HVEM | Notch 3 |
| integrin | CEACAM5 | Hyaluronidase | Notch 4 |
| alpha4beta1 | (CEA) | ICOS | NOV |
| integrin | CEACAM6 | IFNalpha | OSM-R |
| alpha4beta7 | (NCA-90) | IFNbeta | OX-40 |
| integrin | CLAUDIN-3 | IFNgamma | PAR2 |
| AGR2 | CLAUDIN-4 | IgE | PDGF-AA |
| Anti-Lewis-Y | cMet | IgE Receptor | PDGF-BB |
| Apelin J | Collagen | (FceRI) | PDGFRalpha |
| receptor | Cripto | IGF | PDGFRbeta |
| APRIL | CSFR | IGF1R | PD-1 |
| B7-H4 | CSFR-1 | IL1B | PD-L1 |
| BAFF | CTLA-4 | IL1R | PD-L2 |
| BTLA | CTGF | IL2 | Phosphatidyl- |
| C5 | CXCL10 | IL11 | serine |
| complement | CXCL13 | IL12 | P1GF |
| C-242 | CXCR1 | IL12p40 | PSCA |
| CA9 | CXCR2 | IL-12R, | PSMA |
| CA19-9 | CXCR4 | IL-12Rbeta1 | RAAG12 |
| (Lewis a) | CYR61 | IL13 | RAGE |
| Carbonic | DL44 | IL13R | SLC44A4 |
| anhydrase 9 | DLK1 | IL15 | Sphingosine 1 |
| CD2 | DLL4 | IL17 | Phosphate |
| CD3 | DPP-4 | IL18 | STEAP1 |
| CD6 | DSG1 | IL21 | STEAP2 |
| CD9 | EGFR | IL23 | TAG-72 |
| CD11a | EGFRviii | IL23R | TAPA1 |
| CD19 | Endothelin B | IL27/IL27R | TGFbeta |
| CD20 | receptor | (wsx1) | TIGIT |
| CD22 | (ETBR) | IL29 | TIM-3 |
| CD24 | ENPP3 | IL-31R | TLR2 |
| CD25 | EpCAM | IL31/IL31R | TLR4 |
| CD27 | EPHA2 | IL2R | TLR6 |
| CD28 | EPHB2 | IL4 | TLR7 |
| CD30 | ERBB3 | IL4R | TLR8 |
| CD33 | F protein of | IL6, IL6R | TLR9 |
| CD38 | RSV | Insulin | TMEM31 |
| CD40 | FAP | Receptor | TNFalpha |
| CD40L | FGF-2 | Jagged | TNFR |
| CD41 | FGF8 | Ligands | TNFRS12A |
| CD44 | FGFR1 | Jagged 1 | TRAIL-R1 |
| CD44v6 | FGFR2 | Jagged 2 | TRAIL-R2 |
| CD47 | FGFR3 | LAG-3 | Transferrin |
| CD51 | FGFR4 | LIF-R | Transferrin |
| CD52 | Folate | Lewis X | receptor |
| CD56 | receptor | LIGHT | TRK-A |
| CD64 | GAL3ST1 | LRP4 | TRK-B |
| CD70 | G-CSF | LRRC26 | uPAR |
| CD71 | G-CSFR | MCSP | VAP1 |
| CD74 | GD2 | Mesothelin | VCAM-1 |
| CD80 | GITR | MRP4 | VEGF |
| CD81 | GLUT1 | MUC1 | VEGF-A |
| CD86 | GLUT4 | Mucin-16 | VEGF-B |
| CD95 | GM-CSF | (MUC16, | VEGF-C |
| CD117 | GM-CSFR | CA-125) | VEGF-D |
| CD125 | GP IIb/IIIa | Na/K ATPase | VEGFR1 |
| CD132 | receptors | Neutrophil | VEGFR2 |
| (IL-2RG) | Gp130 | elastase | VEGFR3 |
| CD133 | GPIIB/IIIA | NGF | VISTA |
| CD137 | GPNMB | Nicastrin | WISP-1 |
| CD138 | GRP78 | Notch | WISP-2 |
| | | | WISP-3 |

As a non-limiting example, the antibody or antigen-binding fragment and/or the AB of an activatable antibody is or is derived from an antibody listed in Table 2.

TABLE 2
Exemplary sources for Abs

| Antibody Trade Name (antibody name) | Target |
|---|---|
| Avastin ™ (bevacizumab) | VEGF |
| Lucentis ™ (ranibizumab) | VEGF |
| Erbitux ™ (cetuximab) | EGFR |
| Vectibix ™ (panitumumab) | EGFR |
| Remicade ™ (infliximab) | TNFα |
| Humira ™ (adalimumab) | TNFα |
| Tysabri ™ (natalizumab) | Integrinα4 |
| Simulect ™ (basiliximab) | IL2R |
| Soliris ™ (eculizumab) | Complement C5 |
| Raptiva ™ (efalizumab) | CD11a |
| Bexxar ™ (tositumomab) | CD20 |
| Zevalin ™ (ibritumomab tiuxetan) | CD20 |
| Rituxan ™ (rituximab) | CD20 |
| Ocrelizumab | CD20 |
| Arzerra ™ (ofatumumab) | CD20 |
| Obinutuzumab | CD20 |
| Zenapax ™ (daclizumab) | CD25 |
| Adcetris ™ (brentuximab vedotin) | CD30 |
| Myelotarg ™ (gemtuzumab) | CD33 |
| Mylotarg ™ (gemtuzumab ozogamicin) | CD33 |
| Campath ™ (alemtuzumab) | CD52 |
| ReoPro ™ (abiciximab) | Glycoprotein receptor IIb/IIIa |
| Xolair ™ (omalizumab) | IgE |
| Herceptin ™ (trastuzumab) | Her2 |
| Kadcyla ™ (trastuzumab emtansine) | Her2 |
| Synagis ™ (palivizumab) | F protein of RSV |
| (ipilimumab) | CTLA-4 |
| (tremelimumab) | CTLA-4 |
| Hu5c8 | CD40L |
| (pertuzumab) | Her2-neu |
| (ertumaxomab) | CD3/Her2-neu |
| Orencia ™ (abatacept) | CTLA-4 |
| (tanezumab) | NGF |
| (bavituximab) | Phosphatidylserine |
| (zalutumumab) | EGFR |
| (mapatumumab) | EGFR |
| (matuzumab) | EGFR |
| (nimotuzumab) | EGFR |
| ICR62 | EGFR |
| mAb 528 | EGFR |
| CH806 | EGFR |
| MDX-447 | EGFR/CD64 |
| (edrecolomab) | EpCAM |
| RAV12 | RAAG12 |
| huJ591 | PSMA |
| Enbrel ™ (etanercept) | TNF-R |
| Amevive ™ (alefacept) | 1-92-LFA-3 |
| Antril ™, Kineret ™ (ankinra) | IL-1Ra |
| GC1008 | TGFbeta |
| | Notch, e.g., Notch 1 |
| | Jagged 1 or Jagged 2 |
| (adecatumumab) | EpCAM |
| (figitumumab) | IGF1R |
| (tocilizumab) | IL-6 receptor |
| Stelara ™ (ustekinumab) | IL-12/IL-23 |
| Prolia ™ (denosumab) | RANKL |

Exemplary conjugated antibodies and/or activatable antibodies of the disclosure include, for example, antibodies that bind interleukin 6 receptor (IL-6R) and that include a heavy chain and a light chain that are, or are derived from, the antibody referred to herein as the“Av1” antibody, which binds interleukin-6 receptor (IL-6R). The amino acid sequences for the Av1 heavy chain and the Av1 light chain are shown below in SEQ ID NO: 54 and SEQ ID NO: 55, respectively.

Av1 Antibody Heavy Chain Amino Acid Sequence:
(SEQ ID NO: 447)
QVQLQESGPGLVRPSQTLSLTCTVSGYSITSDHAWSWVRQPPGRGLEWIG
YISYSGITTYNPSLKSRVTISRDNSKNTLYLQMNSLRAEDTAVYYCARSL
ARTTAMDYWGQGSLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK
Av1 Antibody Light Chain Amino Acid Sequence:
(SEQ ID NO: 448)
DIQMTQSPSSLSASVGDRVTITCRASQDISSYLNWYQQKPGKAPKLLIYY
TSRLHSGVPSRFSGSGSGTDFTFTISSLQPEDIATYYCQQGNTLPYTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

Exemplary conjugated antibodies and/or activatable antibodies of the disclosure include, for example, antibodies that bind interleukin 6 receptor (IL-6R) and that include a heavy chain and a light chain that are, or are derived from, the Av1 antibody and a masking moiety. Exemplary conjugated antibodies and/or activatable antibodies of the disclosure include an amino acid sequence attached to the N-terminus of the AV1 light chain. These N-terminal amino acid sequences include, for example, YGSCSWNYVHIFMDC (SEQ ID NO: 449); QGDFDIPFPAHWVPIT (SEQ ID NO: 450); MGVPAGCVWNYAHIFMDC (SEQ ID NO: 451); QGQSGQYGSCSWNYVHIFMDC (SEQ ID NO: 452); QGQSGQGDFDIPFPAHWVPIT (SEQ ID NO: 453); or QGQSGQMGVPAGCVWNYAHIFMDC (SEQ ID NO: 454). It is also to be appreciated that such amino acid sequences can be attached to the N-terminus of the AV1 heavy chain or to the C-terminus of the AV1 heavy or light chain.

Exemplary activatable antibodies of the disclosure include, for example, antibodies that bind Epidermal Growth Factor Receptor (EGFR) and that include a heavy chain and a light chain that are, or are derived from, an antibody selected from the group consisting of the antibody referred to herein as the“c225v5” antibody, the antibody referred to herein as the“c225v4” antibody, and the antibody referred to herein as the“c225v6” antibody, each of which binds EGFR. The c225v5 antibody, the c225v4 antibody, and the c225v6 antibody share the same light chain sequence, referred to herein as “c225 light chain.” The amino acid sequences for the c225v5 heavy chain, the c225v4 antibody, the c225v6 antibody, and the c225 light chain are shown below.

C225v5 Antibody Heavy Chain Amino Acid Sequence:
(SEQ ID NO: 455)
QVQLKQSGPGLVQPSQSLSITCTVSGFSLTNYGVHWVRQSPGKGLEWLGV
IWSGGNTDYNTPFTSRLSINKDNSKSQVFFKMNSLQSQDTAIYYCARALT
YYDYEFAYWGQGTLVTVSAASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK*
C225v4 Antibody Heavy Chain Amino Acid Sequence:
(SEQ ID NO: 456)
QVQLKQSGPGLVQPSQSLSITCTVSGFSLTNYGVHWVRQSPGKGLEWLGV
IWSGGNTDYNTPFTSRLSINKDNSKSQVFFKMNSLQSNDTAIYYCARALT
YYDYEFAYWGQGTLVTVSAASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK*
C225v6 Antibody Heavy Chain Amino Acid Sequence:
(SEQ ID NO: 457)
QVQLKQSGPGLVQPSQSLSITCTVSGFSLTNYGVHWVRQSPGKGLEWLGV
IWSGGNTDYNTPFTSRLSINKDNSKSQVFFKMNSLQSQDTAIYYCARALT
YYDYEFAYWGQGTLVTVSAASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYAS
TYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK*
C225 Antibody Light Chain Amino Acid Sequence:
(SEQ ID NO: 458)
QILLTQSPVILSVSPGERVSFSCRASQSIGTNIHWYQQRTNGSPRLLIKY
ASESISGIPSRFSGSGSGTDFTLSINSVESEDIADYYCQQNNNWPTTFGA
GTKLELKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC*

Exemplary activatable antibodies of the disclosure include, for example, antibodies that bind EGFR, that include a heavy chain and a light chain that are, or are derived from, the c225v5 antibody, and that include a masking moiety, a first linking peptide, a cleavable moiety, and a second linking peptide. In some embodiments, the heavy chain and/or the light chain includes a signal peptide. The heavy chain and light chain amino acid sequences for c225v5 without the signal peptide are shown above in SEQ ID NO: 455 (heavy chain without signal peptide) and SEQ ID NO: 458 (light chain without signal peptide). In some embodiments, the activatable anti-EGFR antibody includes a combination of the amino acid sequences shown in SEQ ID NO: 455, SEQ ID NO: 458 and/or the nucleic acid and amino acid sequences shown below:

C225v5 Antibody Heavy Chain Nucleic Acid Sequence with Signal
Peptide:
(SEQ ID NO: 684)
ATGTACAGGATGCAACTCCTGTCTTGCATTGCACTAAGTCTTGCACTTGTCACGAATTCGCAGG
TGCAGCTGAAACAGAGCGGCCCGGGCCTGGTGCAGCCGAGCCAGAGCCTGAGCATTACCTGCAC
CGTGAGCGGCTTTAGCCTGACCAACTATGGCGTGCATTGGGTGCGCCAGAGCCCGGGCAAAGGC
CTGGAATGGCTGGGCGTGATTTGGAGCGGCGGCAACACCGATTATAACACCCCGTTTACCAGCC
GCCTGAGCATTAACAAAGATAACAGCAAAAGCCAGGTGTTTTTTAAAATGAACAGCCTGCAAAG
CCAGGATACCGCGATTTATTATTGCGCGCGCGCGCTGACCTATTATGATTATGAATTTGCGTAT
TGGGGCCAGGGCACCCTGGTGACCGTGAGCGCGGCTAGCACCAAGGGCCCATCGGTCTTCCCCC
TGGCACCCTCCTCCAAGAGCACCTCTGGGGGCACAGCGGCCCTGGGCTGCCTGGTCAAGGACTA
CTTCCCCGAACCGGTGACGGTGTCGTGGAACTCAGGCGCCCTGACCAGCGGCGTGCACACCTTC
CCGGCTGTCCTACAGTCCTCAGGACTCTACTCCCTCAGCAGCGTGGTGACCGTGCCCTCCAGCA
GCTTGGGCACCCAGACCTACATCTGCAACGTGAATCACAAGCCCAGCAACACCAAGGTGGACAA
GAAAGTTGAGCCCAAATCTTGTGACAAAACTCACACATGCCCACCGTGCCCAGCACCTGAACTC
CTGGGGGGACCGTCAGTCTTCCTCTTCCCCCCAAAACCCAAGGACACCCTCATGATCTCCCGGA
CCCCTGAGGTCACATGCGTGGTGGTGGACGTGAGCCACGAAGACCCTGAGGTCAAGTTCAACTG
GTACGTGGACGGCGTGGAGGTGCATAATGCCAAGACAAAGCCGCGGGAGGAGCAGTACAACAGC
ACGTACCGTGTGGTCAGCGTCCTCACCGTCCTGCACCAGGACTGGCTGAATGGCAAGGAGTACA
AGTGCAAGGTCTCCAACAAAGCCCTCCCAGCCCCCATCGAGAAAACCATCTCCAAAGCCAAAGG
GCAGCCCCGAGAACCACAGGTGTACACCCTGCCCCCATCCCGGGAGGAGATGACCAAGAACCAG
GTCAGCCTGACCTGCCTGGTCAAAGGCTTCTATCCCAGCGACATCGCCGTGGAGTGGGAGAGCA
ATGGGCAGCCGGAGAACAACTACAAGACCACGCCTCCCGTGCTGGACTCCGACGGCTCCTTCTT
CCTCTACAGCAAGCTCACCGTGGACAAGAGCAGGTGGCAGCAGGGGAACGTCTTCTCATGCTCC
GTGATGCATGAGGCTCTGCACAACCACTACACGCAGAAGAGCCTCTCCCTGTCTCCGGGTAAAT
GA
Underlined: Signal peptide
C225v5 Antibody Heavy Chain Amino Acid Sequence with Signal
Peptide:
(SEQ ID NO: 685)
MYRMQLLSCIALSLALVTNSQVQLKQSGPGLVQPSQSLSITCTVSGFSLTNYGVHWVRQSPGKG
LEWLGVIWSGGNIDYNTPFTSRLSINKDNSKSQVFFKMNSLQSQDTAIYYCARALTYYDYEFAY
WGQGTLVTVSAASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKKVEPKSCDKTHTCPPCPAPEL
LGGPSVFLFPPKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLTVDKSRWQQGNVFSCS
VMHEALHNHYTQKSLSLSPGK*
Underlined: Signal peptide
3954-2787-c225 Light Chain Nucleic Acid Sequence with Signal
Peptide:
(SEQ ID NO: 686)
ATGTACAGGATGCAACTCCTGTCTTGCATTGCACTAAGTCTTGCACTTGTCACGAATTCGCAAG
GCCAGTCTGGCCAGTGCATCTCACCTCGTGGTTGTCCGGACGGCCCATACGTCATGTACGGCTC
GAGCGGTGGCAGCGGTGGCTCTGGTGGATCCGGTACCTCCACCTCCGGCCGTTCCGCGAACCCG
CGTGGTGGCAGTAGCGGTACCCAGATCTTGCTGACCCAGAGCCCGGTGATTCTGAGCGTGAGCC
CGGGCGAACGTGTGAGCTTTAGCTGCCGCGCGAGCCAGAGCATTGGCACCAACATTCATTGGTA
TCAGCAGCGCACCAACGGCAGCCCGCGCCTGCTGATTAAATATGCGAGCGAAAGCATTAGCGGC
ATTCCGAGCCGCTTTAGCGGCAGCGGCAGCGGCACCGATTTTACCCTGAGCATTAACAGCGTGG
AAAGCGAAGATATTGCGGATTATTATTGCCAGCAGAACAACAACTGGCCGACCACCTTTGGCGC
GGGCACCAAACTGGAACTGAAACGTACGGTGGCTGCACCATCTGTCTTCATCTTCCCGCCATCT
GATGAGCAGTTGAAATCTGGAACTGCCTCTGTTGTGTGCCTGCTGAATAACTTCTATCCCAGAG
AGGCCAAAGTACAGTGGAAGGTGGATAACGCCCTCCAATCGGGTAACTCCCAGGAGAGTGTCAC
AGAGCAGGACAGCAAGGACAGCACCTACAGCCTCAGCAGCACCCTGACGCTGAGCAAAGCAGAC
TACGAGAAACACAAAGTCTACGCCTGCGAAGTCACCCATCAGGGCCTGAGCTCGCCCGTCACAA
AGAGCTTCAACAGGGGAGAGTGTTAG
Underlined: Signal Peptide
3954-2787-c225 Light Chain Amino Acid Sequence with Signal
Peptide:
(SEQ ID NO: 687)
MYRMQLLSCIALSLALVTNSQGQSGQCISPRGCPDGPYVMYGSSGGSGGSGGSGTSTSGRSANP
RGGSSGTQILLTQSPVILSVSPGERVSFSCRASQSIGTNIHWYQQRTNGSPRLLIKYASESISG
IPSRFSGSGSGTDFTLSINSVESEDIADYYCQQNNNWPTTFGAGTKLELKRTVAAPSVFIFPPS
DEQLKSGTASVVCLLNNFYPREAKVQWKVDNALQSGNSQESVTEQDSKDSTYSLSSTLTLSKAD
YEKHKVYACEVTHQGLSSPVTKSFNRGEC*
Underlined: Signal Peptide
3954-2787-c225 Light Chain Nucleic Acid Sequence
(without Signal Peptide):
(SEQ ID NO: 688)
CAAGGCCAGTCTGGCCAGTGCATCTCACCTCGTGGTTGTCCGGACGGCCCATACGTCATGTACG
GCTCGAGCGGTGGCAGCGGTGGCTCTGGTGGATCCGGTACCTCCACCTCCGGCCGTTCCGCGAA
CCCGCGTGGTGGCAGTAGCGGTACCCAGATCTTGCTGACCCAGAGCCCGGTGATTCTGAGCGTG
AGCCCGGGCGAACGTGTGAGCTTTAGCTGCCGCGCGAGCCAGAGCATTGGCACCAACATTCATT
GGTATCAGCAGCGCACCAACGGCAGCCCGCGCCTGCTGATTAAATATGCGAGCGAAAGCATTAG
CGGCATTCCGAGCCGCTTTAGCGGCAGCGGCAGCGGCACCGATTTTACCCTGAGCATTAACAGC
GTGGAAAGCGAAGATATTGCGGATTATTATTGCCAGCAGAACAACAACTGGCCGACCACCTTTG
GCGCGGGCACCAAACTGGAACTGAAACGTACGGTGGCTGCACCATCTGTCTTCATCTTCCCGCC
ATCTGATGAGCAGTTGAAATCTGGAACTGCCTCTGTTGTGTGCCTGCTGAATAACTTCTATCCC
AGAGAGGCCAAAGTACAGTGGAAGGTGGATAACGCCCTCCAATCGGGTAACTCCCAGGAGAGTG
TCACAGAGCAGGACAGCAAGGACAGCACCTACAGCCTCAGCAGCACCCTGACGCTGAGCAAAGC
AGACTACGAGAAACACAAAGTCTACGCCTGCGAAGTCACCCATCAGGGCCTGAGCTCGCCCGTC
ACAAAGAGCTTCAACAGGGGAGAGTGTTAG
3954-2787-c225 Light Chain Amino Acid Sequence
(without Signal Peptide):
(SEQ ID NO: 689)
QGQSGQCISPRGCPDGPYVMYGSSGGSGGSGGSGTSTSGRSANPRGGSSGTQILLTQSPVILSV
SPGERVSFSCRASQSIGTNIHWYQQRTNGSPRLLIKYASESISGIPSRFSGSGSGTDFTLSINS
VESEDIADYYCQQNNNWPTTFGAGTKLELKRTVAAPSVFIFPPSDEQLKSGTASVVCLLNNFYP
REAKVQWKVDNALQSGNSQESVTEQDSKDSTYSLSSTLTLSKADYEKHKVYACEVTHQGLSSPV
TKSFNRGEC*

Exemplary conjugated antibodies and/or activatable antibodies of the disclosure include, for example, antibodies that bind a Jagged target, e.g., Jagged-1, Jagged-2 and/or both Jagged-1 and Jagged-2, and that include a combination of a variable heavy chain region and a variable light chain region that are, or are derived from, the variable heavy chain and variable light chain sequences shown below.

Variable Light Chain Amino Sequence Lc4
(SEQ ID NO: 459)
DIQMTQSPSSLSASVGDRVTITCRASQSISSYLNWYQQKPGKAPKLLIYAASSLQSGVPSRFSG
SGSGTDFTLTISSLQPEDFATYYCQQSVVAPLTFGQGTKVEIKR
Variable Heavy Chain Amino Sequence Hc4
(SEQ ID NO: 460)
EVQLLESGGGLVQPGGSLRLSCAASGFTFSSYAMSWVRQAPGKGLEWVSSIEQMGWQTYYADSV
KGRFTISRDNSKNTLYLQMNSLRAEDTAVYYCAKDIGGRSAFDYWGQGTLVTVSS
Variable Light Chain Amino Sequence Lc5
(SEQ ID NO: 461)
DIQMTQSPSSLSASVGDRVTITCRASQSISSYLNWYQQKPGKAPKLLIYAASSLQSGVPSRFSG
SGSGTDFTLTISSLQPEDFATYYCQQSVVAPLTFGQGTKVEIKR
Variable Heavy Chain Amino Sequence Hc5
(SEQ ID NO: 462)
EVQLLESGGGLVQPGGSLRLSCAASGFTFSSYAMSWVRQAPGKGLEWVSSIEQMGWQTYYADSV
KGRFTISRDNSKNTLYLQMNSLRAEDTAVYYCAKSPPYHGQFDYWGQGTLVTVSS
Variable Light Chain Amino Sequence Lc7
(SEQ ID NO: 463)
DIQMTQSPSSLSASVGDRVTITCRASQSISSYLNWYQQKPGKAPKLLIYAASSLQSGVPSRFSG
SGSGTDFTLTISSLQPEDFATYYCQQSVVAPLTFGQGTKVEIKR
Variable Heavy Chain Amino Sequence Hc7
(SEQ ID NO: 464)
EVQLLESGGGLVQPGGSLRLSCAASGFTFSSYAMSWVRQAPGKGLEWVSSIEQMGWQTYYADSV
KGRFTISRDNSKNTLYLQMNSLRAEDTAVYYCAKSPPFFGQFDYWGQGTLVTVSS
Variable Light Chain Amino Sequence Lc8
(SEQ ID NO: 465)
DIQMTQSPSSLSASVGDRVTITCRASQSISSYLNWYQQKPGKAPKLLIYAASSLQSGVPSRFSG
SGSGTDFTLTISSLQPEDFATYYCQQSVVAPLTFGQGTKVEIKR
Variable Heavy Chain Amino Sequence Hc8
(SEQ ID NO: 466)
EVQLLESGGGLVQPGGSLRLSCAASGFTFSSYAMSWVRQAPGKGLEWVSSIEQMGWQTYYADSV
KGRFTISRDNSKNTLYLQMNSLRAEDTAVYYCAKHIGRTNPFDYWGQGTLVTVSS
Variable Light Chain Amino Sequence Lc13
(SEQ ID NO: 467)
DIQMTQSPSSLSASVGDRVTITCRASQSISSYLNWYQQKPGKAPKLLIYAASSLQSGVPSRFSG
SGSGTDFTLTISSLQPEDFATYYCQQSVVAPLTFGQGTKVEIKR
Variable Heavy Chain Amino Sequence Hc13
(SEQ ID NO: 468)
EVQLLESGGGLVQPGGSLRLSCAASGFTFSSYAMSWVRQAPGKGLEWVSSIEQMGWQTEYADSV
KGRFTISRDNSKNTLYLQMNSLRAEDTAVYYCAKSAAAFDYWGQGTLVTVSS
Variable Light Chain Amino Sequence Lc16
(SEQ ID NO: 469)
DIQMTQSPSSLSASVGDRVTITCRASQSISSYLNWYQQKPGKAPKLLIYAASSLQSGVPSRFSG
SGSGTDFTLTISSLQPEDFATYYCQQSVVAPLTFGQGTKVEIKR
Variable Heavy Chain Amino Sequence Hc16
(SEQ ID NO: 470)
EVQLLESGGGLVQPGGSLRLSCAASGFTFSSYAMSWVRQAPGKGLEWVSSIEQMGWQTYYADSV
KGRFTISRDNSKNTLYLQMNSLRAEDTAVYYCAKSPPYYGQFDYWGQGTLVTVSS
Variable Light Chain Amino Sequence Lc19
(SEQ ID NO: 471)
DIQMTQSPSSLSASVGDRVTITCRASQSISSYLNWYQQKPGKAPKLLIYAASSLQSGVPSRFSG
SGSGTDFTLTISSLQPEDFATYYCQQSVVAPLTFGQGTKVEIKR
Variable Heavy Chain Amino Sequence Hc19
(SEQ ID NO: 472)
EVQLLESGGGLVQPGGSLRLSCAASGFTFSSYAMSWVRQAPGKGLEWVSSIEQMGWQTYYADSV
KGRFTISRDNSKNTLYLQMNSLRAEDTAVYYCAKSPPFFGQFDYWGQGTLVTVSS
Variable Light Chain Amino Sequence Lc21
(SEQ ID NO: 473)
DIQMTQSPSSLSASVGDRVTITCRASQSISSYLNWYQQKPGKAPKLLIYAASSLQSGVPSRFSG
SGSGTDFTLTISSLQPEDFATYYCQQSVVAPLTFGQGTKVEIKR
Variable Heavy Chain Amino Sequence Hc21
(SEQ ID NO: 474)
EVQLLESGGGLVQPGGSLRLSCAASGFTFSSYAMSWVRQAPGKGLEWVSSIEQMGWQTYYADSV
KGRFTISRDNSKNTLYLQMNSLRAEDTAVYYCAKDIGGRSAFDYWGQGTLVTVSS
Variable Light Chain Amino Sequence Lc24
(SEQ ID NO: 475)
DIQMTQSPSSLSASVGDRVTITCRASQSISSYLNWYQQKPGKAPKLLIYAASSLQSGVPSRFSG
SGSGTDFTLTISSLQPEDFATYYCQQSVVAPLTFGQGTKVEIKR
Variable Heavy Chain Amino Sequence Hc24
(SEQ ID NO: 476)
EVQLLESGGGLVQPGGSLRLSCAASGFTFSSYAMSWVRQAPGKGLEWVSSIEEMGWQTLYADSV
KGRFTISRDNSKNTLYLQMNSLRAEDTAVYYCAKSAAAFDYWGQGTLVTVSS
Variable Light Chain Amino Sequence Lc26
(SEQ ID NO: 477)
DIQMTQSPSSLSASVGDRVTITCRASQSISSYLNWYQQKPGKAPKLLIYAASSLQSGVPSRFSG
SGSGTDFTLTISSLQPEDFATYYCQQSVVAPLTFGQGTKVEIKR
Variable Heavy Chain Amino Sequence Hc26
(SEQ ID NO: 478)
EVQLLESGGGLVQPGGSLRLSCAASGFTFSSYAMSWVRQAPGKGLEWVSSIEQMGWQTYYADSV
KGRFTISRDNSKNTLYLQMNSLRAEDTAVYYCAKDIGGRSAFDYWGQGTLVTVSS
Variable Light Chain Amino Sequence Lc27
(SEQ ID NO: 479)
DIQMTQSPSSLSASVGDRVTITCRASQSISSYLNWYQQKPGKAPKLLIYAASSLQSGVPSRFSG
SGSGTDFTLTISSLQPEDFATYYCQQSVVAPLTFGQGTKVEIKR
Variable Heavy Chain Amino Sequence Hc27
(SEQ ID NO: 480)
EVQLLESGGGLVQPGGSLRLSCAASGFTFSSYAMSWVRQAPGKGLEWVSSIEQMGWQTYYADSV
KGRFTISRDNSKNTLYLQMNSLRAEDTAVYYCAKSPPFYGQFDYWGQGTLVTVSS
Variable Light Chain Amino Sequence Lc28
(SEQ ID NO: 481)
DIQMTQSPSSLSASVGDRVTITCRASQSISSYLNWYQQKPGKAPKLLIYAASSLQSGVPSRFSG
SGSGTDFTLTISSLQPEDFATYYCQQSVVAPLTFGQGTKVEIKR
Variable Heavy Chain Amino Sequence Hc28
(SEQ ID NO: 482)
EVQLLESGGGLVQPGGSLRLSCAASGFTFSSYAMSWVRQAPGKGLEWVSSIEQMGWQTYYADSV
KGRFTISRDNSKNTLYLQMNSLRAEDTAVYYCAKSPPFFGQFDYWGQGTLVTVSS
Variable Light Chain Amino Sequence Lc30
(SEQ ID NO: 483)
DIQMTQSPSSLSASVGDRVTITCRASQSISSYLNWYQQKPGKAPKLLIYAASSLQSGVPSRFSG
SGSGTDFTLTISSLQPEDFATYYCQQSVVAPLTFGQGTKVEIKR
Variable Heavy Chain Amino Sequence Hc30
(SEQ ID NO: 484)
EVQLLESGGGLVQPGGSLRLSCAASGFTFSSYAMSWVRQAPGKGLEWVSSIEEMGWQTLYADSV
KGRFTISRDNSKNTLYLQMNSLRAEDTAVYYAKSAAAFDYWGQGTLVTVSS
Variable Light Chain Amino Sequence Lc31
(SEQ ID NO: 485)
DIQMTQSPSSLSASVGDRVTITCRASQSISSYLNWYQQKPGKAPKLLIYAASSLQSGVPSRFSG
SGSGTDFTLTISSLQPEDFATYYCQQSVVAPLTFGQGTKVEIKR
Variable Heavy Chain Amino Sequence Hc31
(SEQ ID NO: 486)
EVQLLESGGGLVQPGGSLRLSCAASGFTFSSYAMSWVRQAPGKGLEWVSSIEQMGWQTYYADSV
KGRFTISRDNSKNTLYLQMNSLRAEDTAVYYCAKDIGGRSAFDYWGQGTLVTVSS
Variable Light Chain Amino Sequence Lc32
(SEQ ID NO: 487)
DIQMTQSPSSLSASVGDRVTITCRASQSISSYLNWYQQKPGKAPKLLIYAASSLQSGVPSRFSG
SGSGTDFTLTISSLQPEDFATYYCQQSVVAPLTFGQGTKVEIKR
Variable Heavy Chain Amino Sequence Hc32
(SEQ ID NO: 488)
EVQLLESGGGLVQPGGSLRLSCAASGFTFSSYAMSWVRQAPGKGLEWVSSIDPEGWQTYYADSV
KGRFTISRDNSKNTLYLQMNSLRAEDTAVYYCAKSAAAFDYWGQGTLVTVSS
Variable Light Chain Amino Sequence Lc37
(SEQ ID NO: 489)
DIQMTQSPSSLSASVGDRVTITCRASQSISSYLNWYQQKPGKAPKLLIYAASSLQSGVPSRFSG
SGSGTDFTLTISSLQPEDFATYYCQQSVVAPLTFGQGTKVEIKR
Variable Heavy Chain Amino Sequence Hc37
(SEQ ID NO: 490)
EVQLLESGGGLVQPGGSLRLSCAASGFTFSSYAMSWVRQAPGKGLEWVSSIEQMGWQTYYADSV
KGRFTISRDNSKNTLYLQMNSLRAEDTAVYYCAKSPPHNGQFDYWGQGTLVTVSS
Variable Light Chain Amino Sequence Lc39
(SEQ ID NO: 491)
DIQMTQSPSSLSASVGDRVTITCRASQSISSYLNWYQQKPGKAPKLLIYAASSLQSGVPSRFSG
SGSGTDFTLTISSLQPEDFATYYCQQSVVAPLTFGQGTKVEIKR
Variable Heavy Chain Amino Sequence Hc39
(SEQ ID NO: 492)
EVQLLESGGGLVQPGGSLRLSCAASGFTFSSYAMSWVRQAPGKGLEWVSSIEQMGWQTEYADSV
KGRFTISRDNSKNTLYLQMNSLRAEDTAVYYCAKSAAAFDYWGQGTLVTVSS
Variable Light Chain Amino Sequence Lc40
(SEQ ID NO: 493)
DIQMTQSPSSLSASVGDRVTITCRASQSISSYLNWYQQKPGKAPKLLIYAASSLQSGVPSRFSG
SGSGTDFTLTISSLQPEDFATYYCQQSVVAPLTFGQGTKVEIKR
Heavy Chain Amino Sequence Hc40
(SEQ ID NO: 494)
EVQLLESGGGLVQPGGSLRLSCAASGFTFSSYAMSWVRQAPGKGLEWVSSIEQMGWQTYYADSV
KGRFTISRDNSKNTLYLQMNSLRAEDTAVYYCAKSPPFFGQFDYWGQGTLVTVSS
Variable Light Chain Amino Sequence Lc47
(SEQ ID NO: 495)
DIQMTQSPSSLSASVGDRVTITCRASQSISSYLNWYQQKPGKAPKLLIYAASSLQSGVPSRFSG
SGSGTDFTLTISSLQPEDFATYYCQQSVVAPLTFGQGTKVEIKR
Variable Heavy Chain Amino Sequence Hc47
(SEQ ID NO: 496)
EVQLLESGGGLVQPGGSLRLSCAASGFTFSSYAMSWVRQAPGKGLEWVSSIDEMGWQTEYADSV
KGRFTISRDNSKNTLYLQMNSLRAEDTAVYYCAKSAAAFDYWGQGTLVTVSS
Variable 4B2 Light Chain
(SEQ ID NO: 497)
DIQMTQSPSSLSASVGDRVTITCRASQSISSYLNWYQQKPGKAPKLLIYAASSLQSGVPSRFSG
SGSGTDFTLTISSLQPEDFATYYCQQTLDAPPQFGQGTKVEIKR
Variable 4B2 Heavy Chain
(SEQ ID NO: 498)
EVQLLESGGGLVQPGGSLRLSCAASGFTFSSYAMSWVRQAPGKGLEWVSSIEQMGWQTYYADSV
KGRFTISRDNSKNTLYLQMNSLRAEDTAVYYCAKDIGGRSAFDYWGQGTLVTVSS
Variable 4D11 Light Chain
(SEQ ID NO: 499)
DIQMTQSPSSLSASVGDRVTITCRASQSISSYLNWYQQKPGKAPKLLIYAASSLQSGVPSRFSG
SGSGTDFTLTISSLQPEDFATYYCQQTVVAPPLFGQGTKVEIKR
Variable 4D11 Heavy Chain
(SEQ ID NO: 500)
EVQLLESGGGLVQPGGSLRLSCAASGFTFSSYAMSWVRQAPGKGLEWVSSIDPEGRQTYYADSV
KGRFTISRDNSKNTLYLQMNSLRAEDTAVYYCAKDIGGRSAFDYWGQGTLVTVSS
Variable 4E7 Light Chain
(SEQ ID NO: 501)
DIQMTQSPSSLSASVGDRVTITCRASQSISSYLNWYQQKPGKAPKLLIYAASSLQSGVPSRFSG
SGSGTDFTLTISSLQPEDFATYYCQQSLVAPLTFGQGTKVEIKR
Variable 4E7 Heavy Chain
(SEQ ID NO: 502)
EVQLLESGGGLVQPGGSLRLSCAASGFTFSSYAMSWVRQAPGKGLEWVSSIEEMGWQTKYADSV
KGRFTISRDNSKNTLYLQMNSLRAEDTAVYYCAKSAAAFDYWGQGTLVTVSS
Variable 4E11 Light Chain
(SEQ ID NO: 503)
DIQMTQSPSSLSASVGDRVTITCRASQSISSYLNWYQQKPGKAPKLLIYAASSLQSGVPSRFSG
SGSGTDFTLTISSLQPEDFATYYCQQALDAPLMFGQGTKVEIKR
Variable 4E11 Heavy Chain
(SEQ ID NO: 504)
EVQLLESGGGLVQPGGSLRLSCAASGFTFSSYAMSWVRQAPGKGLEWVSSIEPMGQLTEYADSV
KGRFTISRDNSKNTLYLQMNSLRAEDTAVYYCAKDIGGRSAFDYWGQGTLVTVSS
Variable 6B7 Light Chain
(SEQ ID NO: 505)
DIQMTQSPSSLSASVGDRVTITCRASQSISSYLNWYQQKPGKAPKLLIYAASSLQSGVPSRFSG
SGSGTDFTLTISSLQPEDFATYYCQQALVAPLTFGQGTKVEIKR
Variable 6B7 Heavy Chain
(SEQ ID NO: 506)
EVQLLESGGGLVQPGGSLRLSCAASGFTFSSYAMSWVRQAPGKGLEWVSSIDEMGWQTYYADSV
KGRFTISRDNSKNTLYLQMNSLRAEDTAVYYCAKSAAAFDYWGQGTLVTVSS
Variable 6F8 Light Chain
(SEQ ID NO: 507)
DIQMTQSPSSLSASVGDRVTITCRASQSISSYLNWYQQKPGKAPKLLIYAASSLQSGVPSRFSG
SGSGTDFTLTISSLQPEDFATYYCQQALVAPLTFGQGTKVEIKR
Variable 6F8 Heavy Chain
(SEQ ID NO: 508)
EVQLLESGGGLVQPGGSLRLSCAASGFTFSSYAMSWVRQAPGKGLEWVSSIDEMGWQTYYADSV
KGRFTISRDNSKNTLYLQMNSLRAEDTAVYYCAKSAAAFDYWGQGTLVTVSS

Exemplary conjugated antibodies and/or activatable antibodies of the disclosure include, for example, antibodies that bind a Jagged target, e.g., Jagged-1, Jagged-2 and/or both Jagged-1 and Jagged-2, and that include a combination of a heavy chain region and a light chain region that are, or are derived from, the heavy chain and light chain sequences shown below.

4D11 Light Chain sequence:
(SEQ ID NO: 509)
DIQMTQSPSSLSASVGDRVTITCRASQSISSYLNWYQQKPGKAPKLLIYA
ASSLQSGVPSRFSGSGSGTDFTLTISSLQPEDFATYYCQQTVVAPPLFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
4D11 Heavy Chain sequence:
(SEQ ID NO: 510)
EVQLLESGGGLVQPGGSLRLSCAASGFTFSSYAMSWVRQAPGKGLEWVSS
IDPEGRQTYYADSVKGRFTISRDNSKNTLYLQMNSLRAEDTAVYYCAKDI
GGRSAFDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK
4D11v2 Heavy Chain sequence
(SEQ ID NO: 511)
EVHLLESGGGLVQPGGSLRLSCAASGFTFSSYAMSWVRQAPGKGLEWVSS
IDPEGRQTYYADSVKGRFTISRDNSKNTLYLQMNSLRAEDTAVYYCAKDI
GGRSAFDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK
4D11v2 Light Chain Sequence
(SEQ ID NO: 512)
DIQMTQSPSSLSASVGDRVTITCRASQSISSYLNWYQQKPGKAPKLLIYA
ASSLQSGVPSRFSGSGSGTDFTLTISSLQPEDFATYYCQQTVVAPPLFGQ
GTKVEIKRTVAAPSVFIFPPSDEQLKSGTASVVCLLNNFYPREAKVQWKV
DNALQSGNSQESVTEQDSKDSTYSLSSTLTLXKADYEKHKVYACEVTHQG
LSSPVTKSFNRGEC

The activatable antibodies provided herein contain at least an antibody or antibody fragment thereof (collectively referred to as AB throughout the disclosure) that specifically binds a target, e.g., a human target, wherein the AB is modified by a masking moiety (MM).

In some embodiments, the masking moiety is selected for use with a specific antibody or antibody fragment. For example, suitable masking moieties for use with antibodies that bind EGFR include MMs that include the sequence CISPRG (SEQ ID NO: 513). By way of non-limiting examples, the MM can include a sequence such as CISPRGC (SEQ ID NO: 690) CISPRGCG (SEQ ID NO: 514); CISPRGCPDGPYVMY (SEQ ID NO: 515); CISPRGCPDGPYVM (SEQ ID NO: 516), CISPRGCEPGTYVPT (SEQ ID NO: 517) and CISPRGCPGQIWHPP (SEQ ID NO: 518). Other suitable masking moieties include any of the EGFR-specific masks disclosed in PCT Publication No. WO 2010/081173, such as, by way of non-limiting example, GSHCLIPINMGAPSC (SEQ ID NO: 519); CISPRGCGGSSASQSGQGSHCLIPINMGAPSC (SEQ ID NO: 520); CNHHYFYTCGCISPRGCPG (SEQ ID NO: 521); ADHVFWGSYGCISPRGCPG (SEQ ID NO: 522); CHHVYWGHCGCISPRGCPG (SEQ ID NO: 523); CPHFTTTSCGCISPRGCPG (SEQ ID NO: 524); CNHHYHYYCGCISPRGCPG (SEQ ID NO: 525); CPHVSFGSCGCISPRGCPG (SEQ ID NO: 526); CPYYTLSYCGCISPRGCPG (SEQ ID NO: 527); CNHVYFGTCGCISPRGCPG (SEQ ID NO: 528); CNHFTLTTCGCISPRGCPG (SEQ ID NO: 529); CHHFTLTTCGCISPRGCPG (SEQ ID NO: 530); YNPCATPMCCISPRGCPG (SEQ ID NO: 531); CNHHYFYTCGCISPRGCG (SEQ ID NO: 532); CNHHYHYYCGCISPRGCG (SEQ ID NO: 533); CNHVYFGTCGCISPRGCG (SEQ ID NO: 534); CHHVYWGHCGCISPRGCG (SEQ ID NO: 535); CPHFTTTSCGCISPRGCG (SEQ ID NO: 536); CNHFTLTTCGCISPRGCG (SEQ ID NO: 537); CHHFTLTTCGCISPRGCG (SEQ ID NO: 538); CPYYTLSYCGCISPRGCG (SEQ ID NO: 539); CPHVSFGSCGCISPRGCG (SEQ ID NO: 540); ADHVFWGSYGCISPRGCG (SEQ ID NO: 541); YNPCATPMCCISPRGCG (SEQ ID NO: 542); CHHVYWGHCGCISPRGCG (SEQ ID NO: 543); C(N/P)H(H/V/F)(Y/T)(F/W/T/L)(Y/G/T/S)(T/S/Y/H)CGCISPRGCG (SEQ ID NO: 544); CISPRGCGQPIPSVK (SEQ ID NO: 545); CISPRGCTQPYHVSR (SEQ ID NO: 546); and/or CISPRGCNAVSGLGS (SEQ ID NO: 547).

Suitable masking moieties for use with antibodies that bind a Jagged target, e.g., Jagged 1 and/or Jagged 2, include, by way of non-limiting example, masking moieties that include a sequence such as QGQSGQCNIWLVGGDCRGWQG (SEQ ID NO: 691); QGQSGQGQQQWCNIWINGGDCRGWNG (SEQ ID NO: 548); PWCMQRQDFLRCPQP (SEQ ID NO: 549); QLGLPAYMCTFECLR (SEQ ID NO: 550); CNLWVSGGDCGGLQG (SEQ ID NO: 551); SCSLWTSGSCLPHSP (SEQ ID NO: 552); YCLQLPHYMQAMCGR (SEQ ID NO: 553); CFLYSCTDVSYWNNT (SEQ ID NO: 554); PWCMQRQDYLRCPQP (SEQ ID NO: 555); CNLWISGGDCRGLAG (SEQ ID NO: 556); CNLWVSGGDCRGVQG (SEQ ID NO: 557); CNLWVSGGDCRGLRG (SEQ ID NO: 558); CNLWISGGDCRGLPG (SEQ ID NO: 559); CNLWVSGGDCRDAPW (SEQ ID NO: 560); CNLWVSGGDCRDLLG (SEQ ID NO: 561); CNLWVSGGDCRGLQG (SEQ ID NO: 562); CNLWLHGGDCRGWQG (SEQ ID NO: 563); CNIWLVGGDCRGWQG (SEQ ID NO: 564); CTTWFCGGDCGVMRG (SEQ ID NO: 565); CNIWGPSVDCGALLG (SEQ ID NO: 566); CNIWVNGGDCRSFEG (SEQ ID NO: 567); YCLNLPRYMQDMCWA (SEQ ID NO: 568); YCLALPHYMQADCAR (SEQ ID NO: 569); CFLYSCGDVSYWGSA (SEQ ID NO: 570); CYLYSCTDSAFWNNR (SEQ ID NO: 571); CYLYSCNDVSYWSNT (SEQ ID NO: 572); CFLYSCTDVSYW (SEQ ID NO: 573); CFLYSCTDVAYWNSA (SEQ ID NO: 574); CFLYSCTDVSYWGDT (SEQ ID NO: 575); CFLYSCTDVSYWGNS (SEQ ID NO: 576); CFLYSCTDVAYWNNT (SEQ ID NO: 577); CFLYSCGDVSYWGNPGLS (SEQ ID NO: 578); CFLYSCTDVAYWSGL (SEQ ID NO: 579); CYLYSCTDGSYWNST (SEQ ID NO: 580); CFLYSCSDVSYWGNI (SEQ ID NO: 581); CFLYSCTDVAYW (SEQ ID NO: 582); CFLYSCTDVSYWGST (SEQ ID NO: 583); CFLYSCTDVAYWGDT (SEQ ID NO: 584); GCNIWLNGGDCRGWVDPLQG (SEQ ID NO: 585); GCNIWLVGGDCRGWIGDTNG (SEQ ID NO: 586); GCNIWLVGGDCRGWIEDSNG (SEQ ID NO: 587); GCNIWANGGDCRGWIDNIDG (SEQ ID NO: 588); GCNIWLVGGDCRGWLGEAVG (SEQ ID NO: 589); GCNIWLVGGDCRGWLEEAVG (SEQ ID NO: 590); GGPALCNIWLNGGDCRGWSG (SEQ ID NO: 591); GAP VFCNIWLNGGDCRGWMG (SEQ ID NO: 592); GQQQWCNIWINGGDCRGWNG (SEQ ID NO: 593); GKSEFCNIWLNGGDCRGWIG (SEQ ID NO: 594); GTPGGCNIWANGGDCRGWEG (SEQ ID NO: 595); GASQYCNLWINGGDCRGWRG (SEQ ID NO: 596); GCNIWLVGGDCRPWVEGG (SEQ ID NO: 597); GCNIWAVGGDCRPFVDGG (SEQ ID NO: 598); GCNIWLNGGDCRAWVDTG (SEQ ID NO: 599); GCNIWIVGGDCRPFINDG (SEQ ID NO: 600); GCNIWLNGGDCRPVVFGG (SEQ ID NO: 601); GCNIWLSGGDCRMFMNEG (SEQ ID NO: 602); GCNIWVNGGDCRSFVYSG (SEQ ID NO: 603); GCNIWLNGGDCRGWEASG (SEQ ID NO: 604); GCNIWAHGGDCRGFIEPG (SEQ ID NO: 605); GCNIWLNGGDCRTFVASG (SEQ ID NO: 606); GCNIWAHGGDCRGFIEPG (SEQ ID NO: 607); GFLENCNIWLNGGDCRTG (SEQ ID NO: 608); GIYENCNIWLNGGDCRMG (SEQ ID NO: 609); and/or GIPDNCNIWINGGDCRYG (SEQ ID NO: 610).

Suitable masking moieties for use with antibodies that bind an interleukin 6 target, e.g., interleukin 6 receptor (IL-6R), include, by way of non-limiting example, masking moieties that include a sequence such as QGQSGQYGSCSWNYVHIFMDC (SEQ ID NO: 611); QGQSGQGDFDIPFPAHWVPIT (SEQ ID NO: 612); QGQSGQMGVPAGCVWNYAHIFMDC (SEQ ID NO: 613); YRSCNWNYVSIFLDC (SEQ ID NO: 614); PGAFDIPFPAHWVPNT (SEQ ID NO: 615); ESSCVWNYVHIYMDC (SEQ ID NO: 616); YPGCKWNYDRIFLDC (SEQ ID NO: 617); YRTCSWNYVGIFLDC (SEQ ID NO: 618); YGSCSWNYVHIFMDC (SEQ ID NO: 619); YGSCSWNYVHIFLDC (SEQ ID NO: 620); YGSCNWNYVHIFLDC (SEQ ID NO: 621); YTSCNWNYVHIFMDC (SEQ ID NO: 622); YPGCKWNYDRIFLDC (SEQ ID NO: 623); WRSCNWNYAHIFLDC (SEQ ID NO: 624); WSNCHWNYVHIFLDC (SEQ ID NO: 625); DRSCTWNYVRISYDC (SEQ ID NO: 626); SGSCKWDYVHIFLDC (SEQ ID NO: 627); SRSCIWNYAHIHLDC (SEQ ID NO: 628); SMSCYWQYERIFLDC (SEQ ID NO: 629); YRSCNWNYVSIFLDC (SEQ ID NO: 630); SGSCKWDYVHIFLDC (SEQ ID NO: 631); YKSCHWDYVHIFLDC (SEQ ID NO: 632); YGSCTWNYVHIFMEC (SEQ ID NO: 633); FSSCNWNYVHIFLDC (SEQ ID NO: 634); WRSCNWNYAHIFLDC (SEQ ID NO: 635); YGSCQWNYVHIFLDC (SEQ ID NO: 636); YRSCNWNYVHIFLDC (SEQ ID NO: 637); NMSCHWDYVHIFLDC (SEQ ID NO: 638); FGPCTWNYARISWDC (SEQ ID NO: 639); XXsCXWXYvhIfXdC (SEQ ID NO: 640); MGVPAGCVWNYAHIFMDC (SEQ ID NO: 641); RDTGGQCRWDYVHIFMDC (SEQ ID NO: 642); AGVPAGCTWNYVHIFMEC (SEQ ID NO: 643); VGVPNGCVWNYAHIFMEC (SEQ ID NO: 644); DGGPAGCSWNYVHIFMEC (SEQ ID NO: 645); AVGPAGCWWNYVHIFMEC (SEQ ID NO: 646); CTWNYVHIFMDCGEGEGP (SEQ ID NO: 647); GGVPEGCTWNYAHIFMEC (SEQ ID NO: 648); AEVPAGCWWNYVHIFMEC (SEQ ID NO: 649); AGVPAGCTWNYVHIFMEC (SEQ ID NO: 650); SGASGGCKWNYVHIFMDC (SEQ ID NO: 651); TPGCRWNYVHIFMECEAL (SEQ ID NO: 652); VGVPNGCVWNYAHIFMEC (SEQ ID NO: 653); PGAFDIPFPAHWVPNT (SEQ ID NO: 654); RGACDIPFPAHWIPNT (SEQ ID NO: 655); QGDFDIPFPAHWVPIT (SEQ ID NO: 656); XGafDIPFPAHWvPnT (SEQ ID NO: 657); RGDGNDSDIPFPAHWVPRT (SEQ ID NO: 658); SGVGRDRDIPFPAHWVPRT (SEQ ID NO: 659); WAGGNDCDIPFPAHWIPNT (SEQ ID NO: 660); WGDGMDVDIPFPAHWVPVT (SEQ ID NO: 661); AGSGNDSDIPFPAHWVPRT (SEQ ID NO: 662); ESRSGYADIPFPAHWVPRT (SEQ ID NO: 663); and/or RECGRCGDIPFPAHWVPRT (SEQ ID NO: 664).

When the AB is modified with a MM and is in the presence of the target, specific binding of the AB to its target is reduced or inhibited, as compared to the specific binding of the AB not modified with an MM or the specific binding of the parental AB to the target.

The K_{d} of the AB modified with a MM towards the target is at least 5, 10, 25, 50, 100, 250, 500, 1,000, 2,500, 5,000, 10,000, 50,000, 100,000, 500,000, 1,000,000, 5,000,000, 10,000,000, 50,000,000 or greater, or between 5-10, 10-100, 10-1,000, 10-10,000, 10-100,000, 10-1,000,000, 10-10,000,000, 100-1,000, 100-10,000, 100-100,000, 100-1,000,000, 100-10,000,000, 1,000-10,000, 1,000-100,000, 1,000-1,000,000, 1,000-10,000,000, 10,000-100,000, 10,000-1,000,000, 10,000-10,000,000, 100,000-1,000,000, or 100,000-10,000,000 times greater than the K_{d} of the AB not modified with an MM or of the parental AB towards the target. Conversely, the binding affinity of the AB modified with a MM towards the target is at least 2, 3, 4, 5, 10, 25, 50, 100, 250, 500, 1,000, 2,500, 5,000, 10,000, 50,000, 100,000, 500,000, 1,000,000, 5,000,000, 10,000,000, 50,000,000 or greater, or between 5-10, 10-100, 10-1,000, 10-10,000, 10-100,000, 10-1,000,000, 10-10,000,000, 100-1,000, 100-10,000, 100-100,000, 100-1,000,000, 100-10,000,000, 1,000-10,000, 1,000-100,000, 1,000-1,000,000, 1,000-10,000,000, 10,000-100,000, 10,000-1,000,000, 10,000-10,000,000, 100,000-1,000,000, or 100,000-10,000,000 times lower than the binding affinity of the AB not modified with an MM or of the parental AB towards the target.

The dissociation constant (K_{d}) of the MM towards the AB is generally greater than the K_{d} of the AB towards the target. The K_{d} of the MM towards the AB can be at least 5, 10, 25, 50, 100, 250, 500, 1,000, 2,500, 5,000, 10,000, 100,000, 1,000,000 or even 10,000,000 times greater than the K_{d} of the AB towards the target. Conversely, the binding affinity of the MM towards the AB is generally lower than the binding affinity of the AB towards the target. The binding affinity of MM towards the AB can be at least 5, 10, 25, 50, 100, 250, 500, 1,000, 2,500, 5,000, 10,000, 100,000, 1,000,000 or even 10,000,000 times lower than the binding affinity of the AB towards the target.

When the AB is modified with a MM and is in the presence of the target specific binding of the AB to its target is reduced or inhibited, as compared to the specific binding of the AB not modified with an MM or the specific binding of the parental AB to the target. When compared to the binding of the AB not modified with an MM or the binding of the parental AB to the target the AB's ability to bind the target when modified with an MM can be reduced by at least 50%, 60%, 70%, 80%, 90%, 92%, 93%, 94%, 95%, 96%, 97%, 98%, 99% and even 100% for at least 2, 4, 6, 8, 12, 28, 24, 30, 36, 48, 60, 72, 84, or 96 hours, or 5, 10, 15, 30, 45, 60, 90, 120, 150, or 180 days, or 1, 2, 3, 4, 5, 6, 7, 8, 9, 10, 11, or 12 months or more when measured in vivo or in an in vitro assay.

The MM inhibits the binding of the AB to the target. The MM binds the antigen binding domain of the AB and inhibits binding of the AB to the target. The MM can sterically inhibit the binding of the AB to the target. The MM can allosterically inhibit the binding of the AB to its target. In these embodiments when the AB is modified or coupled to a MM and in the presence of target there is no binding or substantially no binding of the AB to the target, or no more than 0.001%, 0.01%, 0.1%, 1%, 2%, 3%, 4%, 5%, 6%, 7%, 8%, 9%, 10%, 15%, 20%, 25%, 30%, 35%, 40%, or 50% binding of the AB to the target, as compared to the binding of the AB not modified with an MM, the parental AB, or the AB not coupled to an MM to the target, for at least 2, 4, 6, 8, 12, 28, 24, 30, 36, 48, 60, 72, 84, or 96 hours, or 5, 10, 15, 30, 45, 60, 90, 120, 150, or 180 days, or 1, 2, 3, 4, 5, 6, 7, 8, 9, 10, 11, or 12 months or longer when measured in vivo or in an in vitro assay.

When an AB is coupled to or modified by a MM, the MM ‘masks’ or reduces or otherwise inhibits the specific binding of the AB to the target. When an AB is coupled to or modified by a MM, such coupling or modification can effect a structural change that reduces or inhibits the ability of the AB to specifically bind its target.

An AB coupled to or modified with an MM can be represented by the following formulae (in order from an amino (N) terminal region to carboxyl (C) terminal region:
(MM)-(AB)
(AB)-(MM)
(MM)-L-(AB)
(AB)-L-(MM)
   where MM is a masking moiety, the AB is an antibody or antibody fragment thereof, and the L is a linker. In many embodiments, it may be desirable to insert one or more linkers, e.g., flexible linkers, into the composition so as to provide for flexibility.

In certain embodiments, the MM is not a natural binding partner of the AB. In some embodiments, the MM contains no or substantially no homology to any natural binding partner of the AB. In some embodiments, the MM is no more than 5%, 10%, 15%, 20%, 25%, 30%, 35%, 40%, 45%, 50%, 55%, 60%, 65%, 70%, 75%, or 80% similar to any natural binding partner of the AB. In some embodiments, the MM is no more than 5%, 10%, 15%, 20%, 25%, 30%, 35%, 40%, 45%, 50%, 55%, 60%, 65%, 70%, 75%, or 80% identical to any natural binding partner of the AB. In some embodiments, the MM is no more than 25% identical to any natural binding partner of the AB. In some embodiments, the MM is no more than 50% identical to any natural binding partner of the AB. In some embodiments, the MM is no more than 20% identical to any natural binding partner of the AB. In some embodiments, the MM is no more than 10% identical to any natural binding partner of the AB.

In some embodiments, the activatable antibodies include an AB that is modified by an MM and also includes one or more cleavable moieties (CM). Such activatable antibodies exhibit activatable/switchable binding, to the AB's target. Activatable antibodies generally include an antibody or antibody fragment (AB), modified by or coupled to a masking moiety (MM) and a modifiable or cleavable moiety (CM). In some embodiments, the CM contains an amino acid sequence that serves as a substrate for at least one protease selected from matriptase and uPA.

The elements of the activatable antibodies are arranged so that the MM and CM are positioned such that in a cleaved (or relatively active) state and in the presence of a target, the AB binds a target while in an uncleaved (or relatively inactive) state in the presence of the target, specific binding of the AB to its target is reduced or inhibited. The specific binding of the AB to its target can be reduced due to the inhibition or masking of the AB's ability to specifically bind its target by the MM.

The K_{d} of the AB modified with a MM and a CM towards the target is at least 5, 10, 25, 50, 100, 250, 500, 1,000, 2,500, 5,000, 10,000, 50,000, 100,000, 500,000, 1,000,000, 5,000,000, 10,000,000, 50,000,000 or greater, or between 5-10, 10-100, 10-1,000, 10-10,000, 10-100,000, 10-1,000,000, 10-10,000,000, 100-1,000, 100-10,000, 100-100,000, 100-1,000,000, 100-10,000,000, 1,000-10,000, 1,000-100,000, 1,000-1,000,000, 1,000-10,000,000, 10,000-100,000, 10,000-1,000,000, 10,000-10,000,000, 100,000-1,000,000, or 100,000-10,000,000 times greater than the K_{d} of the AB not modified with an MM and a CM or of the parental AB towards the target. Conversely, the binding affinity of the AB modified with a MM and a CM towards the target is at least 5, 10, 25, 50, 100, 250, 500, 1,000, 2,500, 5,000, 10,000, 50,000, 100,000, 500,000, 1,000,000, 5,000,000, 10,000,000, 50,000,000 or greater, or between 5-10, 10-100, 10-1,000, 10-10,000, 10-100,000, 10-1,000,000, 10-10,000,000, 100-1,000, 100-10,000, 100-100,000, 100-1,000,000, 100-10,000,000, 1,000-10,000, 1,000-100,000, 1,000-1,000,000, 1,000-10,000,000, 10,000-100,000, 10,000-1,000,000, 10,000-10,000,000, 100,000-1,000,000, or 100,000-10,000,000 times lower than the binding affinity of the AB not modified with an MM and a CM or of the parental AB towards the target.

When the AB is modified with a MM and a CM and is in the presence of the target but not in the presence of a modifying agent (for example at least one protease selected from matriptase and uPA), specific binding of the AB to its target is reduced or inhibited, as compared to the specific binding of the AB not modified with an MM and a CM or of the parental AB to the target. When compared to the binding of the parental AB or the binding of an AB not modified with an MM and a CM to its target, the AB's ability to bind the target when modified with an MM and a CM can be reduced by at least 50%, 60%, 70%, 80%, 90%, 92%, 93%, 94%, 95%, 96%, 97%, 98%, 99% and even 100% for at least 2, 4, 6, 8, 12, 28, 24, 30, 36, 48, 60, 72, 84, or 96 hours or 5, 10, 15, 30, 45, 60, 90, 120, 150, or 180 days, or 1, 2, 3, 4, 5, 6, 7, 8, 9, 10, 11, or 12 months or longer when measured in vivo or in an in vitro assay.

As used herein, the term cleaved state refers to the condition of the activatable antibodies following modification of the CM by at least one protease selected from matriptase and uPA. The term uncleaved state, as used herein, refers to the condition of the activatable antibodies in the absence of cleavage of the CM by a protease selected from matriptase and uPA. As discussed above, the term “activatable antibodies” is used herein to refer to an activatable antibody in both its uncleaved (native) state, as well as in its cleaved state. It will be apparent to the ordinarily skilled artisan that in some embodiments a cleaved activatable antibody may lack an MM due to cleavage of the CM by protease, resulting in release of at least the MM (e.g., where the MM is not joined to the activatable antibodies by a covalent bond (e.g., a disulfide bond between cysteine residues).

By activatable or switchable is meant that the activatable antibody exhibits a first level of binding to a target when in a inhibited, masked or uncleaved state (i.e., a first conformation), and a second level of binding to the target in the uninhibited, unmasked and/or cleaved state (i.e., a second conformation), where the second level of target binding is greater than the first level of binding. In general, the access of target to the AB of the activatable antibody is greater in the presence of a cleaving agent capable of cleaving the CM, i.e., a protease selected from matriptase and uPA, than in the absence of such a cleaving agent. Thus, when the activatable antibody is in the uncleaved state, the AB is inhibited from target binding and can be masked from target binding (i.e., the first conformation is such the AB cannot bind the target), and in the cleaved state the AB is not inhibited or is unmasked to target binding.

The CM and AB of the activatable antibodies are selected so that the AB represents a binding moiety for a given target, and the CM represents a substrate for a protease selected from matriptase and uPA. In some embodiments, the protease is co-localized with the target at a treatment site or diagnostic site in a subject. As used herein, co-localized refers to being at the same site or relatively close nearby. In some embodiments, a protease cleaves a CM yielding an activated antibody that binds to a target located nearby the cleavage site. The activatable antibodies disclosed herein find particular use where, for example, a protease capable of cleaving a site in the CM, i.e., a protease selected from matriptase and uPA, is present at relatively higher levels in target-containing tissue of a treatment site or diagnostic site than in tissue of non-treatment sites (for example in healthy tissue). In some embodiments, a CM of the disclosure is also cleaved by one or more other proteases. In some embodiments, it is the one or more other proteases that is co-localized with the target and that is responsible for cleavage of the CM in vivo.

In some embodiments activatable antibodies provide for reduced toxicity and/or adverse side effects that could otherwise result from binding of the AB at non-treatment sites if the AB were not masked or otherwise inhibited from binding to the target.

In general, an activatable antibody can be designed by selecting an AB of interest and constructing the remainder of the activatable antibody so that, when conformationally constrained, the MM provides for masking of the AB or reduction of binding of the AB to its target. Structural design criteria can be to be taken into account to provide for this functional feature.

Activatable antibodies exhibiting a switchable phenotype of a desired dynamic range for target binding in an inhibited versus an uninhibited conformation are provided. Dynamic range generally refers to a ratio of (a) a maximum detected level of a parameter under a first set of conditions to (b) a minimum detected value of that parameter under a second set of conditions. For example, in the context of an activatable antibody, the dynamic range refers to the ratio of (a) a maximum detected level of target protein binding to an activatable antibody in the presence of at least one protease selected from matriptase and uPA capable of cleaving the CM of the activatable antibodies to (b) a minimum detected level of target protein binding to an activatable antibody in the absence of the protease. The dynamic range of an activatable antibody can be calculated as the ratio of the equilibrium dissociation constant of an activatable antibody cleaving agent (e.g., enzyme) treatment to the equilibrium dissociation constant of the activatable antibodies cleaving agent treatment. The greater the dynamic range of an activatable antibody, the better the switchable phenotype of the activatable antibody. Activatable antibodies having relatively higher dynamic range values (e.g., greater than 1) exhibit more desirable switching phenotypes such that target protein binding by the activatable antibodies occurs to a greater extent (e.g., predominantly occurs) in the presence of a cleaving agent (e.g., enzyme) capable of cleaving the CM of the activatable antibodies than in the absence of a cleaving agent.

Activatable antibodies can be provided in a variety of structural configurations. Exemplary formulae for activatable antibodies are provided below. It is specifically contemplated that the N- to C-terminal order of the AB, MM and CM may be reversed within an activatable antibody. It is also specifically contemplated that the CM and MM may overlap in amino acid sequence, e.g., such that the CM is contained within the MM.

For example, activatable antibodies can be represented by the following formula (in order from an amino (N) terminal region to carboxyl (C) terminal region:
(MM)-(CM)-(AB)
(AB)-(CM)-(MM)
   where MM is a masking moiety, CM is a cleavable moiety, and AB is an antibody or fragment thereof. It should be noted that although MM and CM are indicated as distinct components in the formulae above, in all exemplary embodiments (including formulae) disclosed herein it is contemplated that the amino acid sequences of the MM and the CM could overlap, e.g., such that the CM is completely or partially contained within the MM. In addition, the formulae above provide for additional amino acid sequences that may be positioned N-terminal or C-terminal to the activatable antibodies elements.

In certain embodiments, the MM is not a natural binding partner of the AB. In some embodiments, the MM contains no or substantially no homology to any natural binding partner of the AB. In some embodiments, the MM is no more than 5%, 10%, 15%, 20%, 25%, 30%, 35%, 40%, 45%, 50%, 55%, 60%, 65%, 70%, 75%, or 80% similar to any natural binding partner of the AB. In some embodiments, the MM is no more than 5%, 10%, 15%, 20%, 25%, 30%, 35%, 40%, 45%, 50%, 55%, 60%, 65%, 70%, 75%, or 80% identical to any natural binding partner of the AB. In some embodiments, the MM is no more than 50% identical to any natural binding partner of the AB. In some embodiments, the MM is no more than 25% identical to any natural binding partner of the AB. In some embodiments, the MM is no more than 20% identical to any natural binding partner of the AB. In some embodiments, the MM is no more than 10% identical to any natural binding partner of the AB.

In many embodiments it may be desirable to insert one or more linkers, e.g., flexible linkers, into the activatable antibody construct so as to provide for flexibility at one or more of the MM-CM junction, the CM-AB junction, or both. For example, the AB, MM, and/or CM may not contain a sufficient number of residues (e.g., Gly, Ser, Asp, Asn, especially Gly and Ser, particularly Gly) to provide the desired flexibility. As such, the switchable phenotype of such activatable antibody constructs may benefit from introduction of one or more amino acids to provide for a flexible linker. In addition, as described below, where the activatable antibody is provided as a conformationally constrained construct, a flexible linker can be operably inserted to facilitate formation and maintenance of a cyclic structure in the uncleaved activatable antibody.

For example, in certain embodiments an activatable antibody comprises one of the following formulae (where the formula below represent an amino acid sequence in either N- to C-terminal direction or C- to N-terminal direction):
(MM)-L1-(CM)-(AB)
(MM)-(CM)-L2-(AB)
(MM)-L1-(CM)-L2-(AB)
   wherein MM, CM, and AB are as defined above; wherein L1 and L2 are each independently and optionally present or absent, are the same or different flexible linkers that include at least 1 flexible amino acid (e.g., Gly). In addition, the formulae above provide for additional amino acid sequences that may be positioned N-terminal or C-terminal to the activatable antibodies elements. Examples include, but are not limited to, targeting moieties (e.g., a ligand for a receptor of a cell present in a target tissue) and serum half-life extending moieties (e.g., polypeptides that bind serum proteins, such as immunoglobulin (e.g., IgG) or serum albumin (e.g., human serum albumin (HAS)).

The CM is specifically cleaved by at least one protease selected from matriptase and uPA at a rate of about 0.001-1500×10⁴ M⁻¹S⁻¹ or at least 0.001, 0.005, 0.01, 0.05, 0.1, 0.5, 1, 2.5, 5, 7.5, 10, 15, 20, 25, 50, 75, 100, 125, 150, 200, 250, 500, 750, 1000, 1250, or 1500×10⁴ M⁻¹S⁻¹. In some embodiments, the CM is specifically cleaved at a rate of about 100,000 M⁻¹S⁻¹. In some embodiments, the CM is specifically cleaved at a rate from about 1×10E2 to about 1×10E6 M⁻¹S⁻¹ (i.e., from about 1×10² to about 1×10⁶ M⁻¹S⁻¹).

For specific cleavage by an enzyme, contact between the enzyme and CM is made. When the activatable antibody comprising an AB coupled to a MM and a CM is in the presence of target and sufficient enzyme activity, the CM can be cleaved. Sufficient enzyme activity can refer to the ability of the enzyme to make contact with the CM and effect cleavage. It can readily be envisioned that an enzyme may be in the vicinity of the CM but unable to cleave because of other cellular factors or protein modification of the enzyme.

Linkers suitable for use in compositions described herein are generally ones that provide flexibility of the modified AB or the activatable antibodies to facilitate the inhibition of the binding of the AB to the target. Such linkers are generally referred to as flexible linkers. Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, 7, 8, 9, 10, 11, 12, 13, 14, 15, 16, 17, 18, 19, or 20 amino acids in length.

Exemplary flexible linkers include glycine polymers (G)n, glycine-serine polymers (including, for example, (GS)n, (GSGGS)n (SEQ ID NO: 385) and (GGGS)n (SEQ ID NO: 386), where n is an integer of at least one), glycine-alanine polymers, alanine-serine polymers, and other flexible linkers known in the art. Glycine and glycine-serine polymers are relatively unstructured, and therefore may be able to serve as a neutral tether between components. Glycine accesses significantly more phi-psi space than even alanine, and is much less restricted than residues with longer side chains (see Scheraga, Rev. Computational Chem. 11173-142 (1992)). Exemplary flexible linkers include, but are not limited to Gly-Gly-Ser-Gly (SEQ ID NO: 387), Gly-Gly-Ser-Gly-Gly (SEQ ID NO: 388), Gly-Ser-Gly-Ser-Gly (SEQ ID NO: 389), Gly-Ser-Gly-Gly-Gly (SEQ ID NO: 390), Gly-Gly-Gly-Ser-Gly (SEQ ID NO: 391), Gly-Ser-Ser-Ser-Gly (SEQ ID NO: 392), and the like. The ordinarily skilled artisan will recognize that design of an activatable antibodies can include linkers that are all or partially flexible, such that the linker can include a flexible linker as well as one or more portions that confer less flexible structure to provide for a desired activatable antibodies structure.

In some embodiments, the activatable antibodies described herein also include an agent conjugated to the activatable antibody. In some embodiments, the conjugated agent is a therapeutic agent, such as an anti-inflammatory and/or an antineoplastic agent. In such embodiments, the agent is conjugated to a carbohydrate moiety of the activatable antibody, for example, in some embodiments, where the carbohydrate moiety is located outside the antigen-binding region of the antibody or antigen-binding fragment in the activatable antibody. In some embodiments, the agent is conjugated to a sulfhydryl group of the antibody or antigen-binding fragment in the activatable antibody.

In some embodiments, the agent is a cytotoxic agent such as a toxin (e.g., an enzymatically active toxin of bacterial, fungal, plant, or animal origin, or fragments thereof), or a radioactive isotope (i.e., a radioconjugate).

In some embodiments, the agent is a detectable moiety such as, for example, a label or other marker. For example, the agent is or includes a radiolabeled amino acid, one or more biotinyl moieties that can be detected by marked avidin (e.g., streptavidin containing a fluorescent marker or enzymatic activity that can be detected by optical or calorimetric methods), one or more radioisotopes or radionuclides, one or more fluorescent labels, one or more enzymatic labels, and/or one or more chemiluminescent agents. In some embodiments, detectable moieties are attached by spacer molecules.

The disclosure also pertains to immunoconjugates comprising an antibody conjugated to a cytotoxic agent such as a toxin (e.g., an enzymatically active toxin of bacterial, fungal, plant, or animal origin, or fragments thereof), or a radioactive isotope (i.e., a radioconjugate). Suitable cytotoxic agents include, for example, dolastatins and derivatives thereof (e.g. auristatin E, AFP, MMAF, MMAE, MMAD, DMAF, DMAE). For example, the agent is monomethyl auristatin E (MMAE) or monomethyl auristatin D (MMAD). In some embodiments, the agent is an agent selected from the group listed in Table 3. In some embodiments, the agent is a dolastatin. In some embodiments, the agent is an auristatin or derivative thereof. In some embodiments, the agent is auristatin E or a derivative thereof. In some embodiments, the agent is monomethyl auristatin E (MMAE). In some embodiments, the agent is monomethyl auristatin D (MMAD). In some embodiments, the agent is a maytansinoid or maytansinoid derivative. In some embodiments, the agent is DM1 or DM4. In some embodiments, the agent is a duocarmycin or derivative thereof. In some embodiments, the agent is a calicheamicin or derivative thereof. In some embodiments, the agent is a pyrrolobenzodiazepine.

In some embodiments, the agent is linked to the AB using a maleimide caproyl-valine-citrulline linker or a maleimide PEG-valine-citrulline linker. In some embodiments, the agent is linked to the AB using a maleimide caproyl-valine-citrulline linker. In some embodiments, the agent is linked to the AB using a maleimide PEG-valine-citrulline linker In some embodiments, the agent is monomethyl auristatin D (MMAD) linked to the AB using a maleimide PEG-valine-citrulline-para-aminobenzyloxycarbonyl linker, and this linker payload construct is referred to herein as “vc-MMAD.” In some embodiments, the agent is monomethyl auristatin E (MMAE) linked to the AB using a maleimide PEG-valine-citrulline-para-aminobenzyloxycarbonyl linker, and this linker payload construct is referred to herein as “vc-MMAE.” The structures of vc-MMAD and vc-MMAE are shown below:

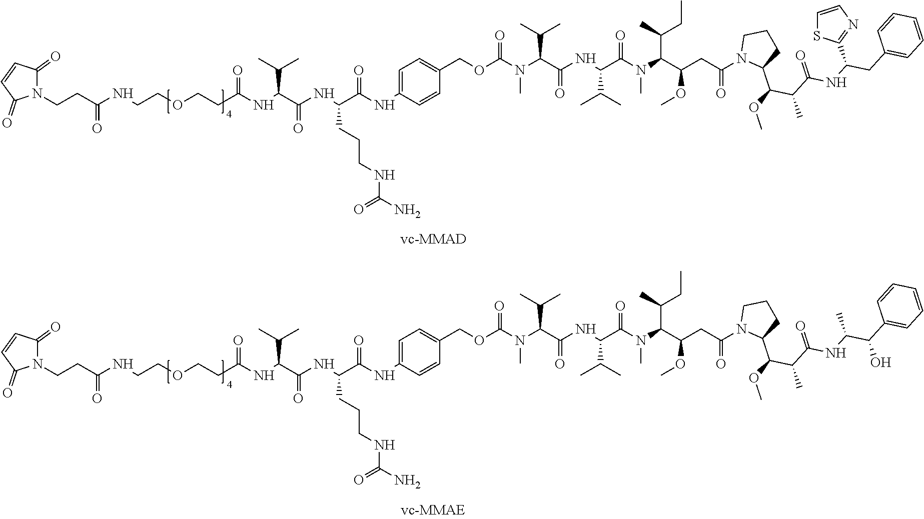

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. (See WO94/11026).

Table 3 lists some of the exemplary pharmaceutical agents that may be employed in the herein described disclosure but in no way is meant to be an exhaustive list.

TABLE 3
Exemplary Pharmaceutical Agents for Conjugation
CYTOTOXIC AGENTS
Auristatins
Auristatin E
Monomethyl auristatin D (MMAD)
Monomethyl auristatin E (MMAE)
Desmethyl auristatin E (DMAE)
Auristatin F
Monomethyl auristatin F (MMAF)
Desmethyl auristatin F (DMAF)
Auristatin derivatives, e.g., amides thereof
Auristatin tyramine
Auristatin quinoline
Dolastatins
Dolastatin derivatives
Dolastatin 16 DmJ
Dolastatin 16 Dpv
Maytansinoids, e.g. DM-1; DM-4
Maytansinoid derivatives
Duocarmycin
Duocarmycin derivatives
Alpha-amanitin
Anthracyclines
Doxorubicin
Daunorubicin
Bryostatins
Camptothecin
Camptothecin derivatives
7-substituted Camptothecin
10,11-
Difluoromethylenedioxycamptothecin
Combretastatins
Debromoaplysiatoxin
Kahalalide-F
Discodermolide
Ecteinascidins
ANTIVIRALS
Acyclovir
Vira A
Symmetrel
ANTIFUNGALS
Nystatin
ADDITIONAL ANTI-NEOPLASTICS
Adriamycin
Cerubidine
Bleomycin
Alkeran
Velban
Oncovin
Fluorouracil
Methotrexate
Thiotepa
Bisantrene
Novantrone
Thioguanine
Procarabizine
Cytarabine
ANTI-BACTERIALS
Aminoglycosides
Streptomycin
Neomycin
Kanamycin
Amikacin
Gentamicin
Tobramycin
Streptomycin B
Spectinomycin
Ampicillin
Sulfanilamide
Polymyxin
Chloramphenicol
Turbostatin
Phenstatins
Hydroxyphenstatin
Spongistatin 5
Spongistatin 7
Halistatin 1
Halistatin 2
Halistatin 3
Modified Bryostatins
Halocomstatins
Pyrrolobenzimidazoles (PBI)
Cibrostatin6
Doxaliform
Anthracyclins analogues
Cemadotin analogue (CemCH2-SH)
Pseudomonas toxin A (PE38) variant
Pseudomonas toxin A (ZZ-PE38) variant
ZJ-101
OSW-1
4-Nitrobenzyloxycarbonyl Derivatives of
O6-Benzylguanine
Topoisomerase inhibitors
Hemiasterlin
Cephalotaxine
Homoharringtonine
Pyrrolobenzodiazepine dimers (PBDs)
Functionalized pyrrolobenzodiazepenes
Calicheamicins
Podophyllotoxins
Taxanes
Vinca alkaloids
CONJUGATABLE DETECTION
REAGENTS
Fluorescein and derivatives thereof
Fluorescein isothiocyanate (FITC)
RADIOPHARMACEUTICALS
¹²⁵I
¹³¹I
⁸⁹Zr
¹¹¹In
¹²³I
¹³¹I
⁹⁹mTc
²⁰¹Tl
¹³³Xe
¹¹C
⁶²Cu
¹⁸F
⁶⁸Ga
¹³N
¹⁵O
³⁸K
⁸²Rb
⁹⁹mTc (Technetium)
HEAVY METALS
Barium
Gold
Platinum
ANTI-MYCOPLASMALS
Tylosine
Spectinomycin

Those of ordinary skill in the art will recognize that a large variety of possible moieties can be coupled to the resultant antibodies of the disclosure. (See, for example, “Conjugate Vaccines”, Contributions to Microbiology and Immunology, J. M. Cruse and R. E. Lewis, Jr (eds), Carger Press, New York, (1989), the entire contents of which are incorporated herein by reference).

Coupling may be accomplished by any chemical reaction that will bind the two molecules so long as the antibody and the other moiety retain their respective activities. This linkage can include many chemical mechanisms, for instance covalent binding, affinity binding, intercalation, coordinate binding and complexation. In some embodiments, the binding is, however, covalent binding. Covalent binding can be achieved either by direct condensation of existing side chains or by the incorporation of external bridging molecules. Many bivalent or polyvalent linking agents are useful in coupling protein molecules, such as the antibodies of the present disclosure, to other molecules. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents. (See Killen and Lindstrom, Jour. Immun. 133:1335-2549 (1984); Jansen et al., Immunological Reviews 62:185-216 (1982); and Vitetta et al., Science 238:1098 (1987).

In some embodiments, in addition to the compositions and methods provided herein, the conjugated activatable antibody can also be modified for site-specific conjugation through modified amino acid sequences inserted or otherwise included in the activatable antibody sequence. These modified amino acid sequences are designed to allow for controlled placement and/or dosage of the conjugated agent within a conjugated activatable antibody. For example, the activatable antibody can be engineered to include cysteine substitutions at positions on light and heavy chains that provide reactive thiol groups and do not negatively impact protein folding and assembly, nor alter antigen binding. In some embodiments, the activatable antibody can be engineered to include or otherwise introduce one or more non-natural amino acid residues within the activatable antibody to provide suitable sites for conjugation. In some embodiments, the activatable antibody can be engineered to include or otherwise introduce enzymatically activatable peptide sequences within the activatable antibody sequence.

Suitable linkers are described in the literature. (See, for example, Ramakrishnan, S. et al., Cancer Res. 44:201-208 (1984) describing use of MBS (M-maleimidobenzoyl-N-hydroxysuccinimide ester). See also, U.S. Pat. No. 5,030,719, describing use of halogenated acetyl hydrazide derivative coupled to an antibody by way of an oligopeptide linker. In some embodiments, suitable linkers include: (i) EDC (1-ethyl-3-(3-dimethylamino-propyl) carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamido]hexanoate (Pierce Chem. Co., Cat #21651G); (iv) Sulfo-LC-SPDP (sulfosuccinimidyl 6 [3-(2-pyridyldithio)-propianamide] hexanoate (Pierce Chem. Co. Cat. #2165-G); and (v) sulfo-NHS (N-hydroxysulfo-succinimide: Pierce Chem. Co., Cat. #24510) conjugated to EDC. Additional linkers include, but are not limited to, SMCC, sulfo-SMCC, SPDB, or sulfo-SPDB.

The linkers described above contain components that have different attributes, thus leading to conjugates with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically hindered disulfide bond, and can form conjugates with increased stability. Disulfide linkages, are in general, less stable than other linkages because the disulfide linkage is cleaved in vitro, resulting in less conjugate available. Sulfo-NHS, in particular, can enhance the stability of carbodimide couplings. Carbodimide couplings (such as EDC) when used in conjunction with sulfo-NHS, forms esters that are more resistant to hydrolysis than the carbodimide coupling reaction alone.

In some embodiments, the linkers are cleavable. In some embodiments, the linkers are non-cleavable. In some embodiments, two or more linkers are present. The two or more linkers are all the same, i.e., cleavable or non-cleavable, or the two or more linkers are different, i.e., at least one cleavable and at least one non-cleavable.

The present disclosure utilizes several methods for attaching agents to ABs: (a) attachment to the carbohydrate moieties of the AB, or (b) attachment to sulfhydryl groups of the AB, or (c) attachment to amino groups of the AB, or (d) attachment to carboxylate groups of the AB. According to the disclosure, ABs may be covalently attached to an agent through an intermediate linker having at least two reactive groups, one to react with AB and one to react with the agent. The linker, which may include any compatible organic compound, can be chosen such that the reaction with AB (or agent) does not adversely affect AB reactivity and selectivity. Furthermore, the attachment of linker to agent might not destroy the activity of the agent. Suitable linkers for reaction with oxidized antibodies or oxidized antibody fragments include those containing an amine selected from the group consisting of primary amine, secondary amine, hydrazine, hydrazide, hydroxylamine, phenylhydrazine, semicarbazide and thiosemicarbazide groups. Such reactive functional groups may exist as part of the structure of the linker, or may be introduced by suitable chemical modification of linkers not containing such groups.

According to the present disclosure, suitable linkers for attachment to reduced ABs include those having certain reactive groups capable of reaction with a sulfhydryl group of a reduced antibody or fragment. Such reactive groups include, but are not limited to: reactive haloalkyl groups (including, for example, haloacetyl groups), p-mercuribenzoate groups and groups capable of Michael-type addition reactions (including, for example, maleimides and groups of the type described by Mitra and Lawton, 1979, J. Amer. Chem. Soc. 101: 3097-3110).

According to the present disclosure, suitable linkers for attachment to neither oxidized nor reduced Abs include those having certain functional groups capable of reaction with the primary amino groups present in unmodified lysine residues in the Ab. Such reactive groups include, but are not limited to, NHS carboxylic or carbonic esters, sulfo-NHS carboxylic or carbonic esters, 4-nitrophenyl carboxylic or carbonic esters, pentafluorophenyl carboxylic or carbonic esters, acyl imidazoles, isocyanates, and isothiocyanates.

According to the present disclosure, suitable linkers for attachment to neither oxidized nor reduced Abs include those having certain functional groups capable of reaction with the carboxylic acid groups present in aspartate or glutamate residues in the Ab, which have been activated with suitable reagents. Suitable activating reagents include EDC, with or without added NHS or sulfo-NHS, and other dehydrating agents utilized for carboxamide formation. In these instances, the functional groups present in the suitable linkers would include primary and secondary amines, hydrazines, hydroxylamines, and hydrazides.

The agent may be attached to the linker before or after the linker is attached to the AB. In certain applications it may be desirable to first produce an AB-linker intermediate in which the linker is free of an associated agent. Depending upon the particular application, a specific agent may then be covalently attached to the linker. In some embodiments, the AB is first attached to the MM, CM and associated linkers and then attached to the linker for conjugation purposes.

Branched Linkers: In specific embodiments, branched linkers that have multiple sites for attachment of agents are utilized. For multiple site linkers, a single covalent attachment to an AB would result in an AB-linker intermediate capable of binding an agent at a number of sites. The sites may be aldehyde or sulfhydryl groups or any chemical site to which agents can be attached.

In some embodiments, higher specific activity (or higher ratio of agents to AB) can be achieved by attachment of a single site linker at a plurality of sites on the AB. This plurality of sites may be introduced into the AB by either of two methods. First, one may generate multiple aldehyde groups and/or sulfhydryl groups in the same AB. Second, one may attach to an aldehyde or sulfhydryl of the AB a “branched linker” having multiple functional sites for subsequent attachment to linkers. The functional sites of the branched linker or multiple site linker may be aldehyde or sulfhydryl groups, or may be any chemical site to which linkers may be attached. Still higher specific activities may be obtained by combining these two approaches, that is, attaching multiple site linkers at several sites on the AB.

Cleavable Linkers: Peptide linkers that are susceptible to cleavage by enzymes of the complement system, such as but not limited to u-plasminogen activator, tissue plasminogen activator, trypsin, plasmin, or another enzyme having proteolytic activity may be used in one embodiment of the present disclosure. According to one method of the present disclosure, an agent is attached via a linker susceptible to cleavage by complement. The antibody is selected from a class that can activate complement. The antibody-agent conjugate, thus, activates the complement cascade and releases the agent at the target site. According to another method of the present disclosure, an agent is attached via a linker susceptible to cleavage by enzymes having a proteolytic activity such as a u-plasminogen activator, a tissue plasminogen activator, plasmin, or trypsin. These cleavable linkers are useful in conjugated activatable antibodies that include an extracellular toxin, e.g., by way of non-limiting example, any of the extracellular toxins shown in Table 3.

Non-limiting examples of cleavable linker sequences are provided in Table 4.

TABLE 4
Exemplary Linker Sequences for Conjugation

| Types of Cleavable Sequences | Amino Acid Sequence |
|---|---|
| Plasmin cleavable sequences | |
| Pro-urokinase | PRFKIIGG (SEQ ID NO: 665) |
| | PRFRIIGG (SEQ ID NO: 666) |
| TGFβ | SSRHRRALD (SEQ ID NO: 667) |
| Plasminogen | RKSSIIIRMRDVVL (SEQ ID NO: 668) |
| Staphylokinase | SSSFDKGKYKKGDDA (SEQ ID NO: 669) |
| | SSSFDKGKYKRGDDA (SEQ ID NO: 670) |
| Factor Xa cleavable sequences | IEGR (SEQ ID NO: 671) |
| | IDGR (SEQ ID NO: 672) |
| | GGSIDGR (SEQ ID NO: 673) |
| MMP cleavable sequences | |
| Gelatinase A | PLGLWA (SEQ ID NO: 674) |
| Collagenase cleavable sequences | |
| Calf skin collagen (α1(I) chain) | GPQGIAGQ (SEQ ID NO: 675) |
| Calf skin collagen (α2(I) chain) | GPQGLLGA (SEQ ID NO: 676) |
| Bovine cartilage collagen (α1(II) chain) | GIAGQ (SEQ ID NO: 677) |
| Human liver collagen (α1(III) chain) | GPLGIAGI (SEQ ID NO: 678) |
| Human α₂M | GPEGLRVG (SEQ ID NO: 679) |
| Human PZP | YGAGLGVV (SEQ ID NO: 680) |
| | AGLGVVER (SEQ ID NO: 681) |
| | AGLGISST (SEQ ID NO: 682) |
| Rat α₁M | EPQALAMS (SEQ ID NO: 683) |
| | QALAMSAI (SEQ ID NO: 312) |
| Rat α₂M | AAYHLVSQ (SEQ ID NO: 315) |
| | MDAFLESS (SEQ ID NO: 316) |
| Rat α₁I₃(2J) | ESLPVVAV (SEQ ID NO: 317) |
| Rat α₁I₃(27J) | SAPAVESE (SEQ ID NO: 318) |
| Human fibroblast collagenase | DVAQFVLT (SEQ ID NO: 319) |
| (autolytic cleavages) | VAQFVLTE (SEQ ID NO: 372) |
| | AQFVLTEG (SEQ ID NO: 373) |
| | PVQPIGPQ (SEQ ID NO: 380) |

In addition, agents may be attached via disulfide bonds (for example, the disulfide bonds on a cysteine molecule) to the AB. Since many tumors naturally release high levels of glutathione (a reducing agent) this can reduce the disulfide bonds with subsequent release of the agent at the site of delivery. In some embodiments, the reducing agent that would modify a CM would also modify the linker of the conjugated activatable antibody.

Spacers and Cleavable Elements: In some embodiments, it may be necessary to construct the linker in such a way as to optimize the spacing between the agent and the AB of the activatable antibody. This may be accomplished by use of a linker of the general structure:

W—(CH₂)n-Q
wherein
W is either —NH—CH₂— or —CH₂—;
Q is an amino acid, peptide; and
n is an integer from 0 to 20.

In some embodiments, the linker may comprise a spacer element and a cleavable element. The spacer element serves to position the cleavable element away from the core of the AB such that the cleavable element is more accessible to the enzyme responsible for cleavage. Certain of the branched linkers described above may serve as spacer elements.

Throughout this discussion, it should be understood that the attachment of linker to agent (or of spacer element to cleavable element, or cleavable element to agent) need not be particular mode of attachment or reaction. Any reaction providing a product of suitable stability and biological compatibility is acceptable.

Serum Complement and Selection of Linkers: According to one method of the present disclosure, when release of an agent is desired, an AB that is an antibody of a class that can activate complement is used. The resulting conjugate retains both the ability to bind antigen and activate the complement cascade. Thus, according to this embodiment of the present disclosure, an agent is joined to one end of the cleavable linker or cleavable element and the other end of the linker group is attached to a specific site on the AB. For example, if the agent has an hydroxy group or an amino group, it may be attached to the carboxy terminus of a peptide, amino acid or other suitably chosen linker via an ester or amide bond, respectively. For example, such agents may be attached to the linker peptide via a carbodimide reaction. If the agent contains functional groups that would interfere with attachment to the linker, these interfering functional groups can be blocked before attachment and deblocked once the product conjugate or intermediate is made. The opposite or amino terminus of the linker is then used either directly or after further modification for binding to an AB that is capable of activating complement.

Linkers (or spacer elements of linkers) may be of any desired length, one end of which can be covalently attached to specific sites on the AB of the activatable antibody. The other end of the linker or spacer element may be attached to an amino acid or peptide linker.

Thus when these conjugates bind to antigen in the presence of complement the amide or ester bond that attaches the agent to the linker will be cleaved, resulting in release of the agent in its active form. These conjugates, when administered to a subject, will accomplish delivery and release of the agent at the target site, and are particularly effective for the in vivo delivery of pharmaceutical agents, antibiotics, antimetabolites, antiproliferative agents and the like as presented in but not limited to those in Table 3.

Linkers for Release without Complement Activation: In yet another application of targeted delivery, release of the agent without complement activation is desired since activation of the complement cascade will ultimately lyse the target cell. Hence, this approach is useful when delivery and release of the agent should be accomplished without killing the target cell. Such is the goal when delivery of cell mediators such as hormones, enzymes, corticosteroids, neurotransmitters, genes or enzymes to target cells is desired. These conjugates may be prepared by attaching the agent to an AB that is not capable of activating complement via a linker that is mildly susceptible to cleavage by serum proteases. When this conjugate is administered to an individual, antigen-antibody complexes will form quickly whereas cleavage of the agent will occur slowly, thus resulting in release of the compound at the target site.

Biochemical Cross Linkers: In some embodiments, the activatable antibody may be conjugated to one or more therapeutic agents using certain biochemical cross-linkers. Cross-linking reagents form molecular bridges that tie together functional groups of two different molecules. To link two different proteins in a step-wise manner, hetero-bifunctional cross-linkers can be used that eliminate unwanted homopolymer formation.

Peptidyl linkers cleavable by lysosomal proteases are also useful, for example, Val-Cit, Val-Ala or other dipeptides. In addition, acid-labile linkers cleavable in the low-pH environment of the lysosome may be used, for example: bis-sialyl ether. Other suitable linkers include cathepsin-labile substrates, particularly those that show optimal function at an acidic pH.

Exemplary hetero-bifunctional cross-linkers are referenced in Table 5.

TABLE 5
Exemplary Hetero-Bifunctional Cross Linkers
HETERO-BIFUNCTIONAL CROSS-LINKERS

| | | | Spacer Arm | |
|---|---|---|---|---|
| | | | Length after | |
| | | Advantages and | cross-linking | |
| Linker | Reactive Toward | Applications | (Angstroms) | |
| SMPT | Primary amines | Greater stability | 11.2 | Å |
| | Sulfhydryls | | | |
| SPDP | Primary amines | Thiolation | 6.8 | Å |
| | Sulfhydryls | Cleavable cross-linking | | |
| LC-SPDP | Primary amines | Extended spacer arm | 15.6 | Å |
| | Sulfhydryls | | | |
| Sulfo-LC- | Primary amines | Extender spacer arm | 15.6 | Å |
| SPDP | Sulfhydryls | Water-soluble | | |
| SMCC | Primary amines | Stable maleimide reactive | 11.6 | Å |
| | | group | | |
| | Sulfhydryls | Enzyme-antibody | | |
| | | conjugation | | |
| | | Hapten-carrier protein | | |
| | | conjugation | | |
| Sulfo- | Primary amines | Stable maleimide reactive | 11.6 | Å |
| SMCC | | group | | |
| | Sulfhydryls | Water-soluble | | |
| | | Enzyme-antibody | | |
| | | conjugation | | |
| MBS | Primary amines | Enzyme-antibody | 9.9 | Å |
| | Sulfhydryls | conjugation | | |
| | | Hapten-carrier protein | | |
| | | conjugation | | |
| Sulfo- | Primary amines | Water-soluble | 9.9 | Å |
| MBS | Sulfhydryls | | | |
| SIAB | Primary amines | Enzyme-antibody | 10.6 | Å |
| | Sulfhydryls | conjugation | | |
| Sulfo- | Primary amines | Water-soluble | 10.6 | Å |
| SIAB | Sulfhydryls | | | |
| SMPB | Primary amines | Extended spacer arm | 14.5 | Å |
| | Sulfhydryls | Enzyme-antibody | | |
| | | conjugation | | |
| Sulfo- | Primary amines | Extended spacer arm | 14.5 | Å |
| SMPB | Sulfhydryls | Water-soluble | | |
| EDE/ | Primary amines | Hapten-Carrier | 0 | |
| Sulfo- | Carboxyl groups | conjugation | | |
| NHS | | | | |
| ABH | Carbohydrates | Reacts with sugar | 11.9 | Å |
| | Nonselective | groups | | |

Non-Cleavable Linkers or Direct Attachment: In some embodiments of the disclosure, the conjugate may be designed so that the agent is delivered to the target but not released. This may be accomplished by attaching an agent to an AB either directly or via a non-cleavable linker.

These non-cleavable linkers may include amino acids, peptides, D-amino acids or other organic compounds that may be modified to include functional groups that can subsequently be utilized in attachment to ABs by the methods described herein. A-general formula for such an organic linker could be

W—(CH₂)n-Q
wherein
W is either —NH—CH₂— or —CH₂—;
Q is an amino acid, peptide; and
n is an integer from 0 to 20.

Non-Cleavable Conjugates: In some embodiments, a compound may be attached to ABs that do not activate complement. When using ABs that are incapable of complement activation, this attachment may be accomplished using linkers that are susceptible to cleavage by activated complement or using linkers that are not susceptible to cleavage by activated complement.

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present disclosure can be conjugated to the liposomes as described in Martin et al., J. Biol. Chem., 257: 286-288 (1982) via a disulfide-interchange reaction.

### Definitions

Unless otherwise defined, scientific and technical terms used in connection with the present disclosure shall have the meanings that are commonly understood by those of ordinary skill in the art. The term “a” entity or “an” entity refers to one or more of that entity. For example, a compound refers to one or more compounds. As such, the terms “a”, “an”, “one or more” and “at least one” can be used interchangeably. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)). The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

As used herein, the term “antibody” refers to immunoglobulin molecules and immunologically active portions of immunoglobulin (Ig) molecules, i.e., molecules that contain an antigen binding site that specifically binds (immunoreacts with) an antigen. By “specifically bind” or “immunoreacts with” or “immunospecifically bind” is meant that the antibody reacts with one or more antigenic determinants of the desired antigen and does not react with other polypeptides or binds at much lower affinity (K_{d}>10⁻⁶). Antibodies include, but are not limited to, polyclonal, monoclonal, chimeric, domain antibody, single chain, Fab, and F(ab′)₂ fragments, scFvs, and an Fab expression library.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. In general, antibody molecules obtained from humans relate to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain.

The term “monoclonal antibody” (mAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs contain an antigen binding site capable of immunoreacting with a particular epitope of the antigen characterized by a unique binding affinity for it.

The term “antigen-binding site” or “binding portion” refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” refers to amino acid sequences that are naturally found between, and adjacent to, hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987), Chothia et al. Nature 342:878-883 (1989).

As used herein, the term “epitope” includes any protein determinant capable of specific binding to an immunoglobulin, an scFv, or a T-cell receptor. The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. For example, antibodies may be raised against N-terminal or C-terminal peptides of a polypeptide. An antibody is said to specifically bind an antigen when the dissociation constant is ≤1 μM; in some embodiments, ≤100 nM and in some embodiments, ≤10 nM.

As used herein, the terms “specific binding,” “immunological binding,” and “immunological binding properties” refer to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See Nature 361:186-87 (1993)). The ratio of K_{off}/Kₒₙ enables the cancellation of all parameters not related to affinity, and is equal to the dissociation constant K_{d}. (See, generally, Davies et al. (1990) Annual Rev Biochem 59:439-473). An antibody of the present disclosure is said to specifically bind to the target, when the equilibrium binding constant (K_{d}) is ≤1 μM, in some embodiments ≤100 nM, in some embodiments ≤10 nM, and in some embodiments ≤100 μM to about 1 μM, as measured by assays such as radioligand binding assays or similar assays known to those skilled in the art.

The term “isolated polynucleotide” as used herein shall mean a polynucleotide of genomic, cDNA, or synthetic origin or some combination thereof, which by virtue of its origin the “isolated polynucleotide” (1) is not associated with all or a portion of a polynucleotide in which the “isolated polynucleotide” is found in nature, (2) is operably linked to a polynucleotide which it is not linked to in nature, or (3) does not occur in nature as part of a larger sequence. Polynucleotides in accordance with the disclosure include the nucleic acid molecules encoding the heavy chain immunoglobulin molecules shown herein, and nucleic acid molecules encoding the light chain immunoglobulin molecules shown herein.

The term “isolated protein” referred to herein means a protein of cDNA, recombinant RNA, or synthetic origin or some combination thereof, which by virtue of its origin, or source of derivation, the “isolated protein” (1) is not associated with proteins found in nature, (2) is free of other proteins from the same source, e.g., free of murine proteins, (3) is expressed by a cell from a different species, or (4) does not occur in nature.

The term “polypeptide” is used herein as a generic term to refer to native protein, fragments, or analogs of a polypeptide sequence. Hence, native protein fragments, and analogs are species of the polypeptide genus. Polypeptides in accordance with the disclosure comprise the heavy chain immunoglobulin molecules shown herein, and the light chain immunoglobulin molecules shown herein, as well as antibody molecules formed by combinations comprising the heavy chain immunoglobulin molecules with light chain immunoglobulin molecules, such as kappa light chain immunoglobulin molecules, and vice versa, as well as fragments and analogs thereof.

The term “naturally-occurring” as used herein as applied to an object refers to the fact that an object can be found in nature. For example, a polypeptide or polynucleotide sequence that is present in an organism (including viruses) that can be isolated from a source in nature and that has not been intentionally modified by man in the laboratory or otherwise is naturally-occurring.

The term “operably linked” as used herein refers to positions of components so described are in a relationship permitting them to function in their intended manner. A control sequence “operably linked” to a coding sequence is ligated in such a way that expression of the coding sequence is achieved under conditions compatible with the control sequences.

The term “control sequence” as used herein refers to polynucleotide sequences that are necessary to effect the expression and processing of coding sequences to which they are ligated. The nature of such control sequences differs depending upon the host organism in prokaryotes, such control sequences generally include promoter, ribosomal binding site, and transcription termination sequence in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences. The term “polynucleotide” as referred to herein means nucleotides of at least 10 bases in length, either ribonucleotides or deoxynucleotides or a modified form of either type of nucleotide. The term includes single and double stranded forms of DNA.

The term oligonucleotide referred to herein includes naturally occurring, and modified nucleotides linked together by naturally occurring, and non-naturally occurring oligonucleotide linkages. Oligonucleotides are a polynucleotide subset generally comprising a length of 200 bases or fewer. In some embodiments, oligonucleotides are 10 to 60 bases in length and in some embodiments, 12, 13, 14, 15, 16, 17, 18, 19, or 20 to 40 bases in length. Oligonucleotides are usually single stranded, e.g., for probes, although oligonucleotides may be double stranded, e.g., for use in the construction of a gene mutant. Oligonucleotides of the disclosure are either sense or antisense oligonucleotides.

The term “naturally occurring nucleotides” referred to herein includes deoxyribonucleotides and ribonucleotides. The term “modified nucleotides” referred to herein includes nucleotides with modified or substituted sugar groups and the like. The term “oligonucleotide linkages” referred to herein includes oligonucleotide linkages such as phosphorothioate, phosphorodithioate, phosphoroselerloate, phosphorodiselenoate, phosphoroanilothioate, phoshoraniladate, phosphoronmidate, and the like. See e.g., LaPlanche et al. Nucl. Acids Res. 14:9081 (1986); Stec et al. J. Am. Chem. Soc. 106:6077 (1984), Stein et al. Nucl. Acids Res. 16:3209 (1988), Zon et al. Anti Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990). An oligonucleotide can include a label for detection, if desired.

As used herein, the twenty conventional amino acids and their abbreviations follow conventional usage. See Immunology—A Synthesis (2nd Edition, E. S. Golub and D. R. Green, Eds., Sinauer Associates, Sunderland, Mass. (1991)). Stereoisomers (e.g., D-amino acids) of the twenty conventional amino acids, unnatural amino acids such as α-, α-disubstituted amino acids, N-alkyl amino acids, lactic acid, and other unconventional amino acids may also be suitable components for polypeptides of the present disclosure. Examples of unconventional amino acids include: 4 hydroxyproline, γ-carboxyglutamate, ε-N,N,N-trimethyllysine, ε-N-acetyllysine, σ-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

Similarly, unless specified otherwise, the left-hand end of single-stranded polynucleotide sequences is the 5′ end the left-hand direction of double-stranded polynucleotide sequences is referred to as the 5′ direction. The direction of 5′ to 3′ addition of nascent RNA transcripts is referred to as the transcription direction sequence regions on the DNA strand having the same sequence as the RNA and that are 5′ to the 5′ end of the RNA transcript are referred to as “upstream sequences”, sequence regions on the DNA strand having the same sequence as the RNA and that are 3′ to the 3′ end of the RNA transcript are referred to as “downstream sequences”.

As applied to polypeptides, the term “substantial identity” means that two peptide sequences, when optimally aligned, such as by the programs GAP or BESTFIT using default gap weights, share at least 80 percent sequence identity, in some embodiments, at least 90 percent sequence identity, in some embodiments, at least 95 percent sequence identity, and in some embodiments, at least 99 percent sequence identity.

In some embodiments, residue positions that are not identical differ by conservative amino acid substitutions.

As discussed herein, minor variations in the amino acid sequences of antibodies or immunoglobulin molecules are contemplated as being encompassed by the present disclosure, providing that the variations in the amino acid sequence maintain at least 75%, in some embodiments, at least 80%, 90%, 95%, and in some embodiments, 99%. In particular, conservative amino acid replacements are contemplated. Conservative replacements are those that take place within a family of amino acids that are related in their side chains. Genetically encoded amino acids are generally divided into families: (1) acidic amino acids are aspartate, glutamate; (2) basic amino acids are lysine, arginine, histidine; (3) non-polar amino acids are alanine, valine, leucine, isoleucine, proline, phenylalanine, methionine, tryptophan, and (4) uncharged polar amino acids are glycine, asparagine, glutamine, cysteine, serine, threonine, tyrosine. The hydrophilic amino acids include arginine, asparagine, aspartate, glutamine, glutamate, histidine, lysine, serine, and threonine. The hydrophobic amino acids include alanine, cysteine, isoleucine, leucine, methionine, phenylalanine, proline, tryptophan, tyrosine and valine. Other families of amino acids include (i) serine and threonine, which are the aliphatic-hydroxy family; (ii) asparagine and glutamine, which are the amide containing family; (iii) alanine, valine, leucine and isoleucine, which are the aliphatic family; and (iv) phenylalanine, tryptophan, and tyrosine, which are the aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. Suitable amino- and carboxy-termini of fragments or analogs occur near boundaries of functional domains. Structural and functional domains can be identified by comparison of the nucleotide and/or amino acid sequence data to public or proprietary sequence databases. In some embodiments, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Thus, the foregoing examples demonstrate that those of skill in the art can recognize sequence motifs and structural conformations that may be used to define structural and functional domains in accordance with the disclosure.

Suitable amino acid substitutions are those that: (1) reduce susceptibility to proteolysis, (2) reduce susceptibility to oxidation, (3) alter binding affinity for forming protein complexes, (4) alter binding affinities, and (5) confer or modify other physicochemical or functional properties of such analogs. Analogs can include various muteins of a sequence other than the naturally-occurring peptide sequence. For example, single or multiple amino acid substitutions (for example, conservative amino acid substitutions) may be made in the naturally-occurring sequence (for example, in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et at. Nature 354:105 (1991).

The term “polypeptide fragment” as used herein refers to a polypeptide that has an amino terminal and/or carboxy-terminal deletion and/or one or more internal deletion(s), but where the remaining amino acid sequence is identical to the corresponding positions in the naturally-occurring sequence deduced, for example, from a full length cDNA sequence. Fragments typically are at least 5, 6, 8 or 10 amino acids long, in some embodiments, at least 14 amino acids long, in some embodiments, at least 20 amino acids long, usually at least 50 amino acids long, and in some embodiments, at least 70 amino acids long. The term “analog” as used herein refers to polypeptides that are comprised of a segment of at least 25 amino acids that has substantial identity to a portion of a deduced amino acid sequence and that has specific binding to the target, under suitable binding conditions. Typically, polypeptide analogs comprise a conservative amino acid substitution (or addition or deletion) with respect to the naturally-occurring sequence. Analogs typically are at least 20 amino acids long, in some embodiments, at least 50 amino acids long or longer, and can often be as long as a full-length naturally-occurring polypeptide.

The term “agent” is used herein to denote a chemical compound, a mixture of chemical compounds, a biological macromolecule, or an extract made from biological materials.

As used herein, the terms “label” or “labeled” refers to incorporation of a detectable marker, e.g., by incorporation of a radiolabeled amino acid or attachment to a polypeptide of biotinyl moieties that can be detected by marked avidin (e.g., streptavidin containing a fluorescent marker or enzymatic activity that can be detected by optical or calorimetric methods). In certain situations, the label or marker can also be therapeutic. Various methods of labeling polypeptides and glycoproteins are known in the art and may be used. Examples of labels for polypeptides include, but are not limited to, the following: radioisotopes ³H, ¹⁴C, ¹⁵N, ³⁵S, ⁹⁰Y, ⁹⁹Tc, ¹¹¹In, ¹²⁵I, ¹³¹I), or radionuclides (e.g., fluorescent labels (e.g., FITC, rhodamine, lanthanide phosphors), enzymatic labels (e.g., horseradish peroxidase, p-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance. The term “pharmaceutical agent or drug” as used herein refers to a chemical compound or composition capable of inducing a desired therapeutic effect when properly administered to a patient.

Other chemistry terms herein are used according to conventional usage in the art, as exemplified by The McGraw-Hill Dictionary of Chemical Terms (Parker, S., Ed., McGraw-Hill, San Francisco (1985)).

As used herein, “substantially pure” means an object species is the predominant species present (i.e., on a molar basis it is more abundant than any other individual species in the composition), and in some embodiments, a substantially purified fraction is a composition wherein the object species comprises at least about 50 percent (on a molar basis) of all macromolecular species present.

Generally, a substantially pure composition will comprise more than about 80 percent of all macromolecular species present in the composition, in some embodiments, more than about 85%, 90%, 95%, and 99%. In some embodiments, the object species is purified to essential homogeneity (contaminant species cannot be detected in the composition by conventional detection methods) wherein the composition consists essentially of a single macromolecular species.

The term patient includes human and veterinary subjects.

Activatable antibodies of the disclosure specifically bind a given target, e.g., a human target protein. Also included in the disclosure are activatable antibodies that bind to the same epitope as the activatable antibodies described herein.

Those skilled in the art will recognize that it is possible to determine, without undue experimentation, if a monoclonal antibody (e.g., a murine monoclonal or humanized antibody) has the same specificity as a monoclonal antibody used in the methods described herein by ascertaining whether the former prevents the latter from binding to the target. If the monoclonal antibody being tested competes with the monoclonal antibody of the disclosure, as shown by a decrease in binding by the monoclonal antibody of the disclosure, then the two monoclonal antibodies bind to the same, or a closely related, epitope. An alternative method for determining whether a monoclonal antibody has the specificity of a monoclonal antibody of the disclosure is to pre-incubate the monoclonal antibody of the disclosure with the target and then add the monoclonal antibody being tested to determine if the monoclonal antibody being tested is inhibited in its ability to bind the target. If the monoclonal antibody being tested is inhibited then, in all likelihood, it has the same, or functionally equivalent, epitopic specificity as the monoclonal antibody of the disclosure.

Multispecific Activatable Antibodies

The disclosure also provides multispecific activatable antibodies. The multispecific activatable antibodies provided herein are multispecific antibodies that recognize two or more different antigens or epitopes and that include at least one masking moiety (MM) linked to at least one antigen- or epitope-binding domain of the multispecific antibody such that coupling of the MM reduces the ability of the antigen- or epitope-binding domain to bind its target. In some embodiments, the MM is coupled to the antigen- or epitope-binding domain of the multispecific antibody via a cleavable moiety (CM) that functions as a substrate for at least one protease selected from uPA and matriptase. The activatable multispecific antibodies provided herein are stable in circulation, activated at intended sites of therapy and/or diagnosis but not in normal, i.e., healthy tissue, and, when activated, exhibit binding to a target that is at least comparable to the corresponding, unmodified multispecific antibody.

In some embodiments, the multispecific activatable antibodies are designed to engage immune effector cells, also referred to herein as immune-effector cell engaging multispecific activatable antibodies. In some embodiments, the multispecific activatable antibodies are designed to engage leukocytes, also referred to herein as leukocyte engaging multispecific activatable antibodies. In some embodiments, the multispecific activatable antibodies are designed to engage T cells, also referred to herein as T-cell engaging multispecific activatable antibodies. In some embodiments, the multispecific activatable antibodies engage a surface antigen on a leukocyte, such as on a T cell, on a natural killer (NK) cell, on a myeloid mononuclear cell, on a macrophage, and/or on another immune effector cell. In some embodiments, the immune effector cell is a leukocyte. In some embodiments, the immune effector cell is a T cell. In some embodiments, the immune effector cell is a NK cell. In some embodiments, the immune effector cell is a mononuclear cell, such as a myeloid mononuclear cell. In some embodiments, the multispecific activatable antibodies are designed to bind or otherwise interact with more than one target and/or more than one epitope, also referred to herein as multi-antigen targeting activatable antibodies. As used herein, the terms “target” and “antigen” are used interchangeably.

In some embodiments, immune effector cell engaging multispecific activatable antibodies of the disclosure include a targeting antibody or antigen-binding fragment thereof and an immune effector cell engaging antibody or antigen-binding portion thereof, where at least one of the targeting antibody or antigen-binding fragment thereof and/or the immune effector cell engaging antibody or antigen-binding portion thereof is masked. In some embodiments, the immune effector cell engaging antibody or antigen binding fragment thereof includes a first antibody or antigen-binding fragment thereof (AB1) that binds a first, immune effector cell engaging target, where the AB1 is attached to a masking moiety (MM1) such that coupling of the MM1 reduces the ability of the AB1 to bind the first target. In some embodiments, the targeting antibody or antigen-binding fragment thereof includes a second antibody or fragment thereof that includes a second antibody or antigen-binding fragment thereof (AB2) that binds a second target, where the AB2 is attached to a masking moiety (MM2) such that coupling of the MM2 reduces the ability of the AB2 to bind the second target. In some embodiments, the immune effector cell engaging antibody or antigen binding fragment thereof includes a first antibody or antigen-binding fragment thereof (AB1) that binds a first, immune effector cell engaging target, where the AB1 is attached to a masking moiety (MM1) such that coupling of the MM1 reduces the ability of the AB1 to bind the first target, and the targeting antibody or antigen-binding fragment thereof includes a second antibody or fragment thereof that includes a second antibody or antigen-binding fragment thereof (AB2) that binds a second target, where the AB2 is attached to a masking moiety (MM2) such that coupling of the MM2 reduces the ability of the AB2 to bind the second target. In some embodiments, the non-immune effector cell engaging antibody is a cancer targeting antibody. In some embodiments the non-immune cell effector antibody is an IgG. In some embodiments the immune effector cell engaging antibody is a scFv. In some embodiments the targeting antibody (e.g., non-immune cell effector antibody) is an IgG and the immune effector cell engaging antibody is a scFv. In some embodiments, the immune effector cell is a leukocyte. In some embodiments, the immune effector cell is a T cell. In some embodiments, the immune effector cell is a NK cell. In some embodiments, the immune effector cell is a myeloid mononuclear cell.

In some embodiments, T-cell engaging multispecific activatable antibodies of the disclosure include a targeting antibody or antigen-binding fragment thereof and a T-cell engaging antibody or antigen-binding portion thereof, where at least one of the targeting antibody or antigen-binding fragment thereof and/or the T-cell engaging antibody or antigen-binding portion thereof is masked. In some embodiments, the T-cell engaging antibody or antigen binding fragment thereof includes a first antibody or antigen-binding fragment thereof (AB1) that binds a first, T-cell engaging target, where the AB1 is attached to a masking moiety (MM1) such that coupling of the MM1 reduces the ability of the AB1 to bind the first target. In some embodiments, the targeting antibody or antigen-binding fragment thereof includes a second antibody or fragment thereof that includes a second antibody or antigen-binding fragment thereof (AB2) that binds a second target, where the AB2 is attached to a masking moiety (MM2) such that coupling of the MM2 reduces the ability of the AB2 to bind the second target. In some embodiments, the T-cell engaging antibody or antigen binding fragment thereof includes a first antibody or antigen-binding fragment thereof (AB1) that binds a first, T-cell engaging target, where the AB1 is attached to a masking moiety (MM1) such that coupling of the MM1 reduces the ability of the AB1 to bind the first target, and the targeting antibody or antigen-binding fragment thereof includes a second antibody or fragment thereof that includes a second antibody or antigen-binding fragment thereof (AB2) that binds a second target, where the AB2 is attached to a masking moiety (MM2) such that coupling of the MM2 reduces the ability of the AB2 to bind the second target.

In some embodiments, the T-cell engaging multispecific activatable antibodies include a cancer targeting antibody or antigen-binding fragment thereof and a T-cell engaging antibody or antigen-binding portion thereof, where at least one of the cancer targeting antibody or antigen-binding fragment thereof and/or the T-cell engaging antibody or antigen-binding portion thereof is masked. In some embodiments, the T-cell engaging antibody or antigen binding fragment thereof includes a first antibody or antigen-binding fragment thereof (AB1) that binds a first, T-cell engaging target, where the AB1 is attached to a masking moiety (MM1) such that coupling of the MIMI reduces the ability of the AB1 to bind the first target. In some embodiments, the cancer targeting antibody or antigen-binding fragment thereof includes a second antibody or fragment thereof that includes a second antibody or antigen-binding fragment thereof (AB2) that binds a second, cancer-related target, where the AB2 is attached to a masking moiety (MM2) such that coupling of the MM2 reduces the ability of the AB2 to bind the second, cancer-related target. In some embodiments, the T-cell engaging antibody or antigen binding fragment thereof includes a first antibody or antigen-binding fragment thereof (AB1) that binds a first, T-cell engaging target, where the AB1 is attached to a masking moiety (MM1) such that coupling of the MM1 reduces the ability of the AB1 to bind the first target, and the cancer targeting antibody or antigen-binding fragment thereof includes a second antibody or fragment thereof that includes a second antibody or antigen-binding fragment thereof (AB2) that binds a second, cancer-related target, where the AB2 is attached to a masking moiety (MM2) such that coupling of the MM2 reduces the ability of the AB2 to bind the second, cancer-related target.

In some embodiments, the T-cell engaging multispecific activatable antibodies include a cancer targeting IgG antibody or antigen-binding fragment thereof and a T-cell engaging scFv, where at least one of the cancer targeting IgG antibody or antigen-binding fragment thereof and/or the T-cell engaging antibody or antigen-binding portion thereof is masked. In some embodiments, the T-cell engaging antibody or antigen binding fragment thereof includes a first antibody or antigen-binding fragment thereof (AB1) that binds a first, T-cell engaging target, where the AB1 is attached to a masking moiety (MM1) such that coupling of the MM1 reduces the ability of the AB1 to bind the first target. In some embodiments, the cancer targeting IgG antibody or antigen-binding fragment thereof includes a second antibody or fragment thereof that includes a second antibody or antigen-binding fragment thereof (AB2) that binds a second, cancer-related target, where the AB2 is attached to a masking moiety (MM2) such that coupling of the MM2 reduces the ability of the AB2 to bind the second, cancer-related target. In some embodiments, the T-cell engaging antibody or antigen binding fragment thereof includes a first antibody or antigen-binding fragment thereof (AB1) that binds a first, T-cell engaging target, where the AB1 is attached to a masking moiety (MM1) such that coupling of the MM1 reduces the ability of the AB1 to bind the first target, and the cancer targeting IgG antibody or antigen-binding fragment thereof includes a second antibody or fragment thereof that includes a second antibody or antigen-binding fragment thereof (AB2) that binds a second, cancer-related target, where the AB2 is attached to a masking moiety (MM2) such that coupling of the MM2 reduces the ability of the AB2 to bind the second, cancer-related target.

In some embodiments of an immune effector cell engaging multi specific activatable antibody, one antigen is typically an antigen present on the surface of a tumor cell or other cell type associated with disease, such as, but not limited to, any target listed in Table 1, such as, but not limited to, EGFR, erbB2, EpCAM, Jagged, PD-L1, B7H3, or CD71 (transferrin receptor), and another antigen is typically a stimulatory or inhibitory receptor present on the surface of a T-cell, natural killer (NK) cell, myeloid mononuclear cell, macrophage, and/or other immune effector cell, such as, but not limited to, B7-H4, BTLA, CD3, CD4, CD8, CD16a, CD25, CD27, CD28, CD32, CD56, CD137, CTLA-4, GITR, HVEM, ICOS, LAG3, NKG2D, OX40, PD-1, TIGIT, TIM3, or VISTA. In some embodiments, the antigen is a stimulatory receptor present on the surface of a T cell or NK cell; examples of such stimulatory receptors include, but are not limited to, CD3, CD27, CD28, CD137 (also referred to as 4-1BB), GITR, HVEM, ICOS, NKG2D, and OX40. In some embodiments, the antigen is an inhibitory receptor present on the surface of a T-cell; examples of such inhibitory receptors include, but are not limited to, BTLA, CTLA-4, LAG3, PD-1, TIGIT, TIM3, and NK-expressed KIRs. The antibody domain conferring specificity to the T-cell surface antigen may also be substituted by a ligand or ligand domain that binds to a T-cell receptor, a NK-cell receptor, a macrophage receptor, and/or other immune effector cell receptor, such as, but not limited to, B7-1, B7-2, B7H3, PD-L1, PD-L2, or TNFSF9.

One embodiment of the disclosure is a multispecific activatable antibody that is activatable in a cancer microenvironment and that includes an antibody, for example a IgG or scFv, directed to a tumor target and an agonist antibody, for example an IgG or scFv, directed to a co-stimulatory receptor expressed on the surface of an activated T cell or NK cell, wherein at least one of the cancer target antibody and/or agonist antibody is masked. Examples of co-stimulatory receptors include, but are not limited to, CD27, CD137, GITR, HVEM, NKG2D, and OX40. In this embodiment, the multispecific activatable antibody, once activated by tumor-associated proteases, would effectively crosslink and activate the T cell or NK cell expressed co-stimulatory receptors in a tumor-dependent manner to enhance the activity of T cells that are responding to any tumor antigen via their endogenous T cell antigen or NK-activating receptors. The activation-dependent nature of these T cell or NK cell costimulatory receptors would focus the activity of the activated multispecific activatable antibody to tumor-specific T cells, without activating all T cells independent of their antigen specificity. In one embodiment, at least the co-stimulatory receptor antibody of the multispecific activatable antibody is masked to prevent activation of auto-reactive T cells that may be present in tissues that also express the antigen recognized by the tumor target-directed antibody in the multispecific activatable antibody, but whose activity is restricted by lack of co-receptor engagement.

One embodiment of the disclosure is a multispecific activatable antibody that is activatable in a disease characterized by T cell overstimulation, such as, but not limited to, an autoimmune disease or inflammatory disease microenvironment. Such a multispecific activatable antibody includes an antibody, for example a IgG or scFv, directed to a target comprising a surface antigen expressed in a tissue targeted by a T cell in autoimmune or inflammatory disease and an antibody, for example a IgG or scFv, directed to an inhibitory receptor expressed on the surface of a T cell or NK cell, wherein at least one of the disease tissue target antibody and/or T cell inhibitory receptor antibody is masked. Examples of inhibitory receptors include, but are not limited to, BTLA, CTLA-4, LAG3, PD-1, TIGIT, TIM3, and NK-expressed KIRs. Examples of a tissue antigen targeted by T cells in autoimmune disease include, but are not limited to, a surface antigen expressed on myelin or nerve cells in multiple sclerosis or a surface antigen expressed on pancreatic islet cells in Type 1 diabetes. In this embodiment, the multispecific activatable antibody when localized in the tissue under autoimmune attack or inflammation is activated and co-engages the T cell or NK cell inhibitory receptor to suppress the activity of autoreactive T cells responding to any disease tissue-targeted antigens via their endogenous TCR or activating receptors. In one embodiment, at least one or multiple antibodies are masked to prevent suppression of T cell responses in non-disease tissues where the target antigen may also be expressed.

In some embodiments, the T-cell engaging multispecific activatable antibody includes an anti-CD3 epsilon (CD3ε, also referred to herein as CD3e and CD3) scFv and a targeting antibody or antigen-binding fragment thereof, where at least one of the anti-CD3ε scFv and/or the targeting antibody or antigen-binding portion thereof is masked. In some embodiments, the CD3ε scFv includes a first antibody or antigen-binding fragment thereof (AB1) that binds CD3ε, where the AB1 is attached to a masking moiety (MM1) such that coupling of the MM1 reduces the ability of the AB1 to bind CD3ε. In some embodiments, the targeting antibody or antigen-binding fragment thereof includes a second antibody or fragment thereof that includes a second antibody or antigen-binding fragment thereof (AB2) that binds a second target, where the AB2 is attached to a masking moiety (MM2) such that coupling of the MM2 reduces the ability of the AB2 to bind the second target. In some embodiments, the CD3ε scFv includes a first antibody or antigen-binding fragment thereof (AB1) that binds CD3ε, where the AB1 is attached to a masking moiety (MM1) such that coupling of the MM1 reduces the ability of the AB1 to bind CD3 ε, and the targeting antibody or antigen-binding fragment thereof includes a second antibody or fragment thereof that includes a second antibody or antigen-binding fragment thereof (AB2) that binds a second target, where the AB2 is attached to a masking moiety (MM2) such that coupling of the MM2 reduces the ability of the AB2 to bind the second target.

In some embodiments, the T-cell engaging multispecific activatable antibody includes an anti-CD3ε scFv and a cancer targeting antibody or antigen-binding fragment thereof, where at least one of the anti-CD3ε scFv and/or the cancer targeting antibody or antigen-binding portion thereof is masked. In some embodiments, the CD3ε scFv includes a first antibody or antigen-binding fragment thereof (AB1) that binds CD3ε, where the AB1 is attached to a masking moiety (MM1) such that coupling of the MM1 reduces the ability of the AB1 to bind CD3ε. In some embodiments, the cancer targeting antibody or antigen-binding fragment thereof includes a second antibody or fragment thereof that includes a second antibody or antigen-binding fragment thereof (AB2) that binds a second, cancer-related target, where the AB2 is attached to a masking moiety (MM2) such that coupling of the MM2 reduces the ability of the AB2 to bind the second, cancer-related target. In some embodiments, the CD3ε scFv includes a first antibody or antigen-binding fragment thereof (AB1) that binds CD3ε, where the AB1 is attached to a masking moiety (MM1) such that coupling of the MM1 reduces the ability of the AB1 to bind CD3ε, and the cancer targeting antibody or antigen-binding fragment thereof includes a second antibody or fragment thereof that includes a second antibody or antigen-binding fragment thereof (AB2) that binds a second, cancer-related target, where the AB2 is attached to a masking moiety (MM2) such that coupling of the MM2 reduces the ability of the AB2 to bind the second, cancer-related target.

In some embodiments, the T-cell engaging multispecific activatable antibody includes an anti-CD3ε scFv and a cancer targeting IgG antibody or antigen-binding fragment thereof, where at least one of the anti-CD3ε scFv and/or the cancer targeting IgG antibody or antigen-binding portion thereof is masked. In some embodiments, the CD3ε scFv includes a first antibody or antigen-binding fragment thereof (AB1) that binds CD3ε, where the AB1 is attached to a masking moiety (MM1) such that coupling of the MM1 reduces the ability of the AB1 to bind CD3ε. In some embodiments, the cancer targeting IgG antibody or antigen-binding fragment thereof includes a second antibody or fragment thereof that includes a second antibody or antigen-binding fragment thereof (AB2) that binds a second, cancer-related target, where the AB2 is attached to a masking moiety (MM2) such that coupling of the MM2 reduces the ability of the AB2 to bind the second, cancer-related target. In some embodiments, the CD3ε scFv includes a first antibody or antigen-binding fragment thereof (AB1) that binds CD3ε, where the AB1 is attached to a masking moiety (MM1) such that coupling of the MM1 reduces the ability of the AB1 to bind CD3ε, and the cancer targeting antibody IgG or antigen-binding fragment thereof includes a second antibody or fragment thereof that includes a second antibody or antigen-binding fragment thereof (AB2) that binds a second, cancer-related target, where the AB2 is attached to a masking moiety (MM2) such that coupling of the MM2 reduces the ability of the AB2 to bind the second, cancer-related target.

In some embodiments, the T-cell engaging multispecific activatable antibody includes an anti-CD3 epsilon (CD3ε) scFv that is derived from OKT3, where at least one of the targeting antibody or antigen-binding fragment thereof and/or the OKT3 scFv or OKT3-derived scFv is masked. In some embodiments, the OKT3 scFv or OKT3-derived scFv includes a first antibody or antigen-binding fragment thereof (AB1) that binds CD3ε, where the AB1 is attached to a masking moiety (MM1) such that coupling of the MM1 reduces the ability of the AB1 to bind CD3ε. In some embodiments, the targeting antibody or antigen-binding fragment thereof includes a second antibody or fragment thereof that includes a second antibody or antigen-binding fragment thereof (AB2) that binds a second target, where the AB2 is attached to a masking moiety (MM2) such that coupling of the MM2 reduces the ability of the AB2 to bind the second target. In some embodiments, the OKT3 scFv or OKT3-derived scFv includes a first antibody or antigen-binding fragment thereof (AB1) that binds CD3ε, where the AB1 is attached to a masking moiety (MM1) such that coupling of the MM1 reduces the ability of the AB1 to bind CD3ε, and the targeting antibody or antigen-binding fragment thereof includes a second antibody or fragment thereof that includes a second antibody or antigen-binding fragment thereof (AB2) that binds a second target, where the AB2 is attached to a masking moiety (MM2) such that coupling of the MM2 reduces the ability of the AB2 to bind the second target.

In some embodiments, the T-cell engaging multispecific activatable antibody includes an OKT3 scFv or OKT3-derived scFv and a cancer targeting antibody or antigen-binding fragment thereof, where at least one of the OKT3 scFv or OKT3-derived scFv and/or the cancer targeting antibody or antigen-binding portion thereof is masked. In some embodiments, the OKT3 scFv or OKT3-derived scFv includes a first antibody or antigen-binding fragment thereof (AB1) that binds CD3ε, where the AB1 is attached to a masking moiety (MM1) such that coupling of the MM1 reduces the ability of the AB1 to bind CD3ε. In some embodiments, the cancer targeting antibody or antigen-binding fragment thereof includes a second antibody or fragment thereof that includes a second antibody or antigen-binding fragment thereof (AB2) that binds a second, cancer-related target, where the AB2 is attached to a masking moiety (MM2) such that coupling of the MM2 reduces the ability of the AB2 to bind the second, cancer-related target. In some embodiments, the OKT3 scFv or OKT3-derived scFv includes a first antibody or antigen-binding fragment thereof (AB1) that binds CD3ε, where the AB1 is attached to a masking moiety (MM1) such that coupling of the MM1 reduces the ability of the AB1 to bind CD3ε, and the cancer targeting antibody or antigen-binding fragment thereof includes a second antibody or fragment thereof that includes a second antibody or antigen-binding fragment thereof (AB2) that binds a second, cancer-related target, where the AB2 is attached to a masking moiety (MM2) such that coupling of the MM2 reduces the ability of the AB2 to bind the second, cancer-related target.

In some embodiments, the T-cell engaging multispecific activatable antibody includes an OKT3 scFv or OKT3-derived scFv and a cancer targeting IgG antibody or antigen-binding fragment thereof, where at least one of the OKT3 scFv or OKT3-derived scFv and/or the cancer targeting IgG antibody or antigen-binding portion thereof is masked. In some embodiments, the OKT3 scFv or OKT3-derived scFv includes a first antibody or antigen-binding fragment thereof (AB1) that binds CD3ε, where the AB1 is attached to a masking moiety (MM1) such that coupling of the MM1 reduces the ability of the AB1 to bind CD3ε. In some embodiments, the cancer targeting IgG antibody or antigen-binding fragment thereof includes a second antibody or fragment thereof that includes a second antibody or antigen-binding fragment thereof (AB2) that binds a second, cancer-related target, where the AB2 is attached to a masking moiety (MM2) such that coupling of the MM2 reduces the ability of the AB2 to bind the second, cancer-related target. In some embodiments, the OKT3 scFv or OKT3-derived scFv includes a first antibody or antigen-binding fragment thereof (AB1) that binds CD3ε, where the AB1 is attached to a masking moiety (MM1) such that coupling of the MM1 reduces the ability of the AB1 to bind CD3ε, and the cancer targeting antibody IgG or antigen-binding fragment thereof includes a second antibody or fragment thereof that includes a second antibody or antigen-binding fragment thereof (AB2) that binds a second, cancer-related target, where the AB2 is attached to a masking moiety (MM2) such that coupling of the MM2 reduces the ability of the AB2 to bind the second, cancer-related target.

In some embodiments, the T-cell engaging multispecific activatable antibody includes an anti-CTLA-4 scFv, where at least one of the targeting antibody or antigen-binding fragment thereof and/or the anti-CTLA-4 scFv is masked. In some embodiments, the anti-CTLA-4 scFv includes a first antibody or antigen-binding fragment thereof (AB1) that binds CTLA-4, where the AB1 is attached to a masking moiety (MM1) such that coupling of the MM1 reduces the ability of the AB1 to bind CTLA-4. In some embodiments, the targeting antibody or antigen-binding fragment thereof includes a second antibody or fragment thereof that includes a second antibody or antigen-binding fragment thereof (AB2) that binds a second target, where the AB2 is attached to a masking moiety (MM2) such that coupling of the MM2 reduces the ability of the AB2 to bind the second target. In some embodiments, the anti-CTLA-4 scFv includes a first antibody or antigen-binding fragment thereof (AB1) that binds CTLA-4, where the AB1 is attached to a masking moiety (MM1) such that coupling of the MM1 reduces the ability of the AB1 to bind CTLA-4, and the targeting antibody or antigen-binding fragment thereof includes a second antibody or fragment thereof that includes a second antibody or antigen-binding fragment thereof (AB2) that binds a second target, where the AB2 is attached to a masking moiety (MM2) such that coupling of the MM2 reduces the ability of the AB2 to bind the second target.

In some embodiments, the T-cell engaging multispecific activatable antibody includes an anti-CTLA-4 scFv and a targeting IgG antibody or antigen-binding fragment thereof, where at least one of the anti-CTLA-4 scFv and/or the targeting IgG antibody or antigen-binding portion thereof is masked. In some embodiments, the anti-CTLA-4 scFv includes a first antibody or antigen-binding fragment thereof (AB1) that binds CTLA-4, where the AB1 is attached to a masking moiety (MM1) such that coupling of the MM1 reduces the ability of the AB1 to bind CTLA-4. In some embodiments, the targeting IgG antibody or antigen-binding fragment thereof includes a second antibody or fragment thereof that includes a second antibody or antigen-binding fragment thereof (AB2) that binds a second target, where the AB2 is attached to a masking moiety (MM2) such that coupling of the MM2 reduces the ability of the AB2 to bind the second target. In some embodiments, the anti-CTLA-4 scFv includes a first antibody or antigen-binding fragment thereof (AB1) that binds CTLA-4, where the AB1 is attached to a masking moiety (MM1) such that coupling of the MM1 reduces the ability of the AB1 to bind CTLA-4, and the targeting antibody IgG or antigen-binding fragment thereof includes a second antibody or fragment thereof that includes a second antibody or antigen-binding fragment thereof (AB2) that binds a second target, where the AB2 is attached to a masking moiety (MM2) such that coupling of the MM2 reduces the ability of the AB2 to bind the second target.

In some embodiments, the multi-antigen targeting antibodies and/or multi-antigen targeting activatable antibodies include at least a first antibody or antigen-binding fragment thereof that binds a first target and/or first epitope and a second antibody or antigen-binding fragment thereof that binds a second target and/or a second epitope. In some embodiments, the multi-antigen targeting antibodies and/or multi-antigen targeting activatable antibodies bind two or more different targets. In some embodiments, the multi-antigen targeting antibodies and/or multi-antigen targeting activatable antibodies bind two or more different epitopes on the same target. In some embodiments, the multi-antigen targeting antibodies and/or multi-antigen targeting activatable antibodies bind a combination of two or more different targets and two or more different epitopes on the same target.

In some embodiments, a multispecific activatable antibody comprising an IgG has the IgG variable domains masked. In some embodiments, a multispecific activatable antibody comprising a scFv has the scFv domains masked. In some embodiments, a multispecific activatable antibody has both IgG variable domains and scFv domains, where at least one of the IgG variable domains is coupled to a masking moiety. In some embodiments, a multispecific activatable antibody has both IgG variable domains and scFv domains, where at least one of the scFv domains is coupled to a masking moiety. In some embodiments, a multispecific activatable antibody has both IgG variable domains and scFv domains, where at least one of the IgG variable domains is coupled to a masking moiety and at least one of the scFv domains is coupled to a masking moiety. In some embodiments, a multispecific activatable antibody has both IgG variable domains and scFv domains, where each of the IgG variable domains and the scFv domains is coupled to its own masking moiety. In some embodiments, one antibody domain of a multispecific activatable antibody has specificity for a target antigen and another antibody domain has specificity for a T-cell surface antigen. In some embodiments, one antibody domain of a multispecific activatable antibody has specificity for a target antigen and another antibody domain has specificity for another target antigen. In some embodiments, one antibody domain of a multispecific activatable antibody has specificity for an epitope of a target antigen and another antibody domain has specificity for another epitope of the target antigen.

In a multispecific activatable antibody, a scFv can be fused to the carboxyl terminus of the heavy chain of an IgG activatable antibody, to the carboxyl terminus of the light chain of an IgG activatable antibody, or to the carboxyl termini of both the heavy and light chains of an IgG activatable antibody. In a multispecific activatable antibody, a scFv can be fused to the amino terminus of the heavy chain of an IgG activatable antibody, to the amino terminus of the light chain of an IgG activatable antibody, or to the amino termini of both the heavy and light chains of an IgG activatable antibody. In a multispecific activatable antibody, a scFv can be fused to any combination of one or more carboxyl termini and one or more amino termini of an IgG activatable antibody. In some embodiments, a masking moiety (MM) linked to a cleavable moiety (CM) is attached to and masks an antigen binding domain of the IgG. In some embodiments, a masking moiety (MM) linked to a cleavable moiety (CM) is attached to and masks an antigen binding domain of at least one scFv. In some embodiments, a masking moiety (MM) linked to a cleavable moiety (CM) is attached to and masks an antigen binding domain of an IgG and a masking moiety (MM) linked to a cleavable moiety (CM) is attached to and masks an antigen binding domain of at least one scFv.

The disclosure provides examples of multispecific activatable antibody structures which include, but are not limited to, the following: (VL-CL)₂:(VH-CH1-CH2-CH3-L4-VH*-L3-VL*-L2-CM-L1-M M)₂; (VL-CL)₂:(VH-CH1-CH2-CH3-L4-VL*-L3-VH*-L2-CM-L1-M M)₂; (MM-L1-CM-L2-VL-CL)₂:(VH-CH1-CH2-CH3-L4-VH*-L3-VL*)₂; (MM-L1-CM-L2-VL-CL)₂:(VH-CH1-CH2-CH3-L4-VL*-L3-VH*)₂; (VL-CL)₂:(MM-L1-CM-L2-VL*-L3-VH*-L4-VH-CH1-CH2-CH3)₂; (VL-CL)₂:(MM-L1-CM-L2-VH*-L3-VL*-L4-VH-CH1-CH2-CH3)₂; (MM-L1-CM-L2-VL-CL)₂:(VL*-L3-VH*-L4-VH-CH1-CH2-CH3)₂; (MM-L1-CM-L2-VL-CL)₂:(VH*-L3-VL*-L4-VH-CH1-CH2-CH3)₂; (VL-CL-L4-VH*-L3-VL*-L2-CM-L1-MM)₂:(VH-CH1-CH2-CH3)₂; (VL-CL-L4-VL*-L3-VH*-L2-CM-L1-M M)₂:(VH-CH1-CH2-CH3)₂; (MM-L1-CM-L2-VL*-L3-VH*-L4-VL-CL)₂:(VH-CH1-CH2-CH3)₂; (MM-L1-CM-L2-VH*-L3-VL*-L4-VL-CL)₂:(VH-CH1-CH2-CH3)₂; (VL-CL-L4-VH*-L3-VL*-L2-CM-L1-MM)₂: (MM-L1-CM-L2-VL*-L3-VH*-L4-VH-CH1-CH2-CH3)₂; (VL-CL-L4-VH*-L3-VL*-L2-CM-L1-MM)₂: (MM-L1-CM-L2-VH*-L3-VL*-L4-VH-CH1-CH2-CH3)₂; (VL-CL-L4-VL*-L3-VH*-L2-CM-L1-M M)₂: (MM-L1-CM-L2-VL*-L3-VH*-L4-VH-CH1-CH2-CH3)₂; (VL-CL-L4-VL*-L3-VH*-L2-CM-L1-MM)₂: (MM-L1-CM-L2-VH*-L3-VL*-L4-VH-CH1-CH2-CH3)₂; (VL-CL-L4-VH*-L3-VL*)₂: (MM-L1-CM-L2-VL*-L3-VH*-L4-VH-CH1-CH2-CH3)₂; (VL-CL-L4-VH*-L3-VL*)₂: (MM-L1-CM-L2-VH*-L3-VL*-L4-VH-CH1-CH2-CH3)₂; (VL-CL-L4-VL*-L3-VH*)₂: (MM-L1-CM-L2-VL*-L3-VH*-L4-VH-CH1-CH2-CH3)₂; (VL-CL-L4-VL*-L3-VH*)₂: (MM-L1-CM-L2-VH*-L3-VL*-L4-VH-CH1-CH2-CH3)₂; (VL-CL-L4-VH*-L3-VL*-L2-CM-L1-MM)₂: (VL*-L3-VH*-L4-VH-CH1-CH2-CH3)₂; (VL-CL-L4-VH*-L3-VL*-L2-CM-L1-MM)₂:(VH*-L3-VL*-L4-VH-CH1-CH2-CH3)₂; (VL-CL-L4-VL*-L3-VH*-L2-CM-L1-MM)₂: (VL*-L3-VH*-L4-VH-CH1-CH2-CH3)₂; or (VL-CL-L4-VL*-L3-VH*-L2-CM-L1-MM)₂:(VH*-L3-VL*-L4-VH-CH1-CH2-CH3)₂, wherein: VL and VH represent the light and heavy variable domains of the first specificity, contained in the IgG; VL* and VH* represent the variable domains of the second specificity, contained in the scFv; L1 is a linker peptide connecting the masking moiety (MM) and the cleavable moiety (CM); L2 is a linker peptide connecting the cleavable moiety (CM), and the antibody; L3 is a linker peptide connecting the variable domains of the scFv; L4 is a linker peptide connecting the antibody of the first specificity to the antibody of the second specificity; CL is the light-chain constant domain; and CH1, CH2, CH3 are the heavy chain constant domains. The first and second specificities may be toward any antigen or epitope.

In some embodiments of a T-cell engaging multispecific activatable antibody, one antigen is typically an antigen present on the surface of a tumor cell or other cell type associated with disease, such as, but not limited to, any target listed in Table 1, such as, but not limited to, EGFR, erbB2, EpCAM, Jagged, PD-L1, B7H3, or CD71 (transferrin receptor), and another antigen is typically a stimulatory (also referred to herein as activating) or inhibitory receptor present on the surface of a T-cell, natural killer (NK) cell, myeloid mononuclear cell, macrophage, and/or other immune effector cell, such as, but not limited to, B7-H4, BTLA, CD3, CD4, CD8, CD16a, CD25, CD27, CD28, CD32, CD56, CD137 (also referred to as TNFRSF9), CTLA-4, GITR, HVEM, ICOS, LAG3, NKG2D, OX40, PD-1, TIGIT, TIM3, or VISTA. The antibody domain conferring specificity to the T-cell surface antigen may also be substituted by a ligand or ligand domain that binds to a T-cell receptor, a NK-cell receptor, a macrophage receptor, and/or other immune effector cell receptor, such as, but not limited to, B7-1, B7-2, B7H3, PD-L1, PD-L2, or TNFSF9. In some embodiments of a multi-antigen targeting activatable antibody, one antigen is selected from the group of targets listed in Table 1, and another antigen is selected from the group of targets listed in Table 1.

In some embodiments, the targeting antibody is an anti-EGFR antibody. In some embodiments, the targeting antibody is C225v5, which is specific for binding to EGFR. In some embodiments, the targeting antibody is C225, which is specific for binding to EGFR. In some embodiments, the targeting antibody is C225v4, which is specific for binding to EGFR. In some embodiments, the targeting antibody is C225v6, which is specific for binding to EGFR. In some embodiments, the targeting antibody is an anti-Jagged antibody. In some embodiments, the targeting antibody is 4D11, which is specific for binding to human and mouse Jagged 1 and Jagged 2. In some embodiments, the targeting antibody is 4D11v2, which is specific for binding to human and mouse Jagged 1 and Jagged 2.

In some embodiments, the targeting antibody can be in the form an activatable antibody. In some embodiments, the scFv(s) can be in the form of a Pro-scFv (see, e.g., WO 2009/025846, WO 2010/081173).

In some embodiments, the scFv is specific for binding CDR, and is or is derived from an antibody or fragment thereof that binds CDR, e.g., CH2527, FN18, H2C, OKT3, 2C11, UCHT1, or V9. In some embodiments, the scFv is specific for binding CTLA-4 (also referred to herein as CTLA and CTLA4).

In some embodiments, the anti-CTLA-4 scFv includes the amino acid sequence:

(SEQ ID NO: 692)
GGGSGGGGSGSGGGSGGGGSGGGEIVLTQSPGTLSLSPGERATLSCRASQ
SVSSSYLAWYQQKPGQAPRLLIYGASSRATGIPDRFSGSGSGTDFTLTIS
RLEPEDFAVYYCQQYGSSPLTFGGGTKVEIKRSGGSTITSYNVYYTKLSS
SGTQVQLVQTGGGVVQPGRSLRLSCAASGSTFSSYAMSWVRQAPGKGLEW
VSAISGSGGSTYYADSVKGRFTISRDNSKNTLYLQMNSLRAEDTAVYYCA
TNSLYWYFDLWGRGTLVTVSSAS

In some embodiments, the anti-CTLA-4 scFv includes the amino acid sequence that is at least 90%, 91%, 92%, 93%, 94%, 95%, 96%, 97%, 98%, 99% or more identical to the amino acid sequence of SEQ ID NO: 692.

In some embodiments, the anti-CD3ε scFv includes the amino acid sequence:

(SEQ ID NO: 693)
GGGSGGGGSGSGGGSGGGGSGGGQVQLQQSGAELARPGASVKMSCKASGY
TFTRYTMHWVKQRPGQGLEWIGYINPSRGYTNYNQKFKDKATLTTDKSSS
TAYMQLSSLTSEDSAVYYCARYYDDHYCLDYWGQGTTLTVSSGGGGSGGG
GSGGGGSQIVLTQSPAIMSASPGEKVTMTCSASSSVSYMNWYQQKSGTSP
KRWIYDTSKLASGVPAHFRGSGSGTSYSLTISGMEAEDAATYYCQQWSSN
PFTFGSGTKLEINR

In some embodiments, the anti-CD3ε scFv includes the amino acid sequence that is at least 90%, 91%, 92%, 93%, 94%, 95%, 96%, 97%, 98%, 99% or more identical to the amino acid sequence of SEQ ID NO: 693.

In some embodiments, the scFv is specific for binding one or more T-cells, one or more NK-cells and/or one or more macrophages. In some embodiments, the scFv is specific for binding a target selected from the group consisting of B7-H4, BTLA, CD3, CD4, CD8, CD16a, CD25, CD27, CD28, CD32, CD56, CD137, CTLA-4, GITR, HVEM, ICOS, LAG3, NKG2D, OX40, PD-1, TIGIT, TIM3, or VISTA.

In some embodiments, the multispecific activatable antibody also includes an agent conjugated to the AB. In some embodiments, the agent is a therapeutic agent. In some embodiments, the agent is an antineoplastic agent. In some embodiments, the agent is a toxin or fragment thereof. In some embodiments, the agent is conjugated to the multispecific activatable antibody via a linker. In some embodiments, the agent is conjugated to the AB via a cleavable linker. In some embodiments, the agent is conjugated to the AB via a linker that includes at least one uPA-cleavable substrate sequence or at least one matriptase-cleavable substrate sequence. In some embodiments, the linker is a non-cleavable linker. In some embodiments, the agent is a microtubule inhibitor. In some embodiments, the agent is a nucleic acid damaging agent, such as a DNA alkylator or DNA intercalator, or other DNA damaging agent. In some embodiments, the linker is a cleavable linker. In some embodiments, the agent is an agent selected from the group listed in Table 4. In some embodiments, the agent is a dolastatin. In some embodiments, the agent is an auristatin or derivative thereof. In some embodiments, the agent is auristatin E or a derivative thereof. In some embodiments, the agent is monomethyl auristatin E (MMAE). In some embodiments, the agent is monomethyl auristatin D (MMAD). In some embodiments, the agent is a maytansinoid or maytansinoid derivative. In some embodiments, the agent is DM1 or DM4. In some embodiments, the agent is a duocarmycin or derivative thereof. In some embodiments, the agent is a calicheamicin or derivative thereof. In some embodiments, the agent is a pyrrolobenzodiazepine.

In some embodiments, the multispecific activatable antibody also includes a detectable moiety. In some embodiments, the detectable moiety is a diagnostic agent.

In some embodiments, the multispecific activatable antibody naturally contains one or more disulfide bonds. In some embodiments, the multispecific activatable antibody can be engineered to include one or more disulfide bonds.

The disclosure also provides an isolated nucleic acid molecule encoding a multispecific activatable antibody described herein, as well as vectors that include these isolated nucleic acid sequences. The disclosure provides methods of producing a multispecific activatable antibody by culturing a cell under conditions that lead to expression of the activatable antibody, wherein the cell comprises such a nucleic acid molecule. In some embodiments, the cell comprises such a vector.

The disclosure also provides a method of manufacturing multispecific activatable antibodies of the disclosure by (a) culturing a cell comprising a nucleic acid construct that encodes the multispecific activatable antibody under conditions that lead to expression of the multispecific activatable, and (b) recovering the multispecific activatable antibody.

The disclosure also provides multispecific activatable antibodies and/or multispecific activatable antibody compositions that include at least a first antibody or antigen-binding fragment thereof (AB1) that specifically binds a first target or first epitope and a second antibody or antigen-biding fragment thereof (AB2) that binds a second target or a second epitope, where at least AB1 is coupled or otherwise attached to a masking moiety (MM1), such that coupling of the MM1 reduces the ability of AB1 to bind its target. In some embodiments, the MM1 is coupled to AB1 via a first cleavable moiety (CM1) sequence that includes a substrate for a protease, for example, a protease that is co-localized with the target of AB1 at a treatment site or a diagnostic site in a subject. The multispecific activatable antibodies provided herein are stable in circulation, activated at intended sites of therapy and/or diagnosis but not in normal, i.e., healthy tissue, and, when activated, exhibit binding to the target of AB1 that is at least comparable to the corresponding, unmodified multispecific antibody.

In some embodiments, the multispecific activatable antibody comprises a linking peptide between the MM1 and the CM1.

In some embodiments, the multispecific activatable antibody comprises a linking peptide between the CM1 and the AB1.

In some embodiments, the activatable antibody comprises a first linking peptide (LP1) and a second linking peptide (LP2), and at least a portion of the multispecific activatable antibody has the structural arrangement from N-terminus to C-terminus as follows in the uncleaved state: MM1-LP1-CM1-LP2-AB1 or AB1-LP2-CM1-LP1-MM1. In some embodiments, the two linking peptides need not be identical to each other.

In some embodiments, at least one of LP1 or LP2 includes an amino acid sequence selected from the group consisting of (GS)ₙ, (GGS)ₙ, (GSGGS)ₙ (SEQ ID NO: 385) and (GGGS)ₙ (SEQ ID NO: 386), where n is an integer of at least one. In some embodiments, at least one of LP1 or LP2 includes an amino acid sequence selected from the group consisting of GGSG (SEQ ID NO: 387), GGSGG (SEQ ID NO: 388), GSGSG (SEQ ID NO: 389), GSGGG (SEQ ID NO: 390), GGGSG (SEQ ID NO: 391), and GSSSG (SEQ ID NO: 392).

In some embodiments, the multispecific activatable antibody includes at least a first antibody or antigen-binding fragment thereof (AB1) that specifically binds a first target or first epitope and a second antibody or antigen-binding fragment thereof (AB2) that specifically binds a second target or second epitope. In some embodiments, each of the AB in the multispecific activatable antibody is independently selected from the group consisting of a monoclonal antibody, domain antibody, single chain, Fab fragment, a F(ab′)₂ fragment, a scFv, a scAb, a dAb, a single domain heavy chain antibody, and a single domain light chain antibody. In some embodiments, each of the AB in the multispecific activatable antibody is a rodent (e.g., mouse or rat), chimeric, humanized or fully human monoclonal antibody.

In some embodiments, each of the AB in the multispecific activatable antibody has an equilibrium dissociation constant of about 100 nM or less for binding to its corresponding target or epitope.

In some embodiments, MM1 has an equilibrium dissociation constant for binding to its corresponding AB that is greater than the equilibrium dissociation constant of the AB to its corresponding target or epitope.

In some embodiments, MM1 has an equilibrium dissociation constant for binding to its corresponding AB that is no more than the equilibrium dissociation constant of the AB to its corresponding target or epitope.

In some embodiments, MM1 does not interfere or compete with its corresponding AB for binding to the corresponding target or epitope when the multispecific activatable antibody is in a cleaved state.

In some embodiments, MM1 is a polypeptide of about 2 to 40 amino acids in length. In some embodiments, each of the MM in the multispecific activatable antibody is a polypeptide of no more than 40 amino acids in length.

In some embodiments, MM1 has a polypeptide sequence that is different from that of target of the corresponding AB.

In some embodiments, MM1 has a polypeptide sequence that is no more than 50% identical to any natural binding partner of the corresponding AB. In some embodiments, MM1 has a polypeptide sequence that is no more than 25% identical to any natural binding partner of the corresponding AB. In some embodiments, MM1 has a polypeptide sequence that is no more than 10% identical to any natural binding partner of the corresponding AB.

In some embodiments, the coupling of MM1 reduces the ability of the corresponding AB to bind its target or epitope such that the dissociation constant (K_{d}) of the AB when coupled to the MM1 towards its corresponding target or epitope is at least 20 times greater than the K_{d} of the AB when not coupled to the MM1 towards its corresponding target or epitope.

In some embodiments, the coupling of MM1 reduces the ability of the corresponding AB to bind its target or epitope such that the dissociation constant (K_{d}) of the AB when coupled to the MM1 towards its corresponding target or epitope is at least 40 times greater than the K_{d} of the AB when not coupled to the MM1 towards its corresponding target or epitope.

In some embodiments, the coupling of MM1 reduces the ability of the corresponding AB to bind its target or epitope such that the dissociation constant (K_{d}) of the AB when coupled to the MM1 towards its corresponding target or epitope is at least 100 times greater than the K_{d} of the AB when not coupled to the MM1 towards its corresponding target or epitope.

In some embodiments, the coupling of MM1 reduces the ability of the corresponding AB to bind its target or epitope such that the dissociation constant (K_{d}) of the AB when coupled to the MM1 towards its corresponding target or epitope is at least 1000 times greater than the K_{d} of the AB when not coupled to the MM1 towards its corresponding target or epitope.

In some embodiments, the coupling of MM1 reduces the ability of the corresponding AB to bind its target or epitope such that the dissociation constant (K_{d}) of the AB when coupled to the MM1 towards its corresponding target or epitope is at least 10,000 times greater than the K_{d} of the AB when not coupled to the MM1 towards its corresponding target or epitope.

In some embodiments, MM1 is an amino acid sequence selected from a MM disclosed herein.

In some embodiments, the multispecific activatable antibody includes at least a second masking moiety (MM2) that inhibits the binding of the AB2 to its target when the multispecific activatable antibody is in an uncleaved state, and a second cleavable moiety (CM2) coupled to the AB2, wherein the CM2 is a polypeptide that functions as a substrate for a second protease. In some embodiments, CM2 is a polypeptide of no more than 15 amino acids long. In some embodiments, the second protease is co-localized with the second target or epitope in a tissue, and wherein the second protease cleaves the CM2 in the multispecific activatable antibody when the multispecific activatable antibody is exposed to the second protease. In some embodiments, the first protease and the second protease are co-localized with the first target or epitope and the second target or epitope in a tissue. In some embodiments, the first protease and the second protease are the same protease. In some embodiments, CM1 and CM2 are different substrates for the same protease. In some embodiments, the protease is selected from the group consisting of those shown in Table 7. In some embodiments, the first protease and the second protease are different proteases. In some embodiments, the first protease and the second protease are different proteases selected from the group consisting of those shown in Table 7.

In some embodiments, each of the MM in the multispecific activatable antibody, e.g., MM1 and at least MM2, has an equilibrium dissociation constant for binding to its corresponding AB that is greater than the equilibrium dissociation constant of the AB to its corresponding target or epitope.

In some embodiments, each of the MM in the multispecific activatable antibody has an equilibrium dissociation constant for binding to its corresponding AB that is no more than the equilibrium dissociation constant of the AB to its corresponding target or epitope.

In some embodiments, each of the MM in the multispecific activatable antibody does not interfere or compete with its corresponding AB for binding to the corresponding target or epitope when the multispecific activatable antibody is in a cleaved state.

In some embodiments, each of the MM in the multispecific activatable antibody is a polypeptide of about 2 to 40 amino acids in length. In some embodiments, each of the MM in the multispecific activatable antibody is a polypeptide of no more than 40 amino acids in length.

In some embodiments, each of the MM in the multispecific activatable antibody has a polypeptide sequence that is different from that of target of the corresponding AB.

In some embodiments, each of the MM in the multispecific activatable antibody has a polypeptide sequence that is no more than 50% identical to any natural binding partner of the corresponding AB. In some embodiments, each of the MM in the multispecific activatable antibody has a polypeptide sequence that is no more than 25% identical to any natural binding partner of the corresponding AB. In some embodiments, each of the MM in the multispecific activatable antibody has a polypeptide sequence that is no more than 10% identical to any natural binding partner of the corresponding AB.

In some embodiments, the coupling of each of the MM reduces the ability of the corresponding AB to bind its target or epitope such that the dissociation constant (K_{d}) of the AB when coupled to the MM towards its corresponding target or epitope is at least 20 times greater than the K_{d} of the AB when not coupled to the MM towards its corresponding target or epitope.

In some embodiments, the coupling of each of the MM reduces the ability of the corresponding AB to bind its target or epitope such that the dissociation constant (K_{d}) of the AB when coupled to the MM towards its corresponding target or epitope is at least 40 times greater than the K_{d} of the AB when not coupled to the MM towards its corresponding target or epitope.

In some embodiments, the coupling of each of the MM reduces the ability of the corresponding AB to bind its target or epitope such that the dissociation constant (K_{d}) of the AB when coupled to the MM towards its corresponding target or epitope is at least 100 times greater than the K_{d} of the AB when not coupled to the MM towards its corresponding target or epitope.

In some embodiments, the coupling of each of the MM reduces the ability of the corresponding AB to bind its target or epitope such that the dissociation constant (K_{d}) of the AB when coupled to the MM towards its corresponding target or epitope is at least 1000 times greater than the K_{d} of the AB when not coupled to the MM towards its corresponding target or epitope.

In some embodiments, the coupling of each of the MM reduces the ability of the corresponding AB to bind its target or epitope such that the dissociation constant (K_{d}) of the AB when coupled to the MM towards its corresponding target or epitope is at least 10,000 times greater than the K_{d} of the AB when not coupled to the MM towards its corresponding target or epitope.

In some embodiments, each of the MM is an amino acid sequence selected from a MM disclosed herein.

In some embodiments, at least one of CM1 and/or CM2 is cleaved by at least one protease selected from uPA and matriptase. In some embodiments, at least one of CM1 and/or CM2 includes an amino acid sequence selected from the group consisting of a core CM consensus sequence shown in Tables 8A-8J, a subgenus of a core CM consensus sequence shown in Tables 8A-8J, an expanded consensus sequence based on one of the core CM consensus sequence shown in Tables 8A-8J, an. In some embodiments, the expanded consensus sequence is a consensus sequence shown in Tables 9A-9J-3, a core CM consensus sequence shown in Tables 10A-10D, a subgenus of a core CM consensus sequence shown in Tables 10A-10D, and a consensus sequence shown in Tables 11A-11D.

In some embodiments, at least one of CM1 and/or CM2 includes an amino acid sequence selected from the group consisting of SEQ ID NOs: 163-267.

In some embodiments, the protease that cleaves the first cleavable moiety (CM1) sequence is co-localized with the target of the AB1 in the multispecific activatable antibody in a tissue, and the protease cleaves the CM1 in the multispecific activatable antibody when the multispecific activatable antibody is exposed to the protease.

In some embodiments, the multispecific activatable antibody includes more than one cleavable moiety sequence, and the protease that cleaves at least one cleavable moiety sequence is co-localized with the target of at least one of the AB regions in the multispecific activatable antibody in a tissue, and the protease cleaves the CM in the multispecific activatable antibody when the multispecific activatable antibody is exposed to the protease.

In some embodiments, each CM, e.g., CM1 and at least CM2, is positioned in the multispecific activatable antibody such that in the uncleaved state, binding of the multispecific activatable antibody to a target of one of the AB regions is reduced to occur with an equilibrium dissociation constant that is at least twofold greater than the equilibrium dissociation constant of an unmodified AB binding to its target, and whereas in the cleaved state, the AB binds its target.

In some embodiments, each CM, e.g., CM1 and at least CM2, is positioned in the multispecific activatable antibody such that in the uncleaved state, binding of the multispecific activatable antibody to a target of one of the AB regions is reduced to occur with an equilibrium dissociation constant that is at least threefold greater than the equilibrium dissociation constant of an unmodified AB binding to its target, and whereas in the cleaved state, the AB binds its target.

In some embodiments, each CM, e.g., CM1 and at least CM2, is positioned in the multispecific activatable antibody such that in the uncleaved state, binding of the multispecific activatable antibody to a target of one of the AB regions is reduced to occur with an equilibrium dissociation constant that is at least fourfold greater than the equilibrium dissociation constant of an unmodified AB binding to its target, and whereas in the cleaved state, the AB binds its target.

In some embodiments, each CM, e.g., CM1 and at least CM2, is positioned in the multispecific activatable antibody such that in the uncleaved state, binding of the multispecific activatable antibody to a target of one of the AB regions is reduced to occur with an equilibrium dissociation constant that is at least fivefold greater than the equilibrium dissociation constant of an unmodified AB binding to its target, and whereas in the cleaved state, the AB binds its target.

In some embodiments, each CM, e.g., CM1 and at least CM2, is positioned in the multispecific activatable antibody such that in the uncleaved state, binding of the multispecific activatable antibody to a target of one of the AB regions is reduced to occur with an equilibrium dissociation constant that is at least tenfold greater than the equilibrium dissociation constant of an unmodified AB binding to its target, and whereas in the cleaved state, the AB binds its target.

In some embodiments, each CM, e.g., CM1 and at least CM2, is positioned in the multispecific activatable antibody such that in the uncleaved state, binding of the multispecific activatable antibody to a target of one of the AB regions is reduced to occur with an equilibrium dissociation constant that is at least 20-fold greater than the equilibrium dissociation constant of an unmodified AB binding to its target, and whereas in the cleaved state, the AB binds its target.

In some embodiments, each CM is positioned in the multispecific activatable antibody such that in the uncleaved state, binding of the multispecific activatable antibody to a target of one of the AB regions is reduced to occur with an equilibrium dissociation constant that is at least 40-fold greater than the equilibrium dissociation constant of an unmodified AB binding to its target, and whereas in the cleaved state, the AB binds its target.

In some embodiments, each CM is positioned in the multispecific activatable antibody such that in the uncleaved state, binding of the multispecific activatable antibody to a target of one of the AB regions is reduced to occur with an equilibrium dissociation constant that is at least 50-fold greater than the equilibrium dissociation constant of an unmodified AB binding to its target, and whereas in the cleaved state, the AB binds its target.

In some embodiments, each CM is positioned in the multispecific activatable antibody such that in the uncleaved state, binding of the multispecific activatable antibody to a target of one of the AB regions is reduced to occur with an equilibrium dissociation constant that is at least 100-fold greater than the equilibrium dissociation constant of an unmodified AB binding to its target, and whereas in the cleaved state, the AB binds its target.

In some embodiments, each CM is positioned in the multispecific activatable antibody such that in the uncleaved state, binding of the multispecific activatable antibody to a target of one of the AB regions is reduced to occur with an equilibrium dissociation constant that is at least 200-fold greater than the equilibrium dissociation constant of an unmodified AB binding to its target, and whereas in the cleaved state, the AB binds its target.

In some embodiments, each CM in the multispecific activatable antibody is a polypeptide of up to 15 amino acids in length.

In some embodiments, at least one CM in the multispecific activatable antibody includes an amino acid sequence selected from the group consisting of SEQ ID NOs: 163-267 and the other CM includes the amino acid sequence LSGRSDNH (SEQ ID NO: 406). In some embodiments, at least one CM includes the amino acid sequence LSGRSDNH (SEQ ID NO: 406). In some embodiments, at least one cleavable moiety is selected for use with a specific protease, for example a protease that is known to be co-localized with at least one target of the multispecific activatable antibody. For example, suitable cleavable moieties for use in the multispecific activatable antibodies of the disclosure are cleaved by at least a protease such as urokinase, legumain, and/or matriptase (also referred to herein as MT-SP1 or MTSP1). In some embodiments, a suitable cleavable moiety includes at least one amino acid sequence selected from the group consisting of SEQ ID NO: 163-267.

In some embodiments, one CM is a substrate for at least one protease selected from uPA and matriptase, and the other CM in the multispecific activatable antibody is a substrate for a protease selected from the group consisting of those shown in Table 7. In some embodiments, the protease is selected from the group consisting of uPA, legumain, matriptase, ADAM17, BMP-1, TMPRSS3, TMPRSS4, neutrophil elastase, MMP-7, MMP-9, MMP-12, MMP-13, and MMP-14. In some embodiments, the protease is a cathepsin, such as, but not limited to, cathepsin S. In some embodiments, each CM in the multispecific activatable antibody is a substrate for a protease selected from the group consisting of uPA (urokinase plasminogen activator), legumain and matriptase. In some embodiments, the protease comprises uPA. In some embodiments, the protease comprises legumain. In some embodiments, the protease comprises matriptase.

In some embodiments, at least one CM in the multispecific activatable antibody is a substrate for at least two proteases. In some embodiments, at least one CM in the multispecific activatable antibody is a substrate for at least two proteases, wherein one of the proteases is selected from the group consisting of uPA and matriptase and the other protease is selected from the group consisting of those shown in Table 7. In some embodiments, at least one CM in the multispecific activatable antibody is a substrate for at least two proteases selected from the group consisting of uPA, legumain and matriptase.

In some embodiments, the multispecific activatable antibody includes at least a first CM (CM1) and a second CM (CM2). In some embodiments, CM1 and CM2 are part of a single cleavable linker that joins an MM to an AB. In some embodiments, CM1 is part of a cleavable linker that joins MM1 to AB1, and CM2 is part of a separate cleavable linker that joins an MM2 to AB2. In some embodiments, a multispecific activatable antibody comprises more than two CMs. In some embodiments, such a multispecific activatable antibody comprises more than two CMs and more than two MMs. In some embodiments, CM1 and CM2 are each polypeptides of no more than 15 amino acids long. In some embodiments, at least one of the first CM and the second CM is a polypeptide that functions as a substrate for a protease selected from the group consisting of those listed in Table 7. In some embodiments, at least one of the first CM and the second CM is a polypeptide that functions as a substrate for a protease selected from the group consisting of uPA, legumain, and matriptase. In some embodiments, the first CM is cleaved by a first cleaving agent selected from the group consisting of uPA, legumain, and matriptase in a target tissue and the second CM is cleaved by a second cleaving agent in a target tissue. In some embodiments, the other protease is selected from the group consisting of those shown in Table 7. In some embodiments, the first cleaving agent and the second cleaving agent are the same protease selected from the group consisting of those listed in Table 7, and the first CM and the second CM are different substrates for the enzyme. In some embodiments, the first cleaving agent and the second cleaving agent are the same protease selected from the group consisting of uPA, legumain, and matriptase, and the first CM and the second CM are different substrates for the enzyme. In some embodiments, the first cleaving agent and the second cleaving agent are the same protease selected from the group listed in Table 7, and the first CM and the second CM are the same substrate. In some embodiments, the first cleaving agent and the second cleaving agent are different proteases. In some embodiments, the first cleaving agent and the second cleaving agent are different proteases selected from the group consisting of those shown in Table 7. In some embodiments, the first cleaving agent and the second cleaving agent are co-localized in the target tissue. In some embodiments, the first CM and the second CM are cleaved by at least one cleaving agent in the target tissue.

In some embodiments, the multispecific activatable antibody is exposed to and cleaved by a protease such that, in the activated or cleaved state, the activated multispecific activatable antibody includes a light chain amino acid sequence that includes at least a portion of LP2 and/or CM sequence after the protease has cleaved the CM.

The disclosure also provides compositions and methods that include a multispecific activatable antibody that includes at least a first antibody or antibody fragment (AB1) that specifically binds a target and a second antibody or antibody fragment (AB2), where at least the first AB in the multispecific activatable antibody is coupled to a masking moiety (MM1) that decreases the ability of AB1 to bind its target. In some embodiments, each AB is coupled to a MM that decreases the ability of its corresponding AB to each target. For example, in bispecific activatable antibody embodiments, AB1 is coupled to a first masking moiety (MM1) that decreases the ability of AB1 to bind its target, and AB2 is coupled to a second masking moiety (MM2) that decreases the ability of AB2 to bind its target. In some embodiments, the multispecific activatable antibody comprises more than two AB regions; in such embodiments, AB1 is coupled to a first masking moiety (MM1) that decreases the ability of AB1 to bind its target, AB2 is coupled to a second masking moiety (MM2) that decreases the ability of AB2 to bind its target, AB3 is coupled to a third masking moiety (MM3) that decreases the ability of AB3 to bind its target, and so on for each AB in the multispecific activatable antibody.

In some embodiments, the multispecific activatable antibody further includes at least one cleavable moiety (CM) that is a substrate for a protease, where the CM links a MM to an AB. For example, in some embodiments, the multispecific activatable antibody includes at least a first antibody or antibody fragment (AB1) that specifically binds a target and a second antibody or antibody fragment (AB2), where at least the first AB in the multispecific activatable antibody is coupled via a first cleavable moiety (CM1) to a masking moiety (MM1) that decreases the ability of AB1 to bind its target. In some bispecific activatable antibody embodiments, AB1 is coupled via CM1 to MM1, and AB2 is coupled via a second cleavable moiety (CM2) to a second masking moiety (MM2) that decreases the ability of AB2 to bind its target. In some embodiments, the multispecific activatable antibody comprises more than two AB regions; in some of these embodiments, AB1 is coupled via CM1 to MM1, AB2 is coupled via CM2 to MM2, and AB3 is coupled via a third cleavable moiety (CM3) to a third masking moiety (MM3) that decreases the ability of AB3 to bind its target, and so on for each AB in the multispecific activatable antibody.

Activatable Antibodies Having Non-Binding Steric Moieties or Binding Partners for Non-Binding Steric Moieties

The disclosure also provides activatable antibodies that include non-binding steric moieties (NB) or binding partners (BP) for non-binding steric moieties, where the BP recruits or otherwise attracts the NB to the activatable antibody. The activatable antibodies provided herein include, for example, an activatable antibody that includes a non-binding steric moiety (NB), a cleavable linker (CL) and antibody or antibody fragment (AB) that binds a target; an activatable antibody that includes a binding partner for a non-binding steric moiety (BP), a CL and an AB; and an activatable antibody that includes a BP to which an NB has been recruited, a CL and an AB that binds the target. Activatable antibodies in which the NB is covalently linked to the CL and AB of the activatable antibody or is associated by interaction with a BP that is covalently linked to the CL and AB of the activatable antibody are referred to herein as “NB-containing activatable antibodies.” By activatable or switchable is meant that the activatable antibody exhibits a first level of binding to a target when the activatable antibody is in an inhibited, masked or uncleaved state (i.e., a first conformation), and a second level of binding to the target when the activatable antibody is in an uninhibited, unmasked and/or cleaved state (i.e., a second conformation, i.e., activated antibody), where the second level of target binding is greater than the first level of target binding. The activatable antibody compositions can exhibit increased bioavailability and more favorable biodistribution compared to conventional antibody therapeutics.

In some embodiments, activatable antibodies provide for reduced toxicity and/or adverse side effects that could otherwise result from binding of the at non-treatment sites and/or non-diagnostic sites if the AB were not masked or otherwise inhibited from binding to such a site.

In one embodiment, the activatable antibody includes a non-binding steric moiety (NB); a cleavable linker (CL); and an antibody or antibody fragment (AB) that binds specifically to the target, wherein the NB is a polypeptide that does not bind specifically to the AB; the CL is a polypeptide that includes a substrate (S) for an enzyme; the CL is positioned such that in an uncleaved state, the NB interferes with binding of the AB to the target and in a cleaved state, the NB does not interfere with binding of the AB to the target; and the NB does not inhibit cleavage of the CL by the enzyme. As used herein and throughout, the term polypeptide refers to any polypeptide that includes at least two amino acid residues, including larger polypeptides, full-length proteins and fragments thereof, and the term polypeptide is not limited to single-chain polypeptides and can include multi-unit, e.g., multi-chain, polypeptides. In cases where the polypeptide is of a shorter length, for example, less than 50 amino acids total, the terms peptide and polypeptide are used interchangeably herein, and in cases where the polypeptide is of a longer length, e.g., 50 amino acids or greater, the terms polypeptide and protein are used interchangeably herein.

In one embodiment, the activatable antibody includes a non-binding steric moiety (NB); a cleavable linker (CL); and an antibody or antibody fragment (AB) that binds specifically to the target, wherein (i) the NB includes a polypeptide that does not bind specifically to the AB; (ii) CL is a polypeptide of up to 50 amino acids in length that includes a substrate (S) for an enzyme, e.g., a protease selected from matriptase and uPA; (iii) the CL is positioned such that in an uncleaved state, the NB interferes with binding of the AB to the target and in a cleaved state, the NB does not interfere with binding of the AB to the target; and (iv) the NB does not inhibit cleavage of the CL by the enzyme. For example, the CL has a length of up to 15 amino acids, a length of up to 20 amino acids, a length of up to 25 amino acids, a length of up to 30 amino acids, a length of up to 35 amino acids, a length of up to 40 amino acids, a length of up to 45 amino acids, a length of up to 50 amino acids, a length in the range of 10-50 amino acids, a length in the range of 15-50 amino acids, a length in the range of 20-50 amino acids, a length in the range of 25-50 amino acids, a length in the range of 30-50 amino acids, a length in the range of 35-50 amino acids, a length in the range of 40-50 amino acids, a length in the range of 45-50 amino acids, a length in the range of 10-40 amino acids, a length in the range of 15-40 amino acids, a length in the range of 20-40 amino acids, a length in the range of 25-40 amino acids, a length in the range of 30-40 amino acids, a length in the range of 35-40 amino acids, a length in the range of 10-30 amino acids, a length in the range of 15-30 amino acids, a length in the range of 20-30 amino acids, a length in the range of 25-30 amino acids, a length in the range of 10-20 amino acids, or a length in the range of 10-15 amino acids.

In one embodiment, the activatable antibody includes a non-binding steric moiety (NB); a cleavable linker (CL); and an antibody or antibody fragment (AB) that binds specifically to the target, wherein (i) the NB includes a polypeptide that does not bind specifically to the AB; (ii) the CL is a polypeptide that includes a substrate (S) for an enzyme, e.g., a protease selected from matriptase and uPA; (iii) the CL is positioned such that in an uncleaved state, the NB interferes with binding of the AB to the target and in a cleaved state, the NB does not interfere with binding of the AB to the target; (iv) the NB does not inhibit cleavage of the CL by the enzyme; and (v) the activatable antibody has the structural arrangement from N-terminus to C-terminus as follows in the uncleaved state: NB-CL-AB or AB-CL-NB.

In one embodiment, the activatable antibody includes a non-binding steric moiety (NB); a cleavable linker (CL); and an antibody or antibody fragment (AB) that binds specifically to the target, wherein (i) the NB includes a polypeptide that does not bind specifically to the AB; (ii) the CL is a polypeptide that includes a substrate (S) for an enzyme, e.g., a protease selected from matriptase and uPA; (iii) the CL is positioned such that in an uncleaved state, the NB interferes with binding of the AB to the target and in a cleaved state, the NB does not interfere with binding of the AB to the target, and wherein the NB in the uncleaved activatable antibody reduces the ability of the AB to bind the target by at least 50%, for example, by at least 60%, by at least 70%, by at least 75%, by at least 80%, by at least 85%, by at least 90%, by at least 95%, by at least 96%, by at least 97%, by at least 98%, by at least 99%, by at least 100% as compared to the ability of the cleaved AB to bind the target; and (iv) the NB does not inhibit cleavage of the CL by the enzyme. The reduction in the ability of the AB to bind the target is determined, e.g., using an assay as described herein or an in vitro target displacement assay such as, for example, the assay described in PCT Publication Nos. WO 2009/025846 and WO 2010/081173.

In one embodiment, the activatable antibody includes a binding partner (BP) for a non-binding steric moiety (NB); a cleavable linker (CL); and an antibody or antibody fragment (AB) that binds specifically to the target, wherein the BP is a polypeptide that binds to the NB when exposed thereto; the NB does not bind specifically to the AB; the CL is a polypeptide that includes a substrate (S) for an enzyme, e.g., a protease selected from matriptase and uPA; the CL is positioned such that in an uncleaved state in the presence of the NB, the NB interferes with binding of the AB to the target and in a cleaved state, the NB does not interfere with binding of the AB to the target and the BP does not interfere with binding of the AB to the target; and the NB and the BP do not inhibit cleavage of the CL by the enzyme. In some examples of this embodiment, the BP of the activatable antibody is optionally bound to the NB. In one embodiment, the NB is recruited by the BP of the activatable antibody in vivo.

In some examples of any of these activatable antibody embodiments, the activatable antibody is formulated as a composition. In some of these embodiments, the composition also includes the NB, where the NB is co-formulated with the activatable antibody that includes the BP, the CL, and the AB. In some examples of this embodiment, the BP is selected from the group consisting of an albumin binding peptide, a fibrinogen binding peptide, a fibronectin binding peptide, a hemoglobin binding peptide, a transferrin binding peptide, an immunoglobulin domain binding peptide, and other serum protein binding peptides.

In some examples of any of these activatable antibody embodiments, the NB is a soluble, globular protein. In some examples of any of these activatable antibody embodiments, the NB is a protein that circulates in the bloodstream. In some examples of any of these activatable antibody embodiments, the NB is selected from the group consisting of albumin, fibrinogen, fibronectin, hemoglobin, transferrin, an immunoglobulin domain, and other serum proteins.

In some examples of any of these activatable antibody embodiments, the CL is a polypeptide that includes a substrate (S) for a protease selected from matriptase and uPA. In some examples of any of these activatable antibody embodiments, the protease is co-localized with the in a tissue, and the protease cleaves the CL in the activatable antibody when the activatable antibody is exposed to the protease. In some examples of any of these activatable antibody embodiments, the CL is a polypeptide of up to 50 amino acids in length. In some examples of any of these activatable antibody embodiments, the CL is a polypeptide that includes a substrate (S) having a length of up to 15 amino acids, e.g., 3 amino acids long, 4 amino acids long, 5 amino acids long, 6 amino acids long, 7 amino acids long, 8 amino acids long, 9 amino acids long, 10 amino acids long, 11 amino acids long, 12 amino acids long, 13 amino acids long, 14 amino acids long, or 15 amino acids long.

In some examples of any of these activatable antibody embodiments, the activatable antibody has the structural arrangement from N-terminus to C-terminus as follows in the uncleaved state: NB-CL-AB, AB-CL-NB, BP-CL-AB or AB-CL-BP. In embodiments where the activatable antibody includes a BP and the activatable antibody is in the presence of the corresponding NB, the activatable antibody has a structural arrangement from N-terminus to C-terminus as follows in the uncleaved state: NB:BP-CM-AB or AB-CM-BP:NB, where “:” represents an interaction, e.g., binding, between the NB and BP.

In some examples of any of these activatable antibody embodiments, the activatable antibody includes an antibody or antigen-binding fragment thereof that specifically binds a given target and is a monoclonal antibody, domain antibody, single chain, Fab fragment, a F(ab′)₂ fragment, a scFv, a scab, a dAb, a single domain heavy chain antibody, or a single domain light chain antibody. In some embodiments, such an antibody or immunologically active fragment thereof that binds the target a mouse, other rodent, chimeric, humanized or fully human monoclonal antibody.

In some examples of any of these activatable antibody embodiments, the activatable antibody includes a combination of a variable heavy chain region comprising an amino acid sequence presented herein and a variable light chain region comprising an amino acid sequence presented herein. In some embodiments, the activatable antibody includes a combination of a variable heavy chain region comprising an amino acid sequence that is at least 90%, 91%, 92%, 93%, 94%, 95%, 96%, 97%, 98%, 99% or more identical to an amino acid sequence presented herein, and a variable light chain region comprising an amino acid sequence that is at least 90%, 91%, 92%, 93%, 94%, 95%, 96%, 97%, 98%, 99% or more identical to an amino acid sequence presented herein.

In some examples of any of these activatable antibody embodiments, the activatable antibody also includes an agent conjugated to the AB. In some embodiments, the agent is a therapeutic agent. In some embodiments, the agent is an antineoplastic agent. In some embodiments, the agent is a toxin or fragment thereof. In some embodiments, the agent is conjugated to the AB via a linker. In some embodiments, the linker is a cleavable linker. In some embodiments, the agent is conjugated to the AB via a noncleavable linker. In some embodiments, the agent is an agent selected from the group listed in Table 3. In some embodiments, the agent is a microtubule inhibitor. In some embodiments, the agent is a nucleic acid damaging agent, such as a DNA alkylator or DNA intercalator, or other DNA damaging agent. In some embodiments, the agent is a dolastatin. In some embodiments, the agent is an auristatin or derivative thereof. In some embodiments, the agent is auristatin E or a derivative thereof. In some embodiments, the agent is monomethyl auristatin E (MMAE). In some embodiments, the agent is monomethyl auristatin D (MMAD). In some embodiments, the agent is a maytansinoid or maytansinoid derivative. In some embodiments, the agent is DM1 or DM4. In some embodiments, the agent is a duocarmycin or derivative thereof. In some embodiments, the agent is a calicheamicin or derivative thereof. In some embodiments, the agent is a pyrrolobenzodiazepine.

In some examples of any of these activatable antibody embodiments, the activatable antibody also includes a detectable moiety. In some embodiments, the detectable moiety is a diagnostic agent.

In some examples of any of these activatable antibody embodiments, the activatable antibody also includes a spacer. In some examples of any of these activatable antibody embodiments, the activatable antibody also includes a signal peptide. In some embodiments, the signal peptide is conjugated to the activatable antibody via a spacer. In some examples of any of these activatable antibody embodiments, the spacer is joined directly to the MM of the activatable antibody.

In some embodiments, the serum half-life of the activatable antibody is longer than that of the corresponding antibody; e.g., the pK of the activatable antibody is longer than that of the corresponding antibody. In some embodiments, the serum half-life of the activatable antibody is similar to that of the corresponding antibody. In some embodiments, the serum half-life of the activatable antibody is at least 15 days when administered to an organism. In some embodiments, the serum half-life of the activatable antibody is at least 12 days when administered to an organism. In some embodiments, the serum half-life of the activatable antibody is at least 11 days when administered to an organism. In some embodiments, the serum half-life of the activatable antibody is at least 10 days when administered to an organism. In some embodiments, the serum half-life of the activatable antibody is at least 9 days when administered to an organism. In some embodiments, the serum half-life of the activatable antibody is at least 8 days when administered to an organism. In some embodiments, the serum half-life of the activatable antibody is at least 7 days when administered to an organism. In some embodiments, the serum half-life of the activatable antibody is at least 6 days when administered to an organism. In some examples of any of these activatable antibody embodiments, the serum half-life of the activatable antibody is at least 5 days when administered to an organism. In some embodiments, the serum half-life of the activatable antibody is at least 4 days when administered to an organism. In some embodiments, the serum half-life of the activatable antibody is at least 3 days when administered to an organism. In some embodiments, the serum half-life of the activatable antibody is at least 2 days when administered to an organism. In some embodiments, the serum half-life of the activatable antibody is at least 24 hours when administered to an organism. In some embodiments, the serum half-life of the activatable antibody is at least 20 hours when administered to an organism. In some embodiments, the serum half-life of the activatable antibody is at least 18 hours when administered to an organism. In some embodiments, the serum half-life of the activatable antibody is at least 16 hours when administered to an organism. In some embodiments, the serum half-life of the activatable antibody is at least 14 hours when administered to an organism. In some embodiments, the serum half-life of the activatable antibody is at least 12 hours when administered to an organism. In some embodiments, the serum half-life of the activatable antibody is at least 10 hours when administered to an organism. In some embodiments, the serum half-life of the activatable antibody is at least 8 hours when administered to an organism. In some embodiments, the serum half-life of the activatable antibody is at least 6 hours when administered to an organism. In some embodiments, the serum half-life of the activatable antibody is at least 4 hours when administered to an organism. In some embodiments, the serum half-life of the activatable antibody is at least 3 hours when administered to an organism.

The disclosure also provides an isolated nucleic acid molecule encoding any of these activatable antibodies, as well as vectors that include these isolated nucleic acid sequences. The disclosure provides methods of producing an activatable antibody by culturing a cell under conditions that lead to expression of the activatable antibody, wherein the cell comprises such a nucleic acid sequence. In some embodiments, the cell comprises such a vector.

The dissociation constant (K_{d}) of the NB-containing activatable antibody toward the target is greater than the K_{d} of the AB towards the target when it is not associated with the NB or NB:BP. The dissociation constant (K_{d}) of the NB-containing activatable antibody toward the target is greater than the K_{d} of the parental AB towards the target. For example, the K_{d} of the NB-containing activatable antibody toward the target is at least 5, 10, 25, 50, 100, 250, 500, 1,000, 2,500, 5,000, 10,000, 50,000, 100,000, 500,000, 1,000,000, 5,000,000, 10,000,000, 50,000,000 or greater, or between 5-10, 10-100, 10-1,000, 10-10,000, 10-100,000, 10-1,000,000, 10-10,000,000, 100-1,000, 100-10,000, 100-100,000, 100-1,000,000, 100-10,000,000, 1,000-10,000, 1,000-100,000, 1,000-1,000,000, 1,000-10,000,000, 10,000-100,000, 10,000-1,000,000, 10,000-10,000,000, 100,000-1,000,000, or 100,000-10,000,000 times greater than the K_{d} of the AB when it is not associated with the NB or NB:BP or the K_{d} of the parental AB towards the target. Conversely, the binding affinity of the NB-containing activatable antibody towards the target is lower than the binding affinity of the AB when it is not associated with the NB or NB:BP or lower than the binding affinity of the parental AB towards the target. For example, the binding affinity of the NB-containing activatable antibody toward the target is at least 5, 10, 25, 50, 100, 250, 500, 1,000, 2,500, 5,000, 10,000, 50,000, 100,000, 500,000, 1,000,000, 5,000,000, 10,000,000, 50,000,000 or greater, or between 5-10, 10-100, 10-1,000, 10-10,000, 10-100,000, 10-1,000,000, 10-10,000,000, 100-1,000, 100-10,000, 100-100,000, 100-1,000,000, 100-10,000,000, 1,000-10,000, 1,000-100,000, 1,000-1,000,000, 1,000-10,000,000, 10,000-100,000, 10,000-1,000,000, 10,000-10,000,000, 100,000-1,000,000, or 100,000-10,000,000 times lower than the binding affinity of the AB when it is not associated with the NB or NB:BP or lower than the binding affinity of the parental AB towards the target.

When the NB-containing activatable antibody is in the presence of the target, specific binding of the AB to the target is reduced or inhibited, as compared to the specific binding of the AB when it is not associated with the NB or NB:BP. When the NB-containing activatable antibody is in the presence of the target, specific binding of the AB to the target is reduced or inhibited, as compared to the specific binding of the parental AB to the target. When compared to the binding of the AB not associated with an NB or NB:BP or the binding of the parental AB to the target, the ability of the NB-containing activatable antibody to bind the target is reduced, for example, by at least 50%, 60%, 70%, 80%, 90%, 92%, 93%, 94%, 95%, 96%, 97%, 98%, 99%, or even 100% for at least 2, 4, 6, 8, 12, 28, 24, 30, 36, 48, 60, 72, 84, or 96 hours, or 5, 10, 15, 30, 45, 60, 90, 120, 150, or 180 days, or 1, 2, 3, 4, 5, 6, 7, 8, 9, 10, 11, or 12 months or longer when measured in vitro and/or in vivo.

When the NB-containing activatable antibody is in the presence of the target but not in the presence of a modifying agent (for example a protease or other enzyme), specific binding of the AB to the target is reduced or inhibited, as compared to the specific binding of the AB when it is not associated with the NB or NB:BP. When the NB-containing activatable antibody is in the presence of the target but not in the presence of a modifying agent (for example a protease, other enzyme, reduction agent, or light), specific binding of the AB to the target is reduced or inhibited, as compared to the specific binding of the parental AB to the target. When compared to the binding of the AB not associated with an NB or NB:BP or the binding of the parental AB to the target, the ability of the NB-containing activatable antibody to bind the target is reduced, for example, by at least 50%, 60%, 70%, 80%, 90%, 92%, 93%, 94%, 95%, 96%, 97%, 98%, 99%, or even 100% for at least 2, 4, 6, 8, 12, 28, 24, 30, 36, 48, 60, 72, 84, or 96 hours, or 5, 10, 15, 30, 45, 60, 90, 120, 150, or 180 days, or 1, 2, 3, 4, 5, 6, 7, 8, 9, 10, 11, or 12 months or longer when measured in vitro and/or in vivo.

In some examples of any of these activatable antibody embodiments, the activatable antibody includes an agent conjugated to the AB to produce an activatable antibody conjugate. In some embodiments of the activatable antibody conjugate, the agent is a therapeutic agent. In some embodiments, the agent is a diagnostic agent. In some embodiments, the agent is a detectable marker. In some embodiments of the activatable antibody conjugate, the agent is an antineoplastic agent. In some embodiments of the activatable antibody conjugate, the agent is a toxin or fragment thereof. In some embodiments of the activatable antibody conjugate, the agent is conjugated to the AB via a linker. In some embodiments of the activatable antibody conjugate, the linker is a cleavable linker. In some embodiments, the agent is conjugated to the AB via a noncleavable linker. In some embodiments, the agent is a microtubule inhibitor. In some embodiments, the agent is a nucleic acid damaging agent, such as a DNA alkylator or DNA intercalator, or other DNA damaging agent. In some embodiments, the agent is an agent selected from the group listed in Table 3. In some embodiments, the agent is a dolastatin. In some embodiments, the agent is an auristatin or derivative thereof. In some embodiments, the agent is auristatin E or a derivative thereof. In some embodiments, the agent is monomethyl auristatin E (MMAE). In some embodiments, the agent is monomethyl auristatin D (MMAD). In some embodiments, the agent is a maytansinoid or maytansinoid derivative. In some embodiments, the agent is DM1 or DM4. In some embodiments, the agent is a duocarmycin or derivative thereof. In some embodiments, the agent is a calicheamicin or derivative thereof. In some embodiments, the agent is a pyrrolobenzodiazepine.

In some examples of any of these activatable antibody embodiments, the activatable antibodies are dual-target binding activatable antibodies. Such dual target binding activatable antibodies contain two Abs that may bind the same or different targets. In specific embodiments, dual-targeting activatable antibodies contain bispecific antibodies or antibody fragments.

Dual target binding activatable antibodies are designed so as to have a CL cleavable by a cleaving agent, e.g., a protease selected from matriptase and uPA, that is co-localized in a target tissue with one or both of the targets capable of binding to the ABs of the activatable antibodies. Dual target binding activatable antibodies with more than one AB to the same or different targets can be designed so as to have more than one CL, wherein the first CL is cleavable by a cleaving agent in a first target tissue and wherein the second CL is cleavable by a cleaving agent in a second target tissue, with one or more of the targets binding to the ABs of the activatable antibodies. In one embodiment, the first and second target tissues are spatially separated, for example, at different sites in the organism. In one embodiment, the first and second target tissues are the same tissue temporally separated, for example the same tissue at two different points in time, for example the first time point is when the tissue is an early stage tumor, and the second time point is when the tissue is a late stage tumor.

The disclosure also provides nucleic acid molecules encoding the activatable antibodies described herein. The disclosure also provides vectors that include these nucleic acids. The activatable antibodies described herein are produced by culturing a cell under conditions that lead to expression of the activatable antibody, wherein the cell includes these nucleic acid molecules or vectors.

The disclosure also provides methods of manufacturing activatable antibodies. In one embodiment, the method includes the steps of (a) culturing a cell that includes a nucleic acid construct that encodes the activatable antibody under conditions that lead to expression of the activatable antibody, wherein the activatable antibody includes (i) a non-binding steric moiety (NB); (ii) a cleavable linker (CL); and (iii) an antibody or an antigen binding fragment thereof (AB) that specifically binds a target, wherein (1) the NB does not bind specifically to the AB; (2) the CL is a polypeptide that includes a substrate (S) for an enzyme, e.g., a protease selected from matriptase and uPA; (3) the CL is positioned such that in an uncleaved state, the NB interferes with binding of the AB to the target and in a cleaved state, the NB does not interfere with binding of the AB to the target; and (4) the NB does not inhibit cleavage of the CL by the enzyme; and (b) recovering the activatable antibody.

In some embodiments, the method includes the steps of (a) culturing a cell that includes a nucleic acid construct that encodes the activatable antibody under conditions that lead to expression of the activatable antibody, wherein the activatable antibody includes (i) a binding partner (BP) for a non-binding steric moiety (NB); (ii) a cleavable linker (CL); and (iii) an antibody or an antigen binding fragment thereof (AB) that specifically binds a target, wherein (1) the NB does not bind specifically to the AB; (2) the CL is a polypeptide that includes a substrate (S) for an enzyme, e.g., a protease selected from matriptase and uPA; (3) the CL is positioned such that in an uncleaved state in the presence of the NB, the NB interferes with binding of the AB to the target and in a cleaved state, the NB does not interfere with binding of the AB to the target and the BP does not interfere with binding of the AB to the target; and (4) the NB and the BP do not inhibit cleavage of the CL by the enzyme; and (b) recovering the activatable antibody. In some examples of this embodiment, the BP of the activatable antibody is bound to the NB.

Use of CM-Containing Molecules Including Conjugated Antibodies and Activatable Antibodies

It will be appreciated that administration of therapeutic entities in accordance with the disclosure will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed, Mack Publishing Company, Easton, PA (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present disclosure, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203(1-2):1-60 (2000), Charman WN “Lipids, lipophilic drugs, and oral drug delivery-some emerging concepts.” J Pharm Sci.89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

Therapeutic formulations of the disclosure, which include a CM-containing molecule, such as by way of non-limiting example, a conjugated antibody, an activatable antibody and/or a conjugated activatable antibody, are used to prevent, treat or otherwise ameliorate a disease or disorder associated with aberrant target expression and/or activity. For example, therapeutic formulations of the disclosure, which include a CM-containing molecule, e.g., a conjugated antibody, an activatable antibody and/or a conjugated activatable antibody, are used to treat or otherwise ameliorate inflammation, an inflammatory disorder, an autoimmune disease and/or a cancer or other neoplastic condition. In some embodiments, the cancer is a solid tumor or a hematologic malignancy where the target is expressed. In some embodiments, the cancer is a solid tumor where the target is expressed. In some embodiments, the cancer is a hematologic malignancy where the target is expressed. In some embodiments, the target is expressed on parenchyma (e.g., in cancer, the portion of an organ or tissue that often carries out function(s) of the organ or tissue). In some embodiments, the target is expressed on a cell, tissue, or organ. In some embodiments, the target is expressed on stroma (i.e., the connective supportive framework of a cell, tissue, or organ). In some embodiments, the target is expressed on an osteoblast. In some embodiments, the target is expressed on the endothelium (vasculature). In some embodiments, the target is expressed on a cancer stem cell. In some embodiments, the agent to which the activatable antibody is conjugated is a microtubule inhibitor. In some embodiments, the agent to which the activatable antibody is conjugated is a nucleic acid damaging agent.

Efficaciousness of prevention, amelioration or treatment is determined in association with any known method for diagnosing or treating the disease or disorder associated with target expression and/or activity, such as, for example, aberrant target expression and/or activity. Prolonging the survival of a subject or otherwise delaying the progression of the disease or disorder associated with target expression and/or activity, e.g., aberrant target expression and/or activity, in a subject indicates that the conjugated antibody, activatable antibody and/or conjugated activatable antibody confers a clinical benefit.

A CM-containing molecule, e.g., a conjugated antibody, an activatable antibody and/or a conjugated activatable antibody can be administered in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

In some embodiments where antibody fragments are used, the smallest fragment that specifically binds to the binding domain of the target protein is selected. For example, based upon the variable-region sequences of an antibody, peptide molecules can be designed that retain the ability to bind the target protein sequence. Such peptides can be synthesized chemically and/or produced by recombinant DNA technology. (See, e.g., Marasco et al., Proc. Natl. Acad. Sci. USA, 90: 7889-7893 (1993)). The formulation can also contain more than one active compounds as necessary for the particular indication being treated, for example, in some embodiments, those with complementary activities that do not adversely affect each other. In some embodiments, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

In some embodiments, the CM-containing molecule, e.g., the conjugated antibody, activatable antibody and/or conjugated activatable antibody contains a detectable label. An intact antibody, or a fragment thereof (e.g., Fab, scFv, or F(ab)₂) is used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the disclosure can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, immunochemical staining, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, N J, 1995; “Immunoassay”, E. Diamandis and T. Christopoulus, Academic Press, Inc., San Diego, CA, 1996; and “Practice and Theory of Enzyme Immunoassays”, P. Tijssen, Elsevier Science Publishers, Amsterdam, 1985. Furthermore, in vivo techniques for detection of an analyte protein include introducing into a subject a labeled anti-analyte protein antibody. For example, the antibody can be labeled with a radioactive marker whose presence and location in a subject can be detected by standard imaging techniques.

The conjugated antibodies, activatable antibodies and/or conjugated activatable antibodies of the disclosure are also useful in a variety of diagnostic and prophylactic formulations. In one embodiment, a conjugated antibody, an activatable antibody and/or a conjugated activatable antibody is administered to patients that are at risk of developing one or more of the aforementioned disorders. A patient's or organ's predisposition to one or more of the aforementioned disorders can be determined using genotypic, serological or biochemical markers.

In some embodiments of the disclosure, a conjugated antibody, an activatable antibody and/or a conjugated activatable antibody is administered to human individuals diagnosed with a clinical indication associated with one or more of the aforementioned disorders. Upon diagnosis, a conjugated antibody, an activatable antibody and/or a conjugated activatable antibody is administered to mitigate or reverse the effects of the clinical indication.

A conjugated antibody, an activatable antibody and/or a conjugated activatable antibody of the disclosure is also useful in the detection of a target in patient samples and accordingly are useful as diagnostics. For example, the antibodies and/or activatable antibodies, and conjugated versions thereof, of the disclosure are used in in vitro assays, e.g., ELISA, to detect target levels in a patient sample.

In one embodiment, a conjugated antibody, an activatable antibody and/or a conjugated activatable antibody of the disclosure is immobilized on a solid support (e.g., the well(s) of a microtiter plate). The immobilized conjugated antibody, activatable antibody and/or conjugated activatable antibody serves as a capture antibody for any target that may be present in a test sample. Prior to contacting the immobilized antibody with a patient sample, the solid support is rinsed and treated with a blocking agent such as milk protein or albumin to prevent nonspecific adsorption of the analyte.

Subsequently the wells are treated with a test sample suspected of containing the antigen, or with a solution containing a standard amount of the antigen. Such a sample is, e.g., a serum sample from a subject suspected of having levels of circulating antigen considered to be diagnostic of a pathology. After rinsing away the test sample or standard, the solid support is treated with a second antibody that is detectably labeled. The labeled second antibody serves as a detecting antibody. The level of detectable label is measured, and the concentration of target antigen in the test sample is determined by comparison with a standard curve developed from the standard samples.

It will be appreciated that based on the results obtained using the antibodies of the disclosure, and conjugated versions thereof, in an in vitro diagnostic assay, it is possible to stage a disease in a subject based on expression levels of the target antigen. For a given disease, samples of blood are taken from subjects diagnosed as being at various stages in the progression of the disease, and/or at various points in the therapeutic treatment of the disease. Using a population of samples that provides statistically significant results for each stage of progression or therapy, a range of concentrations of the antigen that may be considered characteristic of each stage is designated.

A conjugated antibody, an activatable antibody and/or a conjugated activatable antibody can also be used in diagnostic and/or imaging methods. In some embodiments, such methods are in vitro methods. In some embodiments, such methods are in vivo methods. In some embodiments, such methods are in situ methods. In some embodiments, such methods are ex vivo methods. For example, activatable antibodies having an enzymatically cleavable CM can be used to detect the presence or absence of an enzyme that is capable of cleaving the CM. Such activatable antibodies can be used in diagnostics, which can include in vivo detection (e.g., qualitative or quantitative) of enzyme activity (or, in some embodiments, an environment of increased reduction potential such as that which can provide for reduction of a disulfide bond) through measured accumulation of activated antibodies (i.e., antibodies resulting from cleavage of an activatable antibody) in a given cell or tissue of a given host organism. Such accumulation of activated antibodies indicates not only that the tissue expresses enzymatic activity (or an increased reduction potential depending on the nature of the CM) but also that the tissue expresses target to which the activated antibody binds.

For example, the CM can be selected to be substrate for at least one protease selected from matriptase and uPA found at the site of a tumor, at the site of a viral or bacterial infection at a biologically confined site (e.g., such as in an abscess, in an organ, and the like), and the like. The AB can be one that binds a target antigen. Using methods as disclosed herein, or when appropriate, methods familiar to one skilled in the art, a detectable label (e.g., a fluorescent label or radioactive label or radiotracer) can be conjugated to an AB or other region of an antibody and/or activatable antibody. Suitable detectable labels are discussed in the context of the above screening methods and additional specific examples are provided below. Using an AB specific to a protein or peptide of the disease state, along with at least one protease selected from matriptase and uPA whose activity is elevated in the disease tissue of interest, activatable antibodies will exhibit an increased rate of binding to disease tissue relative to tissues where the CM specific enzyme is not present at a detectable level or is present at a lower level than in disease tissue or is inactive (e.g., in zymogen form or in complex with an inhibitor). Since small proteins and peptides are rapidly cleared from the blood by the renal filtration system, and because the enzyme specific for the CM is not present at a detectable level (or is present at lower levels in non-disease tissues or is present in inactive conformation), accumulation of activated antibodies in the disease tissue is enhanced relative to non-disease tissues.

In another example, activatable antibodies can be used to detect the presence or absence of a cleaving agent in a sample. For example, where the activatable antibodies contain a CM susceptible to cleavage by an enzyme, the activatable antibodies can be used to detect (either qualitatively or quantitatively) the presence of an enzyme in the sample. In another example, where the activatable antibodies contain a CM susceptible to cleavage by reducing agent, the activatable antibodies can be used to detect (either qualitatively or quantitatively) the presence of reducing conditions in a sample. To facilitate analysis in these methods, the activatable antibodies can be detectably labeled, and can be bound to a support (e.g., a solid support, such as a slide or bead). The detectable label can be positioned on a portion of the activatable antibody that is not released following cleavage, for example, the detectable label can be a quenched fluorescent label or other label that is not detectable until cleavage has occurred. The assay can be conducted by, for example, contacting the immobilized, detectably labeled activatable antibodies with a sample suspected of containing an enzyme and/or reducing agent for a time sufficient for cleavage to occur, then washing to remove excess sample and contaminants. The presence or absence of the cleaving agent (e.g., enzyme or reducing agent) in the sample is then assessed by a change in detectable signal of the activatable antibodies prior to contacting with the sample e.g., the presence of and/or an increase in detectable signal due to cleavage of the activatable antibody by the cleaving agent in the sample.

Such detection methods can be adapted to also provide for detection of the presence or absence of a target that is capable of binding the AB of the activatable antibodies when cleaved. Thus, the assays can be adapted to assess the presence or absence of a cleaving agent and the presence or absence of a target of interest. The presence or absence of the cleaving agent can be detected by the presence of and/or an increase in detectable label of the activatable antibodies as described above, and the presence or absence of the target can be detected by detection of a target-AB complex e.g., by use of a detectably labeled anti-target antibody.

Activatable antibodies are also useful in in situ imaging for the validation of activatable antibody activation, e.g., by protease cleavage, and binding to a particular target. In situ imaging is a technique that enables localization of proteolytic activity and target in biological samples such as cell cultures or tissue sections. Using this technique, it is possible to confirm both binding to a given target and proteolytic activity based on the presence of a detectable label (e.g., a fluorescent label).

These techniques are useful with any frozen cells or tissue derived from a disease site (e.g. tumor tissue) or healthy tissues. These techniques are also useful with fresh cell or tissue samples.

In these techniques, an activatable antibody is labeled with a detectable label. The detectable label may be a fluorescent dye, (e.g. a fluorophore, Fluorescein Isothiocyanate (FITC), Rhodamine Isothiocyanate (TRITC), an Alexa Fluor® label), a near infrared (NIR) dye (e.g., Qdot® nanocrystals), a colloidal metal, a hapten, a radioactive marker, biotin and an amplification reagent such as streptavidin, or an enzyme (e.g. horseradish peroxidase or alkaline phosphatase).

Detection of the label in a sample that has been incubated with the labeled, activatable antibody indicates that the sample contains the target and contains a protease selected from matriptase and uPA that is specific for the CM of the activatable antibody. In some embodiments, the presence of the protease selected from matriptase and uPA can be confirmed using broad spectrum protease inhibitors such as those described herein, and/or by using an agent that is specific for the protease, for example, an antibody such as A11, which is specific for the protease matriptase and inhibits the proteolytic activity of matriptase; see e.g., International Publication Number WO 2010/129609, published 11 Nov. 2010. The same approach of using broad spectrum protease inhibitors such as those described herein, and/or by using a more selective inhibitory agent can be used to identify a protease selected from matriptase and uPA that is specific for the CM of the activatable antibody. In some embodiments, the presence of the target can be confirmed using an agent that is specific for the target, e.g., another antibody, or the detectable label can be competed with unlabeled target. In some embodiments, unlabeled activatable antibody could be used, with detection by a labeled secondary antibody or more complex detection system.

Similar techniques are also useful for in vivo imaging where detection of the fluorescent signal in a subject, e.g., a mammal, including a human, indicates that the disease site contains the target and contains a protease selected from matriptase and uPA that is specific for the CM of the activatable antibody.

These techniques are also useful in kits and/or as reagents for the detection, identification or characterization of protease activity in a variety of cells, tissues, and organisms based on the protease-specific CM in the activatable antibody.

The disclosure provides methods of using the antibodies and/or activatable antibodies in a variety of diagnostic and/or prophylactic indications. For example, the disclosure provides methods of detecting presence or absence of a cleaving agent and a target of interest in a subject or a sample by (i) contacting a subject or sample with an activatable antibody, wherein the activatable antibody comprises a masking moiety (MM), a cleavable moiety (CM) that is cleaved by the cleaving agent, e.g., a protease selected from matriptase and uPA, and an antigen binding domain or fragment thereof (AB) that specifically binds the target of interest, wherein the activatable antibody in an uncleaved, non-activated state comprises a structural arrangement from N-terminus to C-terminus as follows: MM-CM-AB or AB-CM-MM; (a) wherein the MM is a peptide that inhibits binding of the AB to the target, and wherein the MM does not have an amino acid sequence of a naturally occurring binding partner of the AB and is not a modified form of a natural binding partner of the AB; and (b) wherein, in an uncleaved, non-activated state, the MM interferes with specific binding of the AB to the target, and in a cleaved, activated state the MA/I does not interfere or compete with specific binding of the AB to the target; and (ii) measuring a level of activated activatable antibody in the subject or sample, wherein a detectable level of activated activatable antibody in the subject or sample indicates that the cleaving agent and the target are present in the subject or sample and wherein no detectable level of activated activatable antibody in the subject or sample indicates that the cleaving agent, the target or both the cleaving agent and the target are absent and/or not sufficiently present in the subject or sample. In some embodiments, the activatable antibody is an activatable antibody to which a therapeutic agent is conjugated. In some embodiments, the activatable antibody is not conjugated to an agent. In some embodiments, the activatable antibody comprises a detectable label. In some embodiments, the detectable label is positioned on the AB. In some embodiments, measuring the level of activatable antibody in the subject or sample is accomplished using a secondary reagent that specifically binds to the activated antibody, wherein the reagent comprises a detectable label. In some embodiments, the secondary reagent is an antibody comprising a detectable label.

The disclosure also provides methods of detecting presence or absence of a cleaving agent in a subject or a sample by (i) contacting a subject or sample with an activatable antibody in the presence of a target of interest, e.g., the target, wherein the activatable antibody comprises a masking moiety (MM), a cleavable moiety (CM) that is cleaved by the cleaving agent, e.g., a protease selected from matriptase and uPA, and an antigen binding domain or fragment thereof (AB) that specifically binds the target of interest, wherein the activatable antibody in an uncleaved, non-activated state comprises a structural arrangement from N-terminus to C-terminus as follows: MM-CM-AB or AB-CM-MM; (a) wherein the MM is a peptide that inhibits binding of the AB to the target, and wherein the MM does not have an amino acid sequence of a naturally occurring binding partner of the AB and is not a modified form of a natural binding partner of the AB; and (b) wherein, in an uncleaved, non-activated state, the MM interferes with specific binding of the AB to the target, and in a cleaved, activated state the MM does not interfere or compete with specific binding of the AB to the target; and (ii) measuring a level of activated activatable antibody in the subject or sample, wherein a detectable level of activated activatable antibody in the subject or sample indicates that the cleaving agent is present in the subject or sample and wherein no detectable level of activated activatable antibody in the subject or sample indicates that the cleaving agent is absent and/or not sufficiently present in the subject or sample. In some embodiments, the activatable antibody is an activatable antibody to which a therapeutic agent is conjugated. In some embodiments, the activatable antibody is not conjugated to an agent. In some embodiments, the activatable antibody comprises a detectable label. In some embodiments, the detectable label is positioned on the AB. In some embodiments, measuring the level of activatable antibody in the subject or sample is accomplished using a secondary reagent that specifically binds to the activated antibody, wherein the reagent comprises a detectable label. In some embodiments, the secondary reagent is an antibody comprising a detectable label.

The disclosure also provides kits for use in methods of detecting presence or absence of a cleaving agent and the target in a subject or a sample, where the kits include at least an activatable antibody comprises a masking moiety (MINI), a cleavable moiety (CM) that is cleaved by the cleaving agent, e.g., a protease selected from matriptase and uPA, and an antigen binding domain or fragment thereof (AB) that specifically binds the target of interest, wherein the activatable antibody in an uncleaved, non-activated state comprises a structural arrangement from N-terminus to C-terminus as follows: MM-CM-AB or AB-CM-MINI; (a) wherein the MM is a peptide that inhibits binding of the AB to the target, and wherein the MM does not have an amino acid sequence of a naturally occurring binding partner of the AB and is not a modified form of a natural binding partner of the AB; and (b) wherein, in an uncleaved, non-activated state, the MM interferes with specific binding of the AB to the target, and in a cleaved, activated state the MINI does not interfere or compete with specific binding of the AB to the target; and (ii) measuring a level of activated activatable antibody in the subject or sample, wherein a detectable level of activated activatable antibody in the subject or sample indicates that the cleaving agent is present in the subject or sample and wherein no detectable level of activated activatable antibody in the subject or sample indicates that the cleaving agent is absent and/or not sufficiently present in the subject or sample. In some embodiments, the activatable antibody is an activatable antibody to which a therapeutic agent is conjugated. In some embodiments, the activatable antibody is not conjugated to an agent. In some embodiments, the activatable antibody comprises a detectable label. In some embodiments, the detectable label is positioned on the AB. In some embodiments, measuring the level of activatable antibody in the subject or sample is accomplished using a secondary reagent that specifically binds to the activated antibody, wherein the reagent comprises a detectable label. In some embodiments, the secondary reagent is an antibody comprising a detectable label.

The disclosure also provides methods of detecting presence or absence of a cleaving agent in a subject or a sample by (i) contacting a subject or sample with an activatable antibody, wherein the activatable antibody comprises a masking moiety (MM), a cleavable moiety (CM) that is cleaved by the cleaving agent, e.g., a protease selected from matriptase and uPA, an antigen binding domain (AB) that specifically binds the target, and a detectable label, wherein the activatable antibody in an uncleaved, non-activated state comprises a structural arrangement from N-terminus to C-terminus as follows: MINI-CM-AB or AB-CM-MINI; wherein the MM is a peptide that inhibits binding of the AB to the target, and wherein the MM does not have an amino acid sequence of a naturally occurring binding partner of the AB and is not a modified form of a natural binding partner of the AB; wherein, in an uncleaved, non-activated state, the MM interferes with specific binding of the AB to the target, and in a cleaved, activated state the MM does not interfere or compete with specific binding of the AB to the target; and wherein the detectable label is positioned on a portion of the activatable antibody that is released following cleavage of the CM; and (ii) measuring a level of detectable label in the subject or sample, wherein a detectable level of the detectable label in the subject or sample indicates that the cleaving agent is absent and/or not sufficiently present in the subject or sample and wherein no detectable level of the detectable label in the subject or sample indicates that the cleaving agent is present in the subject or sample. In some embodiments, the activatable antibody is an activatable antibody to which a therapeutic agent is conjugated. In some embodiments, the activatable antibody is not conjugated to an agent. In some embodiments, the activatable antibody comprises a detectable label. In some embodiments, the detectable label is positioned on the AB. In some embodiments, measuring the level of activatable antibody in the subject or sample is accomplished using a secondary reagent that specifically binds to the activated antibody, wherein the reagent comprises a detectable label. In some embodiments, the secondary reagent is an antibody comprising a detectable label.

The disclosure also provides kits for use in methods of detecting presence or absence of a cleaving agent and the target in a subject or a sample, where the kits include at least an activatable antibody and/or conjugated activatable antibody (e.g., an activatable antibody to which a therapeutic agent is conjugated) described herein for use in contacting a subject or biological sample and means for detecting the level of activated activatable antibody and/or conjugated activatable antibody in the subject or biological sample, wherein a detectable level of activated activatable antibody in the subject or biological sample indicates that the cleaving agent and the target are present in the subject or biological sample and wherein no detectable level of activated activatable antibody in the subject or biological sample indicates that the cleaving agent, the target or both the cleaving agent and the target are absent and/or not sufficiently present in the subject or biological sample, such that the target binding and/or protease cleavage of the activatable antibody cannot be detected in the subject or biological sample.

The disclosure also provides methods of detecting presence or absence of a cleaving agent in a subject or a sample by (i) contacting a subject or biological sample with an activatable antibody in the presence of the target, and (ii) measuring a level of activated activatable antibody in the subject or biological sample, wherein a detectable level of activated activatable antibody in the subject or biological sample indicates that the cleaving agent is present in the subject or biological sample and wherein no detectable level of activated activatable antibody in the subject or biological sample indicates that the cleaving agent is absent and/or not sufficiently present in the subject or biological sample at a detectable level, such that protease cleavage of the activatable antibody cannot be detected in the subject or biological sample. Such an activatable antibody includes a masking moiety (MM), a cleavable moiety (CM) that is cleaved by the cleaving agent, e.g., a protease selected from matriptase and uPA, and an antigen binding domain or fragment thereof (AB) that specifically binds the target, wherein the activatable antibody in an uncleaved (i.e., non-activated) state comprises a structural arrangement from N-terminus to C-terminus as follows: MM-CM-AB or AB-CM-MM; (a) wherein the MM is a peptide that inhibits binding of the AB to the target, and wherein the MM does not have an amino acid sequence of a naturally occurring binding partner of the AB; and (b) wherein the MM of the activatable antibody in an uncleaved state interferes with specific binding of the AB to the target, and wherein the MM of an activatable antibody in a cleaved (i.e., activated) state does not interfere or compete with specific binding of the AB to the target. In some embodiments, the activatable antibody is an activatable antibody to which a therapeutic agent is conjugated. In some embodiments, the activatable antibody is not conjugated to an agent. In some embodiments, the detectable label is attached to the masking moiety. In some embodiments, the detectable label is attached to the cleavable moiety N-terminal to the protease cleavage site. In some embodiments, a single antigen binding site of the AB is masked. In some embodiments wherein an antibody of the disclosure has at least two antigen binding sites, at least one antigen binding site is masked and at least one antigen binding site is not masked. In some embodiments all antigen binding sites are masked. In some embodiments, the measuring step includes use of a secondary reagent comprising a detectable label.

The disclosure also provides kits for use in methods of detecting presence or absence of a cleaving agent and the target in a subject or a sample, where the kits include at least an activatable antibody and/or conjugated activatable antibody described herein for use in contacting a subject or biological sample with an activatable antibody in the presence of the target, and measuring a level of activated activatable antibody in the subject or biological sample, wherein a detectable level of activated activatable antibody in the subject or biological sample indicates that the cleaving agent is present in the subject or biological sample and wherein no detectable level of activated activatable antibody in the subject or biological sample indicates that the cleaving agent is absent and/or not sufficiently present in the subject or biological sample at a detectable level, such that protease cleavage of the activatable antibody cannot be detected in the subject or biological sample. Such an activatable antibody includes a masking moiety (MM), a cleavable moiety (CM) that is cleaved by the cleaving agent, e.g., a protease selected from matriptase and uPA, and an antigen binding domain or fragment thereof (AB) that specifically binds the target, wherein the activatable antibody in an uncleaved (i.e., non-activated) state comprises a structural arrangement from N-terminus to C-terminus as follows: MM-CM-AB or AB-CM-MM; (a) wherein the MM is a peptide that inhibits binding of the AB to the target, and wherein the MM does not have an amino acid sequence of a naturally occurring binding partner of the AB; and (b) wherein the MM of the activatable antibody in an uncleaved state interferes with specific binding of the AB to the target, and wherein the MM of an activatable antibody in a cleaved (i.e., activated) state does not interfere or compete with specific binding of the AB to the target. In some embodiments, the activatable antibody is an activatable antibody to which a therapeutic agent is conjugated. In some embodiments, the activatable antibody is not conjugated to an agent. In some embodiments, the detectable label is attached to the masking moiety. In some embodiments, the detectable label is attached to the cleavable moiety N-terminal to the protease cleavage site. In some embodiments, a single antigen binding site of the AB is masked. In some embodiments wherein an antibody of the disclosure has at least two antigen binding sites, at least one antigen binding site is masked and at least one antigen binding site is not masked. In some embodiments all antigen binding sites are masked. In some embodiments, the measuring step includes use of a secondary reagent comprising a detectable label.

The disclosure also provides kits for use in methods of detecting presence or absence of a cleaving agent in a subject or a sample, where the kits include at least an activatable antibody and/or conjugated activatable antibody described herein for use in contacting a subject or biological sample and means for detecting the level of activated activatable antibody and/or conjugated activatable antibody in the subject or biological sample, wherein the activatable antibody includes a detectable label that is positioned on a portion of the activatable antibody that is released following cleavage of the CM, wherein a detectable level of activated activatable antibody in the subject or biological sample indicates that the cleaving agent is absent and/or not sufficiently present in the subject or biological sample such that the target binding and/or protease cleavage of the activatable antibody cannot be detected in the subject or biological sample, and wherein no detectable level of activated activatable antibody in the subject or biological sample indicates that the cleaving agent is present in the subject or biological sample at a detectable level.

The disclosure provides methods of detecting presence or absence of a cleaving agent and the target in a subject or a sample by (i) contacting a subject or biological sample with an activatable antibody, wherein the activatable antibody includes a detectable label that is positioned on a portion of the activatable antibody that is released following cleavage of the CM and (ii) measuring a level of activated activatable antibody in the subject or biological sample, wherein a detectable level of activated activatable antibody in the subject or biological sample indicates that the cleaving agent, the target or both the cleaving agent and the target are absent and/or not sufficiently present in the subject or biological sample, such that the target binding and/or protease cleavage of the activatable antibody cannot be detected in the subject or biological sample, and wherein a reduced detectable level of activated activatable antibody in the subject or biological sample indicates that the cleaving agent and the target are present in the subject or biological sample. A reduced level of detectable label is, for example, a reduction of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% and/or about 100%. Such an activatable antibody includes a masking moiety (MM), a cleavable moiety (CM) that is cleaved by the cleaving agent, and an antigen binding domain or fragment thereof (AB) that specifically binds the target, wherein the activatable antibody in an uncleaved (i.e., non-activated) state comprises a structural arrangement from N-terminus to C-terminus as follows: MM-CM-AB or AB-CM-MM; (a) wherein the MM is a peptide that inhibits binding of the AB to the target, and wherein the MM does not have an amino acid sequence of a naturally occurring binding partner of the AB; and (b) wherein the MM of the activatable antibody in an uncleaved state interferes with specific binding of the AB to the target, and wherein the MM of an activatable antibody in a cleaved (i.e., activated) state does not interfere or compete with specific binding of the AB to the target. In some embodiments, the activatable antibody is an activatable antibody to which a therapeutic agent is conjugated. In some embodiments, the activatable antibody is not conjugated to an agent. In some embodiments, the activatable antibody comprises a detectable label. In some embodiments, the detectable label is positioned on the AB. In some embodiments, measuring the level of activatable antibody in the subject or sample is accomplished using a secondary reagent that specifically binds to the activated antibody, wherein the reagent comprises a detectable label. In some embodiments, the secondary reagent is an antibody comprising a detectable label.

The disclosure also provides kits for use in methods of detecting presence or absence of a cleaving agent and the target in a subject or a sample, where the kits include at least an activatable antibody and/or conjugated activatable antibody described herein for use in contacting a subject or biological sample and means for detecting the level of activated activatable antibody and/or conjugated activatable antibody in the subject or biological sample, wherein a detectable level of activated activatable antibody in the subject or biological sample indicates that the cleaving agent, the target or both the cleaving agent and the target are absent and/or not sufficiently present in the subject or biological sample, such that the target binding and/or protease cleavage of the activatable antibody cannot be detected in the subject or biological sample, and wherein a reduced detectable level of activated activatable antibody in the subject or biological sample indicates that the cleaving agent and the target are present in the subject or biological sample. A reduced level of detectable label is, for example, a reduction of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% and/or about 100%.

The disclosure also provides methods of detecting presence or absence of a cleaving agent in a subject or a sample by (i) contacting a subject or biological sample with an activatable antibody, wherein the activatable antibody includes a detectable label that is positioned on a portion of the activatable antibody that is released following cleavage of the CM; and (ii) measuring a level of detectable label in the subject or biological sample, wherein a detectable level of the detectable label in the subject or biological sample indicates that the cleaving agent is absent and/or not sufficiently present in the subject or biological sample at a detectable level, such that protease cleavage of the activatable antibody cannot be detected in the subject or biological sample, and wherein a reduced detectable level of the detectable label in the subject or biological sample indicates that the cleaving agent is present in the subject or biological sample. A reduced level of detectable label is, for example, a reduction of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% and/or about 100%. Such an activatable antibody includes a masking moiety (MM), a cleavable moiety (CM) that is cleaved by the cleaving agent, and an antigen binding domain or fragment thereof (AB) that specifically binds the target, wherein the activatable antibody in an uncleaved (i.e., non-activated) state comprises a structural arrangement from N-terminus to C-terminus as follows: MM-CM-AB or AB-CM-MM; (a) wherein the MM is a peptide that inhibits binding of the AB to the target, and wherein the MM does not have an amino acid sequence of a naturally occurring binding partner of the AB; and (b) wherein the MM of the activatable antibody in an uncleaved state interferes with specific binding of the AB to the target, and wherein the MM of an activatable antibody in a cleaved (i.e., activated) state does not interfere or compete with specific binding of the AB to the target. In some embodiments, the activatable antibody is an activatable antibody to which a therapeutic agent is conjugated. In some embodiments, the activatable antibody is not conjugated to an agent. In some embodiments, the activatable antibody comprises a detectable label. In some embodiments, the detectable label is positioned on the AB. In some embodiments, measuring the level of activatable antibody in the subject or sample is accomplished using a secondary reagent that specifically binds to the activated antibody, wherein the reagent comprises a detectable label. In some embodiments, the secondary reagent is an antibody comprising a detectable label.

The disclosure also provides kits for use in methods of detecting presence or absence of a cleaving agent of interest in a subject or a sample, where the kits include at least an activatable antibody and/or conjugated activatable antibody described herein for use in contacting a subject or biological sample and means for detecting the level of activated activatable antibody and/or conjugated activatable antibody in the subject or biological sample, wherein the activatable antibody includes a detectable label that is positioned on a portion of the activatable antibody that is released following cleavage of the CM, wherein a detectable level of the detectable label in the subject or biological sample indicates that the cleaving agent, the target, or both the cleaving agent and the target are absent and/or not sufficiently present in the subject or biological sample, such that the target binding and/or protease cleavage of the activatable antibody cannot be detected in the subject or biological sample, and wherein a reduced detectable level of the detectable label in the subject or biological sample indicates that the cleaving agent and the target are present in the subject or biological sample. A reduced level of detectable label is, for example, a reduction of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% and/or about 100%.

In some embodiments of these methods and kits, the activatable antibody includes a detectable label. In some embodiments of these methods and kits, the detectable label includes an imaging agent, a contrasting agent, an enzyme, a fluorescent label, a chromophore, a dye, one or more metal ions, or a ligand-based label. In some embodiments of these methods and kits, the imaging agent comprises a radioisotope. In some embodiments of these methods and kits, the radioisotope is indium or technetium. In some embodiments of these methods and kits, the contrasting agent comprises iodine, gadolinium or iron oxide. In some embodiments of these methods and kits, the enzyme comprises horseradish peroxidase, alkaline phosphatase, or β-galactosidase. In some embodiments of these methods and kits, the fluorescent label comprises yellow fluorescent protein (YFP), cyan fluorescent protein (CFP), green fluorescent protein (GFP), modified red fluorescent protein (mRFP), red fluorescent protein tdimer2 (RFP tdimer2), HCRED, or a europium derivative. In some embodiments of these methods and kits, the luminescent label comprises an N-methylacrydium derivative. In some embodiments of these methods, the label comprises an Alexa Fluor® label, such as Alex Fluor® 680 or Alexa Fluor® 750. In some embodiments of these methods and kits, the ligand-based label comprises biotin, avidin, streptavidin or one or more haptens.

In some embodiments of these methods and kits, the subject is a mammal. In some embodiments of these methods and kits, the subject is a human. In some embodiments, the subject is a non-human mammal, such as a non-human primate, companion animal (e.g., cat, dog, horse), farm animal, work animal, or zoo animal. In some embodiments, the subject is a rodent.

In some embodiments of these methods, the method is an in vivo method. In some embodiments of these methods, the method is an in situ method. In some embodiments of these methods, the method is an ex vivo method. In some embodiments of these methods, the method is an in vitro method.

In some embodiments, in situ imaging and/or in vivo imaging are useful in methods to identify which patients to treat. For example, in in situ imaging, the activatable antibodies are used to screen patient samples to identify those patients having the appropriate protease(s) and target(s) at the appropriate location, e.g., at a tumor site.

In some embodiments in situ imaging is used to identify or otherwise refine a patient population suitable for treatment with an activatable antibody of the disclosure. For example, patients that test positive for both the target (e.g., the target) and a protease that cleaves the substrate in the cleavable moiety (CM) of the activatable antibody being tested (e.g., accumulate activated antibodies at the disease site) are identified as suitable candidates for treatment with such an activatable antibody comprising such a CM. Likewise, patients that test negative for either or both of the target (e.g., the target) and the protease that cleaves the substrate in the CM in the activatable antibody being tested using these methods might be identified as suitable candidates for another form of therapy. In some embodiments, such patients that test negative with respect to a first activatable antibody can be tested with other activatable antibodies comprising different CMs until a suitable activatable antibody for treatment is identified (e.g., an activatable antibody comprising a CM that is cleaved by the patient at the site of disease). In some embodiments, the patient is then administered a therapeutically effective amount of the conjugated activatable antibody for which the patient tested positive.

In some embodiments in vivo imaging is used to identify or otherwise refine a patient population suitable for treatment with an activatable antibody of the disclosure. For example, patients that test positive for both the target (e.g., the target) and a protease that cleaves the substrate in the cleavable moiety (CM) of the activatable antibody being tested (e.g., accumulate activated antibodies at the disease site) are identified as suitable candidates for treatment with such an activatable antibody comprising such a CM. Likewise, patients that test negative might be identified as suitable candidates for another form of therapy. In some embodiments, such patients that test negative with respect to a first activatable antibody can be tested with other activatable antibodies comprising different CMs until a suitable activatable antibody for treatment is identified (e.g., an activatable antibody comprising a CM that is cleaved by the patient at the site of disease). In some embodiments, the patient is then administered a therapeutically effective amount of the conjugated activatable antibody for which the patient tested positive.

In some embodiments of the methods and kits, the method or kit is used to identify or otherwise refine a patient population suitable for treatment with an activatable antibody of the disclosure. For example, patients that test positive for both the target (e.g., the target) and a protease that cleaves the substrate in the cleavable moiety (CM) of the activatable antibody being tested in these methods are identified as suitable candidates for treatment with such an activatable antibody comprising such a CM. Likewise, patients that test negative for both of the targets (e.g., the target) and the protease that cleaves the substrate in the CM in the activatable antibody being tested using these methods might be identified as suitable candidates for another form of therapy. In some embodiments, such patients can be tested with other activatable antibodies until a suitable activatable antibody for treatment is identified (e.g., an activatable antibody comprising a CM that is cleaved by the patient at the site of disease). In some embodiments, patients that test negative for either of the target (e.g., the target) are identified as suitable candidates for treatment with such an activatable antibody comprising such a CM. In some embodiments, patients that test negative for either of the target (e.g., the target) are identified as not being suitable candidates for treatment with such an activatable antibody comprising such a CM. In some embodiments, such patients can be tested with other activatable antibodies until a suitable activatable antibody for treatment is identified (e.g., an activatable antibody comprising a CM that is cleaved by the patient at the site of disease). In some embodiments, the activatable antibody is an activatable antibody to which a therapeutic agent is conjugated. In some embodiments, the activatable antibody is not conjugated to an agent. In some embodiments, the activatable antibody comprises a detectable label. In some embodiments, the detectable label is positioned on the AB. In some embodiments, measuring the level of activatable antibody in the subject or sample is accomplished using a secondary reagent that specifically binds to the activated antibody, wherein the reagent comprises a detectable label. In some embodiments, the secondary reagent is an antibody comprising a detectable label.

In some embodiments, a method or kit is used to identify or otherwise refine a patient population suitable for treatment with an anti-the target activatable antibody and/or conjugated activatable antibody (e.g., activatable antibody to which a therapeutic agent is conjugated) of the disclosure, followed by treatment by administering that activatable antibody and/or conjugated activatable antibody to a subject in need thereof. For example, patients that test positive for both the targets (e.g., the target) and a protease that cleaves the substrate in the cleavable moiety (CM) of the activatable antibody and/or conjugated activatable antibody being tested in these methods are identified as suitable candidates for treatment with such antibody and/or such a conjugated activatable antibody comprising such a CM, and the patient is then administered a therapeutically effective amount of the activatable antibody and/or conjugated activatable antibody that was tested. Likewise, patients that test negative for either or both of the target (e.g., the target) and the protease that cleaves the substrate in the CM in the activatable antibody being tested using these methods might be identified as suitable candidates for another form of therapy. In some embodiments, such patients can be tested with other antibody and/or conjugated activatable antibody until a suitable antibody and/or conjugated activatable antibody for treatment is identified (e.g., an activatable antibody and/or conjugated activatable antibody comprising a CM that is cleaved by the patient at the site of disease). In some embodiments, the patient is then administered a therapeutically effective amount of the activatable antibody and/or conjugated for which the patient tested positive.

In some embodiments of these methods and kits, the MM is a peptide having a length from about 4 to 40 amino acids. In some embodiments of these methods and kits, the activatable antibody comprises a linker peptide, wherein the linker peptide is positioned between the MM and the CM. In some embodiments of these methods and kits, the activatable antibody comprises a linker peptide, where the linker peptide is positioned between the AB and the CM. In some embodiments of these methods and kits, the activatable antibody comprises a first linker peptide (L1) and a second linker peptide (L2), wherein the first linker peptide is positioned between the MM and the CM and the second linker peptide is positioned between the AB and the CM. In some embodiments of these methods and kits, each of L1 and L2 is a peptide of about 1 to 20 amino acids in length, and wherein each of L1 and L2 need not be the same linker. In some embodiments of these methods and kits, one or both of L1 and L2 comprises a glycine-serine polymer. In some embodiments of these methods and kits, at least one of L1 and L2 comprises an amino acid sequence selected from the group consisting of (GS)n, (GSGGS)n (SEQ ID NO: 385) and (GGGS)n (SEQ ID NO: 386), where n is an integer of at least one. In some embodiments of these methods and kits, at least one of L1 and L2 comprises an amino acid sequence having the formula (GGS)n, where n is an integer of at least one. In some embodiments of these methods and kits, at least one of L1 and L2 comprises an amino acid sequence selected from the group consisting of Gly-Gly-Ser-Gly (SEQ ID NO: 387), Gly-Gly-Ser-Gly-Gly (SEQ ID NO: 388), Gly-Ser-Gly-Ser-Gly (SEQ ID NO: 389), Gly-Ser-Gly-Gly-Gly (SEQ ID NO: 390), Gly-Gly-Gly-Ser-Gly (SEQ ID NO: 391), and Gly-Ser-Ser-Ser-Gly (SEQ ID NO: 392).

In some embodiments of these methods and kits, the AB comprises an antibody or antibody fragment sequence selected from the cross-reactive antibody sequences presented herein. In some embodiments of these methods and kits, the AB comprises a Fab fragment, a scFv or a single chain antibody (scAb).

In some embodiments of these methods and kits, the cleaving agent is a protease that is co-localized in the subject or sample with the target and the CM is a polypeptide that functions as a substrate for the protease, wherein the protease cleaves the CM in the activatable antibody when the activatable antibody is exposed to the protease. In some embodiments of these methods and kits, the CM is a polypeptide of up to 15 amino acids in length. In some embodiments of these methods and kits, the CM is coupled to the N-terminus of the AB. In some embodiments of these methods and kits, the CM is coupled to the C-terminus of the AB. In some embodiments of these methods and kits, the CM is coupled to the N-terminus of a VL chain of the AB.

The activatable antibodies and/or conjugated activatable antibodies of the disclosure are used in diagnostic and prophylactic formulations. In one embodiment, an activatable antibody is administered to patients that are at risk of developing one or more of the aforementioned inflammation, inflammatory disorders, cancer or other disorders.

A patient's or organ's predisposition to one or more of the aforementioned disorders can be determined using genotypic, serological or biochemical markers.

In some embodiments of the disclosure, an activatable antibody and/or conjugated activatable antibodies is administered to human individuals diagnosed with a clinical indication associated with one or more of the aforementioned disorders. Upon diagnosis, an activatable antibody and/or conjugated activatable antibodies is administered to mitigate or reverse the effects of the clinical indication.

Activatable antibodies and/or conjugated activatable antibodies of the disclosure are also useful in the detection of the target in patient samples and accordingly are useful as diagnostics. For example, the activatable antibodies and/or conjugated activatable antibodies of the disclosure are used in in vitro assays, e.g., ELISA, to detect target levels in a patient sample.

In one embodiment, an activatable antibody of the disclosure is immobilized on a solid support (e.g., the well(s) of a microtiter plate). The immobilized activatable antibody serves as a capture antibody for any target that may be present in a test sample. Prior to contacting the immobilized antibody with a patient sample, the solid support is rinsed and treated with a blocking agent such as milk protein or albumin to prevent nonspecific adsorption of the analyte.

Subsequently the wells are treated with a test sample suspected of containing the antigen, or with a solution containing a standard amount of the antigen. Such a sample is, e.g., a serum sample from a subject suspected of having levels of circulating antigen considered to be diagnostic of a pathology. After rinsing away the test sample or standard, the solid support is treated with a second antibody that is detectably labeled. The labeled second antibody serves as a detecting antibody. The level of detectable label is measured, and the concentration of target antigen in the test sample is determined by comparison with a standard curve developed from the standard samples.

It will be appreciated that based on the results obtained using the antibodies of the disclosure in an in vitro diagnostic assay, it is possible to stage a disease in a subject based on expression levels of the Target antigen. For a given disease, samples of blood are taken from subjects diagnosed as being at various stages in the progression of the disease, and/or at various points in the therapeutic treatment of the disease. Using a population of samples that provides statistically significant results for each stage of progression or therapy, a range of concentrations of the antigen that may be considered characteristic of each stage is designated.

Activatable antibodies and/or conjugated activatable antibodies can also be used in diagnostic and/or imaging methods. In some embodiments, such methods are in vitro methods. In some embodiments, such methods are in vivo methods. In some embodiments, such methods are in situ methods. In some embodiments, such methods are ex vivo methods. For example, activatable antibodies having an enzymatically cleavable CM can be used to detect the presence or absence of an enzyme that is capable of cleaving the CM. Such activatable antibodies can be used in diagnostics, which can include in vivo detection (e.g., qualitative or quantitative) of enzyme activity (or, in some embodiments, an environment of increased reduction potential such as that which can provide for reduction of a disulfide bond) through measured accumulation of activated antibodies (i.e., antibodies resulting from cleavage of an activatable antibody) in a given cell or tissue of a given host organism. Such accumulation of activated antibodies indicates not only that the tissue expresses enzymatic activity (or an increased reduction potential depending on the nature of the CM) but also that the tissue expresses target to which the activated antibody binds.

For example, the CM can be selected to be a protease substrate for a protease found at the site of a tumor, at the site of a viral or bacterial infection at a biologically confined site (e.g., such as in an abscess, in an organ, and the like), and the like. The AB can be one that binds a target antigen. Using methods familiar to one skilled in the art, a detectable label (e.g., a fluorescent label or radioactive label or radiotracer) can be conjugated to an AB or other region of an activatable antibody. Suitable detectable labels are discussed in the context of the above screening methods and additional specific examples are provided below. Using an AB specific to a protein or peptide of the disease state, along with a protease whose activity is elevated in the disease tissue of interest, activatable antibodies will exhibit an increased rate of binding to disease tissue relative to tissues where the CM specific enzyme is not present at a detectable level or is present at a lower level than in disease tissue or is inactive (e.g., in zymogen form or in complex with an inhibitor). Since small proteins and peptides are rapidly cleared from the blood by the renal filtration system, and because the enzyme specific for the CM is not present at a detectable level (or is present at lower levels in non-disease tissues or is present in inactive conformation), accumulation of activated antibodies in the disease tissue is enhanced relative to non-disease tissues.

In another example, activatable antibodies can be used to detect the presence or absence of a cleaving agent in a sample. For example, where the activatable antibodies contain a CM susceptible to cleavage by an enzyme, the activatable antibodies can be used to detect (either qualitatively or quantitatively) the presence of an enzyme in the sample. In another example, where the activatable antibodies contain a CM susceptible to cleavage by reducing agent, the activatable antibodies can be used to detect (either qualitatively or quantitatively) the presence of reducing conditions in a sample. To facilitate analysis in these methods, the activatable antibodies can be detectably labeled, and can be bound to a support (e.g., a solid support, such as a slide or bead). The detectable label can be positioned on a portion of the activatable antibody that is not released following cleavage, for example, the detectable label can be a quenched fluorescent label or other label that is not detectable until cleavage has occurred. The assay can be conducted by, for example, contacting the immobilized, detectably labeled activatable antibodies with a sample suspected of containing an enzyme and/or reducing agent for a time sufficient for cleavage to occur, then washing to remove excess sample and contaminants. The presence or absence of the cleaving agent (e.g., enzyme or reducing agent) in the sample is then assessed by a change in detectable signal of the activatable antibodies prior to contacting with the sample e.g., the presence of and/or an increase in detectable signal due to cleavage of the activatable antibody by the cleaving agent in the sample.

Such detection methods can be adapted to also provide for detection of the presence or absence of a target that is capable of binding the AB of the activatable antibodies when cleaved. Thus, the assays can be adapted to assess the presence or absence of a cleaving agent and the presence or absence of a target of interest. The presence or absence of the cleaving agent can be detected by the presence of and/or an increase in detectable label of the activatable antibodies as described above, and the presence or absence of the target can be detected by detection of a target-AB complex e.g., by use of a detectably labeled anti-target antibody.

Activatable antibodies are also useful in in situ imaging for the validation of activatable antibody activation, e.g., by protease cleavage, and binding to a particular target. In situ imaging is a technique that enables localization of proteolytic activity and target in biological samples such as cell cultures or tissue sections. Using this technique, it is possible to confirm both binding to a given target and proteolytic activity based on the presence of a detectable label (e.g., a fluorescent label).

These techniques are useful with any frozen cells or tissue derived from a disease site (e.g. tumor tissue) or healthy tissues. These techniques are also useful with fresh cell or tissue samples.

In these techniques, an activatable antibody is labeled with a detectable label. The detectable label may be a fluorescent dye, (e.g. Fluorescein Isothiocyanate (FITC), Rhodamine Isothiocyanate (TRITC), a near infrared (NIR) dye (e.g., Qdot® nanocrystals), a colloidal metal, a hapten, a radioactive marker, biotin and an amplification reagent such as streptavidin, or an enzyme (e.g. horseradish peroxidase or alkaline phosphatase).

Detection of the label in a sample that has been incubated with the labeled, activatable antibody indicates that the sample contains the target and contains a protease that is specific for the CM of the activatable antibody. In some embodiments, the presence of the protease can be confirmed using broad spectrum protease inhibitors such as those described herein, and/or by using an agent that is specific for the protease, for example, an antibody such as A11, which is specific for the protease matriptase and inhibits the proteolytic activity of matriptase; see e.g., International Publication Number WO 2010/129609, published 11 Nov. 2010. The same approach of using broad spectrum protease inhibitors such as those described herein, and/or by using a more selective inhibitory agent can be used to identify a protease or class of proteases specific for the CM of the activatable antibody. In some embodiments, the presence of the target can be confirmed using an agent that is specific for the target, e.g., another antibody, or the detectable label can be competed with unlabeled target. In some embodiments, unlabeled activatable antibody could be used, with detection by a labeled secondary antibody or more complex detection system.

Similar techniques are also useful for in vivo imaging where detection of the fluorescent signal in a subject, e.g., a mammal, including a human, indicates that the disease site contains the target and contains a protease that is specific for the CM of the activatable antibody.

These techniques are also useful in kits and/or as reagents for the detection, identification or characterization of protease activity in a variety of cells, tissues, and organisms based on the protease-specific CM in the activatable antibody.

In some embodiments, in situ imaging and/or in vivo imaging are useful in methods to identify which patients to treat. For example, in in situ imaging, the activatable antibodies are used to screen patient samples to identify those patients having the appropriate protease(s) and target(s) at the appropriate location, e.g., at a tumor site.

In some embodiments in situ imaging is used to identify or otherwise refine a patient population suitable for treatment with an activatable antibody of the disclosure. For example, patients that test positive for both the target and a protease that cleaves the substrate in the cleavable moiety (CM) of the activatable antibody being tested (e.g., accumulate activated antibodies at the disease site) are identified as suitable candidates for treatment with such an activatable antibody comprising such a CM. Likewise, patients that test negative for either or both of the target and the protease that cleaves the substrate in the CM in the activatable antibody being tested using these methods are identified as suitable candidates for another form of therapy (i.e., not suitable for treatment with the activatable antibody being tested). In some embodiments, such patients that test negative with respect to a first activatable antibody can be tested with other activatable antibodies comprising different CMs until a suitable activatable antibody for treatment is identified (e.g., an activatable antibody comprising a CM that is cleaved by the patient at the site of disease).

In some embodiments in vivo imaging is used to identify or otherwise refine a patient population suitable for treatment with an activatable antibody of the disclosure. For example, patients that test positive for both the target and a protease that cleaves the substrate in the cleavable moiety (CM) of the activatable antibody being tested (e.g., accumulate activated antibodies at the disease site) are identified as suitable candidates for treatment with such an activatable antibody comprising such a CM. Likewise, patients that test negative are identified as suitable candidates for another form of therapy (i.e., not suitable for treatment with the activatable antibody being tested). In some embodiments, such patients that test negative with respect to a first activatable antibody can be tested with other activatable antibodies comprising different CMs until a suitable activatable antibody for treatment is identified (e.g., an activatable antibody comprising a CM that is cleaved by the patient at the site of disease).

Pharmaceutical Compositions

The conjugated antibodies, activatable antibodies and/or conjugated activatable antibodies of the disclosure (also referred to herein as “active compounds”), and derivatives, fragments, analogs and homologs thereof, can be incorporated into pharmaceutical compositions suitable for administration. Such compositions typically comprise the conjugated antibody, activatable antibody and/or conjugated activatable antibody and a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Suitable examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the disclosure is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In some embodiments, it will be desirable to include isotonic agents, for example, sugars, polyalcohols such as manitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

The invention will be further described in the following examples, which do not limit the scope of the invention described in the claims.

### EXAMPLES

### Example 1. Materials and Methods

Reagents and Strains: Streptavidin-conjugated phycoerythrin (SA-PE) (Invitrogen, Life Technologies) was used without modifications. Human matriptase-1 (Research & Diagnostics Systems, Inc.) was used without modifications. Human plasmin (Haematologic Technologies Inc.) was used without modifications. Human tPA (Molecular Innovations) was used without modifications. YPet fused to the SH3 domain of Mona (monocytic adaptor) was used without modifications. TBST, 50 mM Tris-HCl, 150 mM NaCl, 0.05% Tween20, pH 7.4, was used. E. coli MC1061 (Casadaban et al., JMB 138(2):179-207 (1980) was used. All bacterial growth was performed at 37° C. with vigorous shaking in Luria-Bertani broth (LB) supplemented with 34 μg/mL chloramphenicol, unless another antibiotic is specified.

Substrate Cleavage and Scaffold Stability Analysis: For screening and clone analysis, overnight cultures were subcultured by dilution into fresh medium (1:50) and grown for 1.5-2 hours. Each subculture was then induced with 0.04% arabinose and incubated with shaking at 37° C. for 1 hour. To stop further growth, cells were incubated on ice for 15-30 minutes. Cell aliquots were harvested and washed with PBS (pH 7.4). Cells were pelleted by centrifugation, the supernatant removed, and the cells resuspended in reaction buffer containing the enzyme; the reaction mixture was incubated at 37° C. static. To stop the reaction cells, were removed and diluted 10-fold in PBS, pelleted by centrifugation, and resuspended in PBS containing either SA-PE (20 μg/mL) or YPet-MONA (50 nM). After incubation on ice (30 min), cells were washed with PBS and analyzed using a FACSAria™ cell sorter.

For protease cleavage assays, cultures were induced for 1 hour. The reaction buffer for matriptase-1 was TB ST. Assays for matriptase-1 hydrolysis, were performed after reactions with 200 pM-200 nM matriptase-1 for 1 hr. Background hydrolysis of the regions flanking the substrate site (using platform eCLiPS3.0-NSUB_SP described in PCT patent application PCT/US13/54378, filed Aug. 9, 2013 and published as WO 2014/026136 on Feb. 13, 2014), was measured under each reaction condition to ensure that hydrolysis occurred in the designated substrate region.

For human plasmin stability assays, platform eCLiPS3.0-NSUB_SP was used; cultures were induced for 1 hr. The reaction buffer for plasmin was 50 mM Tris-HCl pH 7.5 supplemented with 100 mM NaCl, 0.01% Tween20 and 1 mM EDTA. Assays for plasmin hydrolysis were performed after reactions with plasmin for 1 hr.

For human tPA stability assays, platform eCLiPS3.0-NSUB_SP was used; cultures were induced for 1 hr. The reaction buffer for tPA was TB ST. Assays for tPA hydrolysis were performed after reactions with tPA for 1 hr.

Amino and Carboxy terminus labeling conditions: Streptavidin conjugated phycoerythrin (SAPE) was used for labeling streptavidin binding affinity ligand on the N-termini of CPX. Fluorescent protein YPet fused to the SH3 domain of Mona was used for labeling the MONA binding affinity ligand on the C-termini of CPX. For optimum labeling of cells without protease reaction, the cells were incubated for 30 min at 4° C. with SAPE (20 μg/mL) or YPet-MONA (50 nM).

Kinetic Data Analysis: The extent of conversion of cell surface displayed peptide substrates was measured directly, using flow cytometry to measure changes in mean fluorescence of clonal cell populations upon protease treatment. Specifically, for each sample, conversion was determined by flow cytometry analyses using the relationship

$$\\ \begin{matrix}Conversion = \frac{{FL}_{-} - {FL}_{+}}{{FL}_{-} - {FL}_{0}} & [ 1 ]\end{matrix}$$

where (FL₋) is the fluorescence after incubating without enzyme, (FL₊) is fluorescence after incubation with enzyme, and (FL₀) is fluorescence of unlabeled cells. Given that the expected substrate concentrations that were used are significantly below the expected K_{M} of the substrate for the target protease, the Michaelis-Menton model simplifies to:

$$\\ \begin{matrix}\frac{d [ S ]}{dt} \approx - \frac{{k}_{cat}}{{k}_{M}} [ S ] [ E ] & [ 2 ]\end{matrix}$$

allowing substrate conversion to be expressed as

$$\\ \begin{matrix}Conversion = 1 - exp ( - \frac{{k}_{cat}}{{k}_{M}} \cdot [ E ] \cdot t ) & [ 3 ]\end{matrix}$$

where [S] is the substrate concentration, [E] is enzyme concentration and t is time. To determine the second order rate constant (k_{cat}/K_{M}), equation [3] was simplified to:

$$\\ \frac{kcat}{Km} = - ln ( 1 - product conversion ) / ( time * [ protease ] )$$

Sequence Data Analysis—Meta Motifs: Substrates were submitted to Ion Torrent™ sequencing (see, e.g., Rothenberg, J M, Nature 475, 348-352). Raw Ion Torrent reads were cropped by invariant vector sequences to obtain just the variable peptide insert. Insert sequences were translated, and sequences with stop codons were excluded from further analysis. The frequency of each sequence was obtained by number of times observed out of all viable peptide reads observed. Enrichment of sequences was obtained by comparison of observed frequency of each sequence post selection to the frequency of each sequence pre-selection. Motif analysis was performed by extracting all possible 2mers, 3mers, 4mers and non-consecutive 1n2mers 2n1mers 2n2mers and 2nn2mers (where the first number represents the first set of invariant positions, the second number represents the second set of invariant positions, and the number of n's between represents the number of variable positions allowed between the two invariant positions). The frequency of each motif was established across all normalized sequences in both the pre-selected and post selected libraries to establish significance of enrichment of each motif. To generate meta-motifs, all sequences containing each motif were aligned, and a Positional Weight Matrix (PWM) was created representing the amino acid propensity at each position in the motif carriers. Profile-profile alignments and scoring were conducted across all motifs, using a Minimum Mutual Information Content (MMIC) scoring function to score each profile-profile aligned register. Registers aligning above a background of incorrect registers of unique formed PWMs were extracted as significant; the individual PWMs were then added to create an average metamotif.

Sequence Data Analysis—Directed Families: Final substrate pools were sequenced using Ion Torrent™ sequencing. Individual sequences were identified and isolated from these data, and sequences were aligned in CLC main lab (CLC Main Workbench 6.6.2, available online). The alignment file was imported to Jalview (see, e.g., Waterhouse, A. M., et al., 2009, Bioinformatics 9, 1189-1191), and an average distance tree was assembled using the BLOSUM62 algorithm (S Henikoff S et al., 1992, Proc Natl Acad Sci USA. 89, 10915-10919). The restricted group of sequences includes members of the cluster closest to the sequence of interest. The extended group of sequences includes the restricted group of sequences those plus of the branch that shares the closest common ancestor (where applicable).

### Example 2. Selection and Characterization of Substrate Pools in a Platform Scaffold

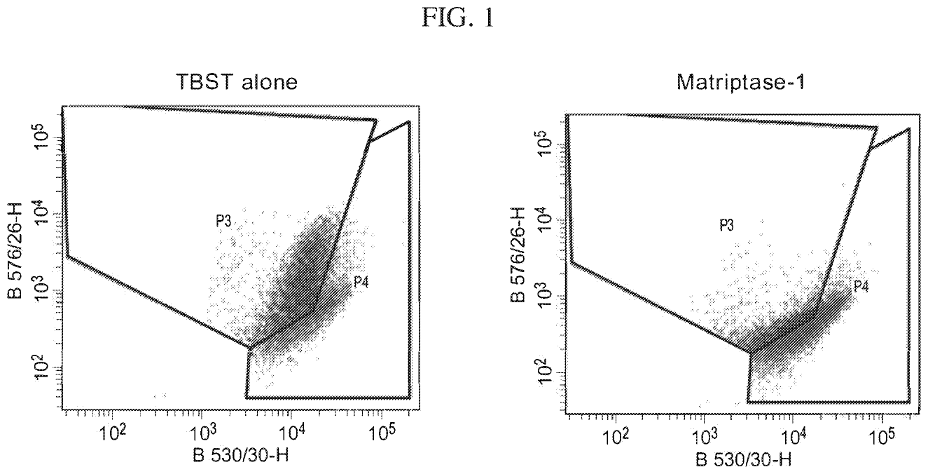

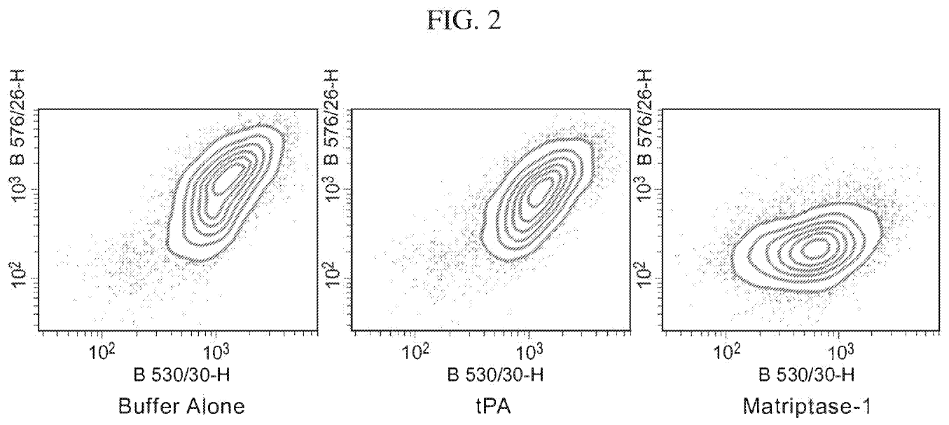

The use of multi-copy substrate display on whole cells enabled selection of populations of substrates cleaved by matriptase-1. Selections were performed as described in U.S. Pat. No. 7,666,817 B, issued Feb. 23, 2010, using recombinant human matriptase-1. Background hydrolysis of the regions flanking the substrate site (using platform eCLiPS3.0-NSUB_SP) was measured under each reaction condition to ensure that hydrolysis occurred in the designated substrate region. Final pools were tested against matriptase-1, plasmin and tPA. The pools were cleaved by matriptase-1 but not by tPA or plasmin. FIG. 1 shows cleavage of pool SMP30 by matriptase-1 in TBST, FIG. 2 shows cleavage of pool SMP17 by matriptase-1 at 50 nM and resistance to tPA, both in TBST. Using similar techniques, a separate library was screened to select substrates cleaved by both matriptase-1 and u-plasminogen activator but not by tPA or plasmin.

### Example 3. Characterization of Substrate Cleavage Kinetics in the Platform Scaffold

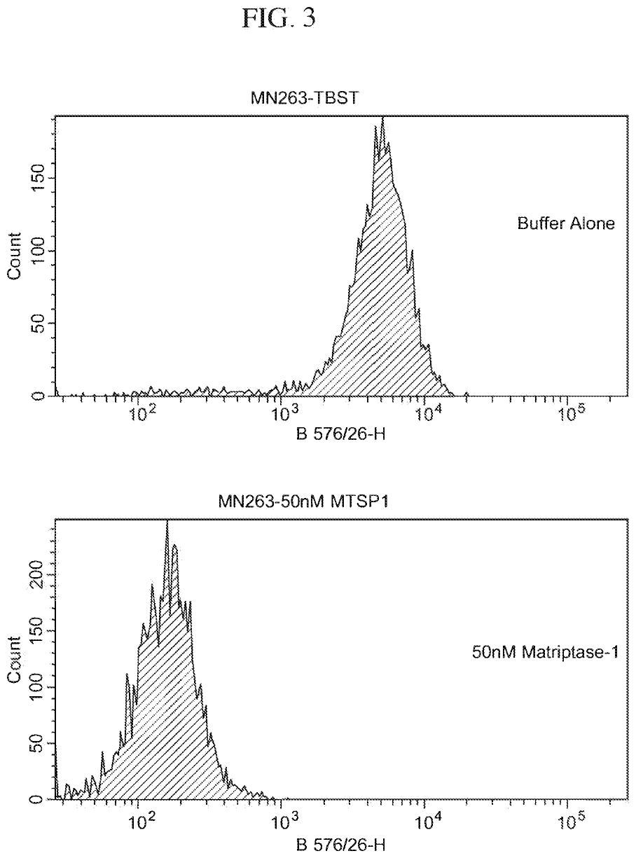

The use of multi-copy substrate display on whole cells enabled simple and direct quantitative characterization of cleavage kinetics. Consequently, flow cytometry was used to rank individual isolated clones on the basis of substrate conversion, and clones were identified by DNA sequencing. In this way, the extent of conversion for each clone could be determined at several different protease concentrations and fit to a Michaelis-Menten model (Kinetic Data Analysis Section). The observed second order rate constant (k_{cat}/K_{M}) was determined for each substrate versus matriptase-1. Background hydrolysis of the regions flanking the substrate site (using platform eCLiPS3.0-NSUB_SP), was measured under each reaction condition to ensure that hydrolysis occurred in the designated substrate region. For example, FIG. 3 shows cleavage of a substrate comprising amino acid sequence VAGRSMRP (SEQ ID NO: 251) by matriptase-1 in TB ST.

### Example 4. In Vitro Substrate Activity in Activatable Antibodies

This Example demonstrates the in vitro activity of substrates of the disclosure when they are incorporated into activatable antibodies.

Several substrates identified in these studies were inserted into Probodies having the 3954 mask and C225v5 variant of cetuximab, which is described in PCT Publication No. WO 2013/163631), which is incorporated herein by reference in its entirety.

The ability of substrates in the resultant activatable antibodies to be cleaved by matriptase-1 or uPA was determined as follows. All protease digests were performed in 50 mM Tris-HCl, 150 mM NaCl, 0.05% Tween-20 pH=7.4. Varying concentrations of active site titrated uPA or matriptase was combined with a fixed activatable antibody concentration to maintain a substrate to protease ratio of at least 50. Samples were incubated at 37° C. for up to 20 h. To stop the reaction, 5 μl of the digest was added to 7 μl of HT Protein Express Sample Buffer (Caliper LifeSciences) containing 20 mM 2-Mercaptoethanol for 10 minutes at 95° C. After heat denaturation, 32 μl of ddH₂O was added and samples analyzed on a LabChip GXII per manufacturer's instructions. The LabChip GXII software was used to quantify light chain peak area. Product conversion was calculated by plugging the light chain peak areas into the following equation: cleaved LC/(cleaved LC+uncleaved LC), LC=light chain. kcat/Km values were determined with the following equation

$$\\ \frac{{k}_{cat}}{{K}_{m}} = - ln ( 1 - C ) / ( {t}^{*} p )$$

where C is product conversion, t is time (s), and p is protease concentration (M), which assumes that the substrate concentration is below the Kₘ and in excess of the protease concentration.

Resultant activatable antibodies comprising substrates selected for cleavage by uPA and matriptase had k_{cat}/K_{M} values ranging from about 400 to 5,000 M⁻¹s⁻¹ for uPA and from about 3,000 to 100,000 M⁻¹s⁻¹ for matriptase (7 substrates tested). Resultant activatable antibodies comprising substrates selected for cleavage by matriptase had k_{cat}/K_{M} values ranging from about 6,500 to 100,000 M⁻¹s⁻¹ for matriptase (5 substrates tested).

### Example 5. Substrate Stability of Activatable Antibodies In Vivo

This Example demonstrates the in vivo stability of substrates of the disclosure when they are incorporated into activatable antibodies and injected into mice.

Activatable antibodies comprising several substrates of the disclosure, produced as described above, were labeled with either AlexaFluor 680 or DyLight 680 using standard NHS ester chemistry. Unreacted dye was removed by purification with a Zeba spin desalting column (40 kDa MWCO, ThermoFisher). Protein concentration was determined by A₂₈₀ using an extinction coefficient calculated from protein sequence and a correction factor that accounted for dye absorbance.

Three nude mice (Crl:NU-Foxn1nu) received a single IP dose of each activatable antibody at 10 mg/kg or 12.5 mg/kg on Day 0. Mice were euthanized on day 4 (about 96 h post-dose) by CO₂ asphyxiation and blood was collected immediately as plasma-EDTA and stored at −80° C.

Plasma samples were prepared for analysis by capillary electrophoresis as described in the k_{cat}/Kₘ section. Briefly, 5 μl of plasma was added to 7 μl Protein Express Sample Buffer with 2-mercaptoethanol. Quantification of circulating stability was identical to quantification of product conversion.

Of 14 activatable antibodies comprising substrates of the disclosure selected for cleavage by uPA or by matriptase, 13 exhibited less than 20% cleavage in the collected plasma samples.

### Example 6. Materials and Methods

Reagents and Strains: Human uPA (catalog no. 1310-SE, Research & Diagnostics Systems, Inc.) was used without modifications. Human matriptase-1 (catalog no. 3946-SE, Research & Diagnostics Systems, Inc.) was used without modifications. Human tPA (catalog no. HTPA-TC, Molecular Innovations) was used without modifications. Human plasmin (catalog no. HCPM-0140, Haematologic Technologies Inc.) was used without modifications. Anti-EE monoclonal antibody (Covance, Princeton, NJ) was labeled with Alexa 647 (Life Sciences) and used with no other modifications (named EE647). E. coli MC1061 or MC1061 derived strains (DH10β) were used for all experiments (Casadaban et al., JMB 138(2):179-207 (1980)). All bacterial growth was performed at 37° C. with vigorous shaking in Luria-Bertani broth (LB) supplemented with 34 μg/mL chloramphenicol (cm), unless another antibiotic is specified.

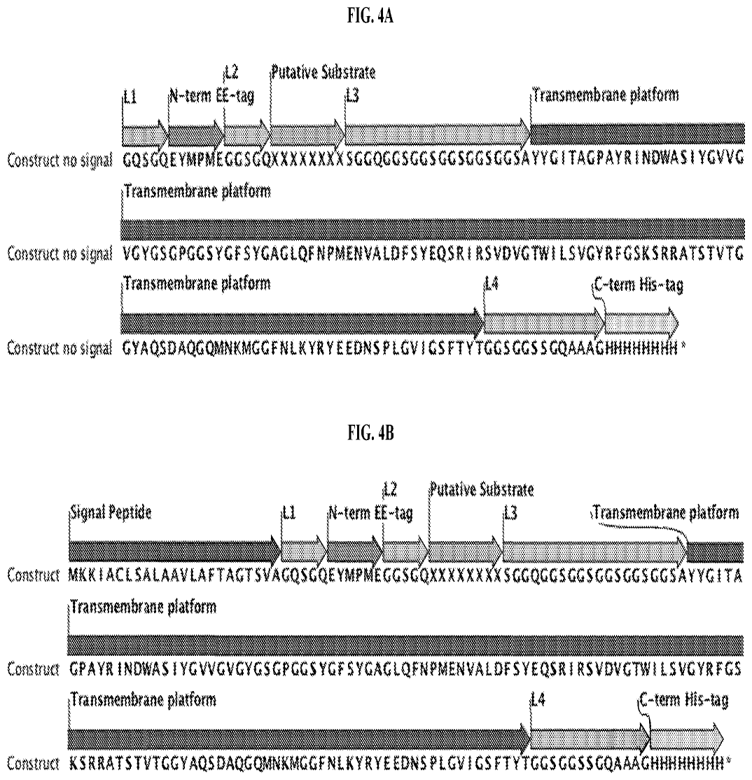

Display Platforms: Display platforms, each engineered to contain an 8-to-12-amino acid substrate of the embodiments, were produced and used as described in International Publication No. WO 2014/026136, published 13 Feb. 2014, the contents of which are hereby incorporated by reference in their entirety. The amino acid sequence of the mature (i.e., without a signal peptide) CYTX-DP-XXXXXXXX display platform (SEQ ID NO: 694) is shown in FIG. 4A. XXXXXXXX indicates the location into which each substrate is inserted. The amino acid sequence of CYTX-DP-XXXXXXXX display platform also including its signal peptide, i.e., SP-CYTX-DP-XXXXXXXX display platform (SEQ ID NO: 695) is shown in FIG. 4B.

CYTX-DP-XXXXXXXX Display Platform:
(SEQ ID NO: 694)
GQSGQEYMPMEGGSGQXXXXXXXXSGGQGGSGGSGGSGGSGGSAYYGITA
GPAYRINDWASIYGVVGVGYGSGPGGSYGFSYGAGLQFNPMENVALDFSY
EQSRIRSVDVGTWILSVGYRFGSKSRRATSTVTGGYAQSDAQGQMNKMGG
FNLKYRYEEDNSPLGVIGSFTYTGGSGGSSGQAAAGHHHHHHHH
SP-CYTX-DP-XXXXXXXX Display Platform:
(SEQ ID NO: 695)
MKKIACLSALAAVLAFTAGTSVAGQSGQEYMPMEGGSGQXXXXXXXXSGG
QGGSGGSGGSGGSGGSAYYGITAGPAYRINDWASIYGVVGVGYGSGPGGS
YGFSYGAGLQFNPMENVALDFSYEQSRIRSVDVGTWILSVGYRFGSKSRR
ATSTVTGGYAQSDAQGQMNKMGGFNLKYRYEEDNSPLGVIGSFTYTGGSG
GSSGQAAAGHHHHHHHH

Substrate Cleavage and Cleavage Kinetics Analysis: For clone analysis, overnight cultures were subcultured by dilution into fresh medium (1:40) and grown for 1.5-2 hours. The subculture was then induced with 0.04% arabinose and incubated with shaking at 37° C. for 40 minutes to 1 hour. To stop further growth, cells were then incubated on ice for 15 minutes to 1 hour. Cell aliquots were harvested and washed with reaction buffer. Cells were pelleted by centrifugation, the supernatant removed and the cells resuspended in reaction buffer containing the enzyme; the reaction mixture was incubated at 37° C. with shaking. To stop the reaction, cells were removed and diluted 10-fold in PBS, pelleted by centrifugation, and resuspended in PBS containing anti-EE647 (20 micrograms per milliliter (also referred to herein as ug/ml or μg/ml)). After incubation on ice (up to 1 hour), cells were washed with PBS and analyzed using an Accuri C6 cell sorter.

For uPA protease cleavage assays, cultures were induced for 40 minutes to 1 hour. The reaction buffer for uPA was 50 mM Tris-HCl, 150 mM NaCl, 0.05% Tween20, pH 7.4 (TBST). Assays for uPA hydrolysis were performed after cleavage with 2 nM-50 nM uPA for 1 hour. Background hydrolysis of the regions flanking the substrate site (using, e.g., CYTX-DP-NSUB, a display platform in which the “Substrate” is non-cleavable linker GGGSGGGS (SEQ ID NO: 696)) was measured under each reaction condition to ensure that hydrolysis occurred in the designated substrate region.

For matriptase-1 protease cleavage assays, cultures were induced for 40 minutes to 1 hour. The reaction buffer for matriptase-1 was 50 mM Tris-HCl, 150 mM NaCl, 0.05% Tween20, pH 7.4 (TBST). Assays for matriptase-1 hydrolysis were performed after cleavage with 2 nM-50 nM matriptase-1 for 1 hr. Background hydrolysis of the regions flanking the substrate site (using, e.g., CYTX-DP-NSUB) was measured under each reaction condition to ensure that hydrolysis occurred in the designated substrate region.

For human plasmin cleavage assays, cultures were induced for 40 minutes to 1 hour. The reaction buffer for plasmin was 50 mM Tris-HCl pH 7.5 supplemented with 100 mM NaCl, 0.01% Tween20 and 1 mM EDTA. Assays for plasmin hydrolysis were performed after cleavage with 20-500 pM plasmin for 1 hr. Background hydrolysis of the regions flanking the substrate site (using, e.g., CYTX-DP-NSTJB) was measured under each reaction condition to ensure that hydrolysis occurred in the designated substrate region.

For tPA protease cleavage assays, cultures were induced for 40 minutes to 1 hour. The reaction buffer for tPA was 50 mM Tris-HCl, 150 mM NaCl, 0.05% Tween20, pH 7.4 (TBST). Assays for tPA hydrolysis were performed after cleavage with 2 nM-50 nM tPA for 1 hour. Background hydrolysis of the regions flanking the substrate site (using, e.g., CYTX-DP-NSUB) was measured under each reaction condition to ensure that hydrolysis occurred in the designated substrate region.

Amino and Carboxyl terminus labeling conditions: Alexa-647 conjugated anti-EE antibody (EE647) was used for labeling the EE binding affinity ligand on the N-termini of the CYTX-DP-XXXXXXXX display platform. Alexa-647 conjugated anti-His antibody (His647) was used for labeling the 8His binding affinity ligand on the C-termini of the CYTX-DP-XXXXXXXX display platform. For optimum labeling of cells without protease reaction, the cells were incubated for 1 hour at 4° C. with EE647 (1 μg/mL) or His647 (2 μg/ml). For the example described below, a 1-hour incubation was used.

Kinetic Data Analysis: The extent of conversion of cell surface displayed peptide substrates was measured directly, using flow cytometry to measure changes in mean fluorescence of clonal cell populations upon protease treatment. Specifically, for each sample, conversion was determined by flow cytometry analyses using the relationship

$$\\ \begin{matrix}{Conversion}_{CLiPS} = \frac{{FL}_{-} - {FL}_{+}}{{FL}_{-} - {FL}_{0}} & [ 1 ]\end{matrix}$$

where (FL₋) is the fluorescence after incubating without enzyme, (FL₊) is fluorescence after incubation with enzyme, and (FL₀) is fluorescence of unlabeled cells. Given that the expected substrate concentrations that were used are significantly below the expected K_{M} of the substrate for the target protease, the Michaelis-Menten model simplifies to

$$\\ \begin{matrix}\frac{d [ S ]}{dt} \approx - \frac{{k}_{cat}}{{k}_{M}} [ S ] [ E ] & [ 2 ]\end{matrix}$$

allowing substrate conversion to be expressed as

$$\\ \begin{matrix}{Conversion}_{MM} = 1 - exp ( - \frac{{k}_{cat}}{{k}_{M}} \cdot [ E ] \cdot t ) & [ 3 ]\end{matrix}$$

where [S] is the substrate concentration, [E] is enzyme concentration and t is time. To determine the second order rate constant (k_{cat}/K_{M}), the time dependent conversion for each substrate was fit to equation [3].

### Example 7. Characterization of Substrate Cleavability in the CYTX-DP Display Platform

This Example demonstrates the ability of substrates of the embodiments to be cleaved by matriptase and/or uPA, but not by plasmin and/or tPA.

The use of multi-copy substrate display on whole cells enabled simple and direct quantitative characterization of cleavage kinetics. Clones encoding substrates were identified by DNA sequencing and subcloned into the CYTX-DP-XXXXXXXX display platform such that the expressed display platform contained the substrate (typically 8 or 12 amino acids) in place of XXXXXXXX. Individual substrate-displaying clones (148 independent substrate-containing display platforms in total) were assessed for cleavage by matriptase and/or uPA (target proteases, i.e., the proteases used to select the substrate) and plasmin and/or tPA (off-target protease); turnover was determined by flow cytometry. Fifty-one of the substrates were selected for cleavage by both matriptase and uPA (i.e., Matriptase- and uPA-selected Substrates). The twenty-eight Matriptase- and uPA-selected Substrates from Pools were selected from the same pools as substrates comprising amino acid sequences SEQ ID NOs: 308, 314, and 361 as well as from substrates comprising amino acid sequences 369-371, 374-379, and 381-384. The twenty-three Matriptase- and uPA-selected Consensus Substrates were selected from substrates comprising amino acid sequences SEQ ID NOs: 307-311, 313-314, and 320-368. Ninety-seven of the substrates were selected for cleavage by matriptase (i.e., Matriptase-selected Substrates). The fifty-two Matriptase-selected Substrates from Pools were selected from substrates in Tables 8A through 8J and in Tables 9A through 9J-3 as well as from substrates comprising amino acid sequences SEQ ID NOs: 250-267. The forty-five Matriptase-selected Consensus Substrates were selected from substrates comprising amino acid sequences SEQ ID NOs: 163-249.

In this way, the extent of cleavage for each clone could be determined and the data aggregated to determine a percent of clones that were cleaved by the target protease and not the off-target protease. Background hydrolysis of the regions flanking the substrate site (using, e.g., the CYTX-DP-NSLTB display platform) was measured under each reaction condition to ensure that hydrolysis occurred in the designated substrate region. Results are presented in Table 12.

TABLE 12
Summary statistics of substrate cleavability

| | | >20% | | | <20% | | |
|---|---|---|---|---|---|---|---|
| | | Cleavage | | | Cleavage | <20% | |
| | | with 50 nM | >20% | >20% | with | Cleavage | <20% |
| | | Matriptase- | Cleavage | Cleavage with | 500 pM | with | Cleavage |
| Discovery | Substrate | 1 or 50 nM | with 50 nM | 50 nM | Plasmin or | 500 pM | with |
| effort | Group | uPA | uPA | Matriptase-1 | 50 nM tPA | Plasmin | 50 nM tPA |
| Matriptase- | All | 100% | 78% | 75% | 76% | 76% | 96% |
| and uPA- | Matriptase- | (51 of 51) | (40 of 51) | (38 of 51) | (39 of 51) | (39 of 51) | (49 of 51) |
| selected | and uPA- | | | | | | |
| Substrates | selected | | | | | | |
| | Substrates | | | | | | |
| | tested | | | | | | |
| | Substrates | 100% | 64% | 89% | 89% | 89% | 93% |
| | from Pools | (28 of 28) | (18 of 28) | (25 of 28) | (25 of 28) | (25 of 28) | (26 of 28) |
| | Consensus | 100% | 96% | 57% | 61% | 61% | 100% |
| | Substrates | (23 of 23) | (22 of 23) | (13 of 23) | (14 of 23) | (14 of 23) | (23 of 23) |
| Matriptase- | All | 86% | 41% | 67% | 70% | 82% | 85% |
| selected | Matriptase- | (83 of 97) | (40 of 97) | (65 of 97) | (68 of 97) | (80 of 97) | (82 of 97) |
| Substrates | selected | | | | | | |
| | Substrates | | | | | | |
| | Substrates | 81% | 35% | 62% | 81% | 94% | 87% |
| | from Pools | (42 of 52) | (18 of 52) | (32 of 52) | (42 of 52) | (49 of 52) | (45 of 52) |
| | Consensus | 91% | 49% | 73% | 58 | 69% | 82% |
| | Substrates | (41 of 45) | (22 of 45) | (33 of 45) | (26 of 45) | (31 of 45) | (37 of 45) |
| Combined | Total | 91% | 54% | 70% | 72% | 80% | 89% |
| substrates | | (134 of 148) | (80 of 148) | (103 of 148) | (107 of 148) | (119 of 148) | (131 of 148) |
| selected for | | | | | | | |
| cleavage by | | | | | | | |
| Matriptase | | | | | | | |
| and/or uPA | | | | | | | |

Table 12 depicts the percentage of Matriptase- and uPA-selected Substrates or Matriptase-selected Substrates tested in the CYTX-DP display platform (a) that exhibited greater than 20% cleavage when incubated with 50 nM human uPA (catalog no. 1310-SE, Research & Diagnostics Systems, Inc.) used without modifications for 1 hour at 37° C. in 50 mM Tris-HCl, 150 mM NaCl, 0.05% Tween20, pH 7.4 (TBST) (>20% cleavage with 50 nM uPA); (b) that exhibited greater than 20% cleavage when incubated with 50 nM human matriptase-1 (catalog no. 3946-SE, Research & Diagnostics Systems, Inc.) used without modifications for 1 hour at 37° C. in 50 mM Tris-HCl, 150 mM NaCl, 0.05% Tween20, pH 7.4 (TBST) (>20% cleavage with 50 nM matriptase-1); (c) that exhibited less than 20% cleavage when incubated with 500 pM human plasmin (catalog no. HCPM-0140, Haematologic Technologies, Inc.) used without modifications for 1 hour at 37° C. in 50 mM Tris-HCl pH 7.5 supplemented with 100 mM NaCl, 0.01% Tween20 and 1 mM EDTA (<20% cleavage with 500 pM plasmin); and (d) that exhibited less than 20% cleavage when incubated with 50 nM human tPA (catalog no. HTPA-TC, Molecular Innovations) used without modifications for 1 hour at 37° C. in 50 mM Tris-HCl, 150 mM NaCl, 0.05% Tween20, pH 7.4 (TBST) (<20% cleavage with 50 nM tPA).

### Example 8. Characterization of Substrate Cleavage Kinetics in the CYTX-DP Display Platform

This Example demonstrates the cleavage kinetics of various substrates of the embodiments.

The use of multi-copy substrate display on whole cells enabled simple and direct quantitative characterization of cleavage kinetics. Clones were identified by DNA sequencing and subcloned into the CYTX-DP-XXXXXXXX display platform as described herein. Ninety individual substrate-displaying clones were assessed for cleavage, and a subset was chosen to assess cleavage kinetics by the clone's target protease. The extent of conversion for each clone could be determined at several different protease concentrations and fit to the Michaelis-Menten model described herein. Background hydrolysis of the regions flanking the substrate site (using, e.g., CYTX-DP-NSUB) was measured under each reaction condition to ensure that hydrolysis occurred in the designated substrate region. Results are presented in Table 13 & Table 14.

TABLE 13
Summary statistics of uPA substrate kinetics

| Discovery | uPA kcat/Km > | uPA kcat/Km > | uPA kcat/Km > | |
|---|---|---|---|---|
| effort | 1 × 10E2 | 1 × 10E3 | 1 × 10E4 | |
| Matriptase- and | 100% (18 of 18) | 100% (18 of 18) | 50% | (9 of 18) |
| uPA-selected | | | | |
| Substrates | | | | |
| Matriptase- | 100% (16 of 16) | 100% (16 of 16) | 6% | (1 of 16) |
| selected | | | | |
| Substrates | | | | |
| Combined | 100% (34 of 34) | 100% (34 of 34) | 29% | (10 of 34) |
| Substrates | | | | |
| Cleaved | | | | |
| by Matriptase | | | | |
| and/or uPA | | | | |

TABLE 14
Summary statistics of Matriptase-1 substrate kinetics

| | Matriptase-1 | Matriptase-1 | Matriptase-1 | |
|---|---|---|---|---|
| Discovery | kcat/Km > | kcat/Km > | kcat/Km > | |
| effort | 1 × 10E2 | 1 × 10E3 | 1 × 10E4 | |
| Matriptase- and | 100% (25 of 25) | 100% (25 of 25) | 16% | (4 of 25) |
| uPA-selected | | | | |
| Substrates | | | | |
| Matriptase- | 100% (31 of 31) | 100% (31 of 31) | 3% | (1 of 31) |
| selected | | | | |
| Substrates | | | | |
| Combined | 100% (56 of 56) | 100% (56 of 56) | 9% | (5 of 56) |
| Substrates | | | | |
| Cleaved | | | | |
| by Matriptase | | | | |
| and/or uPA | | | | |

### Example 9. In Vivo Efficacy and In Situ Activation of Activatable Antibodies Comprising a Substrate Cleavable by Matriptase and/or uPA

This Example demonstrates that activatable antibodies comprising substrates of the embodiments cleavable by matriptase and/or uPA are efficacious in vivo. This Example also demonstrates that such activatable antibodies are activatable in an in situ imaging assay, such as that described in International Publication No. WO 2014/107559, published 10 Jul. 2014, the contents of which are hereby incorporated by reference in their entirety.

Three activatable antibodies, each comprising a different substrate of the embodiments that are cleaved by matriptase and/or uPA, were administered at 10 mg/kg to H292 xenograft tumor-bearing (lung cancer) mice on day 0. All three activatable antibodies also comprised the masking moiety comprising the amino acid sequence CISPRGCPDGPYVMY (SEQ ID NO: 515) and anti-EGFR antibody C225v5 antibody comprising a light chain (SEQ ID NO: 458) and a heavy chain (SEQ ID NO: 455). The configuration of the light chain of the activatable antibody was masking moiety—substrate—light chain of C225v5.

Mice were retro-orbitally bled on day 4 (about 96 hours post-dose). Blood was collected immediately as plasma-EDTA and stored at −80° C. The three activatable antibodies were purified from plasma by anti-human IgG immunoprecipitation using magnetic beads. To analyze by capillary electrophoresis, 5 μl of eluted IgG was added to 7 μl Protein Express Sample Buffer (Caliper LifeSciences) containing 20 mM 2-Mercaptoethanol for 10 minutes at 95° C. After heat denaturation, 32 μl of ddH₂O was added and samples analyzed on a LabChip GXII per manufacturer's instructions. The LabChip GXII software was used to quantify light chain peak area. Product conversion was calculated by plugging the light chain peak areas into the following equation: cleaved LC/(cleaved LC+uncleaved LC), LC=light chain peak area. At day 4, the three activatable antibodies demonstrated mean % activation values ranging from 13% to 30%. Mean % activation is calculated as ((product conversion sum of the test group)*100%)/(number of animals in the test group).

The three activatable antibodies demonstrated tumor growth inhibition ranging from 32% to 59% as measured by mean % Δ inhibition. Mean % Δ inhibition is calculated as (mean(C)−mean(C0))−(mean(T)−mean(T0))/(mean(C)−mean(C0))*100%, wherein T is the current test group value, T0 is the current test group initial value, C is the control group value, and C0 is the control group initial value. The EGFR antibody cetuximab demonstrated 96% inhibition in this study.

The same three activatable antibodies were submitted to in situ imaging assays of mouse xenograft tumor tissues, using the conditions described in the examples of WO 2014/107559, ibid. The three activatable antibodies were activated, demonstrating that substrates were cleaved and the released antibodies bound to EGFR on the tumor tissue. The staining signals ranged from 15% to 85% of the IHC signal intensity of cetuximab.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following.

What is claimed:

1. An isolated polypeptide comprising a cleavable moiety (CM) comprising an amino acid sequence selected from the group consisting of SEQ ID NOs: 269-273, 275-279, 281, 282, 284-286, 270-273, 2-5, 7-9, 11, 12, 14-17, 19, 20, 22-25, 27-30, 32-34, 36-39, 41-44, and 250-267, wherein the cleavable moiety is a substrate for a protease.

2. The isolated polypeptide of claim 1, wherein the CM comprises an amino acid sequence selected from the group consisting of 288-292, 294-298, 300, 301, and 303-306.

3. The isolated polypeptide of claim 1, wherein the CM comprises an amino acid sequence selected from the group consisting of SEQ ID NOs: 307-311, 313, 314, 320-371, 374-379, and 381-384.

4. The isolated polypeptide of claim 1, wherein the CM comprises an amino acid sequence selected from the group consisting of 46-49, 51-53, 55-57, 59-61, 63, 64, 66, 67, 69, 70, 72-75, 77-80, 82-85, 87-89, 91-93, 95-97, 99-102, 104-107, 109-112, 114-117, 119-122, 124-127, 129-132, 134-137, 139-142, 144-147, 149-152, 154-157, and 159-162.

5. The isolated polypeptide of claim 1, wherein the CM comprises an amino acid sequence selected from the group consisting of SEQ ID NOs: 163-249.

6. The isolated polypeptide of claim 1, wherein the CM comprises an amino acid sequence selected from the group consisting of SEQ ID NOs: 250-253.

7. The isolated polypeptide of claim 1, wherein the CM comprises an amino acid sequence selected from the group consisting of SEQ ID NOs: 254-267.

8. The isolated polypeptide of claim 1, wherein the CM is a substrate for at least a matriptase protease or a u-plasminogen activator (uPA) protease.

9. The isolated polypeptide of claim 1, wherein the isolated polypeptide comprises at least one additional moiety (M) selected from the group consisting of a moiety that is located amino (N) terminally to the CM (M_{N}), a moiety that is located carboxyl (C) terminally to the CM (M_{C}), and combinations thereof.

10. The isolated polypeptide of claim 9, wherein the isolated polypeptide comprises at least one M_{N} and at least one M_{C}.

11. The isolated polypeptide of claim 9, wherein M_{N} is selected from the group consisting of a masking moiety, an antibody, a protein, a therapeutic agent, an antineoplastic agent, a toxic agent, a drug, a detectable moiety, a diagnostic agent, and an affinity tag.

12. The isolated polypeptide of claim 9, wherein M_{C} is selected from the group consisting of a masking moiety, an antibody, a protein, a therapeutic agent, an antineoplastic agent, a toxic agent, a drug, a detectable moiety, a diagnostic agent, and an affinity tag.

13. The isolated polypeptide of claim 1, wherein the polypeptide comprises an antibody or antigen binding fragment thereof (AB) that binds a target.

14. The isolated polypeptide of claim 13, wherein the CM is a substrate for a protease that is co-localized in a tissue with the target.

15. The isolated polypeptide of claim 13, wherein the antigen binding fragment thereof is selected from the group consisting of a Fab fragment, a F(ab′)₂ fragment, a scFv, a scab, a dAb, a single domain heavy chain antibody, and a single domain light chain antibody.

16. The isolated polypeptide of claim 13, wherein the AB is linked to the CM.

17. The isolated polypeptide of claim 16, wherein the AB is linked directly to the CM.

18. The isolated polypeptide of claim 16, wherein the AB is linked to the CM via a linking peptide.

19. The isolated polypeptide of claim 13, wherein the isolated polypeptide comprises a masking moiety (MM), wherein the MM has an equilibrium dissociation constant for binding to the AB that is greater than the equilibrium dissociation constant of the AB for binding to the target.

20. The isolated polypeptide of claim 19, wherein the MM is a polypeptide of no more than 40 amino acids in length.

21. The isolated polypeptide of claim 19, wherein the MM is linked to the CM such that the isolated polypeptide in an uncleaved state comprises the structural arrangement from N-terminus to C-terminus as follows: MM-CM-AB or AB-CM-MM.

22. The isolated polypeptide of claim 21, wherein the isolated polypeptide comprises a linking peptide between the MM and the CM.

23. The isolated polypeptide of claim 21, wherein the isolated polypeptide comprises a linking peptide between the CM and the AB.

24. The isolated polypeptide of claim 21, wherein the isolated polypeptide comprises a first linking peptide (LP1) and a second linking peptide (LP2), and wherein the isolated polypeptide has the structural arrangement from N-terminus to C-terminus as follows in the uncleaved state: MM-LP1-CM-LP2-AB or AB-LP2-CM-LP1-MM.

25. The isolated polypeptide of claim 24, wherein the two linking peptides need not be identical to each other.

26. The isolated polypeptide of claim 24, wherein each of LP1 and LP2 is a peptide of about 1 to 20 amino acids in length.

27. The isolated polypeptide of claim 19, wherein the amino acid sequence of the MM is different from that of the target.

28. The isolated polypeptide of claim 19, wherein the MM does not interfere or compete with the AB for binding to the target in a cleaved state.

29. The isolated polypeptide of claim 13 comprising a T cell-engaging scFv linked to the AB.

30. The isolated polypeptide of claim 29, wherein the T cell-engaging scFv comprises a masking moiety.

31. The isolated polypeptide of claim 19 comprising a T cell-engaging scFv linked to the AB.

32. The isolated polypeptide of claim 31, wherein the T cell-engaging scFv comprises a masking moiety.

33. The isolated polypeptide of claim 13 conjugated to an agent.

34. The isolated polypeptide of claim 33, wherein the agent is a toxin or fragment thereof.

35. The isolated polypeptide of claim 33, wherein the agent is selected from the group consisting of a dolastatin or a derivative thereof, an auristatin or a derivative thereof, a maytansinoid or a derivative thereof, a duocarmycin or a derivative thereof, and a calicheamicin or a derivative thereof.

36. The isolated polypeptide of claim 33, wherein the agent is a detectable moiety.

37. The isolated polypeptide of claim 33, wherein the agent is conjugated to the polypeptide via a linker.

38. The isolated polypeptide of claim 37, wherein the linker is a cleavable linker.

39. The isolated polypeptide of claim 19, wherein the AB is conjugated to an agent.

40. The isolated polypeptide of claim 39, wherein the agent is a toxin or fragment thereof.

41. The isolated polypeptide of claim 39, wherein the agent is selected from the group consisting of a dolastatin, an auristatin or a derivative thereof, a maytansinoid or a derivative thereof, a duocarmycin or a derivative thereof, and a calicheamicin or derivative thereof.

42. The isolated polypeptide of claim 39, wherein the agent is auristatin E or a derivative thereof.

43. The isolated polypeptide of claim 39, wherein the agent is monomethyl auristatin E (MMAE).

44. The isolated polypeptide of claim 39, wherein the agent is monomethyl auristatin D (MMAD).

45. The isolated polypeptide of claim 39, wherein the agent is DM1 or DM4.

46. The isolated polypeptide of claim 39, wherein the agent is conjugated to the AB via a linker.

47. The isolated polypeptide of claim 46, wherein the linker is a cleavable linker.

48. The isolated polypeptide of claim 13 comprising a detectable moiety.

49. The isolated polypeptide of claim 48, wherein the detectable moiety is a diagnostic agent.

50. An isolated nucleic acid molecule encoding the isolated polypeptide of claim 1.

51. A vector comprising the isolated nucleic acid molecule of claim 50.

52. An isolated nucleic acid molecule encoding the isolated polypeptide of claim 13.

53. A vector comprising the isolated nucleic acid molecule of claim 52.

54. An isolated nucleic acid molecule encoding the isolated polypeptide of claim 19.

55. A vector comprising the isolated nucleic acid molecule of claim 54.

56. A method of producing a cleavable moiety (CM) containing polypeptide by culturing a cell under conditions that lead to expression of the polypeptide, wherein the cell comprises the vector of claim 51.

57. A method of manufacturing a cleavable moiety (CM) containing polypeptide, the method comprising:
(a) culturing a cell comprising a nucleic acid construct that encodes the isolated polypeptide of claim 1 under conditions that lead to expression of the polypeptide, and
(b) recovering the polypeptide.


SEQUENCE LISTING (696 sequences)

SEQ ID NO: 1-162 - skipped (162 entries)

SEQ ID NO: 163 - protein, 5 aa, synthetic construct
  REGION 1..5: note = Core CM consensus 1 sequence
AAPRS

SEQ ID NO: 164 - protein, 6 aa, synthetic construct
  REGION 1..6: note = Expanded core CM consensus 1 sequence
AAPRSF

SEQ ID NO: 165 - protein, 5 aa, synthetic construct
  REGION 1..5: note = Core CM consensus 2 sequence
SRRVP

SEQ ID NO: 166 - protein, 6 aa, synthetic construct
  REGION 1..6: note = Expanded core CM consensus 2 sequence
QSRRVP

SEQ ID NO: 167 - protein, 6 aa, synthetic construct
  REGION 1..6: note = Expanded core CM consensus 2 sequence
QTRRVP

SEQ ID NO: 168 - protein, 6 aa, synthetic construct
  REGION 1..6: note = Expanded core CM consensus 2 sequence
SRRVPL

SEQ ID NO: 169 - protein, 6 aa, synthetic construct
  REGION 1..6: note = Expanded core CM consensus 2 sequence
SRRVPV

SEQ ID NO: 170 - protein, 7 aa, synthetic construct
  REGION 1..7: note = Expanded core CM consensus 2 sequence
QSRRVPL

SEQ ID NO: 171 - protein, 7 aa, synthetic construct
  REGION 1..7: note = Expanded core CM consensus 2 sequence
QSRRVPV

SEQ ID NO: 172 - protein, 7 aa, synthetic construct
  REGION 1..7: note = Expanded core CM consensus 2 sequence
QTRRVPL

SEQ ID NO: 173 - protein, 7 aa, synthetic construct
  REGION 1..7: note = Expanded core CM consensus 2 sequence
QTRRVPV

SEQ ID NO: 174 - protein, 5 aa, synthetic construct
  REGION 1..5: note = Core CM consensus 3 sequence
PPLGR

SEQ ID NO: 175 - protein, 6 aa, synthetic construct
  REGION 1..6: note = Expanded core CM consensus 3 sequence
GPPLGR

SEQ ID NO: 176 - protein, 6 aa, synthetic construct
  REGION 1..6: note = Expanded core CM consensus 3 sequence
SPPLGR

SEQ ID NO: 177 - protein, 7 aa, synthetic construct
  REGION 1..7: note = Expanded core CM consensus 3 sequence
CGPPLGR

SEQ ID NO: 178 - protein, 7 aa, synthetic construct
  REGION 1..7: note = Expanded core CM consensus 3 sequence
CSPPLGR

SEQ ID NO: 179 - protein, 7 aa, synthetic construct
  REGION 1..7: note = Expanded core CM consensus 3 sequence
GGPPLGR

SEQ ID NO: 180 - protein, 7 aa, synthetic construct
  REGION 1..7: note = Expanded core CM consensus 3 sequence
GSPPLGR

SEQ ID NO: 181 - protein, 7 aa, synthetic construct
  REGION 1..7: note = Expanded core CM consensus 3 sequence
SGPPLGR

SEQ ID NO: 182 - protein, 7 aa, synthetic construct
  REGION 1..7: note = Expanded core CM consensus 3 sequence
SSPPLGR

SEQ ID NO: 183 - protein, 8 aa, synthetic construct
  REGION 1..8: note = Expanded core CM consensus 3 sequence
GCGPPLGR

SEQ ID NO: 184 - protein, 8 aa, synthetic construct
  REGION 1..8: note = Expanded core CM consensus 3 sequence
GCSPPLGR

SEQ ID NO: 185 - protein, 8 aa, synthetic construct
  REGION 1..8: note = Expanded core CM consensus 3 sequence
GGGPPLGR

SEQ ID NO: 186 - protein, 8 aa, synthetic construct
  REGION 1..8: note = Expanded core CM consensus 3 sequence
GGSPPLGR

SEQ ID NO: 187 - protein, 8 aa, synthetic construct
  REGION 1..8: note = Expanded core CM consensus 3 sequence
GSGPPLGR

SEQ ID NO: 188 - protein, 8 aa, synthetic construct
  REGION 1..8: note = Expanded core CM consensus 3 sequence
GSSPPLGR

SEQ ID NO: 189 - protein, 8 aa, synthetic construct
  REGION 1..8: note = Expanded core CM consensus 3 sequence
SCGPPLGR

SEQ ID NO: 190 - protein, 8 aa, synthetic construct
  REGION 1..8: note = Expanded core CM consensus 3 sequence
SCSPPLGR

SEQ ID NO: 191 - protein, 8 aa, synthetic construct
  REGION 1..8: note = Expanded core CM consensus 3 sequence
SGGPPLGR

SEQ ID NO: 192 - protein, 8 aa, synthetic construct
  REGION 1..8: note = Expanded core CM consensus 3 sequence
SGSPPLGR

SEQ ID NO: 193 - protein, 8 aa, synthetic construct
  REGION 1..8: note = Expanded core CM consensus 3 sequence
SSGPPLGR

SEQ ID NO: 194 - protein, 8 aa, synthetic construct
  REGION 1..8: note = Expanded core CM consensus 3 sequence
SSSPPLGR

SEQ ID NO: 195 - protein, 5 aa, synthetic construct
  REGION 1..5: note = Core CM consensus 4 sequence
LRSGW

SEQ ID NO: 196 - protein, 6 aa, synthetic construct
  REGION 1..6: note = Expanded core CM consensus 4
MLRSGW

SEQ ID NO: 197 - protein, 7 aa, synthetic construct
  REGION 1..7: note = Expanded core CM consensus 4
MLRSGWR

SEQ ID NO: 198 - protein, 8 aa, synthetic construct
  REGION 1..8: note = Expanded core CM consensus 4
MLRSGWRG

SEQ ID NO: 199 - protein, 8 aa, synthetic construct
  REGION 1..8: note = Expanded core CM consensus 4
MLRSGWRL

SEQ ID NO: 200 - protein, 8 aa, synthetic construct
  REGION 1..8: note = Expanded core CM consensus 4
MLRSGWRS

SEQ ID NO: 201 - protein, 5 aa, synthetic construct
  REGION 1..5: note = Core CM consensus 5 sequence
VSRSA

SEQ ID NO: 202 - protein, 6 aa, synthetic construct
  REGION 1..6: note = Expanded core CM consensus 5 sequence
IVSRSA

SEQ ID NO: 203 - protein, 7 aa, synthetic construct
  REGION 1..7: note = Expanded core CM consensus 5 sequence
YIVSRSA

SEQ ID NO: 204 - protein, 8 aa, synthetic construct
  REGION 1..8: note = Expanded core CM consensus 5 sequence
QYIVSRSA

SEQ ID NO: 205 - protein, 5 aa, synthetic construct
  REGION 1..5: note = Core CM consensus 6 sequence
ALRAP

SEQ ID NO: 206 - protein, 6 aa, synthetic construct
  REGION 1..6: note = Expanded core CM consensus 6 sequence
RALRAP

SEQ ID NO: 207 - protein, 5 aa, synthetic construct
  REGION 1..5: note = Core CM consensus 7 sequence
PAGRR

SEQ ID NO: 208 - protein, 6 aa, synthetic construct
  REGION 1..6: note = Expanded core CM consensus 7 sequence
PAGRRS

SEQ ID NO: 209 - protein, 7 aa, synthetic construct
  REGION 1..7: note = Expanded core CM consensus 7 sequence
PAGRRSL

SEQ ID NO: 210 - protein, 7 aa, synthetic construct
  REGION 1..7: note = Expanded core CM consensus 7 sequence
VPAGRRS

SEQ ID NO: 211 - protein, 8 aa, synthetic construct
  REGION 1..8: note = Expanded core CM consensus 7 sequence
VPAGRRSL

SEQ ID NO: 212 - protein, 5 aa, synthetic construct
  REGION 1..5: note = Core CM consensus 8 sequence
GRSML

SEQ ID NO: 213 - protein, 6 aa, synthetic construct
  REGION 1..6: note = Expanded core CM consensus 8 sequence
GRSMLL

SEQ ID NO: 214 - protein, 6 aa, synthetic construct
  REGION 1..6: note = Expanded core CM consensus 8 sequence
GRSMLM

SEQ ID NO: 215 - protein, 7 aa, synthetic construct
  REGION 1..7: note = Expanded core CM consensus 8 sequence
GRSMLLG

SEQ ID NO: 216 - protein, 7 aa, synthetic construct
  REGION 1..7: note = Expanded core CM consensus 8 sequence
GRSMLLP

SEQ ID NO: 217 - protein, 7 aa, synthetic construct
  REGION 1..7: note = Expanded core CM consensus 8 sequence
GRSMLLS

SEQ ID NO: 218 - protein, 7 aa, synthetic construct
  REGION 1..7: note = Expanded core CM consensus 8 sequence
GRSMLMG

SEQ ID NO: 219 - protein, 7 aa, synthetic construct
  REGION 1..7: note = Expanded core CM consensus 8 sequence
GRSMLMP

SEQ ID NO: 220 - protein, 7 aa, synthetic construct
  REGION 1..7: note = Expanded core CM consensus 8 sequence
GRSMLMS

SEQ ID NO: 221 - protein, 8 aa, synthetic construct
  REGION 1..8: note = Expanded core CM consensus 8 sequence
GRSMLLGG

SEQ ID NO: 222 - protein, 8 aa, synthetic construct
  REGION 1..8: note = Expanded core CM consensus 8 sequence
GRSMLLPG

SEQ ID NO: 223 - protein, 8 aa, synthetic construct
  REGION 1..8: note = Expanded core CM consensus 8 sequence
GRSMLLSG

SEQ ID NO: 224 - protein, 8 aa, synthetic construct
  REGION 1..8: note = Expanded core CM consensus 8 sequence
GRSMLMGG

SEQ ID NO: 225 - protein, 8 aa, synthetic construct
  REGION 1..8: note = Expanded core CM consensus 8 sequence
GRSMLMPG

SEQ ID NO: 226 - protein, 8 aa, synthetic construct
  REGION 1..8: note = Expanded core CM consensus 8 sequence
GRSMLMSG

SEQ ID NO: 227 - protein, 8 aa, synthetic construct
  REGION 1..8: note = Expanded core CM consensus 8 sequence
GRSMLLGP

SEQ ID NO: 228 - protein, 8 aa, synthetic construct
  REGION 1..8: note = Expanded core CM consensus 8 sequence
GRSMLLPP

SEQ ID NO: 229 - protein, 8 aa, synthetic construct
  REGION 1..8: note = Expanded core CM consensus 8 sequence
GRSMLLSP

SEQ ID NO: 230 - protein, 8 aa, synthetic construct
  REGION 1..8: note = Expanded core CM consensus 8 sequence
GRSMLMGP

SEQ ID NO: 231 - protein, 8 aa, synthetic construct
  REGION 1..8: note = Expanded core CM consensus 8 sequence
GRSMLMPP

SEQ ID NO: 232 - protein, 8 aa, synthetic construct
  REGION 1..8: note = Expanded core CM consensus 8 sequence
GRSMLMSP

SEQ ID NO: 233 - protein, 8 aa, synthetic construct
  REGION 1..8: note = Expanded core CM consensus 8 sequence
GRSMLLGS

SEQ ID NO: 234 - protein, 8 aa, synthetic construct
  REGION 1..8: note = Expanded core CM consensus 8 sequence
GRSMLLPS

SEQ ID NO: 235 - protein, 8 aa, synthetic construct
  REGION 1..8: note = Expanded core CM consensus 8 sequence
GRSMLLSS

SEQ ID NO: 236 - protein, 8 aa, synthetic construct
  REGION 1..8: note = Expanded core CM consensus 8 sequence
GRSMLMGS

SEQ ID NO: 237 - protein, 8 aa, synthetic construct
  REGION 1..8: note = Expanded core CM consensus 8 sequence
GRSMLMPS

SEQ ID NO: 238 - protein, 8 aa, synthetic construct
  REGION 1..8: note = Expanded core CM consensus 8 sequence
GRSMLMSS

SEQ ID NO: 239 - protein, 5 aa, synthetic construct
  REGION 1..5: note = Core CM consensus 9 sequence
LARAG

SEQ ID NO: 240 - protein, 6 aa, synthetic construct
  REGION 1..6: note = Expanded core CM consensus 9 sequence
LARAGI

SEQ ID NO: 241 - protein, 6 aa, synthetic construct
  REGION 1..6: note = Expanded core CM consensus 9 sequence
LARAGL

SEQ ID NO: 242 - protein, 7 aa, synthetic construct
  REGION 1..7: note = Expanded core CM consensus 9 sequence
PLARAGI

SEQ ID NO: 243 - protein, 7 aa, synthetic construct
  REGION 1..7: note = Expanded core CM consensus 9 sequence
PLARAGL

SEQ ID NO: 244 - protein, 8 aa, synthetic construct
  REGION 1..8: note = Expanded core CM consensus 9 sequence
RPLARAGI

SEQ ID NO: 245 - protein, 8 aa, synthetic construct
  REGION 1..8: note = Expanded core CM consensus 9 sequence
RPLARAGL

SEQ ID NO: 246 - protein, 5 aa, synthetic construct
  REGION 1..5: note = Core CM consensus 10 sequence
ESRRW

SEQ ID NO: 247 - protein, 6 aa, synthetic construct
  REGION 1..6: note = Expanded core CM consensus 10 sequence
ESRRWM

SEQ ID NO: 248 - protein, 7 aa, synthetic construct
  REGION 1..7: note = Expanded core CM consensus 10 sequence
ESRRWMP

SEQ ID NO: 249 - protein, 8 aa, synthetic construct
  REGION 1..8: note = Expanded core CM consensus 10 sequence
PESRRWMP

SEQ ID NO: 250 - protein, 8 aa, synthetic construct
  REGION 1..8: note = Cleavable moiety
ILPRSPAF

SEQ ID NO: 251 - protein, 8 aa, synthetic construct
  REGION 1..8: note = Cleavable moiety
VAGRSMRP

SEQ ID NO: 252 - protein, 8 aa, synthetic construct
  REGION 1..8: note = Cleavable moiety
VVPEGRRS

SEQ ID NO: 253 - protein, 8 aa, synthetic construct
  REGION 1..8: note = Cleavable moiety
QGRAITFI

SEQ ID NO: 254 - protein, 8 aa, synthetic construct
  REGION 1..8: note = Cleavable moiety
VLSKQMSF

SEQ ID NO: 255 - protein, 8 aa, synthetic construct
  REGION 1..8: note = Cleavable moiety
LKGRSYYY

SEQ ID NO: 256 - protein, 8 aa, synthetic construct
  REGION 1..8: note = Cleavable moiety
KRMPVQFL

SEQ ID NO: 257 - protein, 8 aa, synthetic construct
  REGION 1..8: note = Cleavable moiety
PQHRIVSF

SEQ ID NO: 258 - protein, 8 aa, synthetic construct
  REGION 1..8: note = Cleavable moiety
YKKFVGSL

SEQ ID NO: 259 - protein, 8 aa, synthetic construct
  REGION 1..8: note = Cleavable moiety
HMMQYARH

SEQ ID NO: 260 - protein, 8 aa, synthetic construct
  REGION 1..8: note = Cleavable moiety
IPFSWSRF

SEQ ID NO: 261 - protein, 8 aa, synthetic construct
  REGION 1..8: note = Cleavable moiety
LSQARWRK

SEQ ID NO: 262 - protein, 8 aa, synthetic construct
  REGION 1..8: note = Cleavable moiety
DISHWRRS

SEQ ID NO: 263 - protein, 8 aa, synthetic construct
  REGION 1..8: note = Cleavable moiety
RKTVQHWW

SEQ ID NO: 264 - protein, 8 aa, synthetic construct
  REGION 1..8: note = Cleavable moiety
RFYRNQFF

SEQ ID NO: 265 - protein, 8 aa, synthetic construct
  REGION 1..8: note = Cleavable moiety
RSLVFAPI

SEQ ID NO: 266 - protein, 8 aa, synthetic construct
  REGION 1..8: note = Cleavable moiety
RSPSRLKC

SEQ ID NO: 267 - protein, 8 aa, synthetic construct
  REGION 1..8: note = Cleavable moiety
RKMPNITV

SEQ ID NO: 268-306 - skipped (39 entries)

SEQ ID NO: 307 - protein, 8 aa, synthetic construct
  REGION 1..8: note = Core CM consensus 11 sequence
LSGRSANH

SEQ ID NO: 308 - protein, 8 aa, synthetic construct
  REGION 1..8: note = Core CM consensus 11 sequence
LSGRSGNH

SEQ ID NO: 309 - protein, 12 aa, synthetic construct
  REGION 1..12: note = Expanded core CM consensus 11 or 12 sequence
DRLSGRSANHKK

SEQ ID NO: 310 - protein, 12 aa, synthetic construct
  REGION 1..12: note = Expanded core CM consensus 11 or 12 sequence
DRLSGRSDNHKK

SEQ ID NO: 311 - protein, 12 aa, synthetic construct
  REGION 1..12: note = Expanded core CM consensus 11 sequence
NTLSGRSGNHGS

SEQ ID NO: 312 - protein, 8 aa, synthetic construct
  REGION 1..8: note = cleavable linker sequence
QALAMSAI

SEQ ID NO: 313 - protein, 8 aa, synthetic construct
  REGION 1..8: note = Core CM consensus 12 sequence
LNGRSDNH

SEQ ID NO: 314 - protein, 8 aa, synthetic construct
  REGION 1..8: note = Core CM consensus 12 sequence
LTGRSDRH

SEQ ID NO: 315 - protein, 8 aa, synthetic construct
  REGION 1..8: note = cleavable linker sequence
AAYHLVSQ

SEQ ID NO: 316 - protein, 8 aa, synthetic construct
  REGION 1..8: note = cleavable linker sequence
MDAFLESS

SEQ ID NO: 317 - protein, 8 aa, synthetic construct
  REGION 1..8: note = cleavable linker sequence
ESLPVVAV

SEQ ID NO: 318 - protein, 8 aa, synthetic construct
  REGION 1..8: note = cleavable linker sequence
SAPAVESE

SEQ ID NO: 319 - protein, 8 aa, synthetic construct
  REGION 1..8: note = cleavable linker sequence
DVAQFVLT

SEQ ID NO: 320 - protein, 12 aa, synthetic construct
  REGION 1..12: note = Expanded core CM consensus 12 sequence
GPLNGRSDNHKA

SEQ ID NO: 321 - protein, 12 aa, synthetic construct
  REGION 1..12: note = Expanded core CM consensus 12 sequence
GPLNGRSDNHKK

SEQ ID NO: 322 - protein, 12 aa, synthetic construct
  REGION 1..12: note = Expanded core CM consensus 12 sequence
GPLNGRSDNHKR

SEQ ID NO: 323 - protein, 12 aa, synthetic construct
  REGION 1..12: note = Expanded core CM consensus 12 sequence
GPLNGRSDNHQA

SEQ ID NO: 324 - protein, 12 aa, synthetic construct
  REGION 1..12: note = Expanded core CM consensus 12 sequence
GPLNGRSDNHQK

SEQ ID NO: 325 - protein, 12 aa, synthetic construct
  REGION 1..12: note = Expanded core CM consensus 12 sequence
GPLNGRSDNHQR

SEQ ID NO: 326 - protein, 12 aa, synthetic construct
  REGION 1..12: note = Expanded core CM consensus 12 sequence
GPLNGRSDNHRA

SEQ ID NO: 327 - protein, 12 aa, synthetic construct
  REGION 1..12: note = Expanded core CM consensus 12 sequence
GPLNGRSDNHRK

SEQ ID NO: 328 - protein, 12 aa, synthetic construct
  REGION 1..12: note = Expanded core CM consensus 12 sequence
GPLNGRSDNHRR

SEQ ID NO: 329 - protein, 12 aa, synthetic construct
  REGION 1..12: note = Expanded core CM consensus 12 sequence
RPLNGRSDNHKA

SEQ ID NO: 330 - protein, 12 aa, synthetic construct
  REGION 1..12: note = Expanded core CM consensus 12 sequence
RPLNGRSDNHKK

SEQ ID NO: 331 - protein, 12 aa, synthetic construct
  REGION 1..12: note = Expanded core CM consensus 12 sequence
RPLNGRSDNHKR

SEQ ID NO: 332 - protein, 12 aa, synthetic construct
  REGION 1..12: note = Expanded core CM consensus 12 sequence
RPLNGRSDNHQA

SEQ ID NO: 333 - protein, 12 aa, synthetic construct
  REGION 1..12: note = Expanded core CM consensus 12 sequence
RPLNGRSDNHQK

SEQ ID NO: 334 - protein, 12 aa, synthetic construct
  REGION 1..12: note = Expanded core CM consensus 12 sequence
RPLNGRSDNHQR

SEQ ID NO: 335 - protein, 12 aa, synthetic construct
  REGION 1..12: note = Expanded core CM consensus 12 sequence
RPLNGRSDNHRA

SEQ ID NO: 336 - protein, 12 aa, synthetic construct
  REGION 1..12: note = Expanded core CM consensus 12 sequence
RPLNGRSDNHRK

SEQ ID NO: 337 - protein, 12 aa, synthetic construct
  REGION 1..12: note = Expanded core CM consensus 12 sequence
RPLNGRSDNHRR

SEQ ID NO: 338 - protein, 12 aa, synthetic construct
  REGION 1..12: note = Expanded core CM consensus 12 sequence
GPLSGRSDNHKA

SEQ ID NO: 339 - protein, 12 aa, synthetic construct
  REGION 1..12: note = Expanded core CM consensus 12 sequence
GPLSGRSDNHKK

SEQ ID NO: 340 - protein, 12 aa, synthetic construct
  REGION 1..12: note = Expanded core CM consensus 12 sequence
GPLSGRSDNHKR

SEQ ID NO: 341 - protein, 12 aa, synthetic construct
  REGION 1..12: note = Expanded core CM consensus 12 sequence
GPLSGRSDNHQA

SEQ ID NO: 342 - protein, 12 aa, synthetic construct
  REGION 1..12: note = Expanded core CM consensus 12 sequence
GPLSGRSDNHQK

SEQ ID NO: 343 - protein, 12 aa, synthetic construct
  REGION 1..12: note = Expanded core CM consensus 12 sequence
GPLSGRSDNHQR

SEQ ID NO: 344 - protein, 12 aa, synthetic construct
  REGION 1..12: note = Expanded core CM consensus 12 sequence
GPLSGRSDNHRA

SEQ ID NO: 345 - protein, 12 aa, synthetic construct
  REGION 1..12: note = Expanded core CM consensus 12 sequence
GPLSGRSDNHRK

SEQ ID NO: 346 - protein, 12 aa, synthetic construct
  REGION 1..12: note = Expanded core CM consensus 12 sequence
GPLSGRSDNHRR

SEQ ID NO: 347 - protein, 12 aa, synthetic construct
  REGION 1..12: note = Expanded core CM consensus 12 sequence
RPLSGRSDNHKA

SEQ ID NO: 348 - protein, 12 aa, synthetic construct
  REGION 1..12: note = Expanded core CM consensus 12 sequence
RPLSGRSDNHKK

SEQ ID NO: 349 - protein, 12 aa, synthetic construct
  REGION 1..12: note = Expanded core CM consensus 12 sequence
RPLSGRSDNHKR

SEQ ID NO: 350 - protein, 12 aa, synthetic construct
  REGION 1..12: note = Expanded core CM consensus 12 sequence
RPLSGRSDNHQA

SEQ ID NO: 351 - protein, 12 aa, synthetic construct
  REGION 1..12: note = Expanded core CM consensus 12 sequence
RPLSGRSDNHQK

SEQ ID NO: 352 - protein, 12 aa, synthetic construct
  REGION 1..12: note = Expanded core CM consensus 12 sequence
RPLSGRSDNHQR

SEQ ID NO: 353 - protein, 12 aa, synthetic construct
  REGION 1..12: note = Expanded core CM consensus 12 sequence
RPLSGRSDNHRA

SEQ ID NO: 354 - protein, 12 aa, synthetic construct
  REGION 1..12: note = Expanded core CM consensus 12 sequence
RPLSGRSDNHRK

SEQ ID NO: 355 - protein, 12 aa, synthetic construct
  REGION 1..12: note = Expanded core CM consensus 12 sequence
RPLSGRSDNHRR

SEQ ID NO: 356 - protein, 12 aa, synthetic construct
  REGION 1..12: note = Expanded core CM consensus 12 sequence
KGLTGRSDRHQA

SEQ ID NO: 357 - protein, 8 aa, synthetic construct
  REGION 1..8: note = Core CM consensus 13 sequence
RIGRSDNH

SEQ ID NO: 358 - protein, 8 aa, synthetic construct
  REGION 1..8: note = Core CM consensus 13 sequence
RLGRSDNN

SEQ ID NO: 359 - protein, 12 aa, synthetic construct
  REGION 1..12: note = Expanded core CM consensus 13 sequence
NHRIGRSDNHRR

SEQ ID NO: 360 - protein, 12 aa, synthetic construct
  REGION 1..12: note = Expanded core CM consensus 13 sequence
TLRLGRSDNNKN

SEQ ID NO: 361 - protein, 8 aa, synthetic construct
  REGION 1..8: note = Core CM consensus 14 sequence
TSGRSANP

SEQ ID NO: 362 - protein, 8 aa, synthetic construct
  REGION 1..8: note = Core CM consensus 14 sequence
TSGRSGNP

SEQ ID NO: 363 - protein, 8 aa, synthetic construct
  REGION 1..8: note = Core CM consensus 14 sequence
LSGRSANP

SEQ ID NO: 364 - protein, 8 aa, synthetic construct
  REGION 1..8: note = Core CM consensus 14 sequence
LSGRSGNP

SEQ ID NO: 365 - protein, 12 aa, synthetic construct
  REGION 1..12: note = Expanded core CM consensus 14 sequence
TSTSGRSANPRG

SEQ ID NO: 366 - protein, 12 aa, synthetic construct
  REGION 1..12: note = Expanded core CM consensus 14 sequence
TSTSGRSGNPRG

SEQ ID NO: 367 - protein, 12 aa, synthetic construct
  REGION 1..12: note = Expanded core CM consensus 14 sequence
TSLSGRSANPRG

SEQ ID NO: 368 - protein, 12 aa, synthetic construct
  REGION 1..12: note = Expanded core CM consensus 14 sequence
TSLSGRSGNPRG

SEQ ID NO: 369 - protein, 8 aa, synthetic construct
  REGION 1..8: note = Cleavable moiety
LSGRSENH

SEQ ID NO: 370 - protein, 8 aa, synthetic construct
  REGION 1..8: note = Cleavable moiety
SIARSDNL

SEQ ID NO: 371 - protein, 8 aa, synthetic construct
  REGION 1..8: note = Cleavable moiety
LSGRSVTQ

SEQ ID NO: 372 - protein, 8 aa, synthetic construct
  REGION 1..8: note = cleavable linker sequence
VAQFVLTE

SEQ ID NO: 373 - protein, 8 aa, synthetic construct
  REGION 1..8: note = cleavable linker sequence
AQFVLTEG

SEQ ID NO: 374 - protein, 8 aa, synthetic construct
  REGION 1..8: note = Cleavable moiety
LYGRSENN

SEQ ID NO: 375 - protein, 8 aa, synthetic construct
  REGION 1..8: note = Cleavable moiety
RLGRSDNN

SEQ ID NO: 376 - protein, 8 aa, synthetic construct
  REGION 1..8: note = Cleavable moiety
TSGRSANP

SEQ ID NO: 377 - protein, 12 aa, synthetic construct
  REGION 1..12: note = Cleavable moiety
NTLSGRSENHSG

SEQ ID NO: 378 - protein, 12 aa, synthetic construct
  REGION 1..12: note = Cleavable moiety
PPSIARSDNLAN

SEQ ID NO: 379 - protein, 12 aa, synthetic construct
  REGION 1..12: note = Cleavable moiety
TGLSGRSVTQTS

SEQ ID NO: 380 - protein, 8 aa, synthetic construct
  REGION 1..8: note = cleavable linker sequence
PVQPIGPQ

SEQ ID NO: 381 - protein, 12 aa, synthetic construct
  REGION 1..12: note = Cleavable moiety
KGLTGRSDRHQA

SEQ ID NO: 382 - protein, 12 aa, synthetic construct
  REGION 1..12: note = Cleavable moiety
KNLYGRSENNGN

SEQ ID NO: 383 - protein, 12 aa, synthetic construct
  REGION 1..12: note = Cleavable moiety
TLRLGRSDNNKN

SEQ ID NO: 384 - protein, 12 aa, synthetic construct
  REGION 1..12: note = Cleavable moiety
TSTSGRSANPRG

SEQ ID NO: 385 - protein, 5 aa, synthetic construct
  REGION 1..5: note = Linking peptide
  REPEAT 1..5: note = May be repeated
GSGGS

SEQ ID NO: 386 - protein, 4 aa, synthetic construct
  REGION 1..4: note = Linking peptide
  REPEAT 1..4: note = May be repeated
GGGS

SEQ ID NO: 387 - protein, 4 aa, synthetic construct
  REGION 1..4: note = Linking peptide
GGSG

SEQ ID NO: 388 - protein, 5 aa, synthetic construct
  REGION 1..5: note = Linking peptide
GGSGG

SEQ ID NO: 389 - protein, 5 aa, synthetic construct
  REGION 1..5: note = Linking peptide
GSGSG

SEQ ID NO: 390 - protein, 5 aa, synthetic construct
  REGION 1..5: note = Linking peptide
GSGGG

SEQ ID NO: 391 - protein, 5 aa, synthetic construct
  REGION 1..5: note = Linking peptide
GGGSG

SEQ ID NO: 392 - protein, 5 aa, synthetic construct
  REGION 1..5: note = Linking peptide
GSSSG

SEQ ID NO: 393 - protein, 13 aa, synthetic construct
  REGION 1..13: note = Linking peptide
GSSGGSGGSGGSG

SEQ ID NO: 394 - protein, 11 aa, synthetic construct
  REGION 1..11: note = Linking peptide
GSSGGSGGSGG

SEQ ID NO: 395 - protein, 12 aa, synthetic construct
  REGION 1..12: note = Linking peptide
GSSGGSGGSGGS

SEQ ID NO: 396 - protein, 16 aa, synthetic construct
  REGION 1..16: note = Linking peptide
GSSGGSGGSGGSGGGS

SEQ ID NO: 397 - protein, 10 aa, synthetic construct
  REGION 1..10: note = Linking peptide
GSSGGSGGSG

SEQ ID NO: 398 - protein, 11 aa, synthetic construct
  REGION 1..11: note = Linking peptide
GSSGGSGGSGS

SEQ ID NO: 399 - protein, 4 aa, synthetic construct
  REGION 1..4: note = Linking peptide
GGGS

SEQ ID NO: 400 - protein, 5 aa, synthetic construct
  REGION 1..5: note = Linking peptide
GSSGT

SEQ ID NO: 401 - protein, 4 aa, synthetic construct
  REGION 1..4: note = Linking peptide
GSSG

SEQ ID NO: 402 - protein, 8 aa, synthetic construct
  REGION 1..8: note = Cleavable moiety
TGRGPSWV

SEQ ID NO: 403 - protein, 8 aa, synthetic construct
  REGION 1..8: note = Cleavable moiety
SARGPSRW

SEQ ID NO: 404 - protein, 8 aa, synthetic construct
  REGION 1..8: note = Cleavable moiety
TARGPSFK

SEQ ID NO: 405 - protein, 7 aa, synthetic construct
  REGION 1..7: note = Cleavable moiety
TARGPSW

SEQ ID NO: 406 - protein, 8 aa, synthetic construct
  REGION 1..8: note = Cleavable moiety
LSGRSDNH

SEQ ID NO: 407 - protein, 8 aa, synthetic construct
  REGION 1..8: note = Cleavable moiety
GGWHTGRN

SEQ ID NO: 408 - protein, 8 aa, synthetic construct
  REGION 1..8: note = Cleavable moiety
HTGRSGAL

SEQ ID NO: 409 - protein, 8 aa, synthetic construct
  REGION 1..8: note = Cleavable moiety
PLTGRSGG

SEQ ID NO: 410 - protein, 7 aa, synthetic construct
  REGION 1..7: note = Cleavable moiety
LTGRSGA

SEQ ID NO: 411 - protein, 8 aa, synthetic construct
  REGION 1..8: note = Cleavable moiety
AARGPAIH

SEQ ID NO: 412 - protein, 8 aa, synthetic construct
  REGION 1..8: note = Cleavable moiety
RGPAFNPM

SEQ ID NO: 413 - protein, 8 aa, synthetic construct
  REGION 1..8: note = Cleavable moiety
SSRGPAYL

SEQ ID NO: 414 - protein, 8 aa, synthetic construct
  REGION 1..8: note = Cleavable moiety
RGPATPIM

SEQ ID NO: 415 - protein, 4 aa, synthetic construct
  REGION 1..4: note = Cleavable moiety
RGPA

SEQ ID NO: 416 - protein, 10 aa, synthetic construct
  REGION 1..10: note = Cleavable moiety
GGQPSGMWGW

SEQ ID NO: 417 - protein, 10 aa, synthetic construct
  REGION 1..10: note = Cleavable moiety
FPRPLGITGL

SEQ ID NO: 418 - protein, 10 aa, synthetic construct
  REGION 1..10: note = Cleavable moiety
VHMPLGFLGP

SEQ ID NO: 419 - protein, 8 aa, synthetic construct
  REGION 1..8: note = Cleavable moiety
SPLTGRSG

SEQ ID NO: 420 - protein, 9 aa, synthetic construct
  REGION 1..9: note = Cleavable moiety
LAPLGLQRR

SEQ ID NO: 421 - protein, 8 aa, synthetic construct
  REGION 1..8: note = Cleavable moiety
SGGPLGVR

SEQ ID NO: 422 - protein, 4 aa, synthetic construct
  REGION 1..4: note = Cleavable moiety
PLGL

SEQ ID NO: 423 - protein, 7 aa, synthetic construct
  REGION 1..7: note = Cleavable moiety
GPRSFGL

SEQ ID NO: 424 - protein, 6 aa, synthetic construct
  REGION 1..6: note = Cleavable moiety
GPRSFG

SEQ ID NO: 425 - protein, 8 aa, synthetic construct
  REGION 1..8: note = Cleavable moiety
ISSGLLSS

SEQ ID NO: 426 - protein, 8 aa, synthetic construct
  REGION 1..8: note = Cleavable moiety
QNQALRMA

SEQ ID NO: 427 - protein, 8 aa, synthetic construct
  REGION 1..8: note = Cleavable moiety
AQNLLGMV

SEQ ID NO: 428 - protein, 8 aa, synthetic construct
  REGION 1..8: note = Cleavable moiety
STFPFGMF

SEQ ID NO: 429 - protein, 8 aa, synthetic construct
  REGION 1..8: note = Cleavable moiety
PVGYTSSL

SEQ ID NO: 430 - protein, 8 aa, synthetic construct
  REGION 1..8: note = Cleavable moiety
DWLYWPGI

SEQ ID NO: 431 - protein, 8 aa, synthetic construct
  REGION 1..8: note = Cleavable moiety
MIAPVAYR

SEQ ID NO: 432 - protein, 8 aa, synthetic construct
  REGION 1..8: note = Cleavable moiety
RPSPMWAY

SEQ ID NO: 433 - protein, 8 aa, synthetic construct
  REGION 1..8: note = Cleavable moiety
WATPRPMR

SEQ ID NO: 434 - protein, 8 aa, synthetic construct
  REGION 1..8: note = Cleavable moiety
FRLLDWQW

SEQ ID NO: 435 - protein, 8 aa, synthetic construct
  REGION 1..8: note = Cleavable moiety
LKAAPRWA

SEQ ID NO: 436 - protein, 8 aa, synthetic construct
  REGION 1..8: note = Cleavable moiety
GPSHLVLT

SEQ ID NO: 437 - protein, 8 aa, synthetic construct
  REGION 1..8: note = Cleavable moiety
LPGGLSPW

SEQ ID NO: 438 - protein, 8 aa, synthetic construct
  REGION 1..8: note = Cleavable moiety
MGLFSEAG

SEQ ID NO: 439 - protein, 8 aa, synthetic construct
  REGION 1..8: note = Cleavable moiety
SPLPLRVP

SEQ ID NO: 440 - protein, 8 aa, synthetic construct
  REGION 1..8: note = Cleavable moiety
RMHLRSLG

SEQ ID NO: 441 - protein, 8 aa, synthetic construct
  REGION 1..8: note = Cleavable moiety
LAAPLGLL

SEQ ID NO: 442 - protein, 8 aa, synthetic construct
  REGION 1..8: note = Cleavable moiety
AVGLLAPP

SEQ ID NO: 443 - protein, 8 aa, synthetic construct
  REGION 1..8: note = Cleavable moiety
LLAPSHRA

SEQ ID NO: 444 - protein, 8 aa, synthetic construct
  REGION 1..8: note = Cleavable moiety
PAGLWLDP

SEQ ID NO: 445 - protein, 7 aa, synthetic construct
  REGION 1..7: note = Cleavable moiety
ISSGLSS

SEQ ID NO: 446 - protein, 6 aa, synthetic construct
  REGION 1..6: note = Spacer peptide
QGQSGQ

SEQ ID NO: 447 - protein, 449 aa, synthetic construct
  REGION 1..449: note = Av1 Antibody Heavy Chain
QVQLQESGPGLVRPSQTLSLTCTVSGYSITSDHAWSWVRQPPGRGLEWIGYISYSGITTYNPSLKSRVTISRDNSKNTLYLQMNSLRAEDTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 448 - protein, 214 aa, synthetic construct [sequence omitted]

SEQ ID NO: 449 - protein, 15 aa, synthetic construct
  REGION 1..15: note = N-terminal sequence attached to AV1 light chain
YGSCSWNYVHIFMDC

SEQ ID NO: 450 - protein, 16 aa, synthetic construct
  REGION 1..16: note = N-terminal sequence attached to AV1 light chain
QGDFDIPFPAHWVPIT

SEQ ID NO: 451 - protein, 18 aa, synthetic construct
  REGION 1..18: note = N-terminal sequence attached to AV1 light chain
MGVPAGCVWNYAHIFMDC

SEQ ID NO: 452 - protein, 21 aa, synthetic construct
  REGION 1..21: note = N-terminal sequence attached to AV1 light chain
QGQSGQYGSCSWNYVHIFMDC

SEQ ID NO: 453 - protein, 21 aa, synthetic construct
  REGION 1..21: note = N-terminal sequence attached to AV1 light chain
QGQSGQGDFDIPFPAHWVPIT

SEQ ID NO: 454 - protein, 24 aa, synthetic construct
  REGION 1..24: note = N-terminal sequence attached to AV1 light chain
QGQSGQMGVPAGCVWNYAHIFMDC

SEQ ID NO: 455 - protein, 449 aa, synthetic construct
  REGION 1..449: note = C225v5 Antibody Heavy Chain
QVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 456 - protein, 449 aa, synthetic construct
  REGION 1..449: note = C225v4 Antibody Heavy Chain
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 457 - protein, 449 aa, synthetic construct
  REGION 1..449: note = C225v6 Antibody Heavy Chain
QVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 458 - protein, 214 aa, synthetic construct
  REGION 1..214: note = C225 Antibody Light Chain
QILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 459 - protein, 108 aa, synthetic construct
  REGION 1..108: note = Variable Light Chain Amino Sequence Lc4
DIQMTQSPSSLSASVGDRVTITCRASQSISSYLNWYQQKPGKAPKLLIYAASSLQSGVPSRFSGSGSGTDFTLTISSLQPEDFATYYCQQSVVAPLTFGQGTKVEIKR

SEQ ID NO: 460 - protein, 119 aa, synthetic construct
  REGION 1..119: note = Variable Heavy Chain Amino Sequence Hc4
EVQLLESGGGLVQPGGSLRLSCAASGFTFSSYAMSWVRQAPGKGLEWVSSIEQMGWQTYYADSVKGRFTISRDNSKNTLYLQMNSLRAEDTAVYYCAKDIGGRSAFDYWGQGTLVTVSS

SEQ ID NO: 461 - protein, 108 aa, synthetic construct; identical to SEQ ID NO: 459
  REGION 1..108: note = Variable Light Chain Amino Sequence Lc5

SEQ ID NO: 462 - protein, 119 aa, synthetic construct
  REGION 1..119: note = Variable Heavy Chain Amino Sequence Hc5
EVQLLESGGGLVQPGGSLRLSCAASGFTFSSYAMSWVRQAPGKGLEWVSSIEQMGWQTYYADSVKGRFTISRDNSKNTLYLQMNSLRAEDTAVYYCAKSPPYHGQFDYWGQGTLVTVSS

SEQ ID NO: 463 - protein, 108 aa, synthetic construct; identical to SEQ ID NO: 459
  REGION 1..108: note = Variable Light Chain Amino Sequence Lc7

SEQ ID NO: 464 - protein, 119 aa, synthetic construct
  REGION 1..119: note = Variable Heavy Chain Amino Sequence Hc7
EVQLLESGGGLVQPGGSLRLSCAASGFTFSSYAMSWVRQAPGKGLEWVSSIEQMGWQTYYADSVKGRFTISRDNSKNTLYLQMNSLRAEDTAVYYCAKSPPFFGQFDYWGQGTLVTVSS

SEQ ID NO: 465 - protein, 108 aa, synthetic construct; identical to SEQ ID NO: 459
  REGION 1..108: note = Variable Light Chain Amino Sequence Lc8

SEQ ID NO: 466 - protein, 119 aa, synthetic construct
  REGION 1..119: note = Variable Heavy Chain Amino Sequence Hc8
EVQLLESGGGLVQPGGSLRLSCAASGFTFSSYAMSWVRQAPGKGLEWVSSIEQMGWQTYYADSVKGRFTISRDNSKNTLYLQMNSLRAEDTAVYYCAKHIGRTNPFDYWGQGTLVTVSS

SEQ ID NO: 467 - protein, 108 aa, synthetic construct; identical to SEQ ID NO: 459
  REGION 1..108: note = Variable Light Chain Amino Sequence Lc13

SEQ ID NO: 468 - protein, 116 aa, synthetic construct
  REGION 1..116: note = Variable Heavy Chain Amino Sequence Hc13
EVQLLESGGGLVQPGGSLRLSCAASGFTFSSYAMSWVRQAPGKGLEWVSSIEQMGWQTEYADSVKGRFTISRDNSKNTLYLQMNSLRAEDTAVYYCAKSAAAFDYWGQGTLVTVSS

SEQ ID NO: 469 - protein, 108 aa, synthetic construct; identical to SEQ ID NO: 459
  REGION 1..108: note = Variable Light Chain Amino Sequence Lc16

SEQ ID NO: 470 - protein, 119 aa, synthetic construct
  REGION 1..119: note = Variable Heavy Chain Amino Sequence Hc16
EVQLLESGGGLVQPGGSLRLSCAASGFTFSSYAMSWVRQAPGKGLEWVSSIEQMGWQTYYADSVKGRFTISRDNSKNTLYLQMNSLRAEDTAVYYCAKSPPYYGQFDYWGQGTLVTVSS

SEQ ID NO: 471 - protein, 108 aa, synthetic construct; identical to SEQ ID NO: 459
  REGION 1..108: note = Variable Light Chain Amino Sequence Lc19

SEQ ID NO: 472 - protein, 119 aa, synthetic construct; identical to SEQ ID NO: 464
  REGION 1..119: note = Variable Heavy Chain Amino Sequence Hc19

SEQ ID NO: 473 - protein, 108 aa, synthetic construct; identical to SEQ ID NO: 459
  REGION 1..108: note = Variable Light Chain Amino Sequence Lc21

SEQ ID NO: 474 - protein, 119 aa, synthetic construct; identical to SEQ ID NO: 460
  REGION 1..119: note = Variable Heavy Chain Amino Sequence Hc21

SEQ ID NO: 475 - protein, 108 aa, synthetic construct; identical to SEQ ID NO: 459
  REGION 1..108: note = Variable Light Chain Amino Sequence Lc24

SEQ ID NO: 476 - protein, 116 aa, synthetic construct
  REGION 1..116: note = Variable Heavy Chain Amino Sequence Hc24
EVQLLESGGGLVQPGGSLRLSCAASGFTFSSYAMSWVRQAPGKGLEWVSSIEEMGWQTLYADSVKGRFTISRDNSKNTLYLQMNSLRAEDTAVYYCAKSAAAFDYWGQGTLVTVSS

SEQ ID NO: 477 - protein, 108 aa, synthetic construct; identical to SEQ ID NO: 459
  REGION 1..108: note = Variable Light Chain Amino Sequence Lc26

SEQ ID NO: 478 - protein, 119 aa, synthetic construct; identical to SEQ ID NO: 460
  REGION 1..119: note = Variable Heavy Chain Amino Sequence Hc26

SEQ ID NO: 479 - protein, 108 aa, synthetic construct; identical to SEQ ID NO: 459
  REGION 1..108: note = Variable Light Chain Amino Sequence Lc27

SEQ ID NO: 480 - protein, 119 aa, synthetic construct
  REGION 1..119: note = Variable Heavy Chain Amino Sequence Hc27
EVQLLESGGGLVQPGGSLRLSCAASGFTFSSYAMSWVRQAPGKGLEWVSSIEQMGWQTYYADSVKGRFTISRDNSKNTLYLQMNSLRAEDTAVYYCAKSPPFYGQFDYWGQGTLVTVSS

SEQ ID NO: 481 - protein, 108 aa, synthetic construct; identical to SEQ ID NO: 459
  REGION 1..108: note = Variable Light Chain Amino Sequence Lc28

SEQ ID NO: 482 - protein, 119 aa, synthetic construct; identical to SEQ ID NO: 464
  REGION 1..119: note = Variable Heavy Chain Amino Sequence Hc28

SEQ ID NO: 483 - protein, 108 aa, synthetic construct; identical to SEQ ID NO: 459
  REGION 1..108: note = Variable Light Chain Amino Sequence Lc30

SEQ ID NO: 484 - protein, 115 aa, synthetic construct
  REGION 1..115: note = Variable Heavy Chain Amino Sequence Hc30
EVQLLESGGGLVQPGGSLRLSCAASGFTFSSYAMSWVRQAPGKGLEWVSSIEEMGWQTLYADSVKGRFTISRDNSKNTLYLQMNSLRAEDTAVYYAKSAAAFDYWGQGTLVTVSS

SEQ ID NO: 485 - protein, 108 aa, synthetic construct; identical to SEQ ID NO: 459
  REGION 1..108: note = Variable Light Chain Amino Sequence Lc31

SEQ ID NO: 486 - protein, 119 aa, synthetic construct; identical to SEQ ID NO: 460
  REGION 1..119: note = Variable Heavy Chain Amino Sequence Hc31

SEQ ID NO: 487 - protein, 108 aa, synthetic construct; identical to SEQ ID NO: 459
  REGION 1..108: note = Variable Light Chain Amino Sequence Lc32

SEQ ID NO: 488 - protein, 116 aa, synthetic construct
  REGION 1..116: note = Variable Heavy Chain Amino Sequence Hc32
EVQLLESGGGLVQPGGSLRLSCAASGFTFSSYAMSWVRQAPGKGLEWVSSIDPEGWQTYYADSVKGRFTISRDNSKNTLYLQMNSLRAEDTAVYYCAKSAAAFDYWGQGTLVTVSS

SEQ ID NO: 489 - protein, 108 aa, synthetic construct; identical to SEQ ID NO: 459
  REGION 1..108: note = Variable Light Chain Amino Sequence Lc37

SEQ ID NO: 490 - protein, 119 aa, synthetic construct
  REGION 1..119: note = Variable Heavy Chain Amino Sequence Hc37
EVQLLESGGGLVQPGGSLRLSCAASGFTFSSYAMSWVRQAPGKGLEWVSSIEQMGWQTYYADSVKGRFTISRDNSKNTLYLQMNSLRAEDTAVYYCAKSPPHNGQFDYWGQGTLVTVSS

SEQ ID NO: 491 - protein, 108 aa, synthetic construct; identical to SEQ ID NO: 459
  REGION 1..108: note = Variable Light Chain Amino Sequence Lc39

SEQ ID NO: 492 - protein, 116 aa, synthetic construct; identical to SEQ ID NO: 468
  REGION 1..116: note = Variable Heavy Chain Amino Sequence Hc39

SEQ ID NO: 493 - protein, 108 aa, synthetic construct; identical to SEQ ID NO: 459
  REGION 1..108: note = Variable Light Chain Amino Sequence Lc40

SEQ ID NO: 494 - protein, 119 aa, synthetic construct; identical to SEQ ID NO: 464
  REGION 1..119: note = Heavy Chain Amino Sequence Hc40

SEQ ID NO: 495 - protein, 108 aa, synthetic construct; identical to SEQ ID NO: 459
  REGION 1..108: note = Variable Light Chain Amino Sequence Lc47

SEQ ID NO: 496 - protein, 116 aa, synthetic construct
  REGION 1..116: note = Variable Heavy Chain Amino Sequence Hc47
EVQLLESGGGLVQPGGSLRLSCAASGFTFSSYAMSWVRQAPGKGLEWVSSIDEMGWQTEYADSVKGRFTISRDNSKNTLYLQMNSLRAEDTAVYYCAKSAAAFDYWGQGTLVTVSS

SEQ ID NO: 497 - protein, 108 aa, synthetic construct
  REGION 1..108: note = Variable 4B2 Light Chain
DIQMTQSPSSLSASVGDRVTITCRASQSISSYLNWYQQKPGKAPKLLIYAASSLQSGVPSRFSGSGSGTDFTLTISSLQPEDFATYYCQQTLDAPPQFGQGTKVEIKR

SEQ ID NO: 498 - protein, 119 aa, synthetic construct; identical to SEQ ID NO: 460
  REGION 1..119: note = Variable 4B2 Heavy Chain

SEQ ID NO: 499 - protein, 108 aa, synthetic construct
  REGION 1..108: note = Variable 4D11 Light Chain
DIQMTQSPSSLSASVGDRVTITCRASQSISSYLNWYQQKPGKAPKLLIYAASSLQSGVPSRFSGSGSGTDFTLTISSLQPEDFATYYCQQTVVAPPLFGQGTKVEIKR

SEQ ID NO: 500 - protein, 119 aa, synthetic construct
  REGION 1..119: note = Variable 4D11 Heavy Chain
EVQLLESGGGLVQPGGSLRLSCAASGFTFSSYAMSWVRQAPGKGLEWVSSIDPEGRQTYYADSVKGRFTISRDNSKNTLYLQMNSLRAEDTAVYYCAKDIGGRSAFDYWGQGTLVTVSS

SEQ ID NO: 501 - protein, 108 aa, synthetic construct
  REGION 1..108: note = Variable 4E7 Light Chain
DIQMTQSPSSLSASVGDRVTITCRASQSISSYLNWYQQKPGKAPKLLIYAASSLQSGVPSRFSGSGSGTDFTLTISSLQPEDFATYYCQQSLVAPLTFGQGTKVEIKR

SEQ ID NO: 502 - protein, 116 aa, synthetic construct
  REGION 1..116: note = Variable 4E7 Heavy Chain
EVQLLESGGGLVQPGGSLRLSCAASGFTFSSYAMSWVRQAPGKGLEWVSSIEEMGWQTKYADSVKGRFTISRDNSKNTLYLQMNSLRAEDTAVYYCAKSAAAFDYWGQGTLVTVSS

SEQ ID NO: 503 - protein, 108 aa, synthetic construct
  REGION 1..108: note = Variable 4E11 Light Chain
DIQMTQSPSSLSASVGDRVTITCRASQSISSYLNWYQQKPGKAPKLLIYAASSLQSGVPSRFSGSGSGTDFTLTISSLQPEDFATYYCQQALDAPLMFGQGTKVEIKR

SEQ ID NO: 504 - protein, 119 aa, synthetic construct
  REGION 1..119: note = Variable 4E11 Heavy Chain
EVQLLESGGGLVQPGGSLRLSCAASGFTFSSYAMSWVRQAPGKGLEWVSSIEPMGQLTEYADSVKGRFTISRDNSKNTLYLQMNSLRAEDTAVYYCAKDIGGRSAFDYWGQGTLVTVSS

SEQ ID NO: 505 - protein, 108 aa, synthetic construct
  REGION 1..108: note = Variable 6B7 Light Chain
DIQMTQSPSSLSASVGDRVTITCRASQSISSYLNWYQQKPGKAPKLLIYAASSLQSGVPSRFSGSGSGTDFTLTISSLQPEDFATYYCQQALVAPLTFGQGTKVEIKR

SEQ ID NO: 506 - protein, 116 aa, synthetic construct
  REGION 1..116: note = Variable 6B7 Heavy Chain
EVQLLESGGGLVQPGGSLRLSCAASGFTFSSYAMSWVRQAPGKGLEWVSSIDEMGWQTYYADSVKGRFTISRDNSKNTLYLQMNSLRAEDTAVYYCAKSAAAFDYWGQGTLVTVSS

SEQ ID NO: 507 - protein, 108 aa, synthetic construct; identical to SEQ ID NO: 505
  REGION 1..108: note = Variable 6F8 Light Chain

SEQ ID NO: 508 - protein, 116 aa, synthetic construct; identical to SEQ ID NO: 506
  REGION 1..116: note = Variable 6F8 Heavy Chain

SEQ ID NO: 509 - protein, 214 aa, synthetic construct
  REGION 1..214: note = 4D11 Light Chain
DIQMTQSPSSLSASVGDRVTITCRASQSISSYLNWYQQKPGKAPKLLIYAASSLQSGVPSRFSGSGSGTDFTLTISSLQPEDFATYYCQQTVVAPPLFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 510 - protein, 449 aa, synthetic construct
  REGION 1..449: note = 4D11 Heavy Chain
EVQLLESGGGLVQPGGSLRLSCAASGFTFSSYAMSWVRQAPGKGLEWVSSIDPEGRQTYYADSVKGRFTISRDNSKNTLYLQMNSLRAEDTAVYYCAKDIGGRSAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 511 - protein, 449 aa, synthetic construct
  REGION 1..449: note = 4D11v2 Heavy Chain
EVHLLESGGGLVQPGGSLRLSCAASGFTFSSYAMSWVRQAPGKGLEWVSSIDPEGRQTYYADSVKGRFTISRDNSKNTLYLQMNSLRAEDTAVYYCAKDIGGRSAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 512 - protein, 214 aa, synthetic construct
  REGION 1..214: note = 4D11v2 Light Chain
  VARIANT 182: note = X may be any amino acid
DIQMTQSPSSLSASVGDRVTITCRASQSISSYLNWYQQKPGKAPKLLIYAASSLQSGVPSRFSGSGSGTDFTLTISSLQPEDFATYYCQQTVVAPPLFGQGTKVEIKRTVAAPSVFIFPPSDEQLKSGTASVVCLLNNFYPREAKVQWKVDNALQSGNSQESVTEQDSKDSTYSLSSTLTLXKADYEKHKVYACEVTHQGLSSPVTKSFNRGEC

SEQ ID NO: 513 - protein, 6 aa, synthetic construct
  REGION 1..6: note = masking moiety
CISPRG

SEQ ID NO: 514 - protein, 8 aa, synthetic construct
  REGION 1..8: note = masking moiety
CISPRGCG

SEQ ID NO: 515 - protein, 15 aa, synthetic construct
  REGION 1..15: note = masking moiety
CISPRGCPDGPYVMY

SEQ ID NO: 516 - protein, 14 aa, synthetic construct
  REGION 1..14: note = masking moiety
CISPRGCPDGPYVM

SEQ ID NO: 517 - protein, 15 aa, synthetic construct
  REGION 1..15: note = masking moiety
CISPRGCEPGTYVPT

SEQ ID NO: 518 - protein, 15 aa, synthetic construct
  REGION 1..15: note = masking moiety
CISPRGCPGQIWHPP

SEQ ID NO: 519 - protein, 15 aa, synthetic construct
  REGION 1..15: note = masking moiety
GSHCLIPINMGAPSC

SEQ ID NO: 520 - protein, 32 aa, synthetic construct
  REGION 1..32: note = masking moiety
CISPRGCGGSSASQSGQGSHCLIPINMGAPSC

SEQ ID NO: 521 - protein, 19 aa, synthetic construct
  REGION 1..19: note = masking moiety
CNHHYFYTCGCISPRGCPG

SEQ ID NO: 522 - protein, 19 aa, synthetic construct
  REGION 1..19: note = masking moiety
ADHVFWGSYGCISPRGCPG

SEQ ID NO: 523 - protein, 19 aa, synthetic construct
  REGION 1..19: note = masking moiety
CHHVYWGHCGCISPRGCPG

SEQ ID NO: 524 - protein, 19 aa, synthetic construct
  REGION 1..19: note = masking moiety
CPHFTTTSCGCISPRGCPG

SEQ ID NO: 525 - protein, 19 aa, synthetic construct
  REGION 1..19: note = masking moiety
CNHHYHYYCGCISPRGCPG

SEQ ID NO: 526 - protein, 19 aa, synthetic construct
  REGION 1..19: note = masking moiety
CPHVSFGSCGCISPRGCPG

SEQ ID NO: 527 - protein, 19 aa, synthetic construct
  REGION 1..19: note = masking moiety
CPYYTLSYCGCISPRGCPG

SEQ ID NO: 528 - protein, 19 aa, synthetic construct
  REGION 1..19: note = masking moiety
CNHVYFGTCGCISPRGCPG

SEQ ID NO: 529 - protein, 19 aa, synthetic construct
  REGION 1..19: note = masking moiety
CNHFTLTTCGCISPRGCPG

SEQ ID NO: 530 - protein, 19 aa, synthetic construct
  REGION 1..19: note = masking moiety
CHHFTLTTCGCISPRGCPG

SEQ ID NO: 531 - protein, 18 aa, synthetic construct
  REGION 1..18: note = masking moiety
YNPCATPMCCISPRGCPG

SEQ ID NO: 532 - protein, 18 aa, synthetic construct
  REGION 1..18: note = masking moiety
CNHHYFYTCGCISPRGCG

SEQ ID NO: 533 - protein, 18 aa, synthetic construct
  REGION 1..18: note = masking moiety
CNHHYHYYCGCISPRGCG

SEQ ID NO: 534 - protein, 18 aa, synthetic construct
  REGION 1..18: note = masking moiety
CNHVYFGTCGCISPRGCG

SEQ ID NO: 535 - protein, 18 aa, synthetic construct
  REGION 1..18: note = masking moiety
CHHVYWGHCGCISPRGCG

SEQ ID NO: 536 - protein, 18 aa, synthetic construct
  REGION 1..18: note = masking moiety
CPHFTTTSCGCISPRGCG

SEQ ID NO: 537 - protein, 18 aa, synthetic construct
  REGION 1..18: note = masking moiety
CNHFTLTTCGCISPRGCG

SEQ ID NO: 538 - protein, 18 aa, synthetic construct
  REGION 1..18: note = masking moiety
CHHFTLTTCGCISPRGCG

SEQ ID NO: 539 - protein, 18 aa, synthetic construct
  REGION 1..18: note = masking moiety
CPYYTLSYCGCISPRGCG

SEQ ID NO: 540 - protein, 18 aa, synthetic construct
  REGION 1..18: note = masking moiety
CPHVSFGSCGCISPRGCG

SEQ ID NO: 541 - protein, 18 aa, synthetic construct
  REGION 1..18: note = masking moiety
ADHVFWGSYGCISPRGCG

SEQ ID NO: 542 - protein, 17 aa, synthetic construct
  REGION 1..17: note = masking moiety
YNPCATPMCCISPRGCG

SEQ ID NO: 543 - protein, 18 aa, synthetic construct
  REGION 1..18: note = masking moiety
CHHVYWGHCGCISPRGCG

SEQ ID NO: 544 - protein, 18 aa, synthetic construct
  REGION 1..18: note = masking moiety
  VARIANT 2: note = X may be Asn or Pro
  VARIANT 4: note = X may be His, Val or Phe
  VARIANT 5: note = X may be Tyr or Thr
  VARIANT 6: note = X may be Phe, Trp, Thr or Leu
  VARIANT 7: note = X may be Tyr, Gly, Thr or Ser
  VARIANT 8: note = X may be Thr, Ser, Tyr or His
CXHXXXXXCGCISPRGCG

SEQ ID NO: 545 - protein, 15 aa, synthetic construct
  REGION 1..15: note = masking moiety
CISPRGCGQPIPSVK

SEQ ID NO: 546 - protein, 15 aa, synthetic construct
  REGION 1..15: note = masking moiety
CISPRGCTQPYHVSR

SEQ ID NO: 547 - protein, 15 aa, synthetic construct
  REGION 1..15: note = masking moiety
CISPRGCNAVSGLGS

SEQ ID NO: 548 - protein, 26 aa, synthetic construct
  REGION 1..26: note = masking moiety
QGQSGQGQQQWCNIWINGGDCRGWNG

SEQ ID NO: 549 - protein, 15 aa, synthetic construct
  REGION 1..15: note = masking moiety
PWCMQRQDFLRCPQP

SEQ ID NO: 550 - protein, 15 aa, synthetic construct
  REGION 1..15: note = masking moiety
QLGLPAYMCTFECLR

SEQ ID NO: 551 - protein, 15 aa, synthetic construct
  REGION 1..15: note = masking moiety
CNLWVSGGDCGGLQG

SEQ ID NO: 552 - protein, 15 aa, synthetic construct
  REGION 1..15: note = masking moiety
SCSLWTSGSCLPHSP

SEQ ID NO: 553 - protein, 15 aa, synthetic construct
  REGION 1..15: note = masking moiety
YCLQLPHYMQAMCGR

SEQ ID NO: 554 - protein, 15 aa, synthetic construct
  REGION 1..15: note = masking moiety
CFLYSCTDVSYWNNT

SEQ ID NO: 555 - protein, 15 aa, synthetic construct
  REGION 1..15: note = masking moiety
PWCMQRQDYLRCPQP

SEQ ID NO: 556 - protein, 15 aa, synthetic construct
  REGION 1..15: note = masking moiety
CNLWISGGDCRGLAG

SEQ ID NO: 557 - protein, 15 aa, synthetic construct
  REGION 1..15: note = masking moiety
CNLWVSGGDCRGVQG

SEQ ID NO: 558 - protein, 15 aa, synthetic construct
  REGION 1..15: note = masking moiety
CNLWVSGGDCRGLRG

SEQ ID NO: 559 - protein, 15 aa, synthetic construct
  REGION 1..15: note = masking moiety
CNLWISGGDCRGLPG

SEQ ID NO: 560 - protein, 15 aa, synthetic construct
  REGION 1..15: note = masking moiety
CNLWVSGGDCRDAPW

SEQ ID NO: 561 - protein, 15 aa, synthetic construct
  REGION 1..15: note = masking moiety
CNLWVSGGDCRDLLG

SEQ ID NO: 562 - protein, 15 aa, synthetic construct
  REGION 1..15: note = masking moiety
CNLWVSGGDCRGLQG

SEQ ID NO: 563 - protein, 15 aa, synthetic construct
  REGION 1..15: note = masking moiety
CNLWLHGGDCRGWQG

SEQ ID NO: 564 - protein, 15 aa, synthetic construct
  REGION 1..15: note = masking moiety
CNIWLVGGDCRGWQG

SEQ ID NO: 565 - protein, 15 aa, synthetic construct
  REGION 1..15: note = masking moiety
CTTWFCGGDCGVMRG

SEQ ID NO: 566 - protein, 15 aa, synthetic construct
  REGION 1..15: note = masking moiety
CNIWGPSVDCGALLG

SEQ ID NO: 567 - protein, 15 aa, synthetic construct
  REGION 1..15: note = masking moiety
CNIWVNGGDCRSFEG

SEQ ID NO: 568 - protein, 15 aa, synthetic construct
  REGION 1..15: note = masking moiety
YCLNLPRYMQDMCWA

SEQ ID NO: 569 - protein, 15 aa, synthetic construct
  REGION 1..15: note = masking moiety
YCLALPHYMQADCAR

SEQ ID NO: 570 - protein, 15 aa, synthetic construct
  REGION 1..15: note = masking moiety
CFLYSCGDVSYWGSA

SEQ ID NO: 571 - protein, 15 aa, synthetic construct
  REGION 1..15: note = masking moiety
CYLYSCTDSAFWNNR

SEQ ID NO: 572 - protein, 15 aa, synthetic construct
  REGION 1..15: note = masking moiety
CYLYSCNDVSYWSNT

SEQ ID NO: 573 - protein, 12 aa, synthetic construct
  REGION 1..12: note = masking moiety
CFLYSCTDVSYW

SEQ ID NO: 574 - protein, 15 aa, synthetic construct
  REGION 1..15: note = masking moiety
CFLYSCTDVAYWNSA

SEQ ID NO: 575 - protein, 15 aa, synthetic construct
  REGION 1..15: note = masking moiety
CFLYSCTDVSYWGDT

SEQ ID NO: 576 - protein, 15 aa, synthetic construct
  REGION 1..15: note = masking moiety
CFLYSCTDVSYWGNS

SEQ ID NO: 577 - protein, 15 aa, synthetic construct
  REGION 1..15: note = masking moiety
CFLYSCTDVAYWNNT

SEQ ID NO: 578 - protein, 18 aa, synthetic construct
  REGION 1..18: note = masking moiety
CFLYSCGDVSYWGNPGLS

SEQ ID NO: 579 - protein, 15 aa, synthetic construct
  REGION 1..15: note = masking moiety
CFLYSCTDVAYWSGL

SEQ ID NO: 580 - protein, 15 aa, synthetic construct
  REGION 1..15: note = masking moiety
CYLYSCTDGSYWNST

SEQ ID NO: 581 - protein, 15 aa, synthetic construct
  REGION 1..15: note = masking moiety
CFLYSCSDVSYWGNI

SEQ ID NO: 582 - protein, 12 aa, synthetic construct
  REGION 1..12: note = masking moiety
CFLYSCTDVAYW

SEQ ID NO: 583 - protein, 15 aa, synthetic construct
  REGION 1..15: note = masking moiety
CFLYSCTDVSYWGST

SEQ ID NO: 584 - protein, 15 aa, synthetic construct
  REGION 1..15: note = masking moiety
CFLYSCTDVAYWGDT

SEQ ID NO: 585 - protein, 20 aa, synthetic construct
  REGION 1..20: note = masking moiety
GCNIWLNGGDCRGWVDPLQG

SEQ ID NO: 586 - protein, 20 aa, synthetic construct
  REGION 1..20: note = masking moiety
GCNIWLVGGDCRGWIGDTNG

SEQ ID NO: 587 - protein, 20 aa, synthetic construct
  REGION 1..20: note = masking moiety
GCNIWLVGGDCRGWIEDSNG

SEQ ID NO: 588 - protein, 20 aa, synthetic construct
  REGION 1..20: note = masking moiety
GCNIWANGGDCRGWIDNIDG

SEQ ID NO: 589 - protein, 20 aa, synthetic construct
  REGION 1..20: note = masking moiety
GCNIWLVGGDCRGWLGEAVG

SEQ ID NO: 590 - protein, 20 aa, synthetic construct
  REGION 1..20: note = masking moiety
GCNIWLVGGDCRGWLEEAVG

SEQ ID NO: 591 - protein, 20 aa, synthetic construct
  REGION 1..20: note = masking moiety
GGPALCNIWLNGGDCRGWSG

SEQ ID NO: 592 - protein, 20 aa, synthetic construct
  REGION 1..20: note = masking moiety
GAPVFCNIWLNGGDCRGWMG

SEQ ID NO: 593 - protein, 20 aa, synthetic construct
  REGION 1..20: note = masking moiety
GQQQWCNIWINGGDCRGWNG

SEQ ID NO: 594 - protein, 20 aa, synthetic construct
  REGION 1..20: note = masking moiety
GKSEFCNIWLNGGDCRGWIG

SEQ ID NO: 595 - protein, 20 aa, synthetic construct
  REGION 1..20: note = masking moiety
GTPGGCNIWANGGDCRGWEG

SEQ ID NO: 596 - protein, 20 aa, synthetic construct
  REGION 1..20: note = masking moiety
GASQYCNLWINGGDCRGWRG

SEQ ID NO: 597 - protein, 18 aa, synthetic construct
  REGION 1..18: note = masking moiety
GCNIWLVGGDCRPWVEGG

SEQ ID NO: 598 - protein, 18 aa, synthetic construct
  REGION 1..18: note = masking moiety
GCNIWAVGGDCRPFVDGG

SEQ ID NO: 599 - protein, 18 aa, synthetic construct
  REGION 1..18: note = masking moiety
GCNIWLNGGDCRAWVDTG

SEQ ID NO: 600 - protein, 18 aa, synthetic construct
  REGION 1..18: note = masking moiety
GCNIWIVGGDCRPFINDG

SEQ ID NO: 601 - protein, 18 aa, synthetic construct
  REGION 1..18: note = masking moiety
GCNIWLNGGDCRPVVFGG

SEQ ID NO: 602 - protein, 18 aa, synthetic construct
  REGION 1..18: note = masking moiety
GCNIWLSGGDCRMFMNEG

SEQ ID NO: 603 - protein, 18 aa, synthetic construct
  REGION 1..18: note = masking moiety
GCNIWVNGGDCRSFVYSG

SEQ ID NO: 604 - protein, 18 aa, synthetic construct
  REGION 1..18: note = masking moiety
GCNIWLNGGDCRGWEASG

SEQ ID NO: 605 - protein, 18 aa, synthetic construct
  REGION 1..18: note = masking moiety
GCNIWAHGGDCRGFIEPG

SEQ ID NO: 606 - protein, 18 aa, synthetic construct
  REGION 1..18: note = masking moiety
GCNIWLNGGDCRTFVASG

SEQ ID NO: 607 - protein, 18 aa, synthetic construct
  REGION 1..18: note = masking moiety
GCNIWAHGGDCRGFIEPG

SEQ ID NO: 608 - protein, 18 aa, synthetic construct
  REGION 1..18: note = masking moiety
GFLENCNIWLNGGDCRTG

SEQ ID NO: 609 - protein, 18 aa, synthetic construct
  REGION 1..18: note = masking moiety
GIYENCNIWLNGGDCRMG

SEQ ID NO: 610 - protein, 18 aa, synthetic construct
  REGION 1..18: note = masking moiety
GIPDNCNIWINGGDCRYG

SEQ ID NO: 611 - protein, 21 aa, synthetic construct
  REGION 1..21: note = masking moiety
QGQSGQYGSCSWNYVHIFMDC

SEQ ID NO: 612 - protein, 21 aa, synthetic construct
  REGION 1..21: note = masking moiety
QGQSGQGDFDIPFPAHWVPIT

SEQ ID NO: 613 - protein, 24 aa, synthetic construct
  REGION 1..24: note = masking moiety
QGQSGQMGVPAGCVWNYAHIFMDC

SEQ ID NO: 614 - protein, 15 aa, synthetic construct
  REGION 1..15: note = masking moiety
YRSCNWNYVSIFLDC

SEQ ID NO: 615 - protein, 16 aa, synthetic construct
  REGION 1..16: note = masking moiety
PGAFDIPFPAHWVPNT

SEQ ID NO: 616 - protein, 15 aa, synthetic construct
  REGION 1..15: note = masking moiety
ESSCVWNYVHIYMDC

SEQ ID NO: 617 - protein, 15 aa, synthetic construct
  REGION 1..15: note = masking moiety
YPGCKWNYDRIFLDC

SEQ ID NO: 618 - protein, 15 aa, synthetic construct
  REGION 1..15: note = masking moiety
YRTCSWNYVGIFLDC

SEQ ID NO: 619 - protein, 15 aa, synthetic construct
  REGION 1..15: note = masking moiety
YGSCSWNYVHIFMDC

SEQ ID NO: 620 - protein, 15 aa, synthetic construct
  REGION 1..15: note = masking moiety
YGSCSWNYVHIFLDC

SEQ ID NO: 621 - protein, 15 aa, synthetic construct
  REGION 1..15: note = masking moiety
YGSCNWNYVHIFLDC

SEQ ID NO: 622 - protein, 15 aa, synthetic construct
  REGION 1..15: note = masking moiety
YTSCNWNYVHIFMDC

SEQ ID NO: 623 - protein, 15 aa, synthetic construct
  REGION 1..15: note = masking moiety
YPGCKWNYDRIFLDC

SEQ ID NO: 624 - protein, 15 aa, synthetic construct
  REGION 1..15: note = masking moiety
WRSCNWNYAHIFLDC

SEQ ID NO: 625 - protein, 15 aa, synthetic construct
  REGION 1..15: note = masking moiety
WSNCHWNYVHIFLDC

SEQ ID NO: 626 - protein, 15 aa, synthetic construct
  REGION 1..15: note = masking moiety
DRSCTWNYVRISYDC

SEQ ID NO: 627 - protein, 15 aa, synthetic construct
  REGION 1..15: note = masking moiety
SGSCKWDYVHIFLDC

SEQ ID NO: 628 - protein, 15 aa, synthetic construct
  REGION 1..15: note = masking moiety
SRSCIWNYAHIHLDC

SEQ ID NO: 629 - protein, 15 aa, synthetic construct
  REGION 1..15: note = masking moiety
SMSCYWQYERIFLDC

SEQ ID NO: 630 - protein, 15 aa, synthetic construct
  REGION 1..15: note = masking moiety
YRSCNWNYVSIFLDC

SEQ ID NO: 631 - protein, 15 aa, synthetic construct
  REGION 1..15: note = masking moiety
SGSCKWDYVHIFLDC

SEQ ID NO: 632 - protein, 15 aa, synthetic construct
  REGION 1..15: note = masking moiety
YKSCHWDYVHIFLDC

SEQ ID NO: 633 - protein, 15 aa, synthetic construct
  REGION 1..15: note = masking moiety
YGSCTWNYVHIFMEC

SEQ ID NO: 634 - protein, 15 aa, synthetic construct
  REGION 1..15: note = masking moiety
FSSCNWNYVHIFLDC

SEQ ID NO: 635 - protein, 15 aa, synthetic construct
  REGION 1..15: note = masking moiety
WRSCNWNYAHIFLDC

SEQ ID NO: 636 - protein, 15 aa, synthetic construct
  REGION 1..15: note = masking moiety
YGSCQWNYVHIFLDC

SEQ ID NO: 637 - protein, 15 aa, synthetic construct
  REGION 1..15: note = masking moiety
YRSCNWNYVHIFLDC

SEQ ID NO: 638 - protein, 15 aa, synthetic construct
  REGION 1..15: note = masking moiety
NMSCHWDYVHIFLDC

SEQ ID NO: 639 - protein, 15 aa, synthetic construct
  REGION 1..15: note = masking moiety
FGPCTWNYARISWDC

SEQ ID NO: 640 - protein, 15 aa, synthetic construct
  REGION 1..15: note = masking moiety
  VARIANT 1..2: note = X may be any amino acid
  VARIANT 5: note = X may be any amino acid
  VARIANT 7: note = X may be any amino acid
  VARIANT 13: note = X may be any amino acid
XXSCXWXYVHIFXDC

SEQ ID NO: 641 - protein, 18 aa, synthetic construct
  REGION 1..18: note = masking moiety
MGVPAGCVWNYAHIFMDC

SEQ ID NO: 642 - protein, 18 aa, synthetic construct
  REGION 1..18: note = masking moiety
RDTGGQCRWDYVHIFMDC

SEQ ID NO: 643 - protein, 18 aa, synthetic construct
  REGION 1..18: note = masking moiety
AGVPAGCTWNYVHIFMEC

SEQ ID NO: 644 - protein, 18 aa, synthetic construct
  REGION 1..18: note = masking moiety
VGVPNGCVWNYAHIFMEC

SEQ ID NO: 645 - protein, 18 aa, synthetic construct
  REGION 1..18: note = masking moiety
DGGPAGCSWNYVHIFMEC

SEQ ID NO: 646 - protein, 18 aa, synthetic construct
  REGION 1..18: note = masking moiety
AVGPAGCWWNYVHIFMEC

SEQ ID NO: 647 - protein, 18 aa, synthetic construct
  REGION 1..18: note = masking moiety
CTWNYVHIFMDCGEGEGP

SEQ ID NO: 648 - protein, 18 aa, synthetic construct
  REGION 1..18: note = masking moiety
GGVPEGCTWNYAHIFMEC

SEQ ID NO: 649 - protein, 18 aa, synthetic construct
  REGION 1..18: note = masking moiety
AEVPAGCWWNYVHIFMEC

SEQ ID NO: 650 - protein, 18 aa, synthetic construct
  REGION 1..18: note = masking moiety
AGVPAGCTWNYVHIFMEC

SEQ ID NO: 651 - protein, 18 aa, synthetic construct
  REGION 1..18: note = masking moiety
SGASGGCKWNYVHIFMDC

SEQ ID NO: 652 - protein, 18 aa, synthetic construct
  REGION 1..18: note = masking moiety
TPGCRWNYVHIFMECEAL

SEQ ID NO: 653 - protein, 18 aa, synthetic construct
  REGION 1..18: note = masking moiety
VGVPNGCVWNYAHIFMEC

SEQ ID NO: 654 - protein, 16 aa, synthetic construct
  REGION 1..16: note = masking moiety
PGAFDIPFPAHWVPNT

SEQ ID NO: 655 - protein, 16 aa, synthetic construct
  REGION 1..16: note = masking moiety
RGACDIPFPAHWIPNT

SEQ ID NO: 656 - protein, 16 aa, synthetic construct
  REGION 1..16: note = masking moiety
QGDFDIPFPAHWVPIT

SEQ ID NO: 657 - protein, 16 aa, synthetic construct
  REGION 1..16: note = masking moiety
  VARIANT 1: note = X may be any amino acid
XGAFDIPFPAHWVPNT

SEQ ID NO: 658 - protein, 19 aa, synthetic construct
  REGION 1..19: note = masking moiety
RGDGNDSDIPFPAHWVPRT

SEQ ID NO: 659 - protein, 19 aa, synthetic construct
  REGION 1..19: note = masking moiety
SGVGRDRDIPFPAHWVPRT

SEQ ID NO: 660 - protein, 19 aa, synthetic construct
  REGION 1..19: note = masking moiety
WAGGNDCDIPFPAHWIPNT

SEQ ID NO: 661 - protein, 19 aa, synthetic construct
  REGION 1..19: note = masking moiety
WGDGMDVDIPFPAHWVPVT

SEQ ID NO: 662 - protein, 19 aa, synthetic construct
  REGION 1..19: note = masking moiety
AGSGNDSDIPFPAHWVPRT

SEQ ID NO: 663 - protein, 19 aa, synthetic construct
  REGION 1..19: note = masking moiety
ESRSGYADIPFPAHWVPRT

SEQ ID NO: 664 - protein, 19 aa, synthetic construct
  REGION 1..19: note = masking moiety
RECGRCGDIPFPAHWVPRT

SEQ ID NO: 665 - protein, 8 aa, synthetic construct
  REGION 1..8: note = cleavable linker sequence
PRFKIIGG

SEQ ID NO: 666 - protein, 8 aa, synthetic construct
  REGION 1..8: note = cleavable linker sequence
PRFRIIGG

SEQ ID NO: 667 - protein, 9 aa, synthetic construct
  REGION 1..9: note = cleavable linker sequence
SSRHRRALD

SEQ ID NO: 668 - protein, 14 aa, synthetic construct
  REGION 1..14: note = cleavable linker sequence
RKSSIIIRMRDVVL

SEQ ID NO: 669 - protein, 15 aa, synthetic construct
  REGION 1..15: note = cleavable linker sequence
SSSFDKGKYKKGDDA

SEQ ID NO: 670 - protein, 15 aa, synthetic construct
  REGION 1..15: note = cleavable linker sequence
SSSFDKGKYKRGDDA

SEQ ID NO: 671 - protein, 4 aa, synthetic construct
  REGION 1..4: note = cleavable linker sequence
IEGR

SEQ ID NO: 672 - protein, 4 aa, synthetic construct
  REGION 1..4: note = cleavable linker sequence
IDGR

SEQ ID NO: 673 - protein, 7 aa, synthetic construct
  REGION 1..7: note = cleavable linker sequence
GGSIDGR

SEQ ID NO: 674 - protein, 6 aa, synthetic construct
  REGION 1..6: note = cleavable linker sequence
PLGLWA

SEQ ID NO: 675 - protein, 8 aa, synthetic construct
  REGION 1..8: note = cleavable linker sequence
GPQGIAGQ

SEQ ID NO: 676 - protein, 8 aa, synthetic construct
  REGION 1..8: note = cleavable linker sequence
GPQGLLGA

SEQ ID NO: 677 - protein, 5 aa, synthetic construct
  REGION 1..5: note = cleavable linker sequence
GIAGQ

SEQ ID NO: 678 - protein, 8 aa, synthetic construct
  REGION 1..8: note = cleavable linker sequence
GPLGIAGI

SEQ ID NO: 679 - protein, 8 aa, synthetic construct
  REGION 1..8: note = cleavable linker sequence
GPEGLRVG

SEQ ID NO: 680 - protein, 8 aa, synthetic construct
  REGION 1..8: note = cleavable linker sequence
YGAGLGVV

SEQ ID NO: 681 - protein, 8 aa, synthetic construct
  REGION 1..8: note = cleavable linker sequence
AGLGVVER

SEQ ID NO: 682 - protein, 8 aa, synthetic construct
  REGION 1..8: note = cleavable linker sequence
AGLGISST

SEQ ID NO: 683 - protein, 8 aa, synthetic construct
  REGION 1..8: note = cleavable linker sequence
EPQALAMS

SEQ ID NO: 684 - DNA, 1410 bp, synthetic construct
  misc_feature 1..1410: note = C225v5 Antibody Heavy Chain with Signal Peptide
  source 1..1410: mol_type = other DNA
atgtacaggatgcaactcctgtcttgcattgcactaagtcttgcacttgtcacgaattcgcaggtgcagctgaaacagagcggcccgggcctggtgcagccgagccagagcctgagcattacctgcaccgtgagcggctttagcctgaccaactatggcgtgcattgggtgcgccagagcccgggcaaaggcctggaatggctgggcgtgatttggagcggcggcaacaccgattataacaccccgtttaccagccgcctgagcattaacaaagataacagcaaaagccaggtgttttttaaaatgaacagcctgcaaagccaggataccgcgatttattattgcgcgcgcgcgctgacctattatgattatgaatttgcgtattggggccagggcaccctggtgaccgtgagcgcggctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 685 - protein, 469 aa, synthetic construct
  REGION 1..469: note = C225v5 Antibody Heavy Chain with Signal Peptide
MYRMQLLSCIALSLALVTNSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 686 - DNA, 858 bp, synthetic construct
  misc_feature 1..858: note = 3954-2787-c225 Light Chain with Signal Peptide
  source 1..858: mol_type = other DNA
atgtacaggatgcaactcctgtcttgcattgcactaagtcttgcacttgtcacgaattcgcaaggccagtctggccagtgcatctcacctcgtggttgtccggacggcccatacgtcatgtacggctcgagcggtggcagcggtggctctggtggatccggtacctccacctccggccgttccgcgaacccgcgtggtggcagtagcggtacccagatcttgctgacccagagcccggtgattctgagcgtgagcccgggcgaacgtgtgagctttagctgccgcgcgagccagagcattggcaccaacattcattggtatcagcagcgcaccaacggcagcccgcgcctgctgattaaatatgcgagcgaaagcattagcggcattccgagccgctttagcggcagcggcagcggcaccgattttaccctgagcattaacagcgtggaaagcgaagatattgcggattattattgccagcagaacaacaactggccgaccacctttggcgcgggcaccaaactggaactgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 687 - protein, 285 aa, synthetic construct
  REGION 1..285: note = 3954-2787-c225 Light Chain with Signal Peptide
MYRMQLLSCIALSLALVTNSQGQSGQCISPRGCPDGPYVMYGSSGGSGGSGGSGTSTSGRSANPRGGSSGTQILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 688 - DNA, 798 bp, synthetic construct
  misc_feature 1..798: note = 3954-2787-c225 Light Chain
  source 1..798: mol_type = other DNA
caaggccagtctggccagtgcatctcacctcgtggttgtccggacggcccatacgtcatgtacggctcgagcggtggcagcggtggctctggtggatccggtacctccacctccggccgttccgcgaacccgcgtggtggcagtagcggtacccagatcttgctgacccagagcccggtgattctgagcgtgagcccgggcgaacgtgtgagctttagctgccgcgcgagccagagcattggcaccaacattcattggtatcagcagcgcaccaacggcagcccgcgcctgctgattaaatatgcgagcgaaagcattagcggcattccgagccgctttagcggcagcggcagcggcaccgattttaccctgagcattaacagcgtggaaagcgaagatattgcggattattattgccagcagaacaacaactggccgaccacctttggcgcgggcaccaaactggaactgaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 689 - protein, 265 aa, synthetic construct
  REGION 1..265: note = 3954-2787-c225 Light Chain
QGQSGQCISPRGCPDGPYVMYGSSGGSGGSGGSGTSTSGRSANPRGGSSGTQILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 690 - protein, 7 aa, synthetic construct
  REGION 1..7: note = masking moiety
CISPRGC

SEQ ID NO: 691 - protein, 21 aa, synthetic construct
  REGION 1..21: note = masking moiety
QGQSGQCNIWLVGGDCRGWQG

SEQ ID NO: 692 - protein, 273 aa, synthetic construct
  REGION 1..273: note = anti-CTLA-4 scFv
GGGSGGGGSGSGGGSGGGGSGGGEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGGGTKVEIKRSGGSTITSYNVYYTKLSSSGTQVQLVQTGGGVVQPGRSLRLSCAASGSTFSSYAMSWVRQAPGKGLEWVSAISGSGGSTYYADSVKGRFTISRDNSKNTLYLQMNSLRAEDTAVYYCATNSLYWYFDLWGRGTLVTVSSAS

SEQ ID NO: 693 - protein, 264 aa, synthetic construct
  REGION 1..264: note = anti-CTLA-4 scFv
GGGSGGGGSGSGGGSGGGGSGGGQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 694 - protein, 194 aa, synthetic construct
  REGION 1..194: note = Display Platform CYTX-DP-XXXXXXXX
  VARIANT 17..24: note = X may be any amino acid
GQSGQEYMPMEGGSGQXXXXXXXXSGGQGGSGGSGGSGGSGGSAYYGITAGPAYRINDWASIYGVVGVGYGSGPGGSYGFSYGAGLQFNPMENVALDFSYEQSRIRSVDVGTWILSVGYRFGSKSRRATSTVTGGYAQSDAQGQMNKMGGFNLKYRYEEDNSPLGVIGSFTYTGGSGGSSGQAAAGHHHHHHHH

SEQ ID NO: 695 - protein, 217 aa, synthetic construct
  REGION 1..217: note = Display Platform SP-CYTX-DP-XXXXXXXX
  VARIANT 40..47: note = X may be any amino acid
MKKIACLSALAAVLAFTAGTSVAGQSGQEYMPMEGGSGQXXXXXXXXSGGQGGSGGSGGSGGSGGSAYYGITAGPAYRINDWASIYGVVGVGYGSGPGGSYGFSYGAGLQFNPMENVALDFSYEQSRIRSVDVGTWILSVGYRFGSKSRRATSTVTGGYAQSDAQGQMNKMGGFNLKYRYEEDNSPLGVIGSFTYTGGSGGSSGQAAAGHHHHHHHH

SEQ ID NO: 696 - protein, 8 aa, synthetic construct
  REGION 1..8: note = linker sequence
GGGSGGGS